(19) United States
(12) Patent Application Publication
(10) Pub. No.: US 2023/0203096 A1
(43) Pub. Date: Jun. 29, 2023

(54) EPHA4 TARGETING COMPOUNDS AND METHODS OF USE THEREOF

(71) Applicants: THE REGENTS OF THE UNIVERSITY OF CALIFORNIA, Oakland, CA (US); RESEARCH INSTITUTE AT NATIONWIDE CHILDREN'S HOSPITAL, Columbus, OH (US)

(72) Inventors: Maurizio PELLECCHIA, San Diego, CA (US); Carlo BAGGIO, Moreno Valley, CA (US); Iryna ETHELL, Riverside, CA (US); Anna KULINICH, Riverside, CA (US); Kathrin Christine MEYER, Columbus, OH (US); Cassandra Nicole DENNYS-RIVERS, Westerville, OH (US)

(73) Assignees: THE REGENTS OF THE UNIVERSITY OF CALIFORNIA, Oakland, CA (US); RESEARCH INSTITUTE AT NATIONWIDE CHILDREN'S HOSPITAL, Columbus, OH (US)

(21) Appl. No.: 17/920,231

(22) PCT Filed: Apr. 23, 2021
(86) PCT No.: PCT/US2021/028914

§ 371 Date: Oct. 20, 2022

Related U.S. Application Data
(60) Provisional application No. 63/014,551, filed on Apr. 23, 2020.

(51) Int. Cl. C07K 7/06; G01N 33/50

(52) U.S. Cl. CPC C07K 7/06; G01N 33/5082; A61K 38/00

(57) ABSTRACT

Certain embodiments of the invention provide EphA4 targeting compounds and compositions comprising a compound described herein. Certain embodiments of the invention also provide methods of treating a neurological disease (e.g., Amyotrophic Lateral Sclerosis, Alzheimer's Disease) or cancer.

### CROSS-REFERENCE TO RELATED APPLICATION

This application claims priority to U.S. Provisional Application No. 63/014,551 that was filed on Apr. 23, 2020. The entire content of the application referenced above is hereby incorporated by reference herein.

### GOVERNMENT FUNDING

This invention was made with government support under NS107479 awarded by the National Institutes of Health. The government has certain rights in the invention.

### BACKGROUND OF THE INVENTION

Amyotrophic lateral sclerosis (ALS) is a progressive neurodegenerative disease of the motor neurons. Mutations in the superoxide dismutase 1 (SOD1) enzyme, as well as in the hexanucleotide repeats in the non-coding region of C9orf72, have been attributed to the onset and progression of the disease (Rosen et al., 1993, Nature 362, 59-62) (DeJesus-Hernandez et al., 2011, Neuron 72, 245-256). Pharmacological inhibition of the EphA4 with an EphA4-blocking peptide (Murai et al., 2003, Molecular and cellular neurosciences 24, 1000-1011), enhanced the recovery and axonal sprouting in models of spinal cord injury (Goldshmit et al., 2004, The Journal of neuroscience: the official journal of the Society for Neuroscience 24, 10064-10073), and it was reported also to prolong survival in ALS animal models harboring the SOD1(G93A) gene (Van Hoecke et al., 2012, Nature medicine 18, 1418-1422) (Wu et al., 2017, Cell Chem Biol 24, 293-305). In ALS patients, EphA4 expression inversely correlates with onset and progression of the disease, (Van Hoecke et al., 2012, Nature medicine 18, 1418-1422). EphA4 signaling has been studied in ALS and other human diseases, including Alzheimer disease (AD) (Fu et al., 2014, PNAS 111, 9959-9964), spinal cord injury (Spanevello et al., 2013, J Neurotrauma 30, 1023-1034), brain injury (Frugier et al., 2012, J Neuropathol Exp Neurol 71, 242-250; Hanell et al., 2012, J Neurotrauma 29, 2660-2671), and some type of cancers (Fukai et al., 2008, Mol Cancer Ther 7, 2768-2778; Iiizumi et al., 2006, Cancer Sci 97, 1211-1216; Miyazaki et al., 2013, BMC Clin Pathol 13, 19.; Oshima et al., 2008, Int J Oncol 33, 573-577).

EphA4 expression is correlated with progression and resistance to chemotherapy of several human cancers including gastric, breast, and pancreatic cancers, as well multiple myeloma, where it also promotes cancer cell invasions. Finally, it has been shown that EphA4 is involved in hippocampal synaptic dysfunctions in mouse models of Alzheimer's disease, suggesting that its modulators can also be useful to threat AD.

These studies suggest that the EphA4 is a potential target for several human conditions and that targeting its ligand-binding domain provides an avenue to novel and effective therapeutics. Thus, there is need for potent EphA4 targeting agents that bind to the EphA4 ligand binding domain (LBD) for the development of novel therapeutics.

### SUMMARY OF THE INVENTION

Certain embodiments of the invention provide a compound comprising a peptide of Formula (I) X₀—X₁—X₂—X₃—X₄ from the N terminal to C terminal, wherein:

X₀ is a residue of an amino acid;

X₁ is a residue of Tryptophan (Trp);

X₂ is a residue of 4-phenyl-Phenylalanine (Bip);

X₃ is a residue of an amino acid;

X₄ is absent, or a residue of an amino acid;

or a salt thereof.

Certain embodiments of the invention provide a compound comprising a peptide of Formula (I) X₀—X₁—X₂—X₃—X₄ from the N terminal to C terminal, wherein:

X₀ is a residue of a beta-amino acid, gamma-amino acid, or delta-amino acid;

X₁ is a residue of Tryptophan (Trp);

X₂ is a residue of 4-phenyl-Phenylalanine (Bip);

X₃ is a residue of an amino acid;

X₄ is a residue of an amino acid;

or a salt thereof.

In certain embodiments, a compound as described herein consists of a peptide of Formula (I) as described herein. For example, certain embodiments of the invention provide a peptide of Formula (I) as described herein.

Certain embodiments of the invention provide a composition (e.g., a pharmaceutical composition) comprising a compound or peptide as described herein, or a pharmaceutically acceptable salt thereof, and a pharmaceutically acceptable carrier.

Certain embodiments of the invention provide a method of modulating EphA4 in vitro or in vivo, comprising contacting EphA4 with an effective amount of a compound or peptide as described herein, or a salt thereof.

Certain embodiments of the invention provide a method of activating EphA4 in a motor neuron, the method comprising contacting EphA4 with an effective amount of a compound or peptide as described herein, or salt thereof, wherein the compound/peptide is an agonist.

Certain embodiments of the invention provide a method of treating a disease associated with EphA4 and/or ephrin-B2 in a mammal in need thereof, comprising administering a therapeutically effective amount of a compound or peptide as described herein, or a pharmaceutically acceptable salt thereof, to the mammal.

Certain embodiments of the invention provide a compound or peptide, or a pharmaceutically acceptable salt thereof, as described herein, for use in medical therapy.

Certain embodiments of the invention provide a compound or peptide, or a pharmaceutically acceptable salt thereof, as described herein, for the prophylactic or therapeutic treatment of a disease associated with EphA4 and/or ephrin-B2 in a mammal in need thereof.

Certain embodiments of the invention provide the use of a compound or peptide, or a pharmaceutically acceptable salt thereof, as described herein, to prepare a medicament for treating a disease associated with EphA4 and/or ephrin-B2 in a mammal in need thereof.

Certain embodiments provide a method of treating or preventing motor neuron degeneration in a mammal in need thereof, comprising administering a therapeutically effective amount of a compound or peptide as described herein, or a pharmaceutically acceptable salt thereof, to the mammal.

Certain embodiments provide a compound or peptide as described herein, or a pharmaceutically acceptable salt thereof, for treating or preventing motor neuron degeneration.

Certain embodiments provide the use of a compound or peptide as described herein, or a pharmaceutically acceptable salt thereof, to prepare a medicament for treating or preventing motor neuron degeneration in a mammal.

Certain embodiments provide a method for identifying an EphA4 agonist, the method comprising isolating primary motor neurons from the spinal cord of an animal, contacting a test compound with the isolated primary motor neurons under conditions suitable for binding between the test compound and EphA4, evaluating axon growth cone morphology of the primary motor neurons, and identifying the test compound as an EphA4 agonist when growth cone collapse is detected.

Certain embodiments provide a compound, or a salt thereof, as described herein. Certain embodiments provide a peptide, or a salt thereof, as described herein.

The invention also provides processes and intermediates disclosed herein that are useful for preparing a compound/peptide, or a salt thereof, as described herein.

### BRIEF DESCRIPTION OF THE FIGURES

FIGS. 1A-1C. FIG. 1A. APY-d3 (reference peptide). FIG. 1B. 2D [¹³C,¹H] correlation spectra with a sample ¹³C^{ε}-Met labeled of the EphA4 ligand binding domain, measured in absence and presence of reference peptide APY-d3 (βA-PYCVYR-βA-SWSC-CONH₂). Changes in the chemical shift of Met 164 are indicative of agonistic binding mode of the agent. The long arrow indicates the change in chemical shift of Met 115 induced by APY-d3. The short arrow indicates the change in chemical shift of Met 164 induced by APY-d3. FIG. 1C. Isothermal titration calorimetry (ITC) data with reference peptide APY-d3.

FIGS. 2A-2C. FIG. 2A. Compound 2. FIG. 2B. 2D [¹³C,¹H] correlation spectra with a sample ¹³C^{ε}-Met labeled of the EphA4 ligand binding domain, measured in absence and presence of compound 2. Changes in the chemical shift of Met 164 are indicative of agonistic binding mode of the agent. The arrow indicates the change in chemical shift of Met 164 induced by compound 2. FIG. 2C. Isothermal titration calorimetry (ITC) data with compound 2 for the determination of the value reported in Table 1.

FIGS. 3A-3C. FIG. 3A. Compound 3. FIG. 3B. 2D [¹³C,¹H] correlation spectra with a sample ¹³C^{ε}-Met labeled of the EphA4 ligand binding domain, measured in absence and presence of compound 3. Changes in the chemical shift of Met 164 are indicative of agonistic binding mode of the agent. The arrow indicates the change in chemical shift of Met 164 induced by compound 3. FIG. 3C. Isothermal titration calorimetry (ITC) data with compound 3 for the determination of the value reported in Table 1.

FIGS. 4A-4C. FIG. 4A. Compound 8. FIG. 4B. 2D [¹³C,¹H] correlation spectra with a sample ¹³C^{ε}-Met labeled of the EphA4 ligand binding domain, measured in absence and presence of compound 8 (150D4). Changes in the chemical shift of Met 164 are indicative of agonistic binding mode of the agent. The arrow indicates the change in chemical shift of Met 164 induced by compound 8. FIG. 4C. Isothermal titration calorimetry (ITC) data with compound 8 for the determination of the value reported in Table 1.

FIGS. 5A-5E. fHTS by NMR summary as deployed against EphA4-LBD. FIG. 5A) Schematic representation of the Ala-XXX positional scanning library made up by 46×3 mixtures each containing 46×46 tetrapeptides. FIG. 5B) Summary of the chemical shift perturbations induced by each mixture. The perturbations were detected using 1D ¹H aliphatic region of the EphA4-LBD as illustrated in panel FIG. 5C). FIG. 5D) Chemical structure of consensus agent 1 and relative perturbations induced (at 20 μM) in the 1D ¹H aliphatic spectrum of EphA4 (20 μM). FIG. 5E) Isothermal titration calorimetry data with EphA4-LBD and compound E1. Fitting of the titration points resulted in a dissociation constant for the complex Kd˜3 μM.

FIGS. 6A-6D. Biophysical studies on EphA4-LBD in the free versus bound state. FIG. 6A) Superposition of the X-ray structures of EphA4-LBD in the apo form (PDB ID 2WO1) and APY-d3 bound (PDB ID 5JR2; stick model for APY-d3). The thickness of the tube is proportional to the pairwise backbone Ca atoms RMSD between the two compared structures. Most notable conformational changes upon antagonist APY-d3 binding are highlighted, together with Met residues that are displayed as stick models. FIG. 6B) Superposition of the X-ray structures of EphA4-LBD in the apo form (PDB ID2WO1) and ephrinA5 bound (PDB ID 4BKA; stick model showing only a peptide region from ephrinA5 that is contact with the EphA4-LBD). Most notable conformational changes upon agonist ephrinA5 binding are highlighted, together with Met residues. FIG. 6C) 2D [¹³C, ¹H] correlation spectra for EphA4-LBD ¹³C^{ε}-Met labeled, measured in absence and in presence of antagonist APY-d3 or agonist 123C4. FIG. 6D) Isothermal titration calorimetry curves for the binding of agents APY-d3 or 123C4 to EphA4-LBD.

FIGS. 7A-7D. Biophysical characterizations of 150D4 binding to EphA4-LBD. FIG. 7A) ITC data for the binding of 150D4 to EphA4-LBD, EphA3-LBD, or EphA2-LBD. FIG. 7B) 150D4 displaces the binding between EphA4-LBD and ephrinA5, as detected by [¹³C,¹H] correlation spectra with EphA4-LBD ¹³C-Met. FIG. 7C) and FIG. 7D) report 1D ¹H NMR and 2D [¹³C,¹H] correlation spectra, respectively, of ¹³C-Met-EphA4-LBD recorded in presence of various concentrations of 150D4.

FIGS. 8A-8D. X-ray and NMR studies with 150D4 in complex with EphA4-LBD. FIG. 8A) Superposition of the structure of EphA4-LBD in complex with 150D4 (sticks model) versus the apo structure of EphA4-LBD (PDB ID 2WO1). The highlighted conformational changes are similar to those induced by the agonistic ligand ephrinA5 (see FIG. 6). FIG. 8B) 2D [¹³C, ¹H] correlation spectra of ¹³C^{ε}-Met-EphA4-LBD collected in absence and presence of 150D4. The large chemical shift changes for residues Met 60 and Met 164 induced by 150D4 are in agreement with the conformational changes observed in loops D-E and J-K, respectively, while and unlike APY-d3, no significant perturbations were observed for Met 115, in the G-H loop. FIG. 8C) Schematic plot to represent the intermolecular interactions between 150D4 and EphA4-LBD. FIG. 8D) Stick model and contour map of the observed electron density for 150D4 when in complex with EphA4-LBD. The ligand molecule is shown superimposed with the refined 2Fo-Fc electron density map contoured at 1.0σ.

FIGS. 9A-9E. EphA4 phosphorylation in primary spinal cord motor neurons. FIG. 9A) Representative western blot images of pEphA4, total EphA4 (after immunoprecipitation, IP) and a motor neuron marker, choline acetyltransferase (ChAT, in cell lysate) in cultures of primary spinal cord motor neurons treated with DMSO, Fe, ephrinA1-Fc (eA1-Fc), APYd3, compound 2, compound 9, compound 3, and 150D4 (1 μM and 10 μM) for 30 min. FIG. 9B-9D) Graphs show average ratio of pEphA4 and total EphA4 in primary motor neuron cultures treated with DMSO, Fc, eA1-Fc, APYd3, compound 2, compound 9, compound 3, and 150D4 (FIG. 9B); DMSO, Fc and eA1-Fc (FIG. 9C); DMSO and 1 μM 150D4 (D). Black solid lines above the graph indicate separate experiments (experiments 1-4, FIG. 9B). Error bars indicate SEM (each experiment was repeated 3 times). Statistical analysis was performed using one-way ANOVA followed by Bonferroni's post-hoc analysis (*p<0.05, FIG. 9C) or two-tailed, unpaired student's t test for comparison of two groups; *p<0.05 (p=0.035, FIG. 9D). FIG. 9E, representative western blot images of pEphA4, total EphA4 and ChAT in primary spinal cord motor neurons treated with DMSO, 1 μM 123C3, 10 μM 123C4, 1 μM 150D4, or 10 μM 150D4.

FIGS. 10A-10E. Growth cone collapse in primary spinal cord motor neurons. FIG. 10A-D, Representative images of 2 DIV primary spinal cord motor neurons treated with DMSO (A), Fc (Fc), eA1-Fc (C), 1 μM 150D4 or 10 μM 150D4. Growth cone morphology was assessed by labeling F-actin with rhodamine-coupled phalloidin. Motor neurons were identified by genetically encoded Hb9-GFP and immunostaining against ChAT. (FIG. 10B, D) High magnification images of growing (FIG. 10B) and collapsed (FIG. 10D) growth cones. Scale bars are 50 m in A, C and 10 m in B, D. (FIG. 10E) Graph shows average percent of collapsed growth cones in primary spinal cord motor neuron cultures treated with DMSO, Fc (Fc), eA1-Fc, 1 μM 150D4, 10 μM 150D4, 1 M 150D4 plus ephrin A1-Fc or 10 μM 150D4 plus ephrin A1-Fc. Error bars indicate SEM (n=4-6 coverslips). Statistical analysis was performed using one-way ANOVA followed by Bonferroni's post-hoc analysis (**p<0.01, ****p<0.0001).

FIGS. 11A-11C. EphA4 agonists protects from iAstrocyte mediated motor neuron death at lower concentrations. FIG. 11A) Schematic illustration of the assay. NPCs were differentiated into induced astrocytes for five days then seeded on a 96 well plate. 10 μM of new ephrin ligand compounds were added 24 hours later at the time of motor neuron addition. 100 μM 123C4 was added to co-culture at time of motor neuron addition as a positive control. FIG. 11B) Representative image of motor neurons following 3 days in co-culture. FIG. 11C) Quantification of motor neuron survival following co-culture. Data was normalized to average motor neuron survival of healthy controls. Data represents a minimum of 2 independent experiments. Statistical analysis was performed using unpaired t-test comparing corresponding treated and untreated iAs.

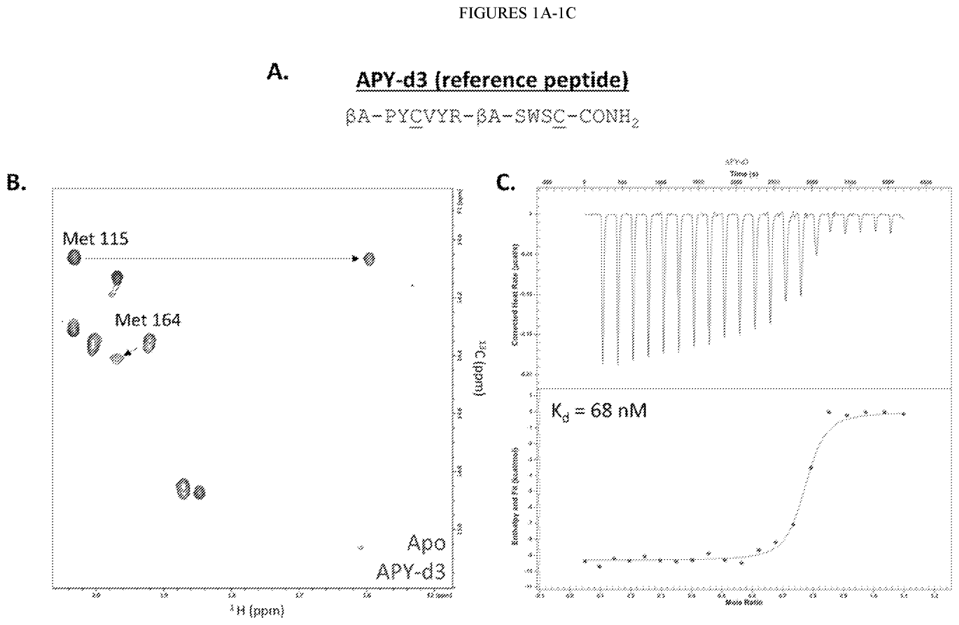

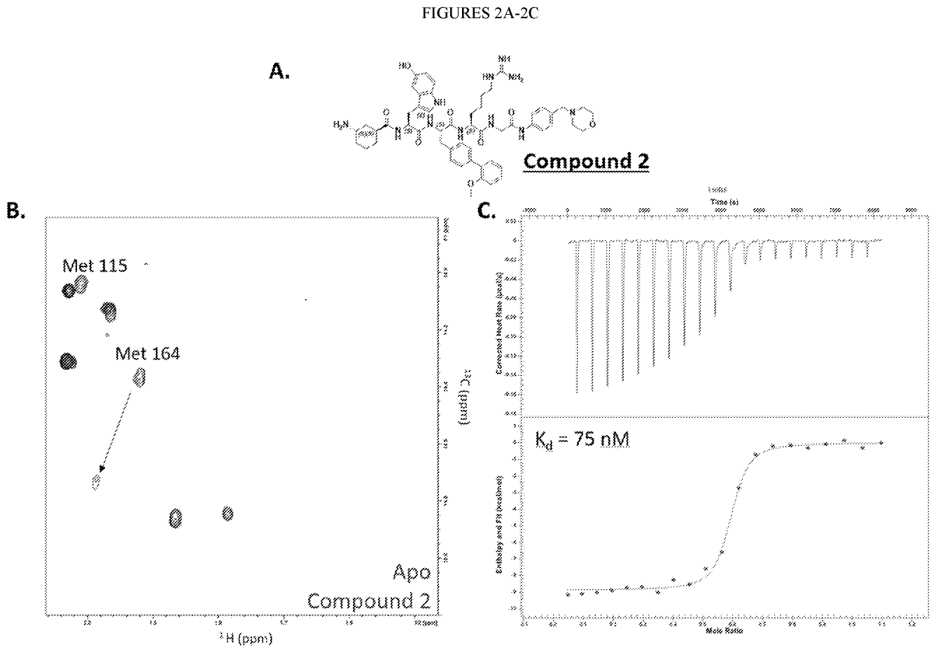

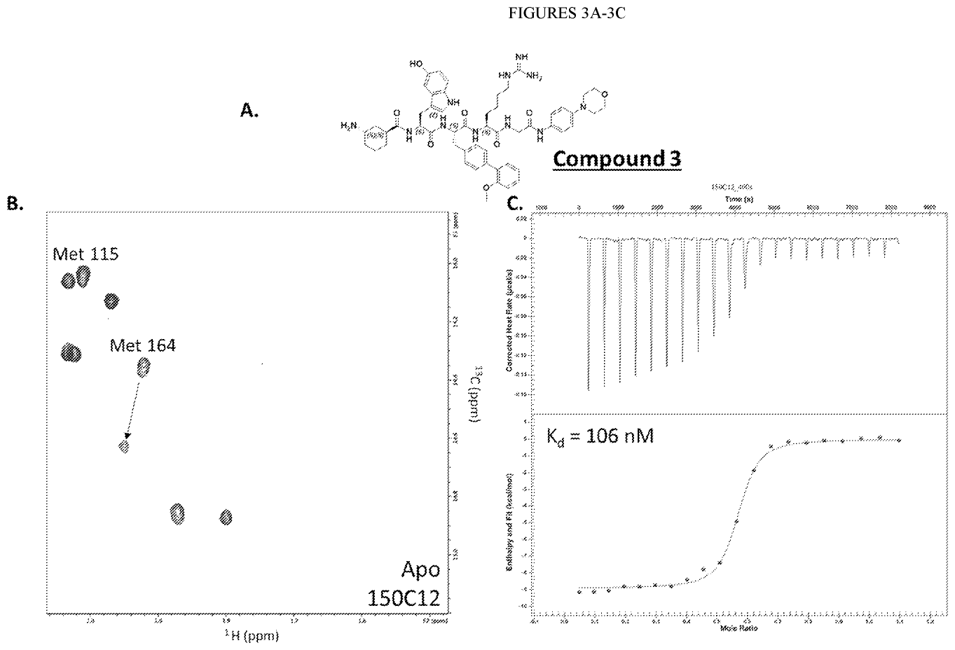

### DETAILED DESCRIPTION

The invention described herein relates to protein-protein interaction (PPI) inhibitors, which target EphA4.

EphA4 belongs to the Eph family of receptor tyrosine kinases, which together with their membrane-bound ligands, the ephrins (Eph receptor-interacting proteins), generate bidirectional signals controlling a multitude of cellular processes. One endogenous ligand for EphA4 is ephrin-B2. Without wanting to be limited by theory, in some examples, ligand-binding of this signaling axis may trigger forward signaling in the EphA4 expressing cells (e.g., neuron) and/or reverse signaling in ephrin-B2 expressing cells (e.g., astrocyte). The uni-directional and/or bidirectional signaling of this axis has been implicated in cancer and neurological diseases. Pharmacological disruption of the EphA4/ephrin-B2 engagement may mitigate neurological pathology and promote the repair and regeneration of neurons (e.g., motor neurons).

Accordingly, certain embodiments of the invention provide EphA4 targeting compounds/peptides that bind the Ligand Binding Domain (LBD) of EphA4 and potently compete with its natural ligand(s) (e.g., a ligand described herein).

In certain embodiments, compounds/peptides of the invention inhibit the EphA4 and ephrin-B2 interaction. In certain embodiments, compounds/peptides of the invention block EphA4 from binding its natural ligand(s) (e.g., a ligand described herein). In certain embodiments, compounds/peptides of the invention irreversibly bind EphA4.

In certain embodiments, compounds/peptides of the invention decrease the level of EphA4 on the cell surface or induce cellular internalization of EphA4. In certain embodiments, a compound/peptide of the invention is an EphA4 agonist. In certain embodiments, a compound/peptide of the invention is an EphA4 partial agonist. In certain embodiments, a compound/peptide of the invention is an EphA4 antagonist. In certain embodiments, compounds/peptides of the invention inhibit EphA4 mediated reverse signaling of ephrin-B2 in ephrin-B2 expressing cells (e.g., astrocyte).

### EphA4 Targeting Compounds Comprising a Peptide of Formula (I)

Certain embodiments of the invention provide a compound comprising a peptide of Formula (I) X₀—X₁—X₂—X₃—X₄ from the N terminal to C terminal, wherein:

X₀ is a residue of an amino acid;

X₁ is a residue of Tryptophan (Trp);

X₂ is a residue of 4-phenyl-Phenylalanine (Bip);

X₃ is a residue of an amino acid;

X₄ is absent or a residue of an amino acid;

or a salt thereof.

In certain embodiments, the peptide of Formula (I) X₀—X₁—X₂—X₃—X₄ is 4 amino acids in length. In certain embodiments, the peptide of Formula (I) X₀—X₁—X₂—X₃—X₄ is 5 amino acids in length.

Certain embodiments of the invention provide a compound comprising a peptide of Formula (I) X₀—X₁—X₂—X₃—X₄ from the N terminal to C terminal, wherein:

X₀ is a residue of a beta-amino acid, gamma-amino acid, or delta-amino acid;

X₁ is a residue of Tryptophan (Trp);

X₂ is a residue of 4-phenyl-Phenylalanine (Bip);

X₃ is a residue of an amino acid;

X₄ is a residue of an amino acid;

or a salt thereof.

In certain embodiments, each residue of an amino acid in the peptide of Formula (I) is independently and optionally substituted.

In certain embodiments, the optional substituent is halo, hydroxy, cyano, carboxyl, —CONH₂, —NO₂, —SH, —SO₃H, —SO₄H, —SO₂NH₂, —OSO₂F, —SO₂F, —NHNH₂, —ONH₂, —NHC(═O)NHNH₂, —NHC(═O)NH₂, —NHC(═O)H, —NHC(═O)OH, —NHOH, (C₁-C₆)alkyl, (C₁-C₆)alkoxy, (C₁-C₆)alkoxycarbonyl, (C₁-C₆)alkanoyl, (C₁-C₆)alkanoyloxy, (C₁-C₆)heteroalkyl, (C₃-C₆)cycloalkyl, heterocycloalkyl, heterocycloalkyl(C₁-C₆)alkyl, aryl, heteroaryl, or —NR^{r}R^{s}, wherein any (C₁-C₆)alkyl, (C₁-C₆)alkoxy, (C₁-C₆)alkoxycarbonyl, (C₁-C₆)alkanoyl, (C₁-C₆)alkanoyloxy, (C₁-C₆)heteroalkyl, (C₃-C₆)cycloalkyl, heterocycloalkyl, heterocycloalkyl(C₁-C₆)alkyl, aryl, and heteroaryl is optionally substituted with one or more groups independently selected from the group consisting of halo, hydroxy, and (C₁-C₆)alkoxy; each R^{r} and R^{s} is independently selected from the group consisting of H, (C₁-C₆)alkyl, aryl, and (C₁-C₆)alkoxyaryl.

In certain embodiments, the optional substituent is halogen, hydroxy, cyano, (C₁-C₆)alkoxy, or (C₁-C₆)alkyl that is optionally substituted with one or more halo.

In certain embodiments, the optional substituent is halogen, —OCH₃, —CN, —OH, —NH₂, —CH₂OH, —CH₂(OH)CH₃, —COOH, —CONH₂, —NO₂, —SH, —SO₃H, —SO₄H, —SO₂NH₂, —OSO₂F, —SO₂F, —NHNH₂, —ONH₂, —NHC(═O)NHNH₂, —NHC(═O)NH₂, —NHC(═O)H, —NHC(═O)OH, —NHOH, —OCCl₃, —OCBr₃, —OCF₃, —OCl₃, —OCH₂Cl, —OCH₂Br, —OCH₂F, —OCH₂I, —OCHCl₂, —OCHBr₂, —OCHF₂, —OCHI₂, an amino acid side chain (e.g., the side chain of a natural amino acid), alkyl, heteroalkyl, cycloalkyl, heterocycloalkyl, heterocycloalkyl(C₁-C₆)alkyl, aryl, or heteroaryl, wherein the alkyl, heteroalkyl, cycloalkyl, heterocycloalkyl, aryl, or heteroaryl is optionally substituted.

In certain embodiments, each residue of an amino acid in the peptide of Formula (I) is independently and optionally substituted. In certain embodiments, Trp at X₁ is optionally substituted on the indole group. For example, in certain embodiments, X₁ is a residue of 5-hydroxy-Trp or 5-methoxy-Trp. In certain embodiments, Bip at X₂ is optionally substituted on one or both phenyl group. For example, in certain embodiments, X₂ is a residue of 4-(2-methoxyphenyl)-Phe.

In certain embodiments, X₁, X₂, X₃ and X₄ are each a residue of an alpha-amino acid.

In certain embodiments, the compound is/consists of a peptide of Formula (I) as described herein. For example, certain embodiments of the invention provide a peptide of Formula (I) X₀—X₁—X₂—X₃—X₄ from the N terminal to C terminal, wherein:

X₀ is a residue of an amino acid, wherein the N-terminus is a primary amine group NH₂—, or a capped amine R^{N}—C(═O)—NH—, wherein R^{N} is H, (C₃-C₆)cycloalkyl, or (C₁-C₄)alkyl;

X₁ is a residue of Trp, wherein Trp is optionally substituted with one or more groups independently selected from the group consisting of halo, hydroxy, cyano, carboxyl, —CONH₂, —NO₂, —SH, —SO₃H, —SO₄H, —SO₂NH₂, —OSO₂F, —SO₂F, —NHNH₂, —ONH₂, —NHC(═O)NHNH₂, —NHC(═O)NH₂, —NHC(═O)H, —NHC(═O)OH, —NHOH, (C₁-C₆)alkyl, (C₁-C₆)alkoxy, (C₁-C₆)alkoxycarbonyl, (C₁-C₆)alkanoyl, (C₁-C₆)alkanoyloxy, (C₁-C₆)heteroalkyl, (C₃-C₆)cycloalkyl, heterocycloalkyl, heterocycloalkyl(C₁-C₆)alkyl, aryl, heteroaryl, and —NR^{r}R^{s}, wherein any (C₁-C₆)alkyl, (C₁-C₆)alkoxy, (C₁-C₆)alkoxycarbonyl, (C₁-C₆)alkanoyl, (C₁-C₆)alkanoyloxy, (C₁-C₆)heteroalkyl, (C₃-C₆)cycloalkyl, heterocycloalkyl, heterocycloalkyl(C₁-C₆)alkyl, aryl, and heteroaryl is optionally substituted with one or more groups independently selected from the group consisting of halo, hydroxy, and (C₁-C₆)alkoxy;

X₂ is a residue of Bip, wherein Bip is optionally substituted on one or both phenyl groups with one or more groups independently selected from the group consisting of halo, hydroxy, cyano, carboxyl, —CONH₂, —NO₂, —SH, —SO₃H, —SO₄H, —SO₂NH₂, —OSO₂F, —SO₂F, —NHNH₂, —ONH₂, —NHC(═O)NHNH₂, —NHC(═O)NH₂, —NHC(═O)H, —NHC(═O)OH, —NHOH, (C₁-C₆)alkyl, (C₁-C₆)alkoxy, (C₁-C₆)alkoxycarbonyl, (C₁-C₆)alkanoyl, (C₁-C₆)alkanoyloxy, (C₁-C₆)heteroalkyl, (C₃-C₆)cycloalkyl, heterocycloalkyl, heterocycloalkyl(C₁-C₆)alkyl, aryl, heteroaryl, and —NR^{r}R^{s}, wherein any (C₁-C₆)alkyl, (C₁-C₆)alkoxy, (C₁-C₆)alkoxycarbonyl, (C₁-C₆)alkanoyl, (C₁-C₆)alkanoyloxy, (C₁-C₆)heteroalkyl, (C₃-C₆)cycloalkyl, heterocycloalkyl, heterocycloalkyl(C₁-C₆)alkyl, aryl, and heteroaryl is optionally substituted with one or more groups independently selected from the group consisting of halo, hydroxy, and (C₁-C₆)alkoxy;

X₃ is a residue of an amino acid;

X₄ is absent, or a residue of an amino acid, wherein the C-terminus is a carboxyl group —COOH, or the C-terminus is amidated to form —C(═O)NR^{x}R^{y};

wherein each R^{x} and R^{y} is independently selected from the group consisting of H, (C₁-C₆)alkyl, (C₃-C₆)cycloalkyl, aryl, heteroaryl, (C₃-C₆)cycloalkyl(C₁-C₆)alkyl, aryl(C₁-C₆)alkyl, heteroaryl(C₁-C₆)alkyl, aryl-heterocycloalkyl-aryl, aryl-heterocycloalkyl-heteroaryl, aryl-heterocycloalkyl-(C₁-C₆)alkyl-aryl, aryl-heterocycloalkyl-(C₁-C₆)alkyl-heteroaryl, and aryl-(C₁-C₆)alkyl-heterocycloalkyl-aryl,

wherein any (C₁-C₆)alkyl, (C₃-C₆)cycloalkyl, aryl, heteroaryl, (C₃-C₆)cycloalkyl(C₁-C₆)alkyl, aryl(C₁-C₆)alkyl, heteroaryl(C₁-C₆)alkyl, aryl-heterocycloalkyl-aryl, aryl-heterocycloalkyl-heteroaryl, aryl-heterocycloalkyl-(C₁-C₆)alkyl-aryl, aryl-heterocycloalkyl-(C₁-C₆)alkyl-heteroaryl, and aryl-(C₁-C₆)alkyl-heterocycloalkyl-aryl is optionally substituted with one or more groups independently selected from the group consisting of halo, hydroxy, cyano, carboxyl, —CONH₂, —NO₂, —SH, —SO₃H, —SO₄H, —SO₂NH₂, —OSO₂F, —SO₂F, —NHNH₂, —ONH₂, —NHC(═O)NHNH₂, —NHC(═O)NH₂, —NHC(═O)H, —NHC(═O)OH, —NHOH, (C₁-C₆)alkyl, (C₁-C₆)alkoxy, (C₁-C₆)alkoxycarbonyl, (C₁-C₆)alkanoyl, (C₁-C₆)alkanoyloxy, (C₁-C₆)heteroalkyl, (C₃-C₆)cycloalkyl, heterocycloalkyl, heterocycloalkyl(C₁-C₆)alkyl, aryl, heteroaryl, and —NR^{r}R^{s}, wherein any (C₁-C₆)alkyl, (C₁-C₆)alkoxy, (C₁-C₆)alkoxycarbonyl, (C₁-C₆)alkanoyl, (C₁-C₆)alkanoyloxy, (C₁-C₆)heteroalkyl, (C₃-C₆)cycloalkyl, heterocycloalkyl, heterocycloalkyl(C₁-C₆)alkyl, aryl, and heteroaryl is optionally substituted with one or more groups independently selected from the group consisting of halo, hydroxy, and (C₁-C₆)alkoxy;

each R^{r} and R^{s} is independently selected from the group consisting of H, (C₁-C₆)alkyl, aryl, and (C₁-C₆)alkoxyaryl;

or a salt thereof.

In certain embodiments, X₀ is a residue of a beta-amino acid, gamma-amino acid, or delta-amino acid, wherein the N-terminus is a primary amine group NH₂—, or a capped amine R^{N}—C(═O)—NH—, wherein R^{N} is H, (C₃-C₆)cycloalkyl, or (C₁-C₄)alkyl. In certain embodiments, X₄ is a residue of an amino acid, wherein the C-terminus is a carboxyl group —COOH, or the C-terminus is amidated to form —C(═O)NR^{x}R^{y}. In certain embodiments, X₀ is beta amino acid, gamma amino acid or delta amino acid, wherein the N-terminus is a primary amine group NH₂—, or a capped amine R^{N}—C(═O)—NH—, wherein R^{N} is H, (C₃-C₆)cycloalkyl, or (C₁-C₄)alkyl; and X₄ is a residue of an amino acid, wherein the C-terminus is a carboxyl group —COOH, or the C-terminus is amidated to form —C(═O)NR^{x}R^{y}.

In certain embodiments, X₄ is absent.

In certain embodiments, X₄ is absent and X₃ is a residue of an amino acid wherein the C-terminus is a carboxyl group —COOH, or the C-terminus is amidated to form —C(═O)NR^{x}R^{y}.

### N-Terminus of a Peptide of Formula (I)

Certain embodiments of the invention provide a compound comprising or consisting of a peptide of Formula (I) X₀—X₁—X₂—X₃—X₄ from the N terminal to C terminal, wherein X₀ is the residue of an amino acid at the N-terminus of the peptide, and X₄ (if present) is the residue of an amino acid at the C-terminus of the peptide.

In certain embodiments, the N-terminus of the peptide of Formula (I) is a primary amine group (NH₂—). In certain embodiments, the N-terminus of the peptide of Formula (I) is not acylated. Namely, in certain embodiments, the primary amine NH₂— of X₀ is not capped (e.g., acylated, or formylated) and may carry a positive charge as H₃N⁺— under suitable conditions (e.g., physiological condition).

In certain embodiments, the N-terminus of the peptide of Formula (I) is capped. In certain embodiments, the N-terminus of the peptide of Formula (I) is acylated or formylated. For example, the N-terminus of the peptide of Formula (I) may be capped as R^{N}—C(═O)—NH—, wherein R^{N} is H, (C₃-C₆)cycloalkyl, or (C₁-C₄)alkyl.

In certain embodiments, X₀ is a residue of an alpha-amino acid (e.g., Ala).

In certain embodiments, X₀ is not a residue of an alpha-amino acid, or X₀ is a residue of a nonalpha-amino acid.

In certain embodiments, X₀ is a residue of a beta-amino acid, gamma-amino acid, or delta-amino acid.

In certain embodiments, the alpha-amino group is a primary amine group. In certain embodiments, the beta-amino group is a primary amine group. In certain embodiments, the gamma-amino group is a primary amine group. In certain embodiments, the delta-amino group is a primary amine group.

In certain embodiments, X₀ is a residue of a beta-amino acid (e.g., beta-alanine).

In certain embodiments, X₀ is a residue of a gamma-amino acid. In certain embodiments, the gamma carbon of the gamma-amino acid is on a linear or branched carbon chain. In certain embodiments, X₀ is a residue of gamma-amino-butyric acid (GABA).

In certain embodiments, the gamma carbon of the gamma-amino acid is on a cycloalkyl group (e.g., cyclopentyl, cyclohexyl or cycloheptyl). In certain embodiments, X₀ is a residue of 3-aminocyclopentane-1-carboxylic acid. In certain embodiments, X₀ is a residue of 3-amino-cyclohexane-1-carboxylic acid (ACHC). In certain embodiments, X₀ is a residue of 3-aminocycloheptane-1-carboxylic acid.

In certain embodiments, the residue of X₀ is optionally substituted.

In certain embodiments, the optional substituent is halogen, hydroxy, cyano, (C₁-C₆)alkoxy, or (C₁-C₆)alkyl that is optionally substituted with one or more halo.

In certain embodiments, the optional substituent is Rₐ and/or R_{b} as defined herein (e.g., in formula (Ib) below).

In certain embodiments, X₀ is a residue of a beta-amino acid, gamma-amino acid, or delta-amino acid, wherein the N-terminus is a primary amine group NH₂—, or a capped amine R^{N}—C(═O)—NH—, wherein R^{N} is H, (C₃-C₆)cycloalkyl, or (C₁-C₄)alkyl.

In certain embodiments, the invention provides a compound comprising a peptide of Formula (I) X₀—X₁—X₂—X₃—X₄ wherein:

X₀ is a residue of a gamma-amino acid (e.g., GABA or ACHC),

X₁ is a residue of Trp,

X₂ is a residue of Bip,

X₃ is a residue of an amino acid,

X₄ is absent, or a residue of an amino acid. In certain embodiments, the compound consists of such a peptide of Formula (I).

In certain embodiments, the invention provides a compound comprising a peptide of Formula (I) X₀—X₁—X₂—X₃—X₄ wherein:

X₀ is a residue of a gamma-amino acid (e.g., GABA or ACHC),

X₁ is a residue of Trp,

X₂ is a residue of Bip,

X₃ is a residue of an amino acid,

X₄ is a residue of an amino acid. In certain embodiments, the compound consists of such a peptide of Formula (I).

In certain embodiments, each residue of an amino acid in the peptide of Formula (I) is independently and optionally substituted. In certain embodiments, Trp at X₁ is optionally substituted on the indole group. For example, in certain embodiments, X₁ is a residue of 5-hydroxy-Trp or 5-methoxy-Trp. In certain embodiments, Bip at X₂ is optionally substituted on one or both phenyl group. For example, in certain embodiments, X₂ is a residue of 4-(2-methoxyphenyl)-Phe.

### X₁ in the Peptide of Formula (I)

In certain embodiments, X₁ is a residue of Trp.

In certain embodiments, the residue of Trp in X₁ is optionally substituted. In certain embodiments, the indole group of the residue of Trp in X₁ is optionally substituted with —OH or —OCH₃. In certain embodiments, X₁ is a residue of 5-hydroxy-Trp. In certain embodiments, X₁ is a residue of 5-methoxy-Trp. In certain embodiments, X₁ is a residue of 6-methoxy-Trp.

In certain embodiments, the residue of Trp in X₁ is optionally substituted with a heteratom (e.g., nitrogen atom) on the six-membered ring of the indole group.

In certain embodiments, the optional substituent is halogen, hydroxy, cyano, (C₁-C₆)alkoxy, or (C₁-C₆)alkyl that is optionally substituted with one or more halo.

In certain embodiments, the optional substituent is R¹ as defined herein (e.g., in formula (Ia) below).

In certain embodiments, the invention provides a compound comprising a peptide of Formula (I) X₀—X₁—X₂—X₃—X₄ wherein:

X₀ is a residue of a beta-amino acid, gamma-amino acid or delta-amino acid,

X₁ is a residue of 5-hydroxy-Trp,

X₂ is a residue of Bip,

X₃ is a residue of an amino acid,

X₄ is a residue of an amino acid. In certain embodiments, the compound consists of such a peptide of Formula (I).

In certain embodiments, the invention provides a compound comprising a peptide of Formula (I) X₀—X₁—X₂—X₃—X₄ wherein:

X₀ is a residue of a beta-amino acid, gamma-amino acid or delta-amino acid,

X₁ is a residue of 5-hydroxy-Trp,

X₂ is a residue of Bip,

X₃ is a residue of an amino acid,

X₄ is absent, or a residue of an amino acid. In certain embodiments, the compound consists of such a peptide of Formula (I).

In certain embodiments, each residue of an amino acid in the peptide of Formula (I) is independently and optionally substituted. In certain embodiments, Trp at X₁ is optionally substituted on the indole group. For example, in certain embodiments, X₁ is a residue of 5-hydroxy-Trp or 5-methoxy-Trp. In certain embodiments, Bip at X₂ is optionally substituted on one or both phenyl group. For example, in certain embodiments, X₂ is a residue of 4-(2-methoxyphenyl)-Phe.

### X₂ in the Peptide of Formula (I)

In certain embodiments, X₂ is a residue of Bip, also referred to as 4-phenyl-L-phenylalanine, or 4-phenyl-Phe, or L-4,4′-Biphenylalanine.

In certain embodiments, the residue of Bip in X₂ is optionally substituted. There are two phenyl rings in Bip: 1) the proximal phenyl ring that is part of the phenylalanine group and 2) the distal 4-phenyl ring. In certain embodiments, each phenyl ring of the residue of Bip in X₂ is optionally and independently substituted.

In certain embodiments, the proximal phenyl ring of the residue of Bip in X₂ is optionally substituted.

In certain embodiments, the optional substituent is halogen, hydroxy, cyano, (C₁-C₆)alkoxy, or (C₁-C₆)alkyl that is optionally substituted with one or more halo.

In certain embodiments, the optional substituent on the proximal phenyl ring is R² as defined herein (e.g., in formula (Ia) below).

In certain embodiments, the 4-phenyl ring of the residue of Bip in X₂ is optionally substituted. In certain embodiments, the 4-phenyl ring of the residue of Bip in X₂ is optionally substituted with —OH or —OCH₃. In certain embodiments, X₂ is a residue of 4-(2-methoxyphenyl)-Phe. In certain embodiments, X₂ is a residue of 4-(2, 6-dimethoxyphenyl)-Phe. In certain embodiments, the 4-phenyl ring of the residue of Bip in X₂ is optionally substituted with halogen or alkyl group. In certain embodiments, X₂ is a residue of 4-(4-chlorophenyl)-Phe. In certain embodiments, X₂ is a residue of 4-(4-methylphenyl)-Phe. In certain embodiments, X₂ is a residue of 4-(2-methylphenyl)-Phe.

In certain embodiments, the optional substituent is halogen, hydroxy, cyano, (C₁-C₆)alkoxy, or (C₁-C₆)alkyl that is optionally substituted with one or more halo.

In certain embodiments, the optional substituent on the 4-phenyl ring is R³ as defined herein (e.g., in formula (Ia) below).

In certain embodiments, X₂ is a residue of Naphthylmethyl glycine. In certain embodiments, X₂ is a residue of N-(1-Naphthylmethyl)glycine. In certain embodiments, X₂ is a residue of N-(2-Naphthylmethyl)glycine.

In certain embodiments, the invention provides a compound comprising a peptide of Formula (I) X₀—X₁—X₂—X₃—X₄ wherein:

X₀ is a residue of a beta-amino acid, gamma-amino acid or delta-amino acid,

X₁ is a residue of Trp,

X₂ is a residue of 4-(2-methoxyphenyl)-Phe,

X₃ is a residue of an amino acid,

X₄ is a residue of an amino acid. In certain embodiments, the compound consists of such a peptide of Formula (I).

In certain embodiments, the invention provides a compound comprising a peptide of Formula (I) X₀—X₁—X₂—X₃—X₄ wherein:

X₀ is a residue of a beta-amino acid, gamma-amino acid or delta-amino acid,

X₁ is a residue of Trp,

X₂ is a residue of 4-(2-methoxyphenyl)-Phe,

X₃ is a residue of an amino acid,

X₄ is absent, or a residue of an amino acid. In certain embodiments, the compound consists of such a peptide of Formula (I).

In certain embodiments, each residue of an amino acid in the peptide of Formula (I) is independently and optionally substituted. In certain embodiments, Trp at X₁ is optionally substituted on the indole group. For example, in certain embodiments, X₁ is a residue of 5-hydroxy-Trp or 5-methoxy-Trp. In certain embodiments, Bip at X₂ is optionally substituted on one or both phenyl group. For example, in certain embodiments, X₂ is a residue of 4-(2-methoxyphenyl)-Phe.

### X₃ in the Peptide of Formula (I)

In certain embodiments, X₃ is a residue of an amino acid. In certain embodiments, X₃ is a residue of an alpha-amino acid. In certain embodiments, X₃ is a positively charged residue of an amino acid comprising a positively charged amino acid side chain (e.g., an amino acid side chain comprising primary amine or guanidino group). For example, in certain embodiments, X₃ is a residue of hArg, Arg, hLys, Lys, Orn, Dab, Dap, or 4-Guanidino Phe. In certain embodiments, X₃ is a residue of hArg, Arg, hLys, Lys, Orn, Dab or Dap. In certain embodiments, X₃ is a residue of hArg. In certain embodiments, X₃ is a residue of L-hArg. In certain embodiments, X₃ is a residue of Arg. In certain embodiments, X₃ is a residue of hLys. In certain embodiments, X₃ is a residue of Lys. In certain embodiments, X₃ is a residue of Orn. In certain embodiments, X₃ is a residue of Dab. In certain embodiments, X₃ is a residue of Dap. In certain embodiments, X₃ is a residue of 4-Guanidino Phe.

In certain embodiments, the side chain of X₃ is R⁴ as defined herein (e.g., in formula (Ia) below).

In certain embodiments, the residue of X₃ is optionally substituted.

In certain embodiments, the optional substituent is halogen, hydroxy, cyano, (C₁-C₆)alkoxy, or (C₁-C₆)alkyl that is optionally substituted with one or more halo.

In certain embodiments, the invention provides a compound comprising a peptide of Formula (I) X₀—X₁—X₂—X₃—X₄ wherein:

X₀ is a residue of a beta-amino acid, gamma-amino acid or delta-amino acid,

X₁ is a residue of Trp,

X₂ is a residue of Bip,

X₃ is a residue of hArg, Arg, hLys, Lys, Orn, Dab or Dap,

X₄ is a residue of an amino acid. In certain embodiments, the compound consists of such a peptide of Formula (I).

In certain embodiments, the invention provides a compound comprising a peptide of Formula (I) X₀—X₁—X₂—X₃—X₄ wherein:

X₀ is a residue of a beta-amino acid, gamma-amino acid or delta-amino acid,

X₁ is a residue of Trp,

X₂ is a residue of Bip,

X₃ is a residue of hArg, Arg, hLys, Lys, Orn, Dab, Dap, or 4-Guanidino Phe,

X₄ is absent, or a residue of an amino acid. In certain embodiments, the compound consists of such a peptide of Formula (I).

In certain embodiments, each residue of an amino acid in the peptide of Formula (I) is independently and optionally substituted. In certain embodiments, Trp at X₁ is optionally substituted on the indole group. For example, in certain embodiments, X₁ is a residue of 5-hydroxy-Trp or 5-methoxy-Trp. In certain embodiments, Bip at X₂ is optionally substituted on one or both phenyl group. For example, in certain embodiments, X₂ is a residue of 4-(2-methoxyphenyl)-Phe.

X₄ in the peptide of Formula (I)

In certain embodiments, X₄ is absent or a residue of an amino acid.

In certain embodiments, X₄ is a residue of an amino acid (e.g., L or D amino acid). In certain embodiments, X₄ is a residue of an alpha-amino acid. In certain embodiments, X₄ is a residue of Gly, Ala, Thr or Ser. In certain embodiments, X₄ is a residue of Gly, or Ala. In certain embodiments, X₄ is a residue of Gly. In certain embodiments, X₄ is a residue of Ala. In certain embodiments, X₄ is a residue of D-Ala. In certain embodiments, X₄ is a residue of L-Ala. In certain embodiments, X₄ is a residue of Thr. In certain embodiments, X₄ is a residue of Ser. In certain embodiments, X₄ is a residue of Leu. In certain embodiments, X₄ is a residue of Glu. In certain embodiments, X₄ is a residue of Lys. In certain embodiments, X₄ is a residue of Phe. In certain embodiments, X₄ is a residue of Trp.

In certain embodiments, X₄ is a residue of a beta-amino acid (e.g., beta Alanine).

In certain embodiments, X₄ is a residue of Trp. In certain embodiments, X₄ is not a residue of substituted or unsubstituted Trp. In certain embodiments, X₄ is a residue of 3-(4-Pyridyl)-alanine. In certain embodiments, X₄ is not a residue of substituted or unsubstituted 3-(4-Pyridyl)-alanine.

In certain embodiments, the side chain of X₄ is R⁵ as defined herein (e.g., in formula (Ia) below).

In certain embodiments, the residue of X₄ is optionally substituted.

In certain embodiments, optional substituent is halogen, hydroxy, cyano, (C₁-C₆)alkoxy, or (C₁-C₆)alkyl that is optionally substituted with one or more halo.

In certain embodiments, X₄ is a residue of an amino acid, wherein the C-terminus is a carboxyl group —COOH, or the C-terminus is amidated to form —C(═O)NR^{x}R^{y}, wherein R^{x}R^{y} are as defined herein.

In certain embodiments, X₄ is absent.

In certain embodiments, the invention provides a compound comprising a peptide of Formula (I) X₀—X₁—X₂—X₃—X₄ wherein:

X₀ is a residue of a beta-amino acid, gamma-amino acid or delta-amino acid,

X₁ is a residue of Trp,

X₂ is a residue of Bip,

X₃ is a residue of an amino acid,

X₄ is a residue of Gly, Ala, Thr or Ser. In certain embodiments, the compound consists of such a peptide of Formula (I).

In certain embodiments, the invention provides a compound comprising a peptide of Formula (I) X₀—X₁—X₂—X₃—X₄ wherein:

X₀ is a residue of a beta-amino acid, gamma-amino acid or delta-amino acid,

X₁ is a residue of Trp,

X₂ is a residue of Bip,

X₃ is a residue of an amino acid,

X₄ is absent, or a residue of Gly, Ala, Thr, Ser, Glu, Phe, Trp, Leu, Lys, or beta-Alanine.

In certain embodiments, the compound consists of such a peptide of Formula (I).

In certain embodiments, each residue of an amino acid in the peptide of Formula (I) is independently and optionally substituted. In certain embodiments, Trp at X₁ is optionally substituted on the indole group. For example, in certain embodiments, X₁ is a residue of 5-hydroxy-Trp or 5-methoxy-Trp. In certain embodiments, Bip at X₂ is optionally substituted on one or both phenyl group. For example, in certain embodiments, X₂ is a residue of 4-(2-methoxyphenyl)-Phe.

### C-Terminus of the Peptide of Formula (I)

In certain embodiments, the C-terminus of the peptide of formula (I) is amidated with an alkyl-amino group. In certain embodiments, the C-terminus of the peptide of formula (I) is amidated with an aryl-amino or heteroaryl-amino group.

In certain embodiments, the aryl-amino group is anilinyl, which may also be referred to as phenylamino.

In certain embodiments, the heteroaryl-amino group is pyridyl-amino group. In certain embodiments, the heteroaryl-amino group is (2-pyridyl)-amino group.

In certain embodiments, the aryl-amino or heteroaryl-amino is optionally substituted.

In certain embodiments, the optional substituent is halogen, hydroxy, cyano, (C₁-C₆)alkoxy, or (C₁-C₆)alkyl that is optionally substituted with one or more halo.

In certain embodiments, the aryl-amino or heteroaryl-amino is optionally substituted with R⁶ as defined herein (e.g., in formula (Ia) below).

In certain embodiments, the aryl-amino group or heteroaryl-amino group is optionally substituted with halo, (C₁-C₆)alkyl, heterocycle (e.g., 4-morpholinyl, 2-Oxa-6-azaspiro[3.3]heptan-6-yl, 1-piperazinyl or 4-piperidinyl), or amino, wherein the (C₁-C₆)alkyl, heterocycle, or amino is further optionally substituted with optionally substituted aryl, heterocycle or arylalkyl.

In certain embodiments, the anilinyl group is substituted with halo (e.g., fluoro).

In certain embodiments, the anilinyl group is substituted with hydroxy group. For example, in certain embodiments, the anilinyl group is 2-hydroxyl anilinyl.

In certain embodiments, the anilinyl group is substituted (e.g., at para-position) with an alkyl that is further optionally substituted with a heterocycle. For example, the anilinyl group is substituted with (4-morpholinyl)-methyl.

In certain embodiments, the anilinyl group is substituted (e.g., at para-position) with a heterocycle (e.g., 4-morpholinyl, 2-Oxa-6-azaspiro[3.3]heptan-6-yl, 1-piperazinyl or 4-piperidinyl), which is further optionally substituted with optionally substituted arylalkyl (e.g., benzyl optionally substituted with sulfonyl halide).

In certain embodiments, the anilinyl group is substituted (e.g., at para-position) with an amino group, which is optionally substituted with optionally substituted aryl. For example, the anilinyl group is substituted at para-position with 4-methoxyanilinyl.

In certain embodiments, the (2-pyridyl)-amino group is substituted with halo (e.g., fluoro). In certain embodiments, the (2-pyridyl)-amino group is substituted with hydroxy group.

In certain embodiments, the (2-pyridyl)-amino group is substituted with an alkyl that is further optionally substituted with an optionally substituted heterocycle. For example, the (2-pyridyl)-amino group is substituted with (4-morpholinyl)-methyl.

In certain embodiments, the (2-pyridyl)-amino group is substituted with a heterocycle (e.g., 4-morpholinyl, 2-Oxa-6-azaspiro[3.3]heptan-6-yl, 1-piperazinyl or 4-piperidinyl), which is further optionally substituted with optionally substituted arylalkyl (e.g., benzyl optionally substituted with sulfonyl halide).

In certain embodiments, the (2-pyridyl)-amino group is substituted with an amino group, which is optionally substituted with optionally substituted aryl. For example, the (2-pyridyl)-amino group is substituted with 4-methoxyanilinyl.

In certain embodiments, the C-terminus of the peptide of formula (I) is amidated with an alkyl-amino group (e.g., C₁₋₃ alkyl-amino group), which is optionally substituted with an aryl or heteroaryl group on the alkyl group. For example, in certain embodiments, the C-terminus of the peptide of formula (I) may be amidated with 3-(2-amino-ethyl)-5-methoxy-1H-indole. In certain embodiments, the C-terminus of the peptide of formula (I) is amidated with a benzylamino group, such as 2-hydroxybenzylamino or 3,5-dimethoxybenzylamino group.

In certain embodiments, the C-terminus of the peptide of formula (I) is amidated to form —C(═O)NR^{x}R^{y}, wherein R^{x}R^{y} are as defined herein.

In certain embodiments, the C-terminus of the peptide of formula (I) is amidated to form —C(═O)NR^{x}R^{y}, and R^{x} is H; and R^{y} is aryl or heteroaryl, wherein the aryl and heteroaryl is optionally substituted with one or more groups independently selected from the group consisting of halo, hydroxy, cyano, carboxyl, —CONH₂, —NO₂, —SH, —SO₃H, —SO₄H, —SO₂NH₂, —OSO₂F, —SO₂F, —NHNH₂, —ONH₂, —NHC(═O)NHNH₂, —NHC(═O)NH₂, —NHC(═O)H, —NHC(═O)OH, —NHOH, (C₁-C₆)alkyl, (C₁-C₆)alkoxy, (C₁-C₆)alkoxycarbonyl, (C₁-C₆)alkanoyl, (C₁-C₆)alkanoyloxy, (C₁-C₆)heteroalkyl, (C₃-C₆)cycloalkyl, heterocycloalkyl, heterocycloalkyl(C₁-C₆)alkyl, aryl, heteroaryl, and —NR^{r}R^{s}, wherein any (C₁-C₆)alkyl, (C₁-C₆)alkoxy, (C₁-C₆)alkoxycarbonyl, (C₁-C₆)alkanoyl, (C₁-C₆)alkanoyloxy, (C₁-C₆)heteroalkyl, (C₃-C₆)cycloalkyl, heterocycloalkyl, heterocycloalkyl(C₁-C₆)alkyl, aryl, and heteroaryl is optionally substituted with one or more groups independently selected from the group consisting of halo, hydroxy, and (C₁-C₆)alkoxy.

In certain embodiments, the C-terminus of the peptide of formula (I) is amidated to form —C(═O)NR^{x}R^{y}, and R^{x} is H; and R^{y} is phenyl that is optionally substituted with one or more groups independently selected from halo, hydroxy, cyano, carboxyl, —CONH₂, —NO₂, —SH, —SO₃H, —SO₄H, —SO₂NH₂, —OSO₂F, —SO₂F, —NHNH₂, —ONH₂, —NHC(═O)NHNH₂, —NHC(═O)NH₂, —NHC(═O)H, —NHC(═O)OH, —NHOH, (C₁-C₆)alkyl, (C₁-C₆)alkoxy, (C₁-C₆)alkoxycarbonyl, (C₁-C₆)alkanoyl, (C₁-C₆)alkanoyloxy, (C₁-C₆)heteroalkyl, (C₃-C₆)cycloalkyl, heterocycloalkyl, heterocycloalkyl(C₁-C₆)alkyl, aryl, heteroaryl, and —NR^{r}R^{s}, wherein any (C₁-C₆)alkyl, (C₁-C₆)alkoxy, (C₁-C₆)alkoxycarbonyl, (C₁-C₆)alkanoyl, (C₁-C₆)alkanoyloxy, (C₁-C₆)heteroalkyl, (C₃-C₆)cycloalkyl, heterocycloalkyl, heterocycloalkyl(C₁-C₆)alkyl, aryl, and heteroaryl is optionally substituted with one or more groups independently selected from the group consisting of halo, hydroxy, and (C₁-C₆)alkoxy.

In certain embodiments, the C-terminus of the peptide of formula (I) is amidated to form —C(═O)NR^{x}R^{y}, and R^{x} is H; and R^{y} is 2-pyridyl that is optionally substituted with one or more groups independently selected from halo, hydroxy, cyano, carboxyl, —CONH₂, —NO₂, —SH, —SO₃H, —SO₄H, —SO₂NH₂, —OSO₂F, —SO₂F, —NHNH₂, —ONH₂, —NHC(═O)NHNH₂, —NHC(═O)NH₂, —NHC(═O)H, —NHC(═O)OH, —NHOH, (C₁-C₆)alkyl, (C₁-C₆)alkoxy, (C₁-C₆)alkoxycarbonyl, (C₁-C₆)alkanoyl, (C₁-C₆)alkanoyloxy, (C₁-C₆)heteroalkyl, (C₃-C₆)cycloalkyl, heterocycloalkyl, heterocycloalkyl(C₁-C₆)alkyl, aryl, heteroaryl, and —NR^{r}R^{s}, wherein any (C₁-C₆)alkyl, (C₁-C₆)alkoxy, (C₁-C₆)alkoxycarbonyl, (C₁-C₆)alkanoyl, (C₁-C₆)alkanoyloxy, (C₁-C₆)heteroalkyl, (C₃-C₆)cycloalkyl, heterocycloalkyl, heterocycloalkyl(C₁-C₆)alkyl, aryl, and heteroaryl is optionally substituted with one or more groups independently selected from the group consisting of halo, hydroxy, and (C₁-C₆)alkoxy.

In certain embodiments, the invention provides a compound comprising a peptide of Formula (I) X₀—X₁—X₂—X₃—X₄ wherein:

X₀ is a residue of a beta-amino acid, gamma-amino acid or delta-amino acid,

X₁ is a residue of Trp,

X₂ is a residue of Bip,

X₃ is a residue of an amino acid,

X₄ is a residue of an amino acid,

wherein the carboxylic acid terminal of X₄ is amidated (e.g., via anilinyl or (2-pyridyl)-amino group). In certain embodiments, the compound consists of such a peptide of Formula (I).

In certain embodiments, the invention provides a compound that comprises a peptide of Formula (I) wherein:

X₀ is a residue of GABA or ACHC,

X₁ is a residue of Trp, 5-hydroxy-Trp or 5-methoxy-Trp,

X₂ is a residue of Bip or 4-(2-methoxyphenyl)-Phe,

X₃ is a residue of hArg, Arg, hLys, Lys, Orn, Dab or Dap,

X₄ is a residue of Gly, Ala, Thr or Ser,

wherein the C-terminus of the peptide of Formula (I) is amidated with anilinyl group or (2-pyridyl)-amino group,

or a salt thereof. In certain embodiments, the compound consists of such a peptide of Formula (I).

In certain embodiments, the anilinyl group or (2-pyridyl)-amino group is optionally substituted.

In certain embodiments, the optional substituent is halogen, hydroxy, cyano, (C₁-C₆)alkoxy, or (C₁-C₆)alkyl that is optionally substituted with one or more halo.

In certain embodiments, the aryl-amino or heteroaryl-amino is optionally substituted with R⁶ as defined herein (e.g., in formula (Ia) below).

In certain embodiments, the invention provides a compound that comprises a peptide of Formula (I) wherein:

X₀ is a residue of GABA or ACHC,

X₁ is a residue of Trp, 5-hydroxy-Trp or 5-methoxy-Trp,

X₂ is a residue of Bip or 4-(2-methoxyphenyl)-Phe,

X₃ is a residue of hArg, Arg, hLys, Lys, Orn, Dab or Dap,

X₄ is a residue of Gly, Ala, Thr or Ser,

wherein the C-terminus of the peptide of Formula (I) is amidated and R^{x} is H; and R^{y} is 2-pyridyl or phenyl that is optionally substituted with one or more groups independently selected from the group consisting of halo, hydroxy, cyano, carboxyl, —CONH₂, —NO₂, —SH, —SO₃H, —SO₄H, —SO₂NH₂, —OSO₂F, —SO₂F, —NHNH₂, —ONH₂, —NHC(═O)NHNH₂, —NHC(═O)NH₂, —NHC(═O)H, —NHC(═O)OH, —NHOH, (C₁-C₆)alkyl, (C₁-C₆)alkoxy, (C₁-C₆)alkoxycarbonyl, (C₁-C₆)alkanoyl, (C₁-C₆)alkanoyloxy, (C₁-C₆)heteroalkyl, (C₃-C₆)cycloalkyl, heterocycloalkyl, heterocycloalkyl(C₁-C₆)alkyl, aryl, heteroaryl, and —NR^{r}R^{s}, wherein any (C₁-C₆)alkyl, (C₁-C₆)alkoxy, (C₁-C₆)alkoxycarbonyl, (C₁-C₆)alkanoyl, (C₁-C₆)alkanoyloxy, (C₁-C₆)heteroalkyl, (C₃-C₆)cycloalkyl, heterocycloalkyl, heterocycloalkyl(C₁-C₆)alkyl, aryl, and heteroaryl is optionally substituted with one or more groups independently selected from the group consisting of halo, hydroxy, and (C₁-C₆)alkoxy;

or a salt thereof. In certain embodiments, X₄ is a residue of Gly or Ala. In certain embodiments, the compound consists of such a peptide of Formula (I).

In certain embodiments, each residue of an amino acid in the peptide of Formula (I) is independently and optionally substituted. In certain embodiments, Trp at X₁ is optionally substituted on the indole group. For example, in certain embodiments, X₁ is a residue of 5-hydroxy-Trp or 5-methoxy-Trp. In certain embodiments, Bip at X₂ is optionally substituted on one or both phenyl group. For example, in certain embodiments, X₂ is a residue of 4-(2-methoxyphenyl)-Phe.

In certain embodiments, the dipeptide segment X₁—X₂ in formula (I) is Trp-Bip.

In certain embodiments, the tripeptide segment X₀—X₁—X₂ in formula (I) is GABA-Trp-Bip.

In certain embodiments, the tripeptide segment X₀—X₁—X₂ in formula (I) is ACHC-Trp-Bip.

In certain embodiments, the tripeptide segment X₁—X₂—X₃ in formula (I) is Trp-Bip-hArg.

In certain embodiments, the tetrapeptide segment X₀—X₁—X₂—X₃ in formula (I) is GABA-Trp-Bip-hArg.

In certain embodiments, the tetrapeptide segment X₀—X₁—X₂—X₃ in formula (I) is ACHC-Trp-Bip-hArg.

In certain embodiments, the tetrapeptide segment X₁—X₂—X₃—X₄ in formula (I) is Trp-Bip-hArg-Gly or Trp-Bip-hArg-Ala.

In certain embodiments, the peptide of formula (I) X₀—X₁—X₂—X₃—X₄ is GABA-Trp-Bip-hArg-Gly or GABA-Trp-Bip-hArg-Ala.

In certain embodiments, the peptide of formula (I) X₀—X₁—X₂—X₃—X₄ is ACHC-Trp-Bip-hArg-Gly or ACHC-Trp-Bip-hArg-Ala.

Each amino acid of the peptide segment or formula (I) described herein is optionally and independently substituted. For example, any peptide segment or formula (I) comprising Trp in X₁ can be 5-hydroxy-Trp in X₁ or 5-methoxy-Trp in X₁. Any peptide segment or formula (I) comprising Bip in X₂ can be 4-(2-methoxyphenyl)-Phe in X₂.

Further embodiments of peptide of formula (I) provided herein include combinations of optionally substituted residues or terminus from one or more of certain embodiments described herein.

Certain embodiments of the invention provide a compound comprising a peptide of Formula (I) as described herein. Certain embodiments of the invention provide a peptide of Formula (I) as described herein.

Certain embodiments of the invention provide a compound comprising a peptide of Formula (I′):

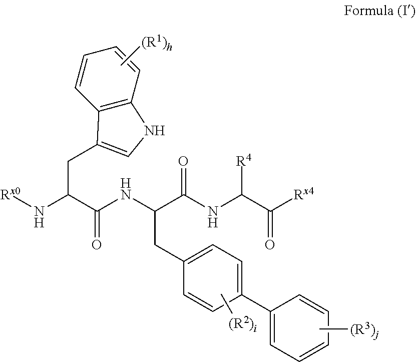

wherein

h, i, and j are each independent 0, 1, 2 or 3;

R¹, R², and R³ are each independently absent, hydrogen, halo, hydroxy, cyano, carboxyl, —CONH₂, —NO₂, —SH, —SO₃H, —SO₄H, —SO₂NH₂, —OSO₂F, —SO₂F, —NHNH₂, —ONH₂, —NHC(═O)NHNH₂, —NHC(═O)NH₂, —NHC(═O)H, —NHC(═O)OH, —NHOH, (C₁-C₆)alkyl, (C₁-C₆)alkoxy, (C₁-C₆)alkoxycarbonyl, (C₁-C₆)alkanoyl, (C₁-C₆)alkanoyloxy, (C₁-C₆)heteroalkyl, (C₃-C₆)cycloalkyl, heterocycloalkyl, heterocycloalkyl(C₁-C₆)alkyl, aryl, heteroaryl, and —NR^{r}R^{s}, wherein any (C₁-C₆)alkyl, (C₁-C₆)alkoxy, (C₁-C₆)alkoxycarbonyl, (C₁-C₆)alkanoyl, (C₁-C₆)alkanoyloxy, (C₁-C₆)heteroalkyl, (C₃-C₆)cycloalkyl, heterocycloalkyl, heterocycloalkyl(C₁-C₆)alkyl, aryl, and heteroaryl is optionally substituted with one or more groups independently selected from the group consisting of halo, hydroxy, and (C₁-C₆)alkoxy;

R⁴ is an amino acid side chain;

R^{x0} is a residue of an amino acid, wherein the N-terminus is a primary amine group NH₂— or capped amine R^{N}—C(═O)—NH—, wherein R^{N} is H, (C₃-C₆)cycloalkyl, or (C₁-C₄)alkyl;

R^{x4} is —OH, —NH₂, or a residue of an amino acid wherein the C-terminus is a free carboxyl group —COOH or the C-terminus of the amino acid is amidated to form —C(═O)NR^{x}R^{y};

wherein each R^{x} and R^{y} is independently selected from the group consisting of H, (C₁-C₆)alkyl, (C₃-C₆)cycloalkyl, aryl, heteroaryl, (C₃-C₆)cycloalkyl(C₁-C₆)alkyl, aryl(C₁-C₆)alkyl, heteroaryl(C₁-C₆)alkyl, aryl-heterocycloalkyl-aryl, aryl-heterocycloalkyl-heteroaryl, aryl-heterocycloalkyl-(C₁-C₆)alkyl-aryl, aryl-heterocycloalkyl-(C₁-C₆)alkyl-heteroaryl, and aryl-(C₁-C₆)alkyl-heterocycloalkyl-aryl,

wherein any (C₁-C₆)alkyl, (C₃-C₆)cycloalkyl, aryl, heteroaryl, (C₃-C₆)cycloalkyl(C₁-C₆)alkyl, aryl(C₁-C₆)alkyl, heteroaryl(C₁-C₆)alkyl, aryl-heterocycloalkyl-aryl, aryl-heterocycloalkyl-heteroaryl, aryl-heterocycloalkyl-(C₁-C₆)alkyl-aryl, aryl-heterocycloalkyl-(C₁-C₆)alkyl-heteroaryl, and aryl-(C₁-C₆)alkyl-heterocycloalkyl-aryl is optionally substituted with one or more groups independently selected from the group consisting of halo, hydroxy, cyano, carboxyl, —CONH₂, —NO₂, —SH, —SO₃H, —SO₄H, —SO₂NH₂, —OSO₂F, —SO₂F, —NHNH₂, —ONH₂, —NHC(═O)NHNH₂, —NHC(═O)NH₂, —NHC(═O)H, —NHC(═O)OH, —NHOH, (C₁-C₆)alkyl, (C₁-C₆)alkoxy, (C₁-C₆)alkoxycarbonyl, (C₁-C₆)alkanoyl, (C₁-C₆)alkanoyloxy, (C₁-C₆)heteroalkyl, (C₃-C₆)cycloalkyl, heterocycloalkyl, heterocycloalkyl(C₁-C₆)alkyl, aryl, heteroaryl, and —NR^{r}R^{s}, wherein any (C₁-C₆)alkyl, (C₁-C₆)alkoxy, (C₁-C₆)alkoxycarbonyl, (C₁-C₆)alkanoyl, (C₁-C₆)alkanoyloxy, (C₁-C₆)heteroalkyl, (C₃-C₆)cycloalkyl, heterocycloalkyl, heterocycloalkyl(C₁-C₆)alkyl, aryl, and heteroaryl is optionally substituted with one or more groups independently selected from the group consisting of halo, hydroxy, and (C₁-C₆)alkoxy;

each R^{r} and R^{s} is independently selected from the group consisting of H, (C₁-C₆)alkyl, aryl, and (C₁-C₆)alkoxyaryl;

or a salt thereof.

Certain embodiments of the invention provide a peptide of Formula (I′) as described herein, or a salt thereof.

In certain embodiments, R^{x0} is X₀ as described herein. For example, in certain embodiments, R^{x0} is a residue of an alpha-amino acid (e.g., Ala). In certain embodiments, R^{x0} is a residue of a beta-amino acid, gamma-amino acid, or delta-amino acid. In certain embodiments, R^{x0} is a residue of a gamma-amino acid (e.g., GABA or ACHC),

In certain embodiments, R^{x4} is a residue of an amino acid. In certain embodiments, the R^{x4} is X₄ as described herein. For example, in certain embodiments, R^{x4} is a residue of Gly, Ala, Thr, Ser, Leu, Glu, Lys, Phe, Trp, or beta alanine. In certain embodiments, the C-terminal of R^{x4} is amidated (e.g., via an alkyl-amino, aryl-amino or heteroaryl amino group as described herein, such as an optionally substituted anilinyl or (2-pyridyl)-amino group).

In certain embodiments, R⁴ is an amino acid side chain that is positively charged. For example, in certain embodiments, R⁴ is an amino acid side chain comprising a guanidino group or a primary amino group. The R⁴ amino acid side chain may have a positive charge under suitable conditions. In certain embodiments, R⁴ is the L-hArg side chain, which is 4-guanidino-butyl or H₂NC(═NH)NH(CH₂)₄—. In certain embodiments, R⁴ is the side chain of hArg, Arg, hLys, Lys, Orn, Dab or Dap. In certain embodiments, R⁴ is the side chain of 4-Guanidino Phe.

Certain embodiments of the invention provide a compound consisting of a peptide of Formula (I) as described herein, or a salt thereof. Certain embodiments of the invention provide a peptide of Formula (I) as described herein, or a salt thereof. Certain embodiments of the invention provide a compound comprising a peptide of Formula (Ia):

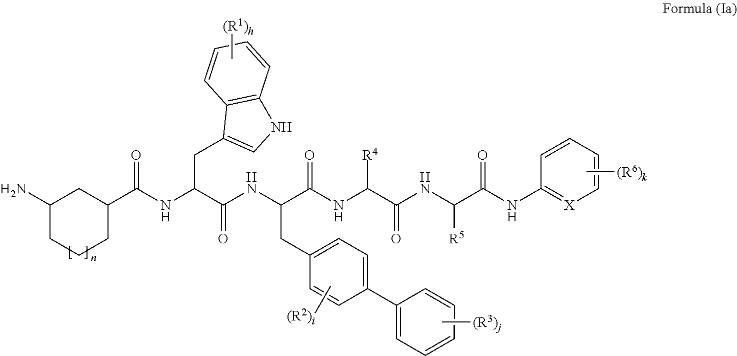

wherein

n is 0, 1, or 2;

X is C or N;

h, i, j and k are each independent 0, 1, 2 or 3;

R¹, R², R³, and R⁶ are each independently selected from the group consisting of absent, halo, hydroxy, cyano, carboxyl, —CONH₂, —NO₂, —SH, —SO₃H, —SO₄H, —SO₂NH₂, —OSO₂F, —SO₂F, —NHNH₂, —ONH₂, —NHC(═O)NHNH₂, —NHC(═O)NH₂, —NHC(═O)H, —NHC(═O)OH, —NHOH, (C₁-C₆)alkyl, (C₁-C₆)alkoxy, (C₁-C₆)alkoxycarbonyl, (C₁-C₆)alkanoyl, (C₁-C₆)alkanoyloxy, (C₁-C₆)heteroalkyl, (C₃-C₆)cycloalkyl, heterocycloalkyl, heterocycloalkyl(C₁-C₆)alkyl, aryl, heteroaryl, and —NR^{r}R^{s}, wherein any (C₁-C₆)alkyl, (C₁-C₆)alkoxy, (C₁-C₆)alkoxycarbonyl, (C₁-C₆)alkanoyl, (C₁-C₆)alkanoyloxy, (C₁-C₆)heteroalkyl, (C₃-C₆)cycloalkyl, heterocycloalkyl, heterocycloalkyl(C₁-C₆)alkyl, aryl, and heteroaryl is optionally substituted with one or more groups independently selected from the group consisting of halo, hydroxy, and (C₁-C₆)alkoxy;

R⁴, and R⁵ are each independently an amino acid side chain;

each R^{r} and R^{s} is independently selected from the group consisting of H, (C₁-C₆)alkyl, aryl, and (C₁-C₆)alkoxyaryl;

or a salt thereof.

Certain embodiments of the invention provide a peptide of Formula (Ia) as described herein, or a salt thereof.

In certain embodiments, R¹, R², R³, R⁴, R⁵, and R⁶ of Formula (Ia) are each independently absent, hydrogen, halogen, —OCH₃, —CN, —OH, —NH₂, —CH₂OH, —CH₂(OH)CH₃, —COOH, —CONH₂, —NO₂, —SH, —SO₃H, —SO₄H, —SO₂NH₂, —OSO₂F, —SO₂F, —NHNH₂, —ONH₂, —NHC(═O)NHNH₂, —NHC(═O)NH₂, —NHC(═O)H, —NHC(═O)OH, —NHOH, —OCCl₃, —OCBr₃, —OCF₃, —OCl₃, —OCH₂Cl, —OCH₂Br, —OCH₂F, —OCH₂I, —OCHCl₂, —OCHBr₂, —OCHF₂, —OCHI₂, an amino acid side chain, alkyl, heteroalkyl, cycloalkyl, heterocycloalkyl, aryl, or heteroaryl.

In certain embodiments, the alkyl, heteroalkyl, cycloalkyl, heterocycloalkyl, aryl, or heteroaryl is optionally substituted.

In certain embodiments, the alkyl, heteroalkyl, cycloalkyl, heterocycloalkyl, aryl, or heteroaryl is optionally substituted with halogen, hydroxy, cyano, (C₁-C₆)alkoxy, or (C₁-C₆)alkyl that is optionally substituted with one or more halo.

In certain embodiments, the alkyl, heteroalkyl, cycloalkyl, heterocycloalkyl, aryl, or heteroaryl is optionally substituted with halogen, hydroxy, (C₁-C₆)alkoxy, —OSO₂F, or —SO₂F.

In certain embodiments, n is 1.

In certain embodiments, X is C. In certain embodiments, X is N.

In certain embodiments, R¹ is —OH. In certain embodiments, R¹ is —OCH₃.

In certain embodiments, R² is absent.

In certain embodiments, R³ is —OCH₃. In certain embodiments, R³ is absent. In certain embodiments, R³ is C₁₋₃ alkyl. In certain embodiments, R³ is —CH₃ or halogen (e.g., chloro).

In certain embodiments, R⁴ is an amino acid side chain comprising a primary amine group (e.g., guanidino group or amino group). The primary amine group may have a positive charge under suitable conditions. In certain embodiments, R⁴ is the L-hArg side chain, which is 4-guanidino-butyl or H₂NC(═NH)NH(CH₂)₄—. In certain embodiments, R⁴ is the side chain of hArg, Arg, hLys, Lys, Orn, Dab or Dap. In certain embodiments, R⁴ is the side chain of 4-Guanidino Phe.

In certain embodiments, R^{s} is H. In certain embodiments, R^{s} is CH₃. In certain embodiments, R^{s} is —H, —CH₃, —CH₂OH, or —CH(OH)CH₃.

In certain embodiments, R⁶ is 4-morpholinyl. In certain embodiments, R⁶ is 2-Oxa-6-azaspiro[3.3]heptan-6-yl. In certain embodiments, R⁶ is (4-morpholinyl)-methyl. In certain embodiments, R⁶ is 1-piperazinyl. In certain embodiments, R⁶ is anilinyl. In certain embodiments, R⁶ is fluoro.

In certain embodiments, Formula (Ia) is Formula (Ia′):

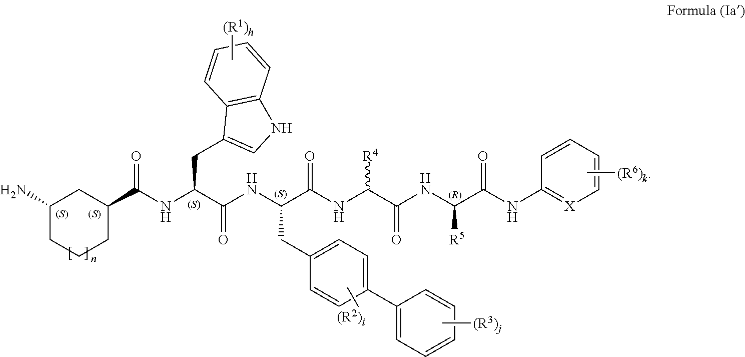

Certain embodiments of the invention provide a compound comprising a peptide of Formula (Ib):

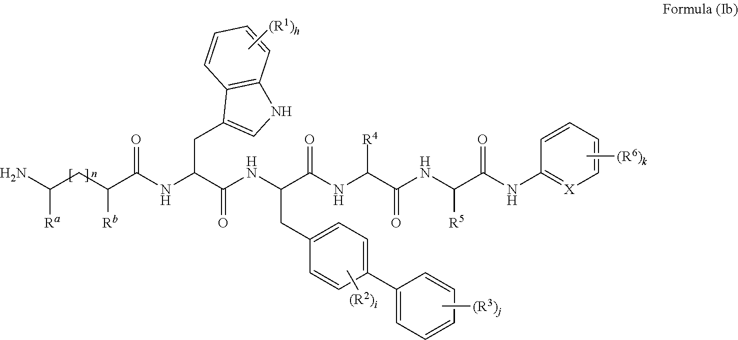

wherein

n is 0, 1, or 2;

X is C or N;

h, i, j and k are each independent 0, 1, 2 or 3;

R^{a}, R^{b}, R¹, R², R³, and R⁶ are each independently selected from the group consisting of absent, hydrogen, halo, hydroxy, cyano, carboxyl, —CONH₂, —NO₂, —SH, —SO₃H, —SO₄H, —SO₂NH₂, —OSO₂F, —SO₂F, —NHNH₂, —ONH₂, —NHC(═O)NHNH₂, —NHC(═O)NH₂, —NHC(═O)H, —NHC(═O)OH, —NHOH, (C₁-C₆)alkyl, (C₁-C₆)alkoxy, (C₁-C₆)alkoxycarbonyl, (C₁-C₆)alkanoyl, (C₁-C₆)alkanoyloxy, (C₁-C₆)heteroalkyl, (C₃-C₆)cycloalkyl, heterocycloalkyl, heterocycloalkyl(C₁-C₆)alkyl, aryl, heteroaryl, and —NR^{r}R^{s}, wherein any (C₁-C₆)alkyl, (C₁-C₆)alkoxy, (C₁-C₆)alkoxycarbonyl, (C₁-C₆)alkanoyl, (C₁-C₆)alkanoyloxy, (C₁-C₆)heteroalkyl, (C₃-C₆)cycloalkyl, heterocycloalkyl, heterocycloalkyl(C₁-C₆)alkyl, aryl, and heteroaryl is optionally substituted with one or more groups independently selected from the group consisting of halo, hydroxy, and (C₁-C₆)alkoxy;

R⁴, and R⁵ are each independently an amino acid side chain;

each R^{r} and R^{s} is independently selected from the group consisting of H, (C₁-C₆)alkyl, aryl, and (C₁-C₆)alkoxyaryl;

or a salt thereof.

Certain embodiments of the invention provide a peptide of Formula (Ib) as described herein, or a salt thereof.

In certain embodiments, R^{a}, R^{b}, R¹, R², R³, R⁴, R⁵, and R⁶ of Formula (Ib) are each independently absent, hydrogen, halogen, —OCH₃, —CN, —OH, —NH₂, —CH₂OH, —CH₂(OH)CH₃, —COOH, —CONH₂, —NO₂, —SH, —SO₃H, —SO₄H, —SO₂NH₂, —OSO₂F, —SO₂F, —NHNH₂, —ONH₂, —NHC(═O)NHNH₂, —NHC(═O)NH₂, —NHC(═O)H, —NHC(═O)OH, —NHOH, —OCCl₃, —OCBr₃, —OCF₃, —OCl₃, —OCH₂Cl, —OCH₂Br, —OCH₂F, —OCH₂I, —OCHCl₂, —OCHBr₂, —OCHF₂, —OCHI₂, an amino acid side chain, alkyl, heteroalkyl, cycloalkyl, heterocycloalkyl, aryl, or heteroaryl.

In certain embodiments, the alkyl, heteroalkyl, cycloalkyl, heterocycloalkyl, aryl, or heteroaryl is optionally substituted.

In certain embodiments, the alkyl, heteroalkyl, cycloalkyl, heterocycloalkyl, aryl, or heteroaryl is optionally substituted with halogen, hydroxy, cyano, (C₁-C₆)alkoxy, or (C₁-C₆)alkyl that is optionally substituted with one or more halo.

In certain embodiments, the alkyl, heteroalkyl, cycloalkyl, heterocycloalkyl, aryl, or heteroaryl is optionally substituted with halogen, hydroxy, (C₁-C₆)alkoxy, —OSO₂F, or —SO₂F.

In certain embodiments, n is 1.

In certain embodiments, R^{a} is H.

In certain embodiments, R^{b} is H.

In certain embodiments, X is C. In certain embodiments, X is N.

In certain embodiments, R¹ is —OH. In certain embodiments, R¹ is —OCH₃.

In certain embodiments, R² is absent.

In certain embodiments, R³ is —OCH₃. In certain embodiments, R³ is absent. In certain embodiments, R³ is C₁₋₃ alkyl. In certain embodiments, R³ is —CH₃ or halogen (e.g., chloro).

In certain embodiments, R⁴ is an amino acid side chain comprising a primary amine group (e.g., guanidino group or amino group). The primary amine group may have a positive charge under suitable conditions. In certain embodiments, R⁴ is the L-hArg side chain. In certain embodiments, R⁴ is the side chain of hArg, Arg, hLys, Lys, Orn, Dab or Dap. In certain embodiments, R⁴ is the side chain of 4-Guanidino Phe.

In certain embodiments, R^{s} is H. In certain embodiments, R^{s} is CH₃. In certain embodiments, R⁵ is —H, —CH₃, —CH₂OH, or —CH(OH)CH₃.

In certain embodiments, R⁶ is 4-morpholinyl. In certain embodiments, R⁶ is 2-Oxa-6-azaspiro[3.3]heptan-6-yl. In certain embodiments, R⁶ is (4-morpholinyl)-methyl. In certain embodiments, R⁶ is 1-piperazinyl. In certain embodiments, R⁶ is anilinyl. In certain embodiments, R⁶ is fluoro. In certain embodiments, R⁶ is —OSO₂F, or —SO₂F.

In certain embodiments, Formula (Ib) is Formula (Ib′):

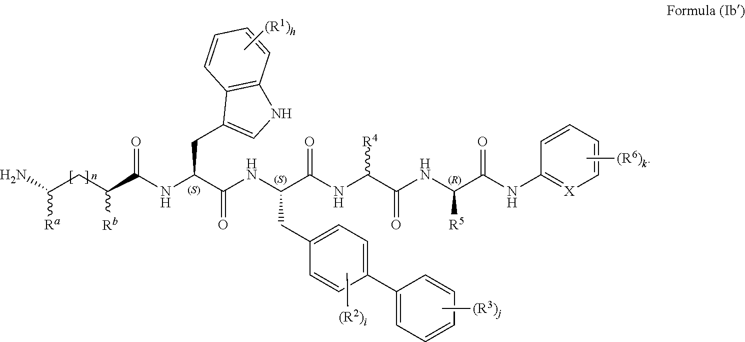

Certain embodiments of the invention provide a compound comprising a peptide of Formula (Ic):

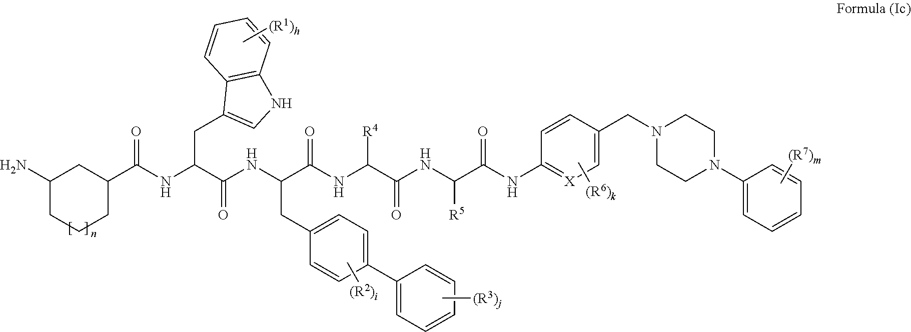

wherein

n is 0, 1, or 2;

X is C or N;

h, i, j, k and m are each independent 0, 1, 2 or 3;

R¹, R², R³, R⁶ and R⁷ are each independently selected from the group consisting of absent, halo, hydroxy, cyano, carboxyl, —CONH₂, —NO₂, —SH, —SO₃H, —SO₄H, —SO₂NH₂, —OSO₂F, —SO₂F, —NHNH₂, —ONH₂, —NHC(═O)NHNH₂, —NHC(═O)NH₂, —NHC(═O)H, —NHC(═O)OH, —NHOH, (C₁-C₆)alkyl, (C₁-C₆)alkoxy, (C₁-C₆)alkoxycarbonyl, (C₁-C₆)alkanoyl, (C₁-C₆)alkanoyloxy, (C₁-C₆)heteroalkyl, (C₃-C₆)cycloalkyl, heterocycloalkyl, heterocycloalkyl(C₁-C₆)alkyl, aryl, heteroaryl, and —NR^{r}R^{s}, wherein any (C₁-C₆)alkyl, (C₁-C₆)alkoxy, (C₁-C₆)alkoxycarbonyl, (C₁-C₆)alkanoyl, (C₁-C₆)alkanoyloxy, (C₁-C₆)heteroalkyl, (C₃-C₆)cycloalkyl, heterocycloalkyl, heterocycloalkyl(C₁-C₆)alkyl, aryl, and heteroaryl is optionally substituted with one or more groups independently selected from the group consisting of halo, hydroxy, and (C₁-C₆)alkoxy;

R⁴, and R⁵ are each independently an amino acid side chain;

each R^{r} and R^{s} is independently selected from the group consisting of H, (C₁-C₆)alkyl, aryl, and (C₁-C₆)alkoxyaryl;

or a salt thereof.

Certain embodiments of the invention provide a peptide of Formula (Ic) as described herein, or a salt thereof.

In certain embodiments, R¹, R², R³, R⁴, R⁵, R⁶ and R⁷ of Formula (Ic) are each independently absent, hydrogen, halogen, —OCH₃, —CN, —OH, —NH₂, —CH₂OH—, —CH₂(OH)CH₃, —COOH, —CONH₂, —NO₂, —SH, —SO₃H, —SO₄H, —SO₂NH₂, —OSO₂F, —SO₂F, —NHNH₂, —ONH₂, —NHC(═O)NHNH₂, —NHC(═O)NH₂, —NHC(═O)H, —NHC(═O)OH, —NHOH, —OCCl₃, —OCBr₃, —OCF₃, —OCl₃, —OCH₂Cl, —OCH₂Br, —OCH₂F, —OCH₂I, —OCHCl₂, —OCHBr₂, —OCHF₂, —OCHI₂, an amino acid side chain, alkyl, heteroalkyl, cycloalkyl, heterocycloalkyl, aryl, or heteroaryl.

In certain embodiments, the alkyl, heteroalkyl, cycloalkyl, heterocycloalkyl, aryl, or heteroaryl is optionally substituted.

In certain embodiments, the alkyl, heteroalkyl, cycloalkyl, heterocycloalkyl, aryl, or heteroaryl is optionally substituted with halogen, hydroxy, cyano, (C₁-C₆)alkoxy, or (C₁-C₆)alkyl that is optionally substituted with one or more halo.

In certain embodiments, the alkyl, heteroalkyl, cycloalkyl, heterocycloalkyl, aryl, or heteroaryl is optionally substituted with halogen, hydroxy, (C₁-C₆)alkoxy, —OSO₂F, or —SO₂F.

In certain embodiments, n is 1.

In certain embodiments, X is C. In certain embodiments, X is N.

In certain embodiments, R¹ is —OH. In certain embodiments, R¹ is —OCH₃.

In certain embodiments, R² is absent.

In certain embodiments, R³ is —OCH₃. In certain embodiments, R³ is absent. In certain embodiments, R³ is C₁₋₃ alkyl. In certain embodiments, R³ is —CH₃ or halogen (e.g., chloro).

In certain embodiments, R⁴ is an amino acid side chain comprising a primary amine group (e.g., guanidino group or amino group). The primary amine group may have a positive charge under suitable conditions. In certain embodiments, R⁴ is the L-hArg side chain. In certain embodiments, R⁴ is the side chain of hArg, Arg, hLys, Lys, Orn, Dab or Dap. In certain embodiments, R⁴ is the side chain of 4-Guanidino Phe.

In certain embodiments, R⁵ is H. In certain embodiments, R⁵ is CH₃. In certain embodiments, R^{s} is —H, —CH₃, —CH₂OH, or —CH(OH)CH₃.

In certain embodiments, R⁶ is fluoro.

In certain embodiments, R⁷ is —OSO₂F. In certain embodiments, R⁷ is —SO₂F.

In certain embodiments, Formula (Ic) is Formula (Ic′):

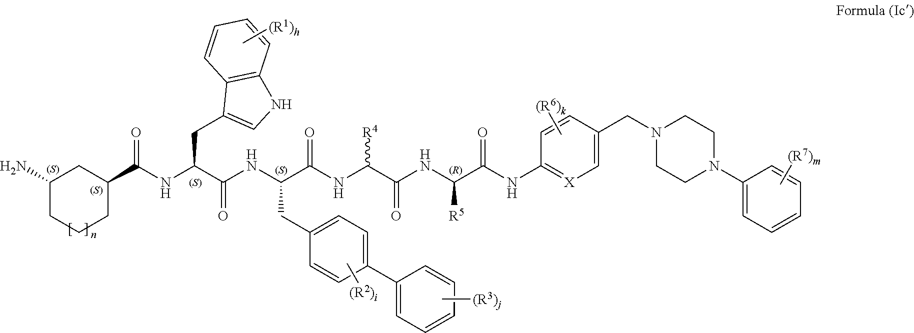

Certain embodiments of the invention provide a compound comprising a peptide of Formula (Id):

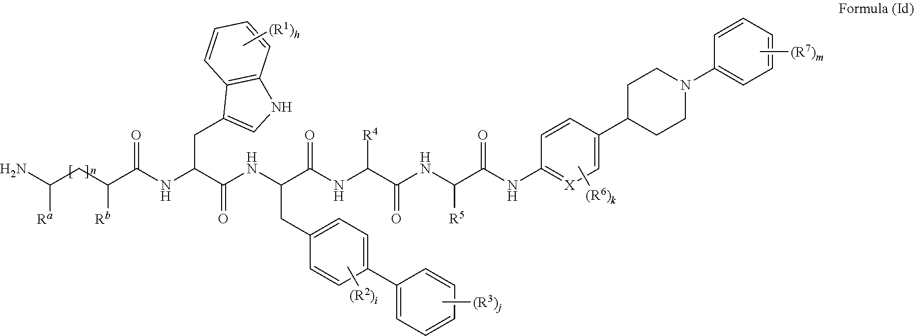

wherein

n is 0, 1, or 2;

X is C or N;

h, i, j, k and m are each independent 0, 1, 2 or 3;

R^{a}, R^{b}, R¹, R², R³, R⁶ and R⁷ are each independently selected from the group consisting of absent, hydrogen, halo, hydroxy, cyano, carboxyl, —CONH₂, —NO₂, —SH, —SO₃H, —SO₄H, —SO₂NH₂, —OSO₂F, —SO₂F, —NHNH₂, —ONH₂, —NHC(═O)NHNH₂, —NHC(═O)NH₂, —NHC(═O)H, —NHC(═O)OH, —NHOH, (C₁-C₆)alkyl, (C₁-C₆)alkoxy, (C₁-C₆)alkoxycarbonyl, (C₁-C₆)alkanoyl, (C₁-C₆)alkanoyloxy, (C₁-C₆)heteroalkyl, (C₃-C₆)cycloalkyl, heterocycloalkyl, heterocycloalkyl(C₁-C₆)alkyl, aryl, heteroaryl, and —NR^{r}R^{s}, wherein any (C₁-C₆)alkyl, (C₁-C₆)alkoxy, (C₁-C₆)alkoxycarbonyl, (C₁-C₆)alkanoyl, (C₁-C₆)alkanoyloxy, (C₁-C₆)heteroalkyl, (C₃-C₆)cycloalkyl, heterocycloalkyl, heterocycloalkyl(C₁-C₆)alkyl, aryl, and heteroaryl is optionally substituted with one or more groups independently selected from the group consisting of halo, hydroxy, and (C₁-C₆)alkoxy;

R⁴, and R⁵ are each independently an amino acid side chain;

each R^{r} and R^{s} is independently selected from the group consisting of H, (C₁-C₆)alkyl, aryl, and (C₁-C₆)alkoxyaryl;

or a salt thereof.

Certain embodiments of the invention provide a peptide of Formula (Id) as described herein, or a salt thereof.

In certain embodiments, R^{a}, R^{b}, R¹, R², R³, R⁴, R⁵, R⁶ and R⁷ of Formula (Id) are each independently absent, hydrogen, halogen, —OCH₃, —CN, —OH, —NH₂, —CH₂OH—, —CH₂(OH)CH₃, —COOH, —CONH₂, —NO₂, —SH, —SO₃H, —SO₄H, —SO₂NH₂, —OSO₂F, —SO₂F, —NHNH₂, —ONH₂, —NHC(═O)NHNH₂, —NHC(═O)NH₂, —NHC(═O)H, —NHC(═O)OH, —NHOH, —OCCl₃, —OCBr₃, —OCF₃, —OCI₃, —OCH₂Cl, —OCH₂Br, —OCH₂F, —OCH₂I, —OCHCl₂, —OCHBr₂, —OCHF₂, —OCHI₂, an amino acid side chain, alkyl, heteroalkyl, cycloalkyl, heterocycloalkyl, aryl, or heteroaryl.

In certain embodiments, the alkyl, heteroalkyl, cycloalkyl, heterocycloalkyl, aryl, or heteroaryl is optionally substituted.

In certain embodiments, the alkyl, heteroalkyl, cycloalkyl, heterocycloalkyl, aryl, or heteroaryl is optionally substituted with halogen, hydroxy, cyano, (C₁-C₆)alkoxy, or (C₁-C₆)alkyl that is optionally substituted with one or more halo.

In certain embodiments, the alkyl, heteroalkyl, cycloalkyl, heterocycloalkyl, aryl, or heteroaryl is optionally substituted with halogen, hydroxy, (C₁-C₆)alkoxy, —OSO₂F, or —SO₂F.

In certain embodiments, n is 1.

In certain embodiments, X is C. In certain embodiments, X is N.

In certain embodiments, R^{a} is H.

In certain embodiments, R^{b} is H.

In certain embodiments, R¹ is —OH. In certain embodiments, R¹ is —OCH₃.

In certain embodiments, R² is absent.

In certain embodiments, R³ is —OCH₃. In certain embodiments, R³ is absent. In certain embodiments, R³ is C₁₋₃ alkyl. In certain embodiments, R³ is —CH₃ or halogen (e.g., chloro).

In certain embodiments, R⁴ is an amino acid side chain comprising a primary amine group (e.g., guanidino group or amino group). The primary amine group may have a positive charge under suitable conditions. In certain embodiments, R⁴ is the L-hArg side chain. In certain embodiments, R⁴ is the side chain of hArg, Arg, hLys, Lys, Orn, Dab or Dap. In certain embodiments, R⁴ is the side chain of 4-Guanidino Phe.

In certain embodiments, R⁵ is H. In certain embodiments, R⁵ is CH₃. In certain embodiments, R⁵ is —H, —CH₃, —CH₂OH, or —CH(OH)CH₃.

In certain embodiments, R⁶ is fluoro.

In certain embodiments, R⁷ is —OSO₂F. In certain embodiments, R⁷ is —SO₂F.

In certain embodiments, Formula (Id) is Formula (Id′):

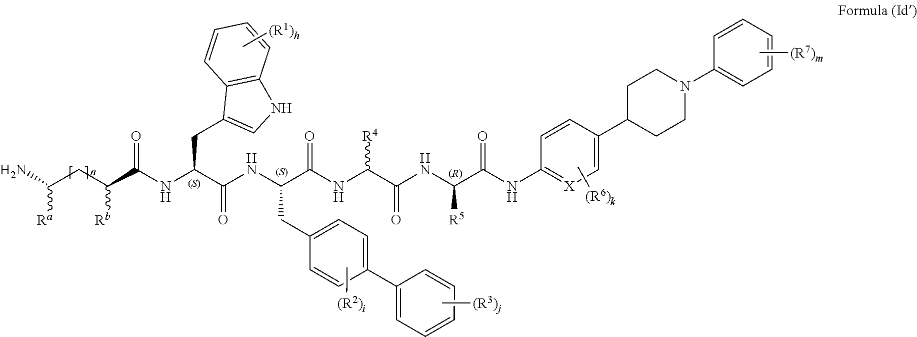

In certain embodiments, the invention provides a compound comprising a peptide of Formula (I), which is:

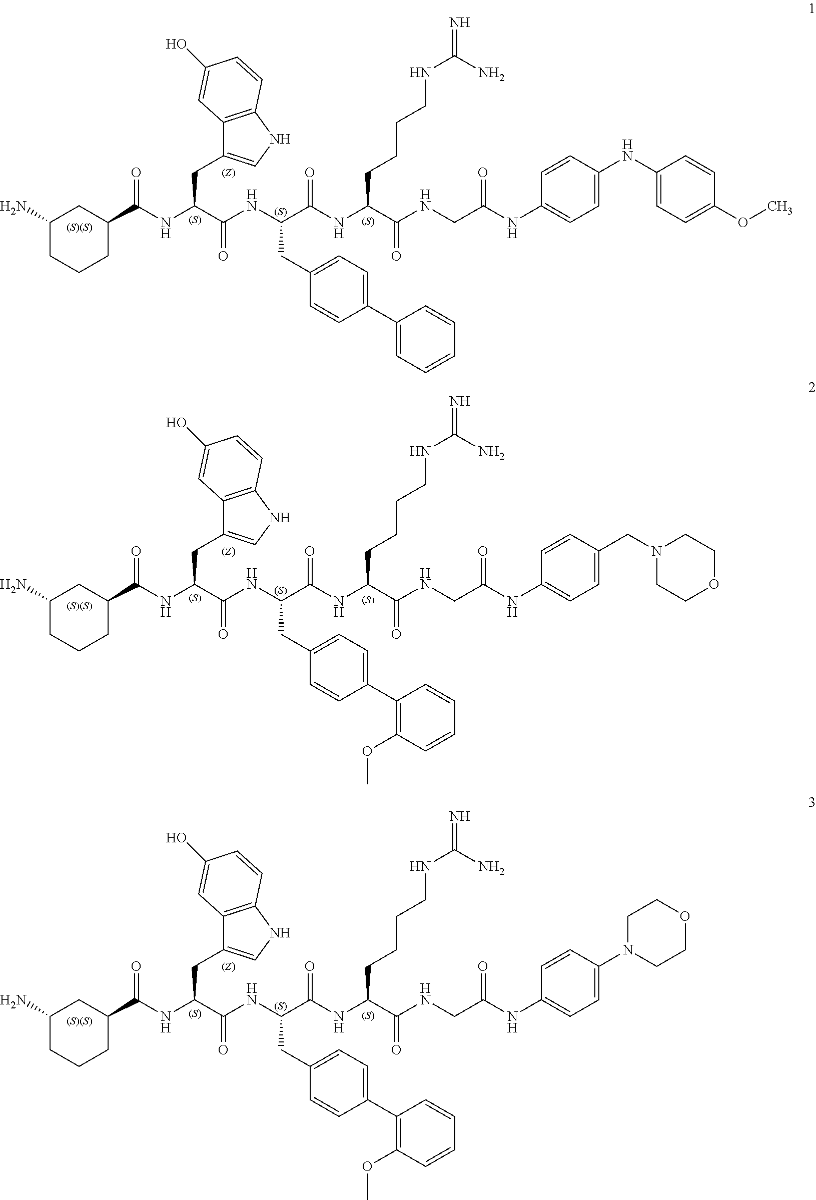

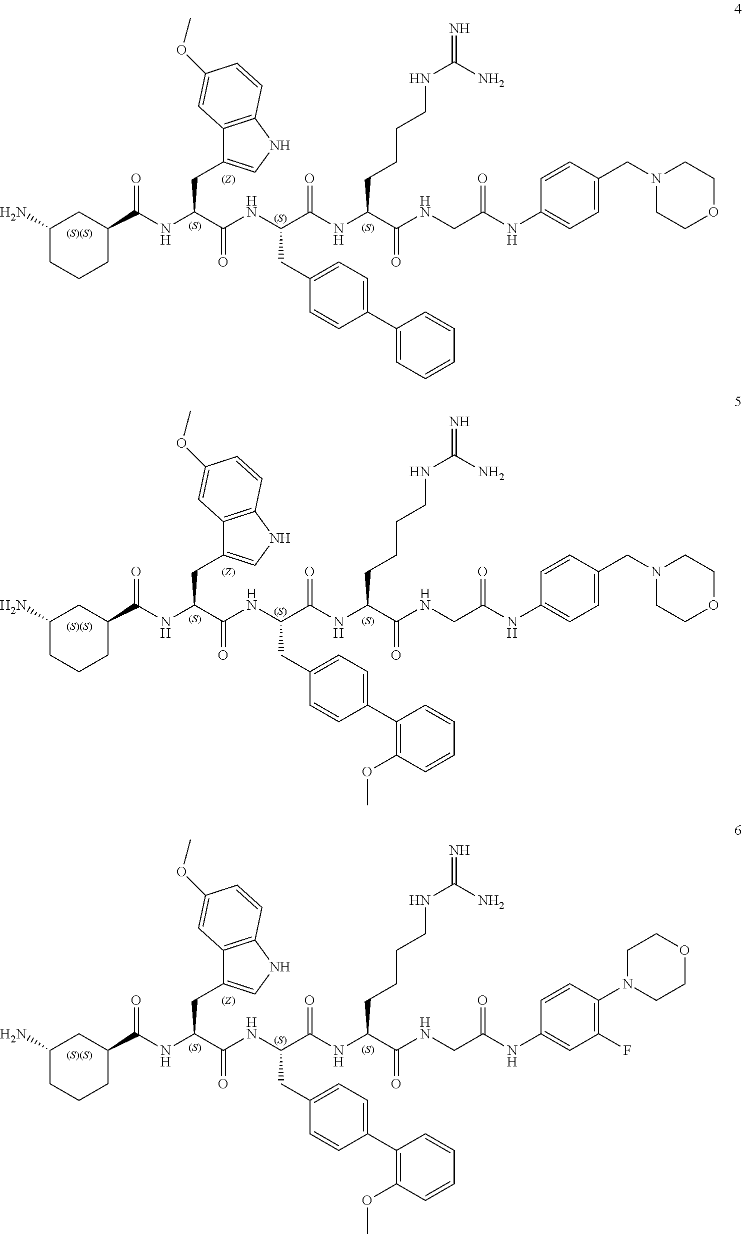

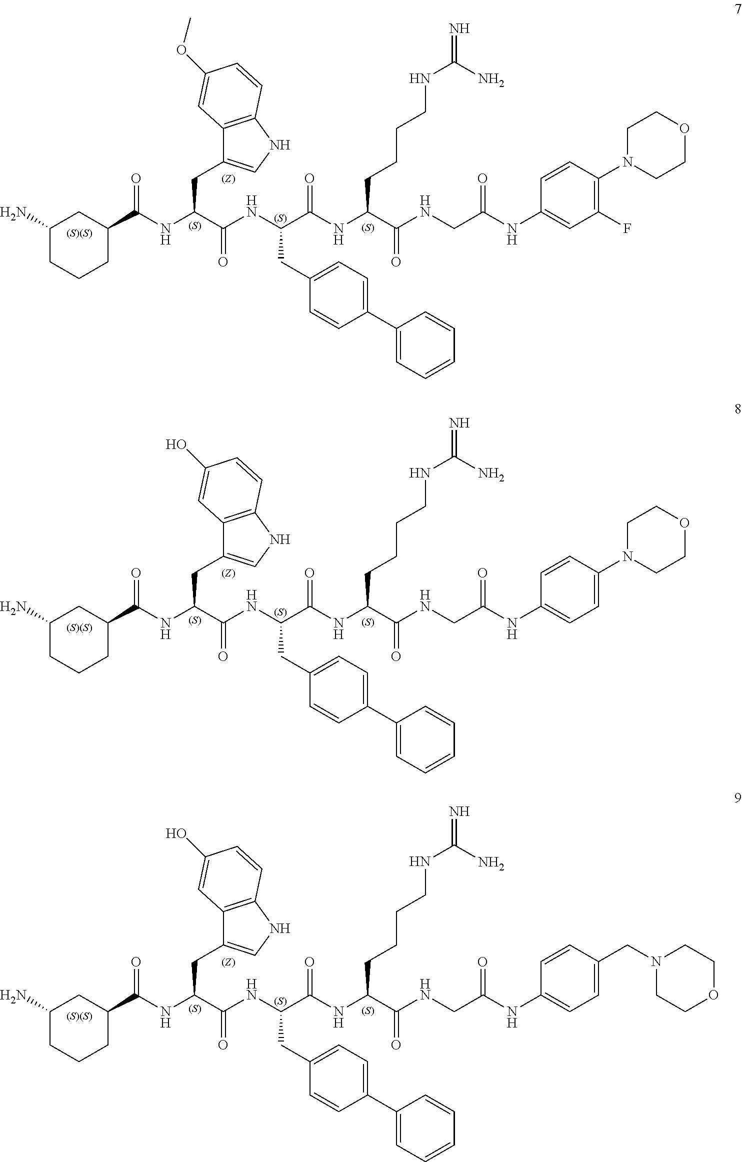

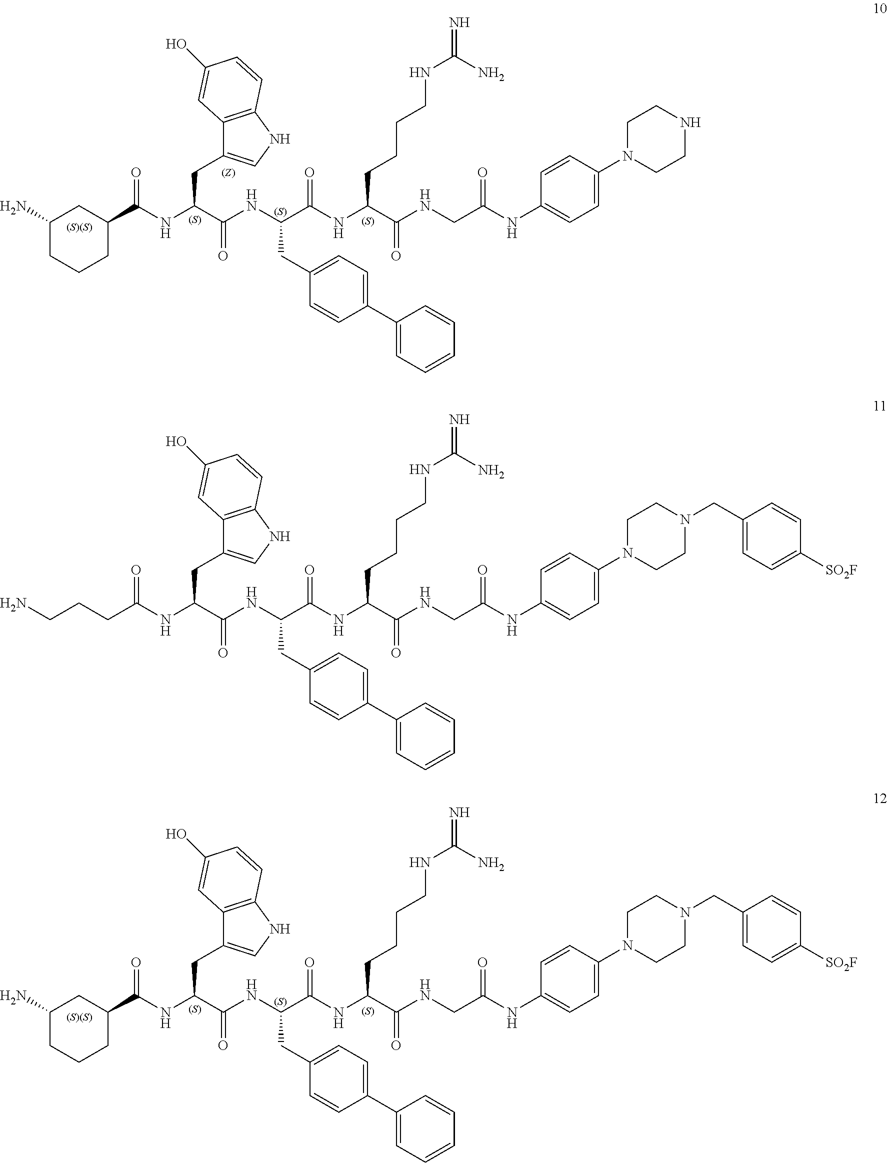

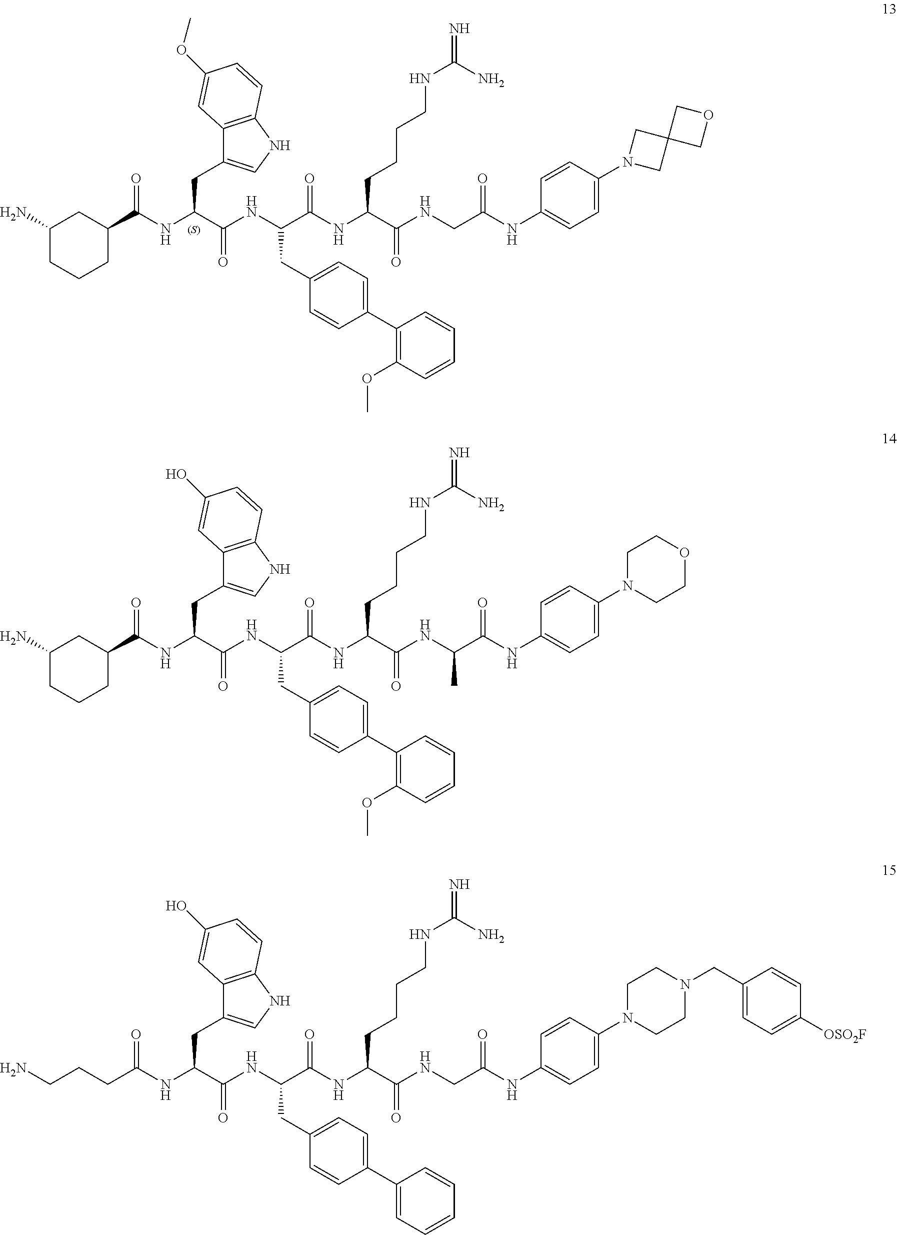

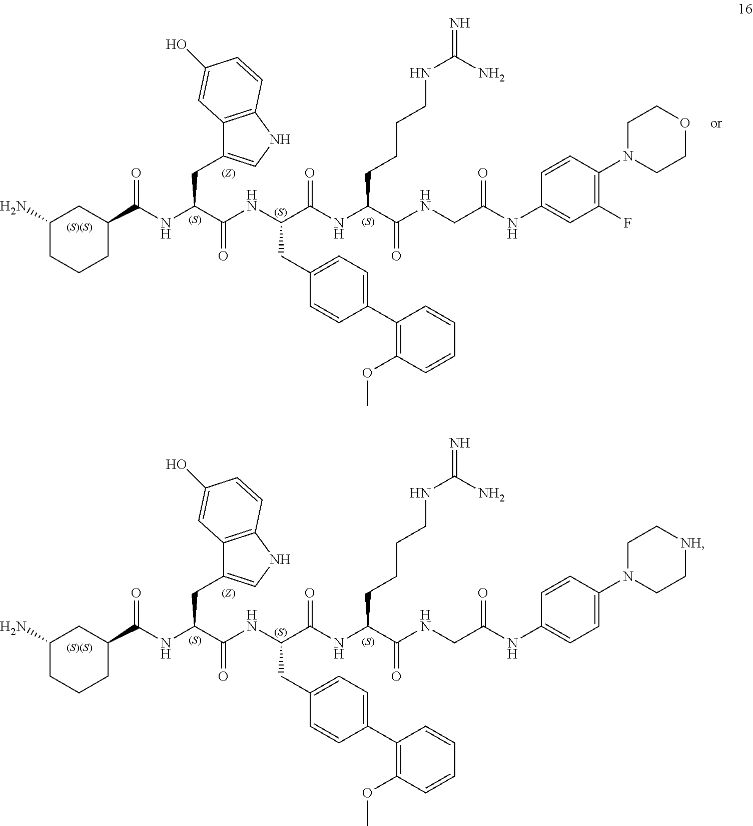

or a salt thereof.

Certain embodiments provide a peptide of Formula (I), which is:

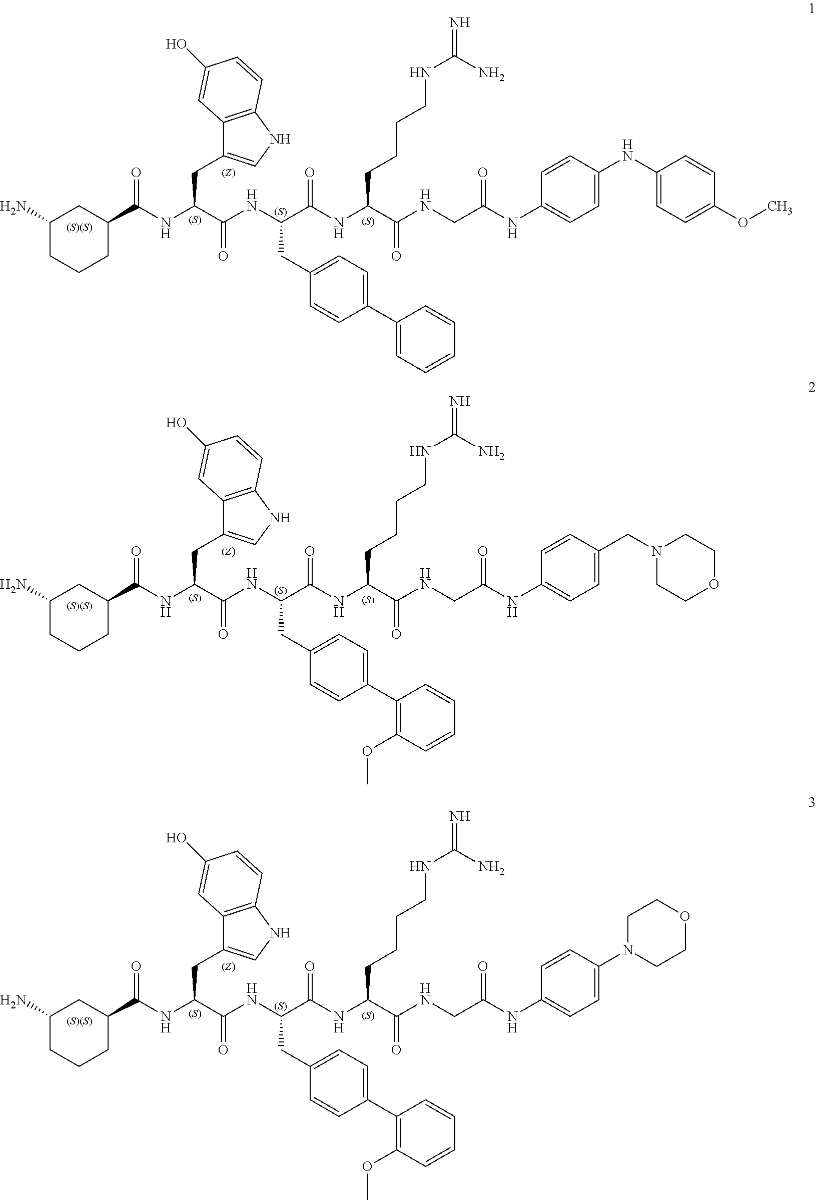

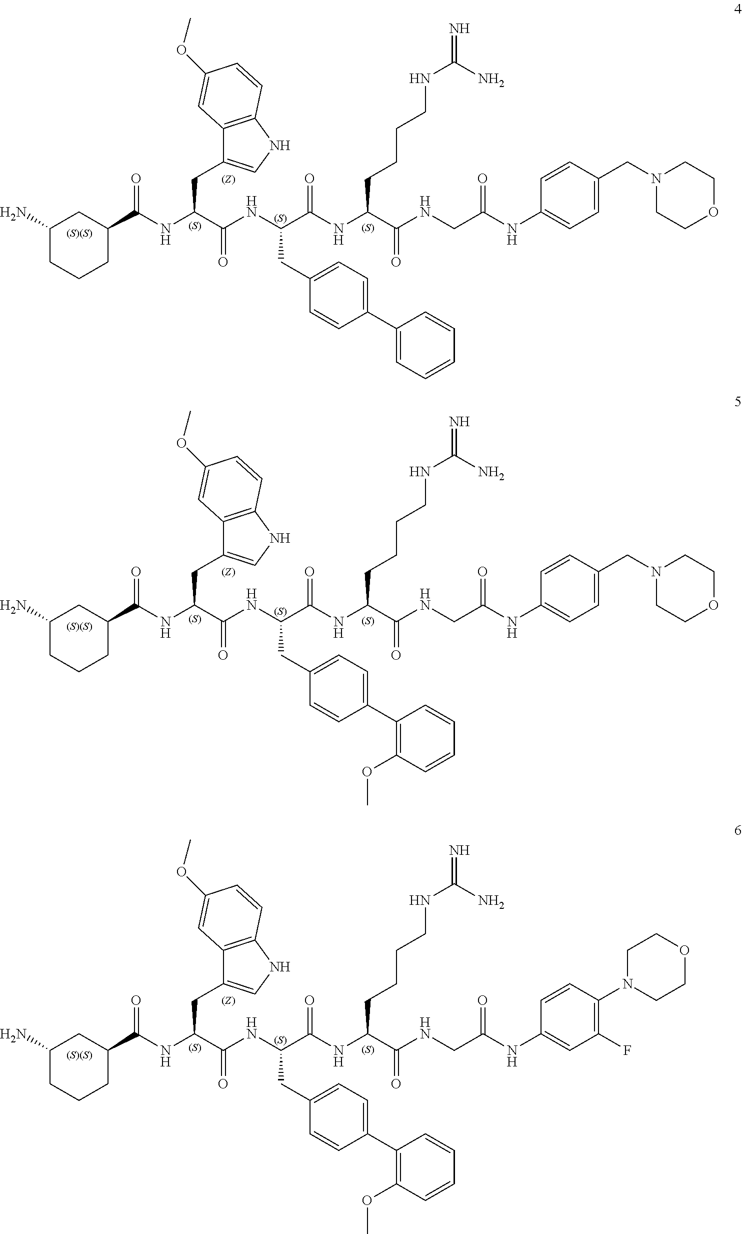

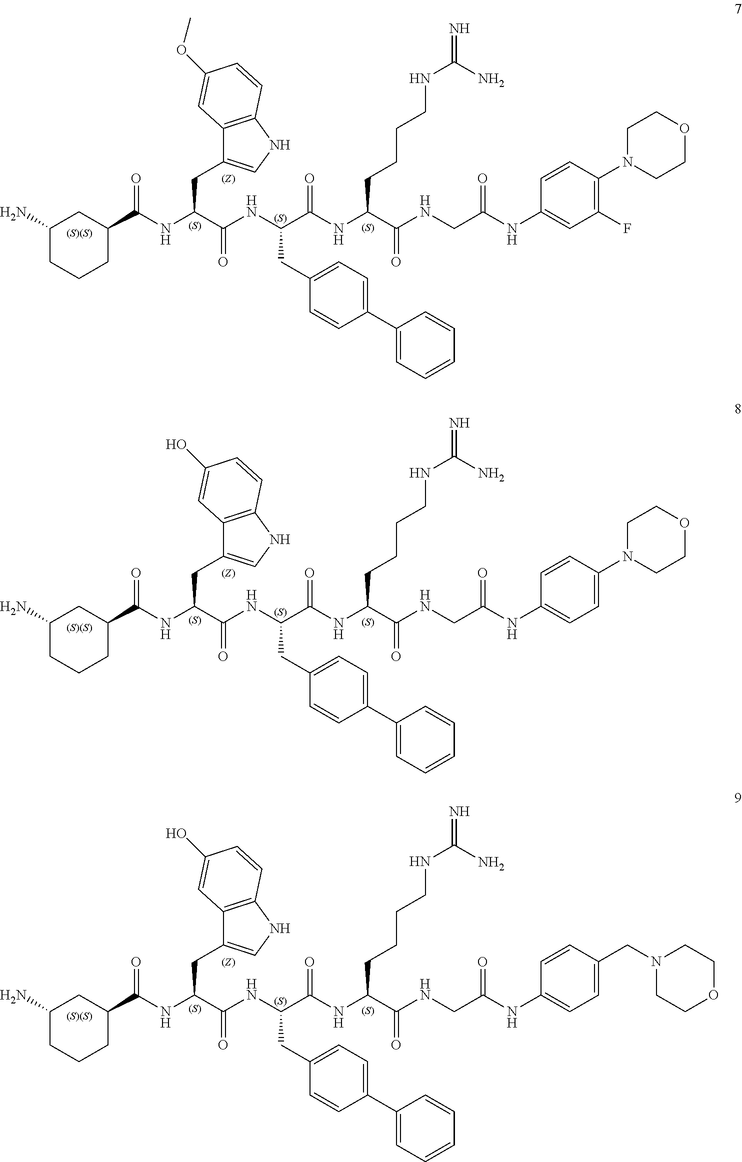

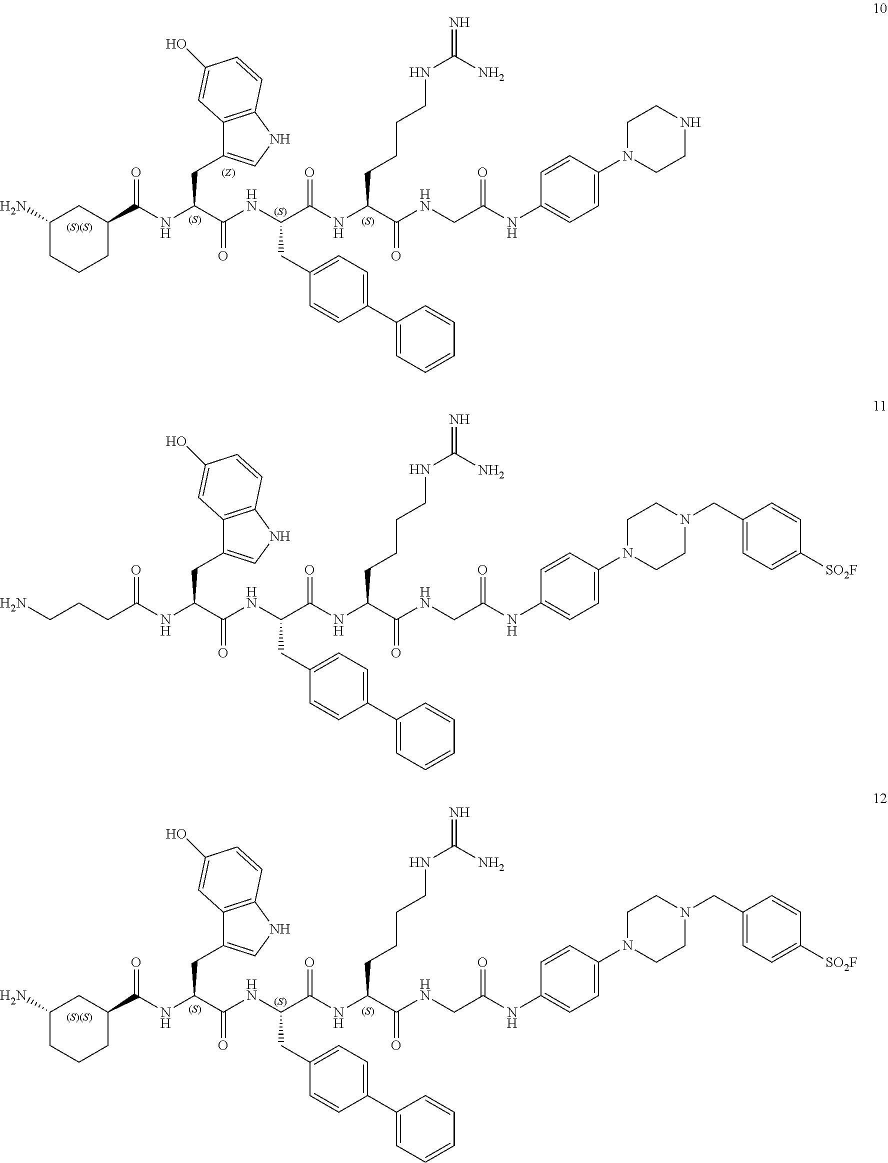

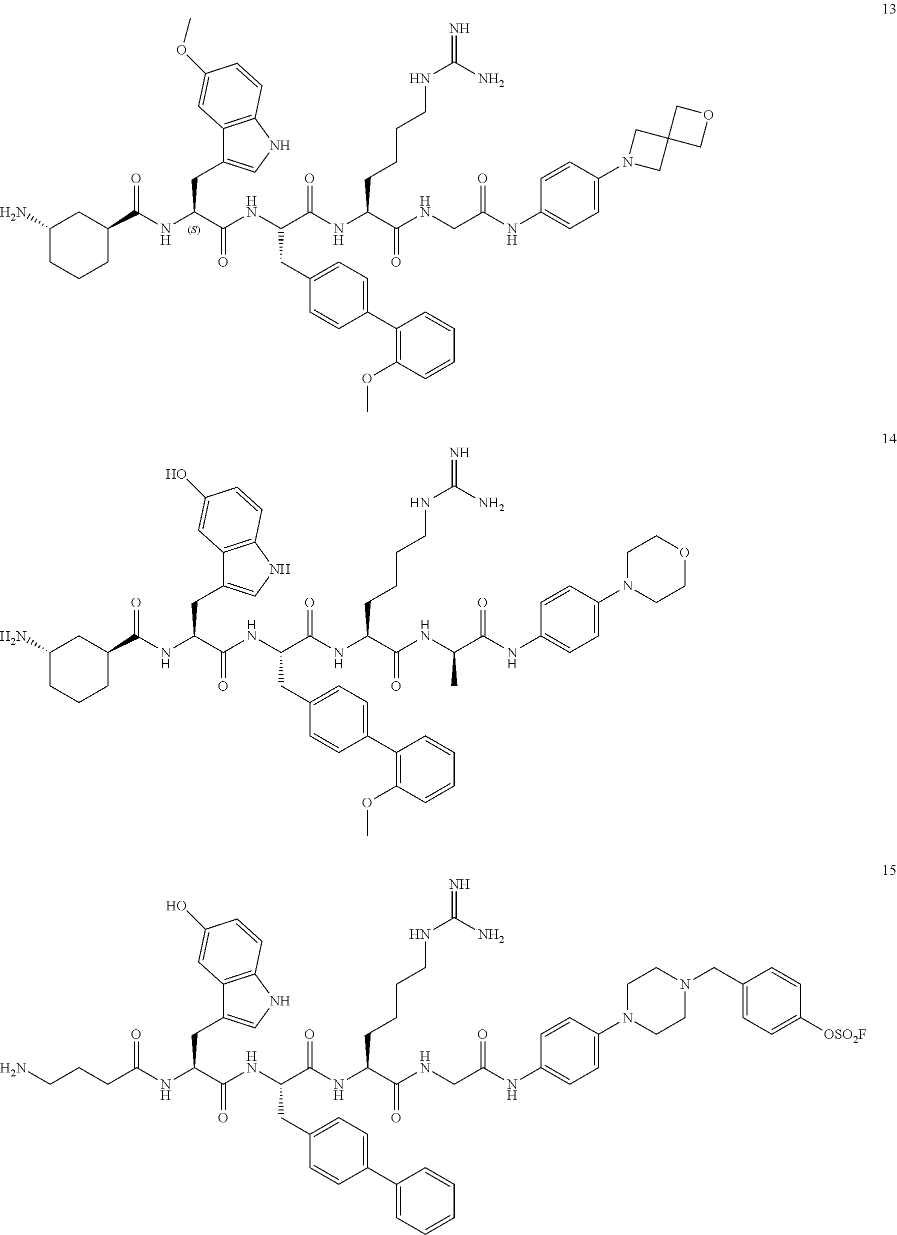

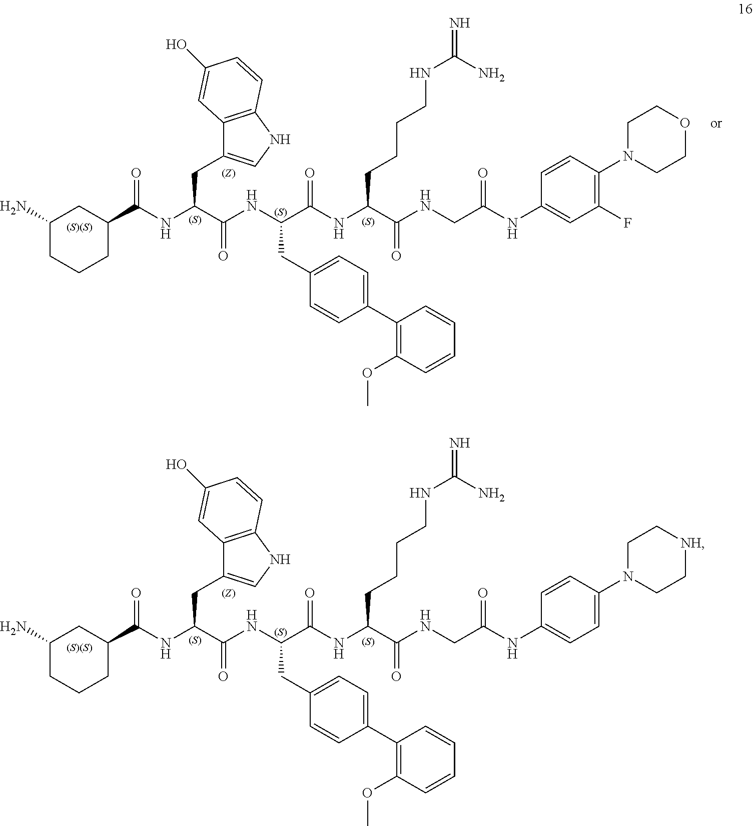

or a salt thereof.

In certain embodiments, the peptide of Formula (I) is:

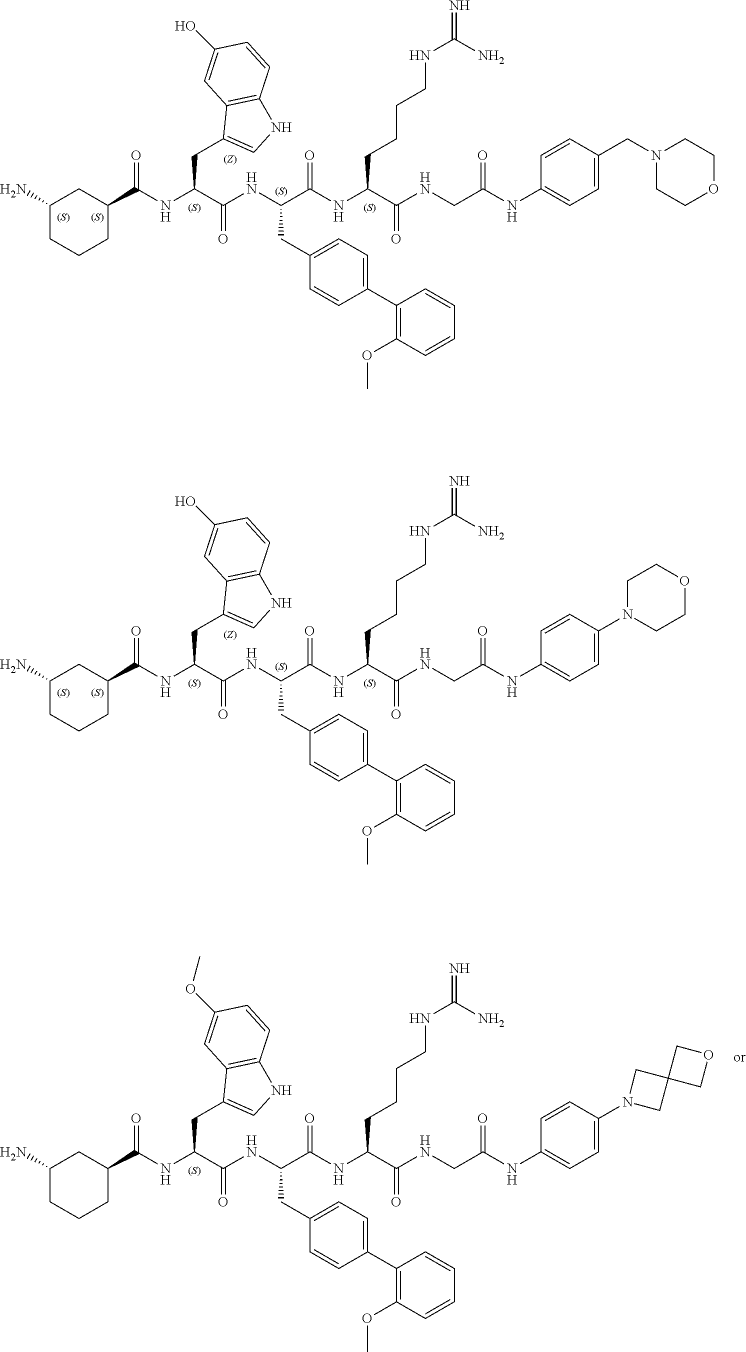

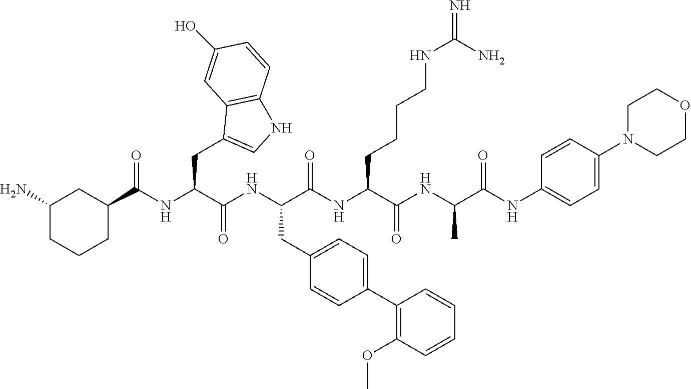 [SMILES: COc1ccccc1-c1ccc(C[C@H](NC(=O)[C@H](Cc2c[nH]c3ccc(O)cc23)NC(=O)[C@H]2CCC[C@H](N)C2)C(=O)N[C@@H](CCCCNC(=N)N)C(=O)N[C@H](C)C(=O)Nc2ccc(N3CCOCC3)cc2)cc1]

or a salt thereof.

In certain embodiments, the peptide of Formula (I) is.

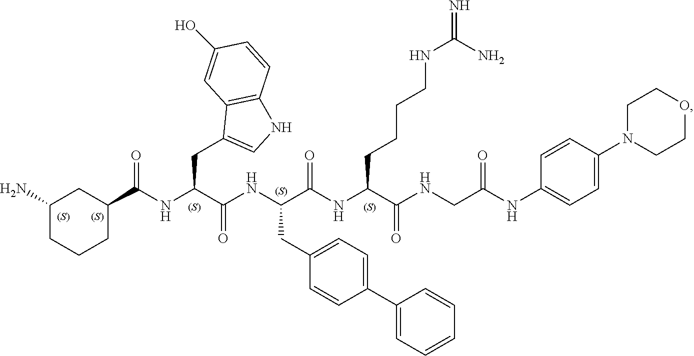

or a salt thereof.

In certain embodiments, the invention provides a compound comprising a peptide of Formula (I) as described in any one of Tables 1-8. In certain embodiments, the invention provides a peptide of Formula (I) as described in any one of Tables 1-8.

### Certain Methods of the Invention

The invention also provides a method of modulating EphA4 in vitro or in vivo, comprising contacting EphA4 with an effective amount of a compound or peptide as described herein, or a salt thereof.

The invention also provides a method of activating EphA4 in vitro or in vivo, comprising contacting EphA4 with an effective amount of a compound or peptide as described herein, or a salt thereof. In certain embodiments, EphA4 is expressed in a motor neuron. In certain embodiments, the compound/peptide is an agonist. Accordingly, certain embodiments provide a method of activating EphA4 in a motor neuron, comprising contacting EphA4 with an effective amount of a compound or peptide as described herein, or salt thereof, wherein the compound/peptide is an agonist. In certain embodiments, EphA4 is activated by at least about 30%, 40%, 50%, or more, when the motor neuron is treated with a given concentration of the compound/peptide (e.g., 10 micromolar or less, such as 1 micromolar or less), compared to a control (e.g., a negative control, such as non-treated cells). In certain embodiments, EphA4 is activated by at least about 30% when contacted with 1 micromolar or less, as compared to a non-treated control. In certain embodiments, EphA4 is activated by at least about 40% when contacted with 1 micromolar or less, as compared to a non-treated control. In certain embodiments, EphA4 is activated by at least about 50% when contacted with 1 micromolar or less, as compared to a non-treated control. In certain embodiments, EphA4 activation is measured using a method described herein.

The invention also provides a method of increasing the cellular internalization of EphA4 in vitro or in vivo, comprising contacting EphA4 with an effective amount of a compound or peptide as described herein, or a salt thereof.

The invention also provides a method of antagonizing EphA4 in vitro or in vivo, comprising contacting EphA4 with an effective amount of a compound or peptide as described herein, or a salt thereof.

The invention also provides a method of blocking EphA4 binding with its natural ligand(s) in vitro or in vivo, comprising contacting EphA4 with an effective amount of a compound or peptide as described herein, or a salt thereof.

The invention also provides a method of inhibiting EphA4 and ephrin-B2 interaction in vitro or in vivo, comprising contacting EphA4 with an effective amount of a compound or peptide as described herein, or a salt thereof.

The invention also provides a method of blocking EphA4-mediated ephrin-B2 signaling in vitro or in vivo, comprising contacting EphA4 with an effective amount of a compound or peptide as described herein, or a salt thereof.

Certain embodiments of the invention provide a method of treating a disease associated with EphA4 and/or ephrin-B2 in a mammal in need thereof, comprising administering a therapeutically effective amount of a compound or peptide as described herein, or a salt thereof (e.g., a pharmaceutically acceptable salt thereof), to the mammal.

The invention also provides a compound or peptide, or a pharmaceutically acceptable salt thereof, as described herein, for the prophylactic or therapeutic treatment of a disease associated with EphA4 and/or ephrin-B2 in a mammal in need thereof.

Certain embodiments of the invention provide the use of a compound or peptide, or a pharmaceutically acceptable salt thereof, as described herein, to prepare a medicament for treating a disease associated with EphA4 and/or ephrin-B2 in a mammal in need thereof.

The invention also provides a compound or peptide, or a pharmaceutically acceptable salt thereof, as described herein, for use in medical therapy.

In certain embodiments, the disease associated with EphA4 is a neurological disorder. In certain embodiments, the disease associated with EphA4 is a neurodegenerative disease (e.g., amyotrophic lateral sclerosis (ALS), Alzheimer's disease (AD) or Parkinson's disease (PD)). In certain embodiments, the disease associated with EphA4 is spinal cord injury. In certain embodiments, the disease associated with EphA4 is traumatic brain injury. In certain embodiments, the disease associated with EphA4 is astrogliosis.

In certain embodiments, the disease associated with EphA4 is cancer. In certain embodiments, the cancer is gastric cancer, breast cancer, pancreatic cancer, multiple myeloma, brain cancer (e.g., glioma), thyroid cancer, urothelial cancer, testis cancer, endometrial cancer, rectal cancer, colon cancer, urothelial cancer, or skin cancer.

In certain embodiments, the disease associated with EphA4 is amyotrophic lateral sclerosis (ALS). In certain embodiments, the disease is familial ALS (fALS). In certain embodiments, the disease is sporadic ALS (sALS). In certain embodiments, motor neuron degeneration is reduced. In certain embodiments, motor neuron degeneration induced by astrocytes is reduced. In certain embodiments, a compound/peptide described herein may be used to protect a motor neuron from degeneration.

In certain embodiments, a method described herein may further comprise identifying a human subject susceptible to ALS (e.g., diagnosing a subject harboring a SOD1 mutation), and administering a compound/peptide described herein therapeutically and/or prophylactically.

In certain embodiments, the compound/peptide described herein is a synthetic agonistic for EphA4. In certain embodiments, the compound/peptide activates EphA4. In certain embodiments, the compound/peptide activates EphA4 in a motor neuron. In certain embodiments, the compound/peptide activates EphA4 in a brain neuron. In certain embodiments, the compound/peptide activates EphA4 in a spinal cord neuron.

Certain embodiments also provide a method of treating (e.g., ameliorating, reducing, or suppressing) or preventing motor neuron degeneration in a mammal in need thereof, comprising administering a therapeutically effective amount of a compound or peptide as described herein, or a pharmaceutically acceptable salt thereof, to the mammal.

In certain embodiments, the motor neuron degeneration is induced by astrocytes.

In certain embodiments, motor neuron degeneration is inhibited by at least about 10%, 20%, 30%, 40%, 50%, 60%, 70%, 80%, 90%, 95%, 96%, 97% 98%, 99% or 100%. In certain embodiments motor neuron degeneration is prevented.

In certain embodiments, the mammal has familial ALS (fALS) or was determined to have a mutation associated with fALS. In certain embodiments, the compound/peptide is administered to the mammal therapeutically. In certain embodiments, the compound/peptide is administered to the mammal prophylactically.

In certain embodiments, the mammal has sporadic ALS (sALS).

In certain embodiments, the compound/peptide is an EphA4 agonist and the compound/peptide activates EphA4.

Certain embodiments provide a compound or peptide, or a pharmaceutically acceptable salt thereof, as described herein, for treating or preventing motor neuron degeneration in a mammal in need thereof.

Certain embodiments provide the use of a compound or peptide, or a pharmaceutically acceptable salt thereof, as described herein, to prepare a medicament for treating or preventing motor neuron degeneration in a mammal in need thereof.

In certain embodiments, the mammal is a human.

Certain embodiments described herein provide methods for predicting whether a patient is likely to respond favorably to a treatment. For example, certain embodiments of the invention provide a method of identifying patient (e.g., a patient having a disorder associated with motor neuron degeneration, such as ALS) that is likely to respond to treatment, the method comprising of a) isolating fibroblasts from the patient; b) culturing the fibroblasts under conditions suitable to generate patient derived astrocytes, c) co-culturing the patient derived astrocytes with mouse motor neurons (MN) in the presence of a compound or peptide as described herein, or a pharmaceutically acceptable salt thereof; and c) identifying the patient as being likely to respond to treatment with the compound/peptide, or pharmaceutically acceptable salt thereof, when MN cell degeneration or MN cell death is inhibited as compared to a control (e.g., a negative control, such as a MN cell that was not contacted with the compound/peptide) or reference value.

Methods for differentiating or reprogramming fibroblasts into neuronal progenitor cells and/or astrocytes are known in the art and are described herein (see, e.g., Example 2 and Meyer et al., Proc Natl Acad Sci USA, 111 (2): 829-832, (2014)). In certain embodiments, the fibroblasts are reprogrammed directly into neuronal progenitor cells (NPCs), and the NPCs are subsequently cultured under conditions suitable to generate astrocytes.

In certain embodiments, the method further comprises administering the compound or peptide to the identified patient.

In certain embodiments, motor neuron degeneration or cell death is inhibited by at least about 10%, 20%, 30%, 40%, 50%, 60%, 70%, 80%, 90%, 95%, 96%, 97%, 98%, 99%, or 100% as compared to a control (e.g., a negative control). In certain embodiments motor neuron degeneration is prevented. In certain embodiments MN cell death prevented.

In certain embodiments, the patient has familial ALS (fALS). In certain embodiments, the patient has sporadic ALS (sALS).

In certain embodiments, the compound/peptide is an EphA4 agonist.

Certain embodiments also provide a method for identifying an EphA4 agonist, the method comprising isolating a primary motor neuron(s) from the spinal cord of an animal, contacting a test compound/peptide with the isolated primary motor neuron(s), under conditions suitable for binding between the test compound/peptide and EphA4, evaluating axon growth cone morphology of the primary motor neuron(s), and identifying the test compound/peptide as an EphA4 agonist when growth cone collapse is detected. In certain embodiments, the animal is a transgenic animal. In certain embodiments, the primary motor neurons express a fluorescent protein, such as GFP.

### Composition and Administration

Certain embodiments of the invention also provide a composition (e.g., a pharmaceutical composition) comprising a compound (e.g., a peptide) as described herein, or a salt (e.g., pharmaceutically acceptable) thereof, and a pharmaceutically acceptable carrier. Compounds described herein (including salt, solvate, stereoisomer or prodrug thereof) can be formulated as pharmaceutical compositions and administered to a mammalian host, such as a human patient in a variety of forms adapted to the chosen route of administration, i.e., orally or parenterally, by intravenous, intramuscular, intrathecal, topical, intranasal, inhalation, suppository, sub dermal osmotic pump, intraperitoneal, intradermal or subcutaneous routes.

Thus, the present compounds may be systemically administered, e.g., orally or intravenously, in combination with a pharmaceutically acceptable vehicle such as an inert diluent or an assimilable edible carrier (pharmaceutically acceptable excipients are well known in the field). The composition may be freeze-dried into lyophilized formulation (e.g., lyophilized cake), may be enclosed in hard or soft shell gelatin capsules, or may be compressed into tablets.

For oral therapeutic administration, the active compound may be combined with one or more excipients and used in the form of ingestible tablets, buccal tablets, troches, capsules, elixirs, suspensions, syrups, wafers, and the like. The capsules, tablets or other oral delivery formulation may have enteric coating for controlled release of the compound at desired intestinal segment. Such compositions and preparations should contain at least 0.1% of active compound. The percentage of the compositions and preparations may, of course, be varied and may conveniently be between about 2 to about 60% of the weight of a given unit dosage form. The amount of active compound in such therapeutically useful compositions is such that an effective dosage level will be obtained.

In certain embodiments, the present compounds may be administered via intrathecal delivery.

Lyophilized formulations may also contain carrier such as bulking agent (e.g., mannitol or glycine) and cryoprotectant/lyoprotectant (e.g., trehalose or sucrose). Lyophilized formulation can be reconstituted into a liquid dosage form using saline, 5% dextrose solution or sterile water before administration. The tablets, troches, pills, capsules, and the like may also contain the following: binders such as gum tragacanth, acacia, corn starch or gelatin; excipients such as dicalcium phosphate; a disintegrating agent such as corn starch, potato starch, alginic acid and the like; a lubricant such as magnesium stearate; and a sweetening agent such as sucrose, fructose, lactose or aspartame or a flavoring agent such as peppermint, oil of wintergreen, or cherry flavoring may be added. When the unit dosage form is a capsule, it may contain, in addition to materials of the above type, a liquid carrier, such as a vegetable oil or a polyethylene glycol. Various other materials may be present as coatings or to otherwise modify the physical form of the solid unit dosage form. For instance, tablets, pills, or capsules may be coated with gelatin, wax, shellac or sugar and the like. A syrup or elixir may contain the active compound, sucrose or fructose as a sweetening agent, methyl and propylparabens as preservatives, a dye and flavoring such as cherry or orange flavor. Of course, any material used in preparing any unit dosage form should be pharmaceutically acceptable and substantially non-toxic in the amounts employed. In addition, the active compound may be incorporated into sustained-release preparations and devices.

The active compound may also be administered intravenously, intradermally, subcutaneously, intrathecally or intraperitoneally by infusion or injection. Solutions of the active compound or its salts can be prepared in water, optionally mixed with a nontoxic surfactant. Dispersions can also be prepared in glycerol, liquid polyethylene glycols, triacetin, and mixtures thereof and in oils. Under ordinary conditions of storage and use, these preparations contain a preservative to prevent the growth of microorganisms.

The pharmaceutical dosage forms suitable for injection or infusion can include sterile aqueous solutions or dispersions or sterile powders comprising the active ingredient which are adapted for the extemporaneous preparation of sterile injectable or infusible solutions or dispersions, optionally encapsulated in liposomes. In all cases, the ultimate dosage form should be sterile, fluid and stable under the conditions of manufacture and storage. The liquid carrier or vehicle can be a solvent or liquid dispersion medium comprising, for example, water, ethanol, a polyol (for example, glycerol, propylene glycol, liquid polyethylene glycols, and the like), vegetable oils, nontoxic glyceryl esters, and suitable mixtures thereof. The proper fluidity can be maintained, for example, by the formation of liposomes, by the maintenance of the required particle size in the case of dispersions or by the use of surfactants. The prevention of the action of microorganisms can be brought about by various antibacterial and antifungal agents, for example, parabens, chlorobutanol, phenol, sorbic acid, thimerosal, and the like. In many cases, it will be preferable to include isotonic agents, for example, sugars, buffers or sodium chloride. Prolonged absorption of the injectable compositions can be brought about by the use in the compositions of agents delaying absorption, for example, aluminum monostearate and gelatin.

Sterile injectable solutions are prepared by incorporating the active compound in the required amount in the appropriate solvent with various other ingredients enumerated above, as required, followed by filter sterilization. In the case of sterile powders for the preparation of sterile injectable solutions, the preferred methods of preparation are vacuum drying and the freeze-drying techniques, which yield a powder of the active ingredient plus any additional desired ingredient present in the previously sterile-filtered solutions.

For topical administration, the present compounds may be applied in pure form, i.e., when they are liquids. However, it will generally be desirable to administer them to the skin as compositions or formulations, in combination with a dermatologically acceptable carrier, which may be a solid or a liquid.

Useful solid carriers include finely divided solids such as talc, clay, microcrystalline cellulose, silica, alumina, starch, starch derivatives and the like. Useful liquid carriers include water, alcohols or glycols or water-alcohol/glycol blends, in which the present compounds can be dissolved or dispersed at effective levels, optionally with the aid of non-toxic surfactants. Adjuvants such as fragrances and additional antimicrobial agents can be added to optimize the properties for a given use. The resultant liquid compositions can be applied from absorbent pads, used to impregnate bandages and other dressings, or sprayed onto the affected area using pump-type or aerosol sprayers.

Thickeners such as synthetic polymers, fatty acids, fatty acid salts and esters, fatty alcohols, modified celluloses or modified mineral materials can also be employed with liquid carriers to form spreadable pastes, gels, ointments, soaps, and the like, for application directly to the skin of the user.

Examples of useful dermatological compositions which can be used to deliver the compounds of formula I to the skin are known to the art; for example, see Jacquet et al. (U.S. Pat. No. 4,608,392), Geria (U.S. Pat. No. 4,992,478), Smith et al. (U.S. Pat. No. 4,559,157) and Wortzman (U.S. Pat. No. 4,820,508).

Useful dosages of the compounds described herein can be determined by comparing their in vitro activity, and in vivo activity in animal models. Methods for the extrapolation of effective dosages in mice, and other animals, to humans are known to the art; for example, see U.S. Pat. No. 4,938,949.

The amount of the compound, or an active salt or derivative thereof, required for use in treatment will vary not only with the particular salt selected but also with the route of administration, the nature of the condition being treated and the age and condition of the patient and will be ultimately at the discretion of the attendant physician or clinician.

The compound may be conveniently formulated in unit dosage form. In one embodiment, the invention provides a composition comprising a compound of the invention formulated in such a unit dosage form.

The desired dose may conveniently be presented in a single dose or as divided doses administered at appropriate intervals, for example, as two, three, four or more sub-doses per day. The sub-dose itself may be further divided, e.g., into a number of discrete loosely spaced administrations.

Compounds or peptides of the invention described herein can also be administered in combination with other therapeutic agent(s). For example, compounds/peptides of the invention, or pharmaceutical salts thereof, may be administered with other agent(s) that are useful for treating diseases associated with EphA4 (e.g., ALS, AD or cancer). Accordingly, in one embodiment the invention also provides a composition comprising a compound/peptide of the invention described herein, or a pharmaceutically acceptable salt thereof, at least one other therapeutic agent, and a pharmaceutically acceptable diluent or carrier.

The invention also provides a kit comprising a compound/peptide of the invention described herein, or a pharmaceutically acceptable salt thereof, and optionally at least one other therapeutic agent, packaging material, and instructions for administering the compound/peptide of the invention described herein or the pharmaceutically acceptable salt thereof and the other optional therapeutic agent or agents to an mammal to modulate EphA4 activity, and/or treat diseases associated with EphA4 (e.g., ALS, AD or cancer).

### CERTAIN EMBODIMENTS

Embodiment 1. A compound comprising a peptide of Formula (I) X₀—X₁—X₂—X₃—X₄ from N terminal to C terminal, wherein:

X₀ is a residue of a beta-amino acid, gamma-amino acid, or delta-amino acid;

X₁ is a residue of Trp;

X₂ is a residue of Bip;

X₃ is a residue of an amino acid;

X₄ is a residue of an amino acid;

or a salt thereof.

Embodiment 2. The compound of Embodiment 1, wherein the N-terminus of the peptide is a primary amine group.

Embodiment 3. The compound of any one of Embodiments 1-2, wherein X₀ is a residue of a gamma-amino acid.

Embodiment 4. The compound of any one of Embodiments 1-3, wherein X₀ is a residue of gamma-amino-butyric acid (GABA).

Embodiment 5. The compound of any one of Embodiments 1-3, wherein X₀ is a residue of 3-amino-cyclohexanecarboxylic acid (ACHC).

Embodiment 6. The compound of any one of Embodiments 1-5, wherein X₁ is a residue of 5-hydroxy-Trp or 5-methoxy-Trp.

Embodiment 7. The compound of any one of Embodiments 1-6, wherein X₂ is a residue of Bip or 4-(2-methoxyphenyl)-Phe.

Embodiment 8. The compound of any one of Embodiments 1-7, wherein X₃ is a residue of hArg, Arg, hLys, Lys, Orn, Dab or Dap.

Embodiment 9. The compound of any one of Embodiments 1-8, wherein X₄ is a residue of Gly, Ala, Thr or Ser.

Embodiment 10. The compound of any one of Embodiments 1-9, wherein the C-terminus of the peptide of Formula (I) is amidated with an amino group.

Embodiment 11. The compound of any one of Embodiments 1-10, wherein the C-terminus of the peptide of Formula (I) is amidated with an aryl-amino or heteroaryl-amino group.

Embodiment 12. The compound of any one of Embodiments 1-11, wherein the C-terminus of the peptide of Formula (I) is amidated with an anilinyl group.

Embodiment 13. The compound of any one of Embodiments 1-11, wherein the C-terminus of the peptide of Formula (I) is amidated with a (2-pyridyl)-amino group.

Embodiment 14. The compound of Embodiment 1, wherein the compound comprises a peptide of Formula (I) wherein:

X₀ is a residue of GABA or ACHC,

X₁ is a residue of Trp, 5-hydroxy-Trp or 5-methoxy-Trp,

X₂ is a residue of Bip or 4-(2-methoxyphenyl)-Phe,

X₃ is a residue of hArg, Arg, hLys, Lys, Orn, Dab or Dap,

X₄ is a residue of Gly, Ala, Thr or Ser,

wherein the C-terminus of the peptide of Formula (I) is amidated with an anilinyl group or a (2-pyridyl)-amino group,

or a salt thereof.

Embodiment 15. The compound of Embodiment 1, wherein the compound consists of the peptide of Formula (I).

Embodiment 16. The compound of Embodiment 1, wherein the peptide has structure of Formula (Ia)

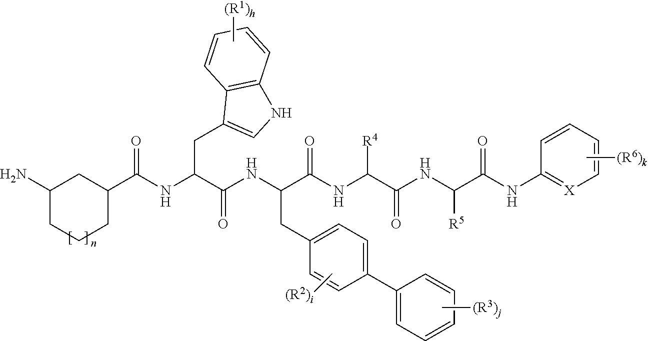

wherein

n is 0, 1, or 2;

X is C or N;

h, i, j and k are each independent 0, 1, 2 or 3;

R¹, R², R³, R⁴, R⁵, and R⁶ are each independently absent, hydrogen, halogen, —OCH₃, —CN, —OH, —NH₂, —CH₂OH—, —CH₂(OH)CH₃, —COOH, —CONH₂, —NO₂, —SH, —SO₃H, —SO₄H, —SO₂NH₂, —OSO₂F, —SO₂F, —NHNH₂, —ONH₂, —NHC(═O)NHNH₂, —NHC(═O)NH₂, —NHC(═O)H, —NHC(═O)OH, —NHOH, —OCCl₃, —OCBr₃, —OCF₃, —OCl₃, —OCH₂Cl, —OCH₂Br, —OCH₂F, —OCH₂I, —OCHCl₂, —OCHBr₂, —OCHF₂, —OCHI₂, an amino acid side chain, alkyl, heteroalkyl, cycloalkyl, heterocycloalkyl, aryl, or heteroaryl,

or a salt thereof.

Embodiment 17. The compound of Embodiment 16, wherein R⁴ is the side chain of hArg, Arg, hLys, Lys, Orn, Dab, or Dap.

Embodiment 18. The compound of Embodiment 16, wherein R⁴ is the side chain of L-hArg.

Embodiment 19. The compound of any one of Embodiments 16-18, wherein R⁵ is —H, —CH₃, —CH₂OH, or —CH(OH)CH₃.

Embodiment 20. The compound of Embodiment 1, wherein the peptide has structure of Formula (Ib)

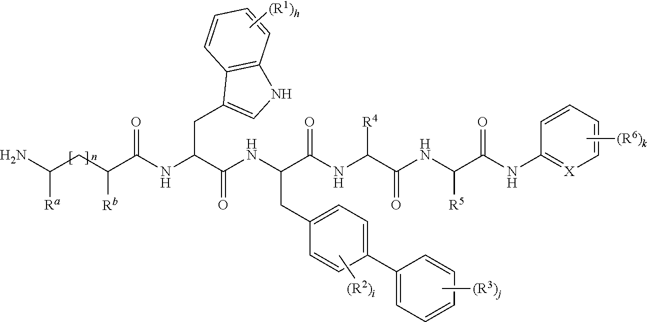

wherein

n is 0, 1, or 2;

X is C or N;

h, i, j and k are each independent 0, 1, 2 or 3;

R^{a}, R^{b}, R¹, R², R³, R⁴, R⁵, and R⁶ are each independently absent, hydrogen, halogen, —OCH₃, —CN, —OH, —NH₂, —CH₂OH—, —CH₂(OH)CH₃, —COOH, —CONH₂, —NO₂, —SH, —SO₃H, —SO₄H, —SO₂NH₂, —OSO₂F, —SO₂F, —NHNH₂, —ONH₂, —NHC(═O)NHNH₂, —NHC(═O)NH₂, —NHC(═O)H, —NHC(═O)OH, —NHOH, —OCCl₃, —OCBr₃, —OCF₃, —OCI₃, —OCH₂Cl, —OCH₂Br, —OCH₂F, —OCH₂I, —OCHCl₂, —OCHBr₂, —OCHF₂, —OCHI₂, an amino acid side chain, alkyl, heteroalkyl, cycloalkyl, heterocycloalkyl, aryl, or heteroaryl,

or a salt thereof.

Embodiment 21. The compound of Embodiment 20, wherein R⁶ is —OSO₂F, or —SO₂F.

Embodiment 22. The compound of Embodiment 1, wherein the peptide has structure of Formula (Ic)

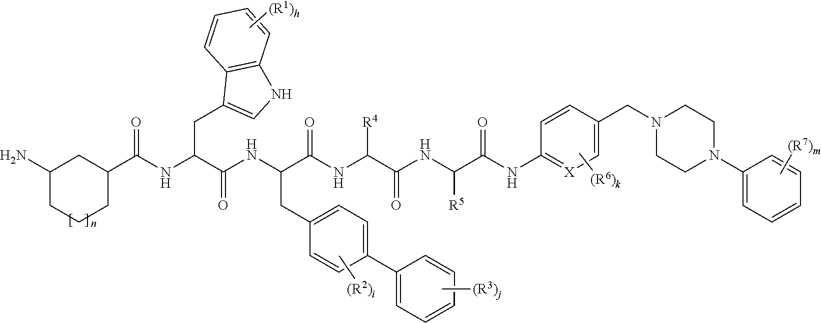

wherein

n is 0, 1, or 2;

X is C or N;

h, i, j, k and m are each independent 0, 1, 2 or 3;

R¹, R², R³, R⁴, R⁵, R⁶ and R⁷ are each independently absent, hydrogen, halogen, —OCH₃, —CN, —OH, —NH₂, —CH₂OH—, —CH₂(OH)CH₃, —COOH, —CONH₂, —NO₂, —SH, —SO₃H, —SO₄H, —SO₂NH₂, —OSO₂F, —SO₂F, —NHNH₂, —ONH₂, —NHC(═O)NHNH₂, —NHC(═O)NH₂, —NHC(═O)H, —NHC(═O)OH, —NHOH, —OCCl₃, —OCBr₃, —OCF₃, —OCl₃, —OCH₂Cl, —OCH₂Br, —OCH₂F, —OCH₂I, —OCHCl₂, —OCHBr₂, —OCHF₂, —OCHI₂, an amino acid side chain, alkyl, heteroalkyl, cycloalkyl, heterocycloalkyl, aryl, or heteroaryl,

or a salt thereof.

Embodiment 23. The compound of Embodiment 22, wherein R⁷ is —OSO₂F, or —SO₂F.

Embodiment 24. The compound of Embodiment 1, wherein the peptide has structure of Formula (Id)

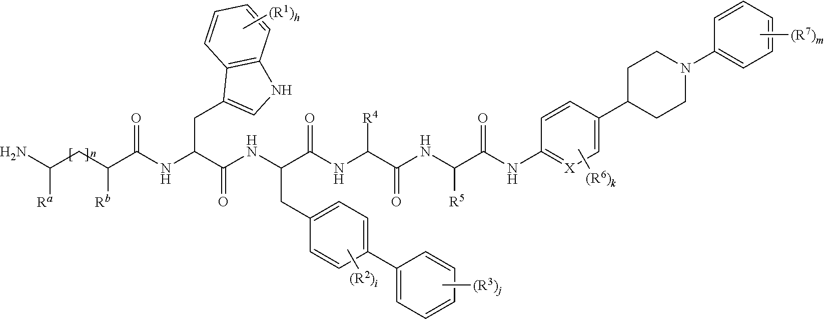

wherein

n is 0, 1, or 2;

X is C or N;

h, i, j, k and m are each independent 0, 1, 2 or 3;

R^{a}, R^{b}, R¹, R², R³, R⁴, R⁵, R⁶ and R⁷ are each independently absent, hydrogen, halogen, —OCH₃, —CN, —OH, —NH₂, —CH₂OH—, —CH₂(OH)CH₃, —COOH, —CONH₂, —NO₂, —SH, —SO₃H, —SO₄H, —SO₂NH₂, —OSO₂F, —SO₂F, —NHNH₂, —ONH₂, —NHC(═O)NHNH₂, —NHC(═O)NH₂, —NHC(═O)H, —NHC(═O)OH, —NHOH, —OCCl₃, —OCBr₃, —OCF₃, —OCl₃, —OCH₂Cl, —OCH₂Br, —OCH₂F, —OCH₂I, —OCHCl₂, —OCHBr₂, —OCHF₂, —OCHI₂, an amino acid side chain, alkyl, heteroalkyl, cycloalkyl, heterocycloalkyl, aryl, or heteroaryl,

or a salt thereof.

Embodiment 25. The compound of Embodiment 24, wherein R⁷ is —OSO₂F, or —SO₂F.

Embodiment 26. The compound of Embodiment 1, wherein the peptide is selected from the group consisting of:

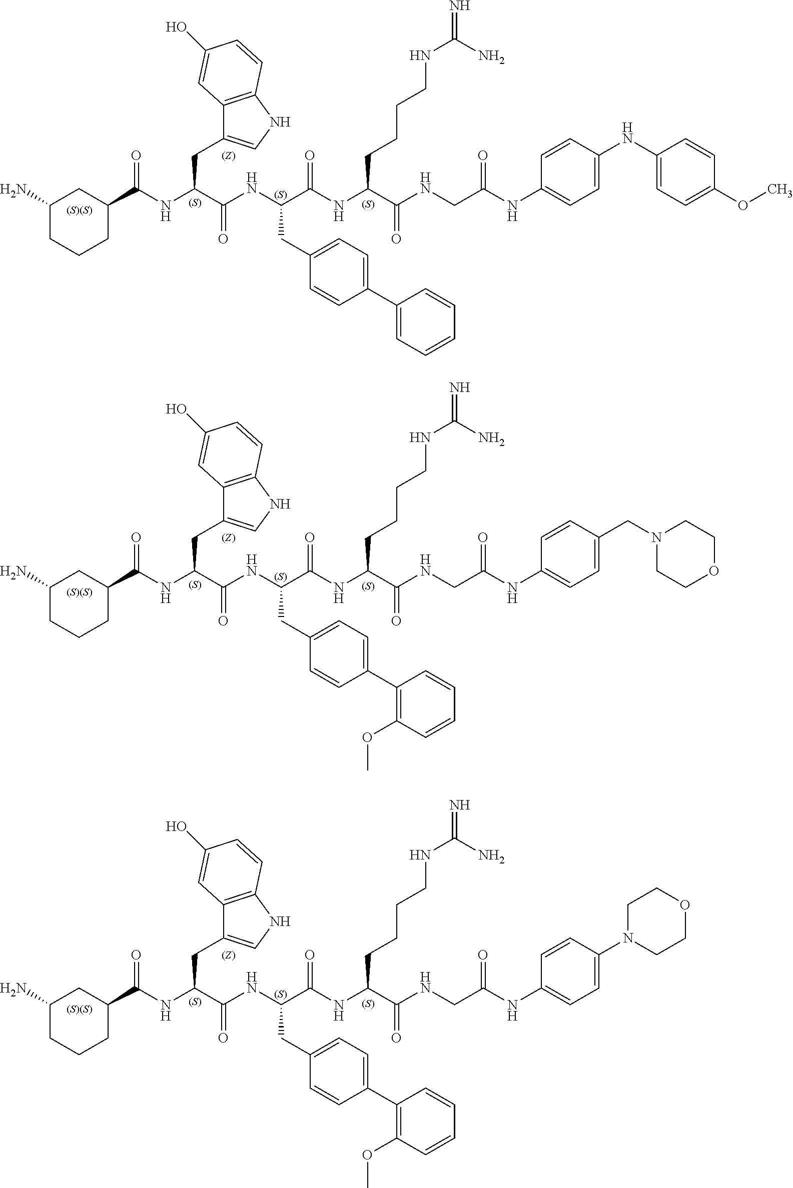

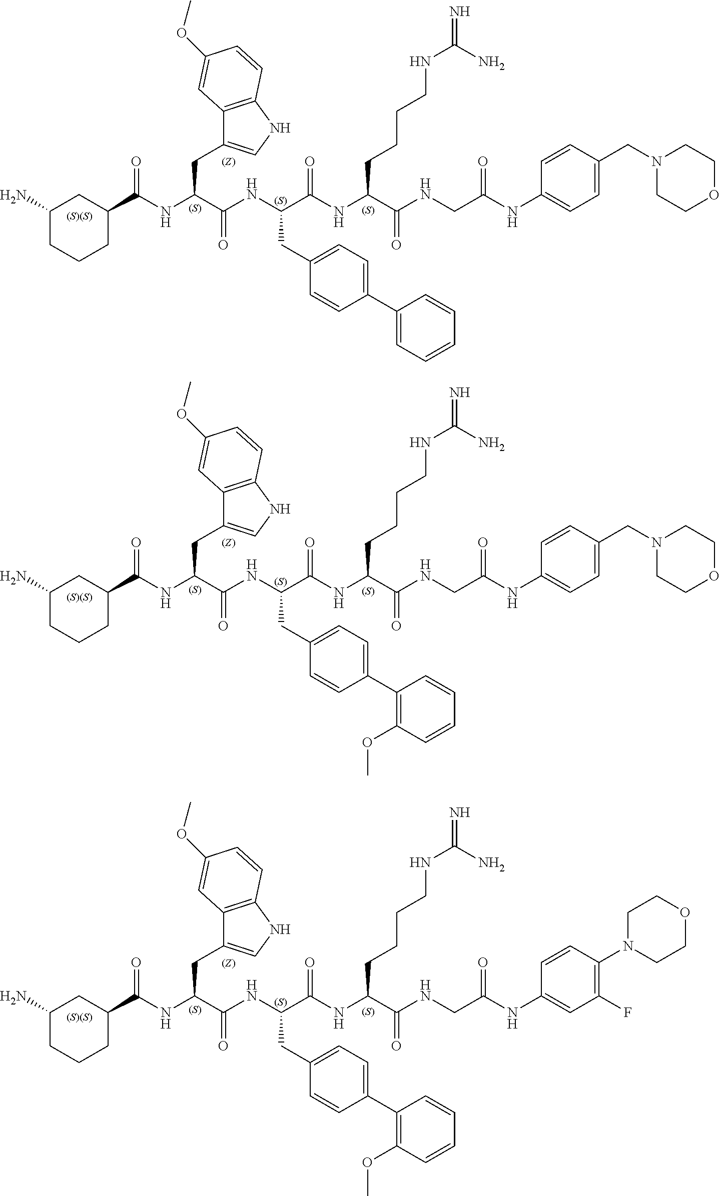

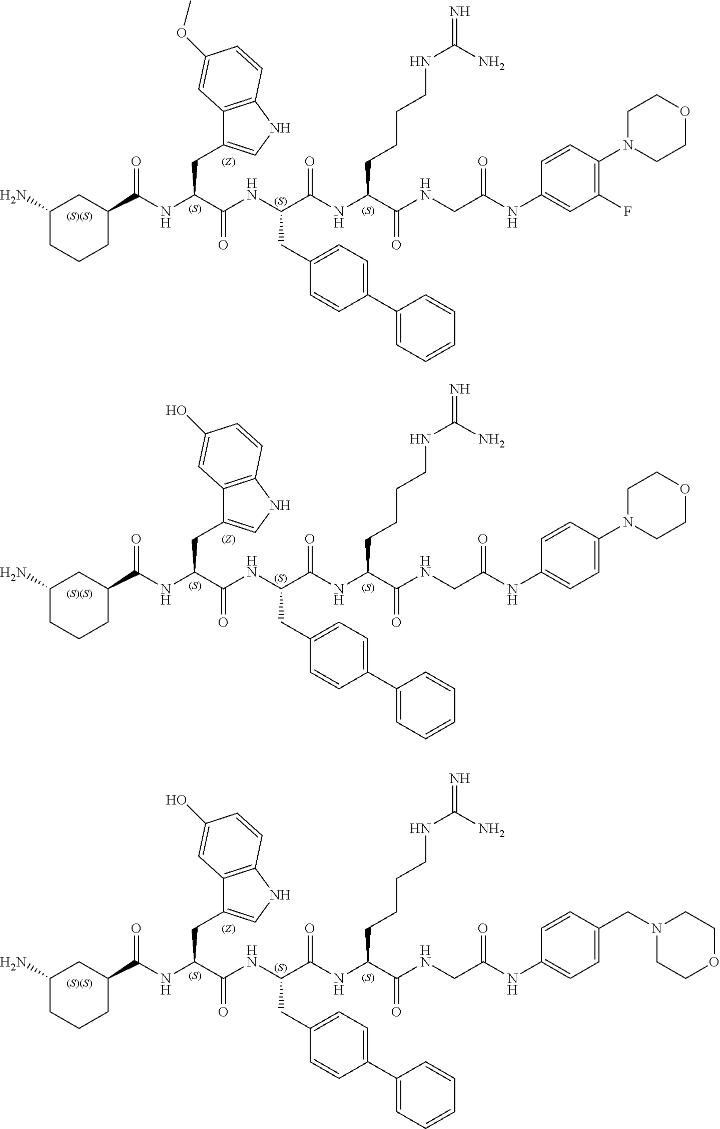

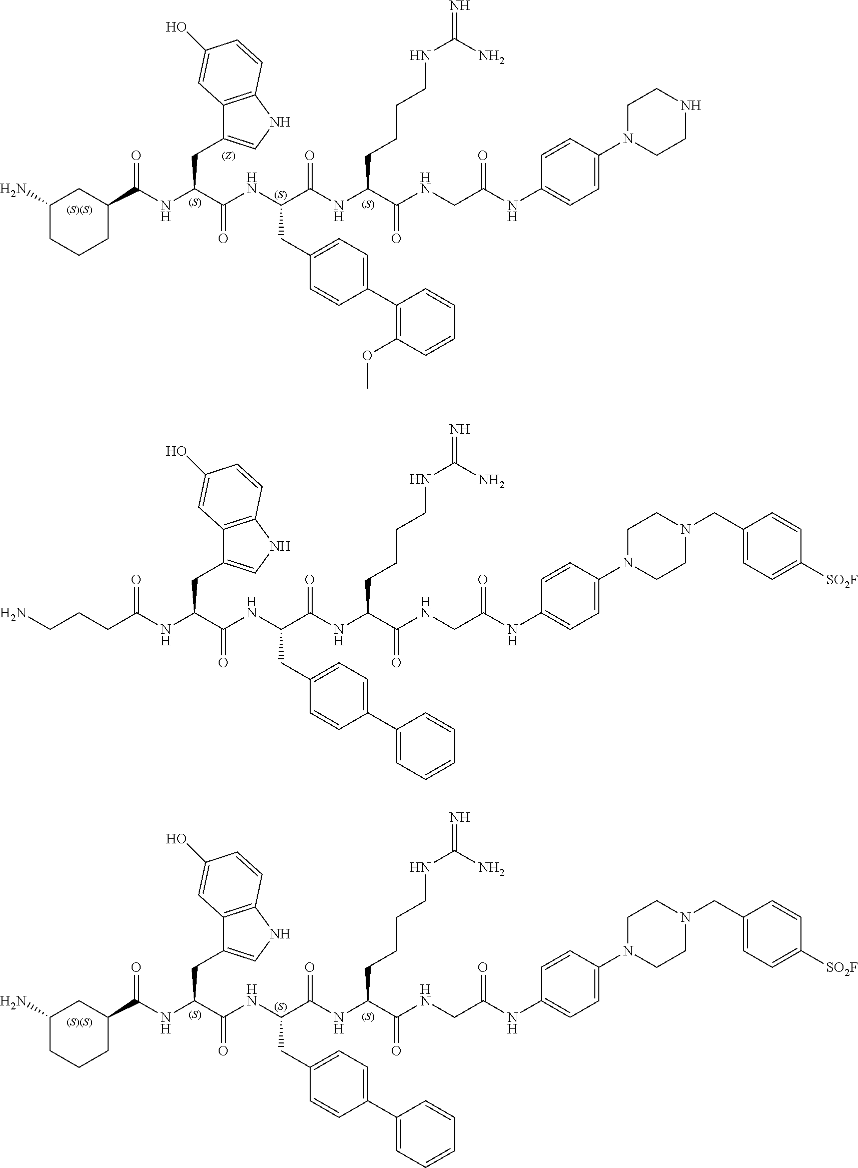

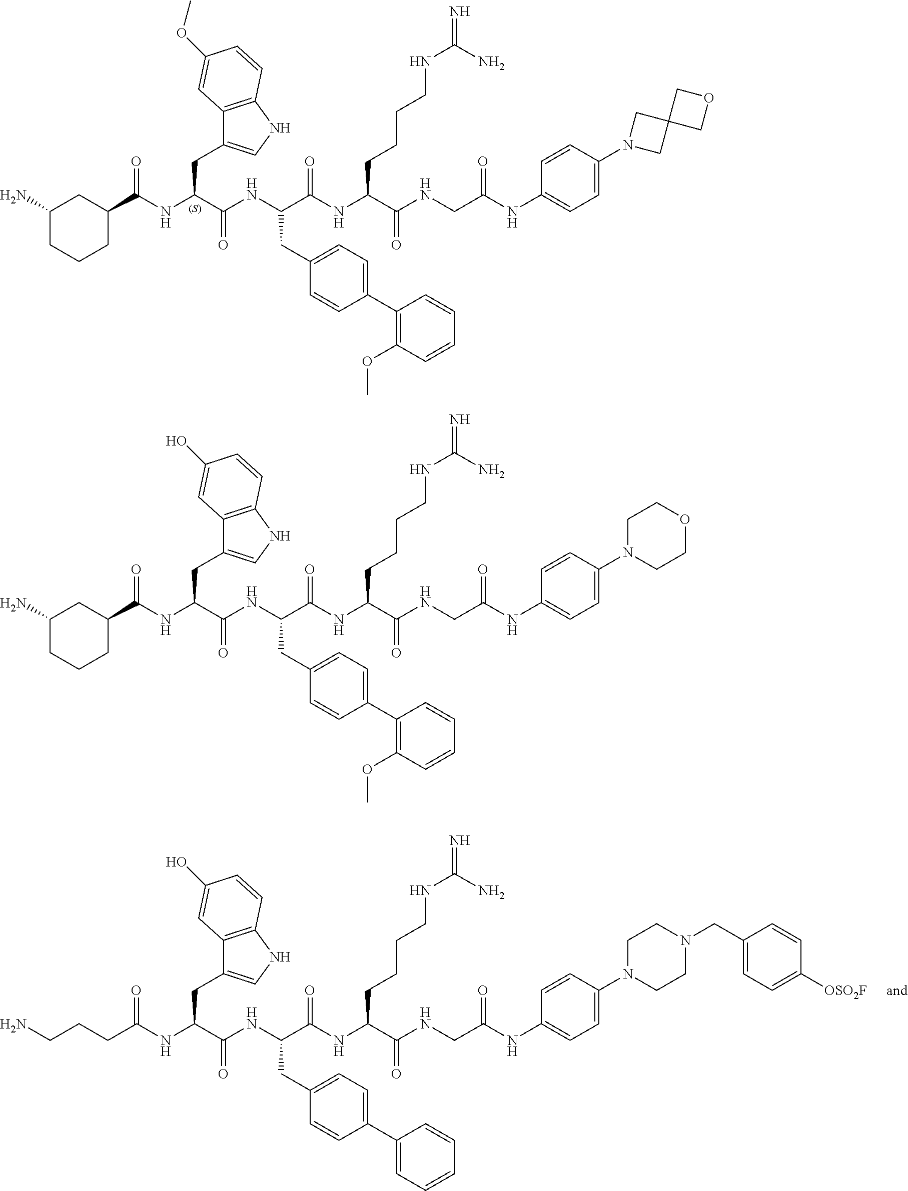

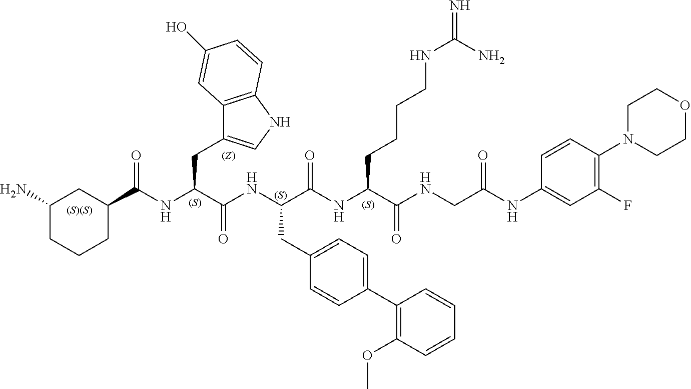

or a salt thereof.

Embodiment 27. The compound of Embodiment 1, wherein the peptide is

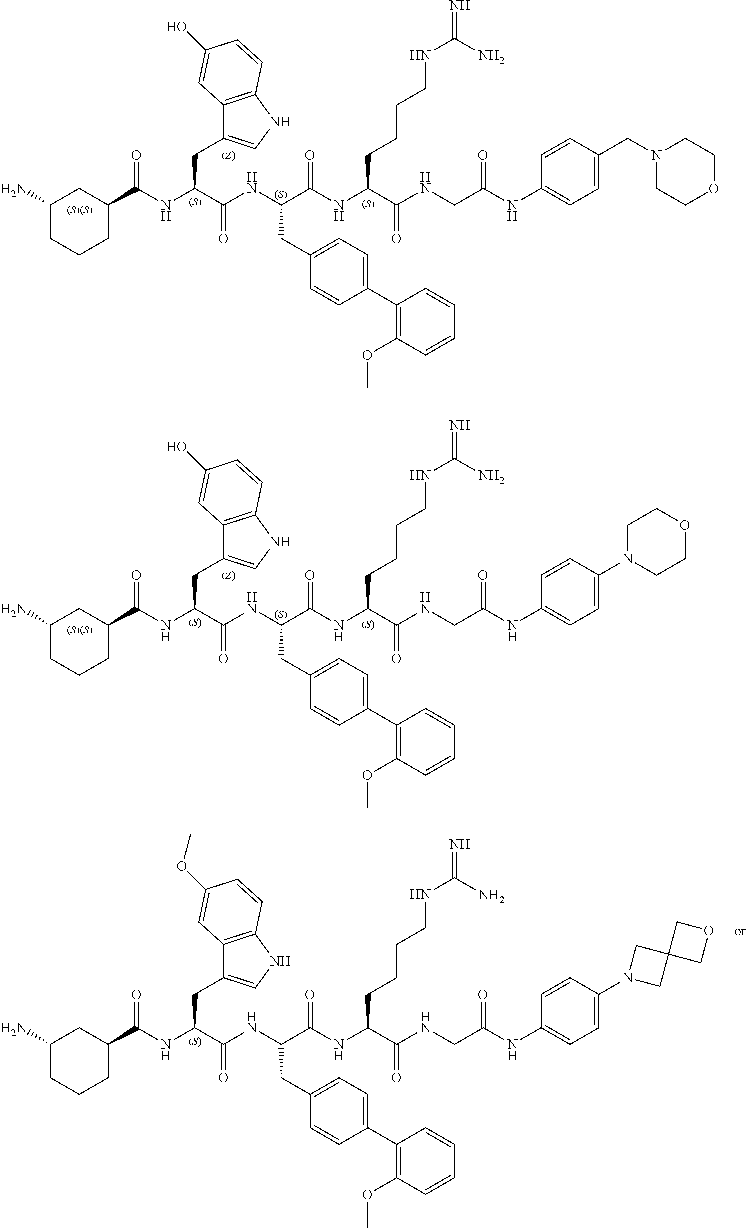

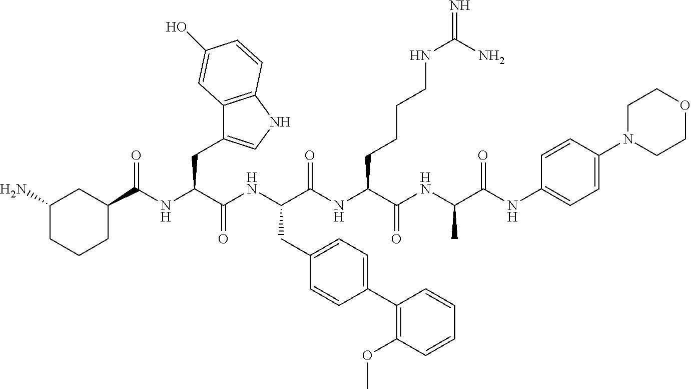 [SMILES: COc1ccccc1-c1ccc(C[C@H](NC(=O)[C@H](Cc2c[nH]c3ccc(O)cc23)NC(=O)[C@H]2CCC[C@H](N)C2)C(=O)N[C@@H](CCCCNC(=N)N)C(=O)N[C@H](C)C(=O)Nc2ccc(N3CCOCC3)cc2)cc1]

or a salt thereof.

Embodiment 28. A composition comprising a compound that comprises a peptide of Formula (I) as described in any one of Embodiments 1-27, or a pharmaceutically acceptable salt thereof, and a pharmaceutically acceptable carrier.

Embodiment 29. A method of treating a disease associated with EphA4 in a mammal in need thereof, comprising administering a therapeutically effective amount of a compound comprising a peptide of Formula (I) as described in any one of Embodiments 1-27, or a pharmaceutically acceptable salt thereof, to the mammal.

Embodiment 30. A compound comprising a peptide of Formula (I), or a pharmaceutically acceptable salt thereof, as described in any one of Embodiments 1-27, for use in medical therapy.

Embodiment 31. A compound comprising a peptide of Formula (I), or a pharmaceutically acceptable salt thereof, as described in any one of Embodiments 1-27, for the prophylactic or therapeutic treatment of a disease associated with EphA4 in a mammal in need thereof.

Embodiment 32. The use of a compound comprising a peptide of Formula (I), or a pharmaceutically acceptable salt thereof, as described in any one of Embodiments 1-27, to prepare a medicament for treating a disease associated with EphA4 in a mammal in need thereof.

Embodiment 101. A peptide of Formula (I) X₀—X₁—X₂—X₃—X₄ from N terminal to C terminal, wherein:

X₀ is a residue of an amino acid, wherein the N-terminus is a primary amine group NH₂—, or a capped amine R^{N}—C(═O)—NH—, wherein R^{N} is H, (C₃-C₆)cycloalkyl, or (C₁-C₄)alkyl;

X₁ is a residue of Trp, wherein Trp is optionally substituted with one or more groups independently selected from the group consisting of halo, hydroxy, cyano, carboxyl, —CONH₂, —NO₂, —SH, —SO₃H, —SO₄H, —SO₂NH₂, —OSO₂F, —SO₂F, —NHNH₂, —ONH₂, —NHC(═O)NHNH₂, —NHC(═O)NH₂, —NHC(═O)H, —NHC(═O)OH, —NHOH, (C₁-C₆)alkyl, (C₁-C₆)alkoxy, (C₁-C₆)alkoxycarbonyl, (C₁-C₆)alkanoyl, (C₁-C₆)alkanoyloxy, (C₁-C₆)heteroalkyl, (C₃-C₆)cycloalkyl, heterocycloalkyl, heterocycloalkyl(C₁-C₆)alkyl, aryl, heteroaryl, and —NR^{r}R^{s}, wherein any (C₁-C₆)alkyl, (C₁-C₆)alkoxy, (C₁-C₆)alkoxycarbonyl, (C₁-C₆)alkanoyl, (C₁-C₆)alkanoyloxy, (C₁-C₆)heteroalkyl, (C₃-C₆)cycloalkyl, heterocycloalkyl, heterocycloalkyl(C₁-C₆)alkyl, aryl, and heteroaryl is optionally substituted with one or more groups independently selected from the group consisting of halo, hydroxy, and (C₁-C₆)alkoxy;

X₂ is a residue of Bip, wherein Bip is optionally substituted on one or both phenyl groups with one or more groups independently selected from the group consisting of halo, hydroxy, cyano, carboxyl, —CONH₂, —NO₂, —SH, —SO₃H, —SO₄H, —SO₂NH₂, —OSO₂F, —SO₂F, —NHNH₂, —ONH₂, —NHC(═O)NHNH₂, —NHC(═O)NH₂, —NHC(═O)H, —NHC(═O)OH, —NHOH, (C₁-C₆)alkyl, (C₁-C₆)alkoxy, (C₁-C₆)alkoxycarbonyl, (C₁-C₆)alkanoyl, (C₁-C₆)alkanoyloxy, (C₁-C₆)heteroalkyl, (C₃-C₆)cycloalkyl, heterocycloalkyl, heterocycloalkyl(C₁-C₆)alkyl, aryl, heteroaryl, and —NR^{r}R^{s}, wherein any (C₁-C₆)alkyl, (C₁-C₆)alkoxy, (C₁-C₆)alkoxycarbonyl, (C₁-C₆)alkanoyl, (C₁-C₆)alkanoyloxy, (C₁-C₆)heteroalkyl, (C₃-C₆)cycloalkyl, heterocycloalkyl, heterocycloalkyl(C₁-C₆)alkyl, aryl, and heteroaryl is optionally substituted with one or more groups independently selected from the group consisting of halo, hydroxy, and (C₁-C₆)alkoxy;

X₃ is a residue of an amino acid;

X₄ is absent or a residue of an amino acid, wherein the C-terminus is a carboxyl group —COOH, or the C-terminus is amidated to form —C(═O)NR^{x}R^{y};

wherein each R^{x} and R^{y} is independently selected from the group consisting of H, (C₁-C₆)alkyl, (C₃-C₆)cycloalkyl, aryl, heteroaryl, (C₃-C₆)cycloalkyl(C₁-C₆)alkyl, aryl(C₁-C₆)alkyl, heteroaryl(C₁-C₆)alkyl, aryl-heterocycloalkyl-aryl, aryl-heterocycloalkyl-heteroaryl, aryl-heterocycloalkyl-(C₁-C₆)alkyl-aryl, aryl-heterocycloalkyl-(C₁-C₆)alkyl-heteroaryl, and aryl-(C₁-C₆)alkyl-heterocycloalkyl-aryl,

wherein any (C₁-C₆)alkyl, (C₃-C₆)cycloalkyl, aryl, heteroaryl, (C₃-C₆)cycloalkyl(C₁-C₆)alkyl, aryl(C₁-C₆)alkyl, heteroaryl(C₁-C₆)alkyl, aryl-heterocycloalkyl-aryl, aryl-heterocycloalkyl-heteroaryl, aryl-heterocycloalkyl-(C₁-C₆)alkyl-aryl, aryl-heterocycloalkyl-(C₁-C₆)alkyl-heteroaryl, and aryl-(C₁-C₆)alkyl-heterocycloalkyl-aryl is optionally substituted with one or more groups independently selected from the group consisting of halo, hydroxy, cyano, carboxyl, —CONH₂, —NO₂, —SH, —SO₃H, —SO₄H, —SO₂NH₂, —OSO₂F, —SO₂F, —NHNH₂, —ONH₂, —NHC(═O)NHNH₂, —NHC(═O)NH₂, —NHC(═O)H, —NHC(═O)OH, —NHOH, (C₁-C₆)alkyl, (C₁-C₆)alkoxy, (C₁-C₆)alkoxycarbonyl, (C₁-C₆)alkanoyl, (C₁-C₆)alkanoyloxy, (C₁-C₆)heteroalkyl, (C₃-C₆)cycloalkyl, heterocycloalkyl, heterocycloalkyl(C₁-C₆)alkyl, aryl, heteroaryl, and —NR^{r}R^{s}, wherein any (C₁-C₆)alkyl, (C₁-C₆)alkoxy, (C₁-C₆)alkoxycarbonyl, (C₁-C₆)alkanoyl, (C₁-C₆)alkanoyloxy, (C₁-C₆)heteroalkyl, (C₃-C₆)cycloalkyl, heterocycloalkyl, heterocycloalkyl(C₁-C₆)alkyl, aryl, and heteroaryl is optionally substituted with one or more groups independently selected from the group consisting of halo, hydroxy, and (C₁-C₆)alkoxy;

each R^{r} and R^{s} is independently selected from the group consisting of H, (C₁-C₆)alkyl, aryl, and (C₁-C₆)alkoxyaryl;

or a salt thereof.

Embodiment 102. The peptide of Embodiment 101, wherein X₀ is a residue of a beta-amino acid, gamma-amino acid, or delta-amino acid, wherein the N-terminus is a primary amine group NH₂—, or a capped amine R^{N}—C(═O)—NH—, wherein R^{N} is H, (C₃-C₆)cycloalkyl, or (C₁-C₄)alkyl.

Embodiment 103. The peptide of any one of Embodiments 101-102, wherein the N-terminus of the peptide is a primary amine group.

Embodiment 104. The peptide of any one of Embodiments 101-103, wherein X₀ is a residue of a gamma-amino acid.

Embodiment 105. The peptide of any one of Embodiments 101-104, wherein X₀ is a residue of gamma-amino-butyric acid (GABA).

Embodiment 106. The peptide of any one of Embodiments 101-104, wherein X₀ is a residue of 3-amino-cyclohexanecarboxylic acid (ACHC).

Embodiment 107. The peptide of any one of Embodiments 101-106, wherein X₁ is a residue of 5-hydroxy-Trp or 5-methoxy-Trp.

Embodiment 108. The peptide of any one of Embodiments 101-107, wherein X₂ is a residue of Bip or 4-(2-methoxyphenyl)-Phe.

Embodiment 109. The peptide of any one of Embodiments 101-108, wherein X₃ is a positively charged amino acid residue.

Embodiment 110. The peptide of any one of Embodiments 101-109, wherein X₃ is a residue of hArg, Arg, hLys, Lys, Orn, Dab, Dap, or 4-guanidino Phe.

Embodiment 111. The peptide of any one of Embodiments 101-110, wherein X₄ is a residue of Gly, Ala, Thr or Ser.

Embodiment 112. The peptide of any one of Embodiments 101-111, wherein X₄ is a residue of an amino acid, wherein the C-terminus is a carboxyl group —COOH, or the C-terminus is amidated to form —C(═O)NR^{x}R^{y}.

Embodiment 113. The peptide of any one of Embodiments 101-110, wherein X₄ is absent.

Embodiment 114. The peptide of any one of Embodiments 101-113, wherein the C-terminus of the peptide of Formula (I) is amidated and R^{x} is H; and R^{y} is aryl or heteroaryl, wherein the aryl and heteroaryl is optionally substituted with one or more groups independently selected from the group consisting of halo, hydroxy, cyano, carboxyl, —CONH₂, —NO₂, —SH, —SO₃H, —SO₄H, —SO₂NH₂, —OSO₂F, —SO₂F, —NHNH₂, —ONH₂, —NHC(═O)NHNH₂, —NHC(═O)NH₂, —NHC(═O)H, —NHC(═O)OH, —NHOH, (C₁-C₆)alkyl, (C₁-C₆)alkoxy, (C₁-C₆)alkoxycarbonyl, (C₁-C₆)alkanoyl, (C₁-C₆)alkanoyloxy, (C₁-C₆)heteroalkyl, (C₃-C₆)cycloalkyl, heterocycloalkyl, heterocycloalkyl(C₁-C₆)alkyl, aryl, heteroaryl, and —NR^{r}R^{s}, wherein any (C₁-C₆)alkyl, (C₁-C₆)alkoxy, (C₁-C₆)alkoxycarbonyl, (C₁-C₆)alkanoyl, (C₁-C₆)alkanoyloxy, (C₁-C₆)heteroalkyl, (C₃-C₆)cycloalkyl, heterocycloalkyl, heterocycloalkyl(C₁-C₆)alkyl, aryl, and heteroaryl is optionally substituted with one or more groups independently selected from the group consisting of halo, hydroxy, and (C₁-C₆)alkoxy.

Embodiment 115. The peptide of any one of Embodiments 101-114, wherein the C-terminus of the peptide of Formula (I) is amidated and R^{x} is H; and R^{y} is phenyl that is optionally substituted with one or more groups independently selected from halo, hydroxy, cyano, carboxyl, —CONH₂, —NO₂, —SH, —SO₃H, —SO₄H, —SO₂NH₂, —OSO₂F, —SO₂F, —NHNH₂, —ONH₂, —NHC(═O)NHNH₂, —NHC(═O)NH₂, —NHC(═O)H, —NHC(═O)OH, —NHOH, (C₁-C₆)alkyl, (C₁-C₆)alkoxy, (C₁-C₆)alkoxycarbonyl, (C₁-C₆)alkanoyl, (C₁-C₆)alkanoyloxy, (C₁-C₆)heteroalkyl, (C₃-C₆)cycloalkyl, heterocycloalkyl, heterocycloalkyl(C₁-C₆)alkyl, aryl, heteroaryl, and —NR^{r}R^{s}, wherein any (C₁-C₆)alkyl, (C₁-C₆)alkoxy, (C₁-C₆)alkoxycarbonyl, (C₁-C₆)alkanoyl, (C₁-C₆)alkanoyloxy, (C₁-C₆)heteroalkyl, (C₃-C₆)cycloalkyl, heterocycloalkyl, heterocycloalkyl(C₁-C₆)alkyl, aryl, and heteroaryl is optionally substituted with one or more groups independently selected from the group consisting of halo, hydroxy, and (C₁-C₆)alkoxy.

Embodiment 116. The peptide of any one of Embodiments 101-114, wherein the C-terminus of the peptide of Formula (I) is amidated and R^{x} is H; and R^{y} is 2-pyridyl that is optionally substituted with one or more groups independently selected from halo, hydroxy, cyano, carboxyl, —CONH₂, —NO₂, —SH, —SO₃H, —SO₄H, —SO₂NH₂, —OSO₂F, —SO₂F, —NHNH₂, —ONH₂, —NHC(═O)NHNH₂, —NHC(═O)NH₂, —NHC(═O)H, —NHC(═O)OH, —NHOH, (C₁-C₆)alkyl, (C₁-C₆)alkoxy, (C₁-C₆)alkoxycarbonyl, (C₁-C₆)alkanoyl, (C₁-C₆)alkanoyloxy, (C₁-C₆)heteroalkyl, (C₃-C₆)cycloalkyl, heterocycloalkyl, heterocycloalkyl(C₁-C₆)alkyl, aryl, heteroaryl, and —NR^{r}R^{s}, wherein any (C₁-C₆)alkyl, (C₁-C₆)alkoxy, (C₁-C₆)alkoxycarbonyl, (C₁-C₆)alkanoyl, (C₁-C₆)alkanoyloxy, (C₁-C₆)heteroalkyl, (C₃-C₆)cycloalkyl, heterocycloalkyl, heterocycloalkyl(C₁-C₆)alkyl, aryl, and heteroaryl is optionally substituted with one or more groups independently selected from the group consisting of halo, hydroxy, and (C₁-C₆)alkoxy.

Embodiment 117. The peptide of any one of Embodiments 101-116, wherein:

X₀ is a residue of GABA or ACHC,

X₁ is a residue of Trp, 5-hydroxy-Trp or 5-methoxy-Trp,

X₂ is a residue of Bip or 4-(2-methoxyphenyl)-Phe,

X₃ is a residue of hArg, Arg, hLys, Lys, Orn, Dab or Dap,

X₄ is a residue of Gly, Ala, Thr or Ser,

wherein the C-terminus of the peptide of Formula (I) is amidated and R^{x} is H; and R^{y} is 2-pyridyl or phenyl that is optionally substituted with one or more groups independently selected from the group consisting of halo, hydroxy, cyano, carboxyl, —CONH₂, —NO₂, —SH, —SO₃H, —SO₄H, —SO₂NH₂, —OSO₂F, —SO₂F, —NHNH₂, —ONH₂, —NHC(═O)NHNH₂, —NHC(═O)NH₂, —NHC(═O)H, —NHC(═O)OH, —NHOH, (C₁-C₆)alkyl, (C₁-C₆)alkoxy, (C₁-C₆)alkoxycarbonyl, (C₁-C₆)alkanoyl, (C₁-C₆)alkanoyloxy, (C₁-C₆)heteroalkyl, (C₃-C₆)cycloalkyl, heterocycloalkyl, heterocycloalkyl(C₁-C₆)alkyl, aryl, heteroaryl, and —NR^{r}R^{s}, wherein any (C₁-C₆)alkyl, (C₁-C₆)alkoxy, (C₁-C₆)alkoxycarbonyl, (C₁-C₆)alkanoyl, (C₁-C₆)alkanoyloxy, (C₁-C₆)heteroalkyl, (C₃-C₆)cycloalkyl, heterocycloalkyl, heterocycloalkyl(C₁-C₆)alkyl, aryl, and heteroaryl is optionally substituted with one or more groups independently selected from the group consisting of halo, hydroxy, and (C₁-C₆)alkoxy;

or a salt thereof.

Embodiment 118. The peptide of any one of Embodiments 101-117, wherein X₄ is a residue of Gly or Ala.

Embodiment 119. The peptide of any one of Embodiments 101-118, that has structure of Formula (Ia)

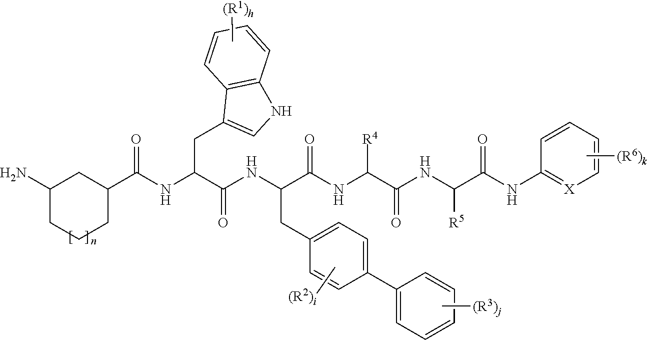

wherein

n is 0, 1, or 2;

X is C or N;

h, i, j and k are each independent 0, 1, 2 or 3;

R¹, R², R³, and R⁶ are each independently selected from the group consisting of absent, halo, hydroxy, cyano, carboxyl, —CONH₂, —NO₂, —SH, —SO₃H, —SO₄H, —SO₂NH₂, —OSO₂F, —SO₂F, —NHNH₂, —ONH₂, —NHC(═O)NHNH₂, —NHC(═O)NH₂, —NHC(═O)H, —NHC(═O)OH, —NHOH, (C₁-C₆)alkyl, (C₁-C₆)alkoxy, (C₁-C₆)alkoxycarbonyl, (C₁-C₆)alkanoyl, (C₁-C₆)alkanoyloxy, (C₁-C₆)heteroalkyl, (C₃-C₆)cycloalkyl, heterocycloalkyl, heterocycloalkyl(C₁-C₆)alkyl, aryl, heteroaryl, and —NR^{r}R^{s}, wherein any (C₁-C₆)alkyl, (C₁-C₆)alkoxy, (C₁-C₆)alkoxycarbonyl, (C₁-C₆)alkanoyl, (C₁-C₆)alkanoyloxy, (C₁-C₆)heteroalkyl, (C₃-C₆)cycloalkyl, heterocycloalkyl, heterocycloalkyl(C₁-C₆)alkyl, aryl, and heteroaryl is optionally substituted with one or more groups independently selected from the group consisting of halo, hydroxy, and (C₁-C₆)alkoxy;

R⁴, and R⁵ are each independently an amino acid side chain;

or a salt thereof.

Embodiment 120. The peptide of Embodiment 119, wherein R⁴ is the side chain of hArg, Arg, hLys, Lys, Orn, Dab, Dap, or 4-guanidino Phe.

Embodiment 121. The peptide of Embodiment 119, wherein R⁴ is the side chain of L-hArg.

Embodiment 122. The peptide of any one of Embodiments 119-121, wherein R^{s} is —H, —CH₃, —CH₂OH, or —CH(OH)CH₃.

Embodiment 123. The peptide of any one of Embodiments 101-118, that has structure of Formula (Ib)

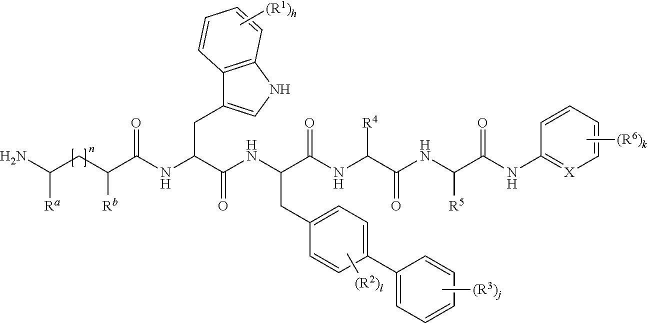

wherein

n is 0, 1, or 2;

X is C or N;

h, i, j and k are each independent 0, 1, 2 or 3;

R^{a}, R^{b}, R¹, R², R³, and R⁶ are each independently selected from the group consisting of absent, hydrogen, halo, hydroxy, cyano, carboxyl, —CONH₂, —NO₂, —SH, —SO₃H, —SO₄H, —SO₂NH₂, —OSO₂F, —SO₂F, —NHNH₂, —ONH₂, —NHC(═O)NHNH₂, —NHC(═O)NH₂, —NHC(═O)H, —NHC(═O)OH, —NHOH, (C₁-C₆)alkyl, (C₁-C₆)alkoxy, (C₁-C₆)alkoxycarbonyl, (C₁-C₆)alkanoyl, (C₁-C₆)alkanoyloxy, (C₁-C₆)heteroalkyl, (C₃-C₆)cycloalkyl, heterocycloalkyl, heterocycloalkyl(C₁-C₆)alkyl, aryl, heteroaryl, and —NR^{r}R^{s}, wherein any (C₁-C₆)alkyl, (C₁-C₆)alkoxy, (C₁-C₆)alkoxycarbonyl, (C₁-C₆)alkanoyl, (C₁-C₆)alkanoyloxy, (C₁-C₆)heteroalkyl, (C₃-C₆)cycloalkyl, heterocycloalkyl, heterocycloalkyl(C₁-C₆)alkyl, aryl, and heteroaryl is optionally substituted with one or more groups independently selected from the group consisting of halo, hydroxy, and (C₁-C₆)alkoxy;

R⁴, and R⁵ are each independently an amino acid side chain;

or a salt thereof.

Embodiment 124. The peptide of Embodiment 123, wherein R⁶ is —OSO₂F, or —SO₂F.

Embodiment 125. The peptide of any one of Embodiments 101-118, that has structure of Formula (Ic)

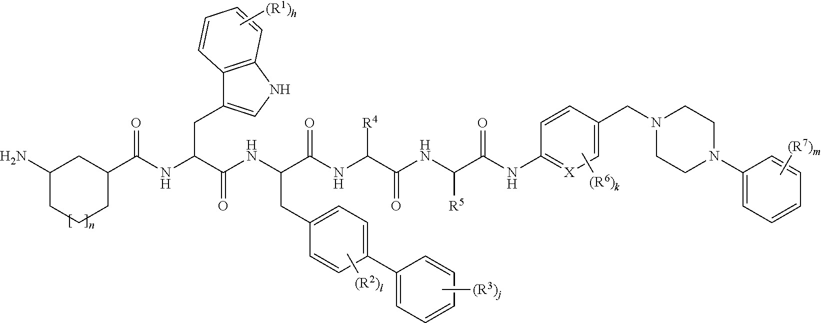

wherein

n is 0, 1, or 2;

X is C or N;

h, i, j, k and m are each independent 0, 1, 2 or 3;

R¹, R², R³, R⁶ and R⁷ are each independently selected from the group consisting of absent, halo, hydroxy, cyano, carboxyl, —CONH₂, —NO₂, —SH, —SO₃H, —SO₄H, —SO₂NH₂, —OSO₂F, —SO₂F, —NHNH₂, —ONH₂, —NHC(═O)NHNH₂, —NHC(═O)NH₂, —NHC(═O)H, —NHC(═O)OH, —NHOH, (C₁-C₆)alkyl, (C₁-C₆)alkoxy, (C₁-C₆)alkoxycarbonyl, (C₁-C₆)alkanoyl, (C₁-C₆)alkanoyloxy, (C₁-C₆)heteroalkyl, (C₃-C₆)cycloalkyl, heterocycloalkyl, heterocycloalkyl(C₁-C₆)alkyl, aryl, heteroaryl, and —NR^{r}R^{s}, wherein any (C₁-C₆)alkyl, (C₁-C₆)alkoxy, (C₁-C₆)alkoxycarbonyl, (C₁-C₆)alkanoyl, (C₁-C₆)alkanoyloxy, (C₁-C₆)heteroalkyl, (C₃-C₆)cycloalkyl, heterocycloalkyl, heterocycloalkyl(C₁-C₆)alkyl, aryl, and heteroaryl is optionally substituted with one or more groups independently selected from the group consisting of halo, hydroxy, and (C₁-C₆)alkoxy;

R⁴, and R⁵ are each independently an amino acid side chain;

or a salt thereof.

Embodiment 126. The peptide of Embodiment 125, wherein R⁷ is —OSO₂F, or —SO₂F.

Embodiment 127. The peptide of any one of Embodiments 101-118, that has structure of Formula (Id)

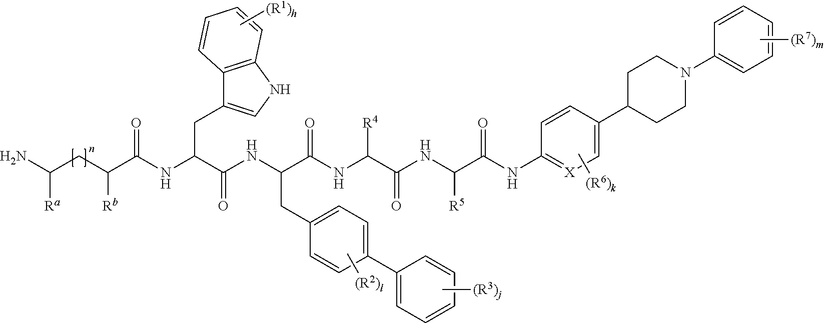

wherein

n is 0, 1, or 2;

X is C or N;

h, i, j, k and m are each independent 0, 1, 2 or 3;

R^{a}, R^{b}, R¹, R², R³, R⁶ and R⁷ are each independently selected from the group consisting of absent, hydrogen, halo, hydroxy, cyano, carboxyl, —CONH₂, —NO₂, —SH, —SO₃H, —SO₄H, —SO₂NH₂, —OSO₂F, —SO₂F, —NHNH₂, —ONH₂, —NHC(═O)NHNH₂, —NHC(═O)NH₂, —NHC(═O)H, —NHC(═O)OH, —NHOH, (C₁-C₆)alkyl, (C₁-C₆)alkoxy, (C₁-C₆)alkoxycarbonyl, (C₁-C₆)alkanoyl, (C₁-C₆)alkanoyloxy, (C₁-C₆)heteroalkyl, (C₃-C₆)cycloalkyl, heterocycloalkyl, heterocycloalkyl(C₁-C₆)alkyl, aryl, heteroaryl, and —NR^{r}R^{s}, wherein any (C₁-C₆)alkyl, (C₁-C₆)alkoxy, (C₁-C₆)alkoxycarbonyl, (C₁-C₆)alkanoyl, (C₁-C₆)alkanoyloxy, (C₁-C₆)heteroalkyl, (C₃-C₆)cycloalkyl, heterocycloalkyl, heterocycloalkyl(C₁-C₆)alkyl, aryl, and heteroaryl is optionally substituted with one or more groups independently selected from the group consisting of halo, hydroxy, and (C₁-C₆)alkoxy;

R⁴, and R⁵ are each independently an amino acid side chain;

or a salt thereof.

Embodiment 128. The peptide of Embodiment 127, wherein R⁷ is —OSO₂F, or —SO₂F.

Embodiment 129. The peptide of Embodiment 101, that is selected from the group consisting of:

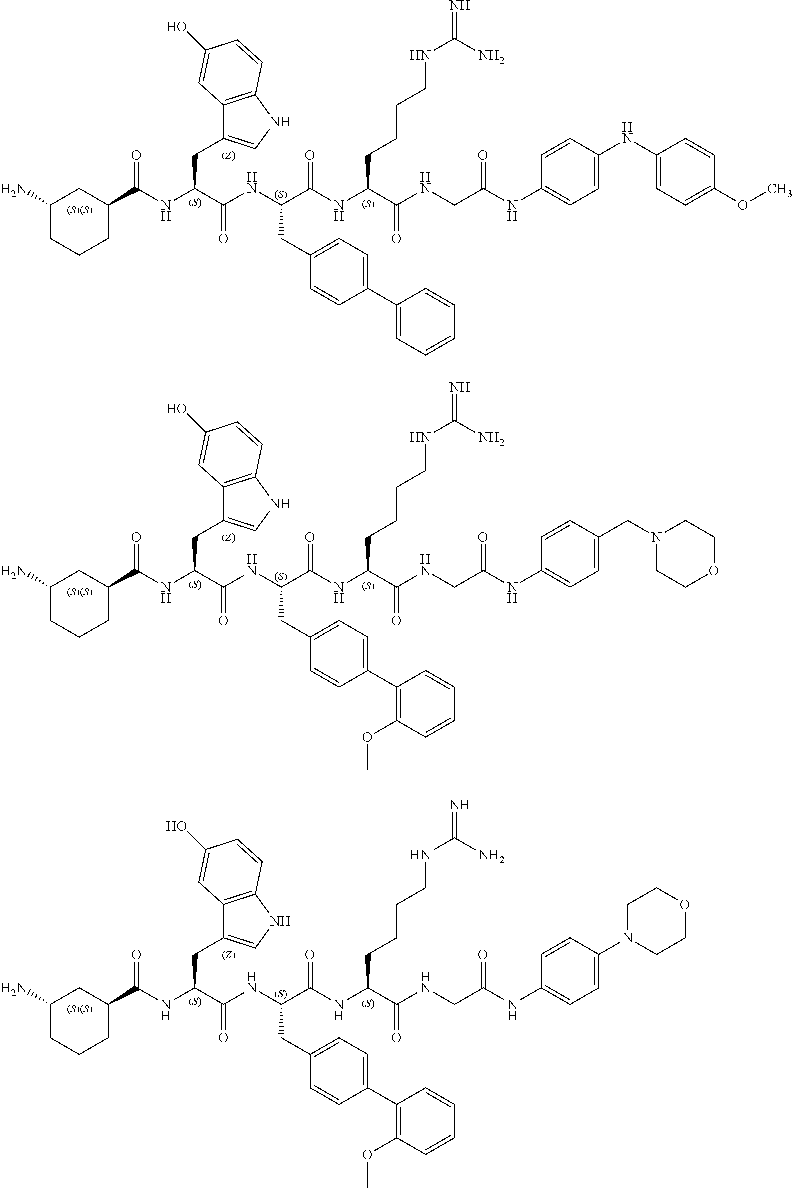

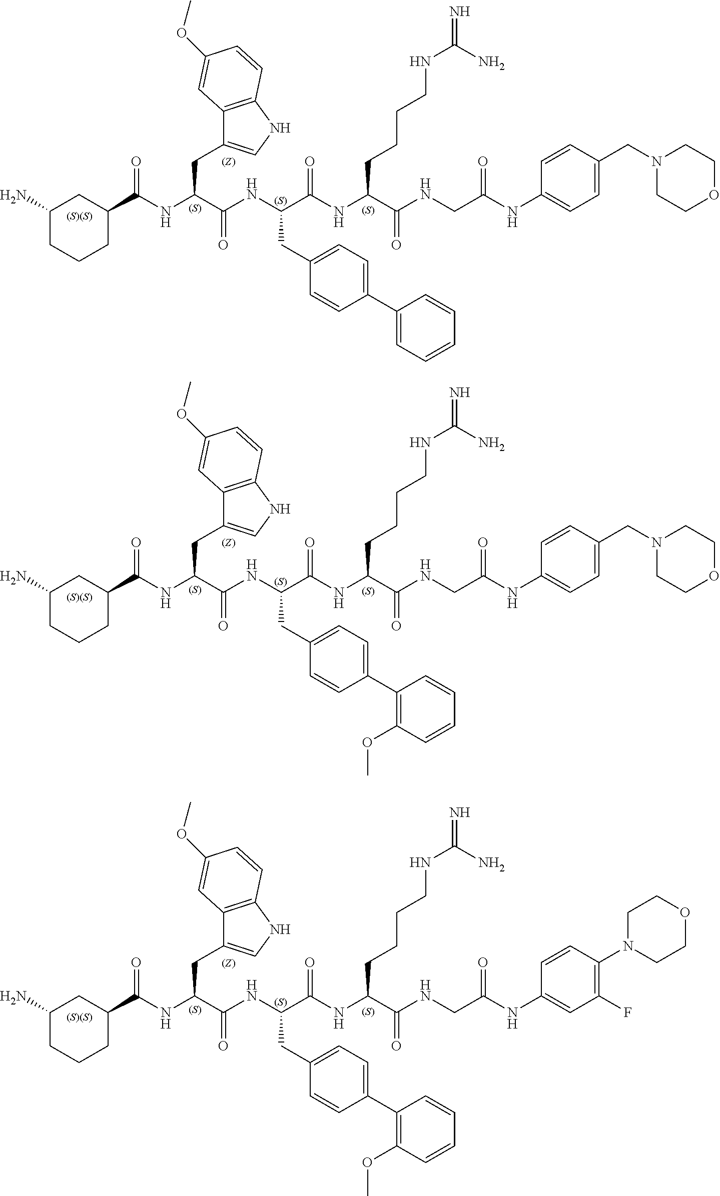

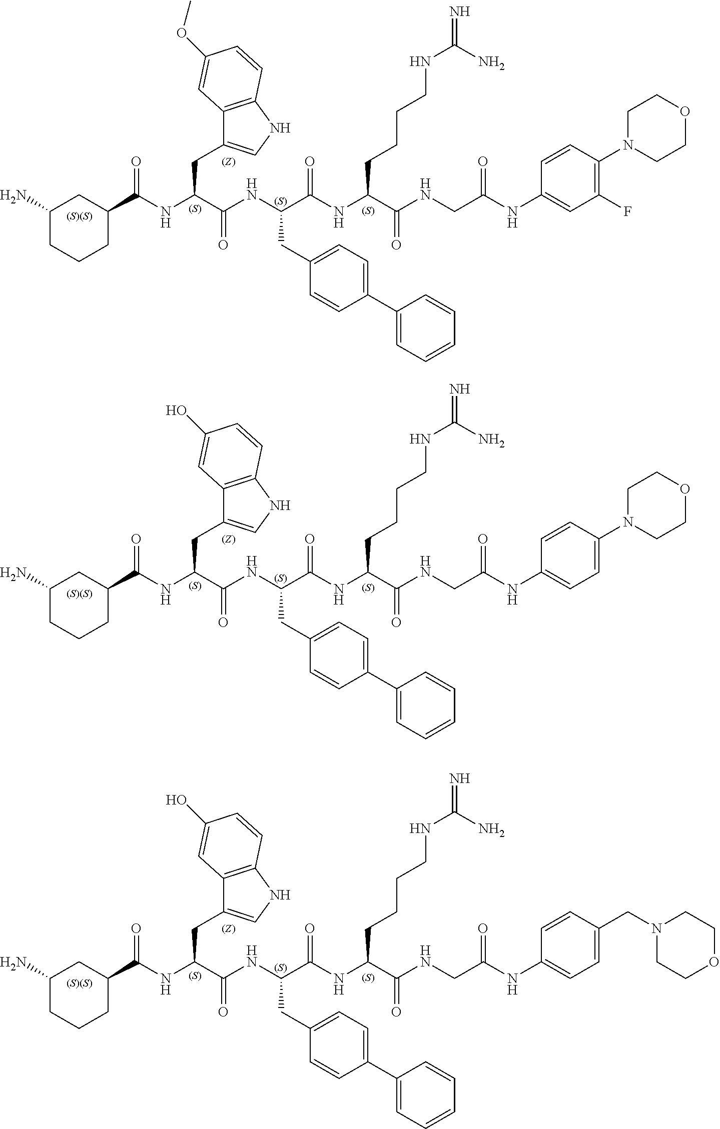

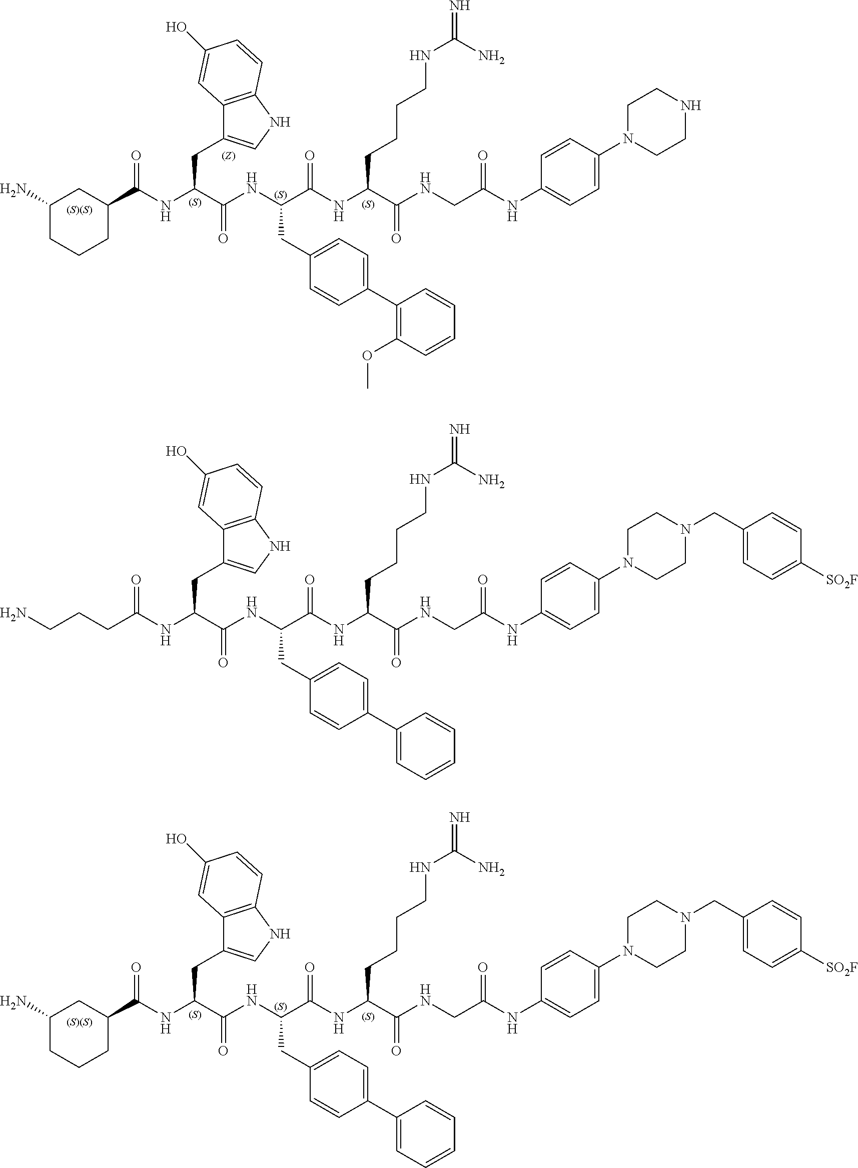

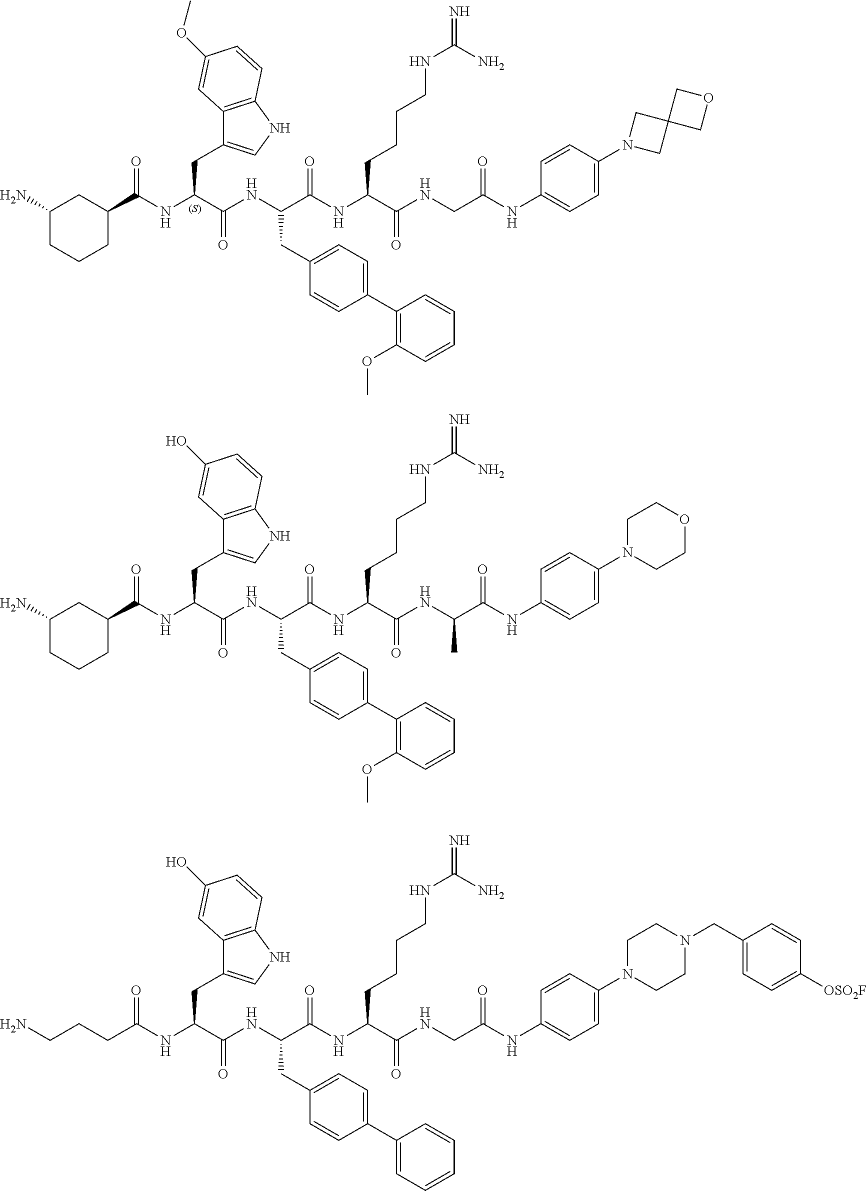

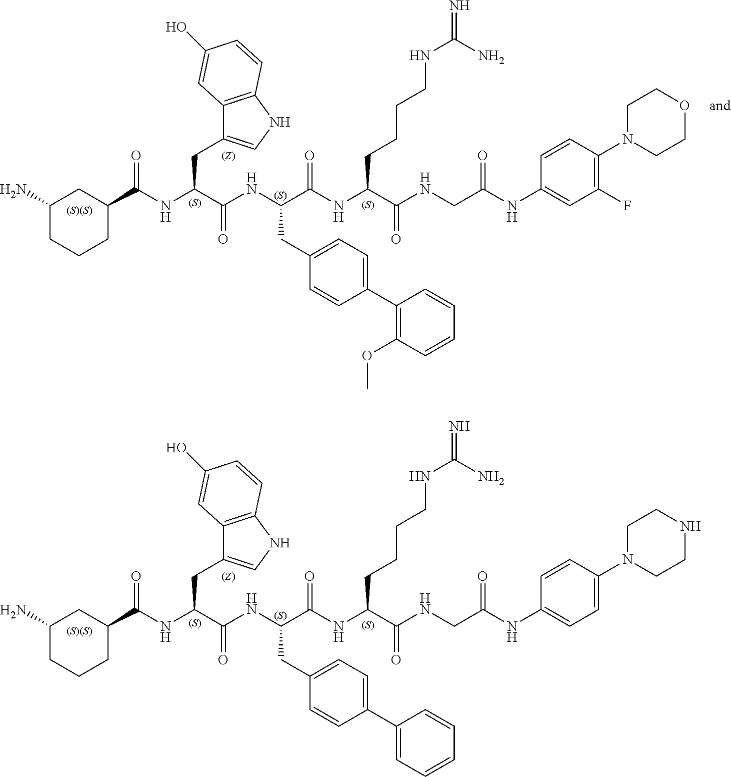

or a salt thereof.

Embodiment 130. The peptide of Embodiment 101, that is

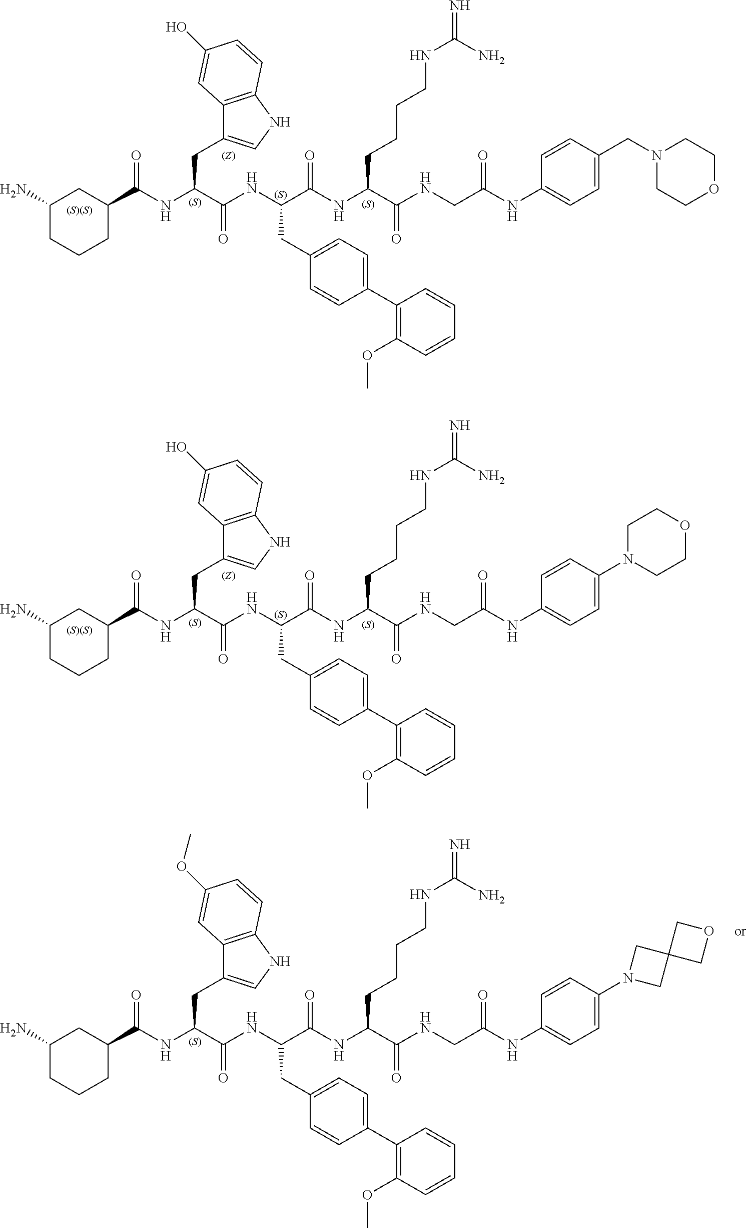

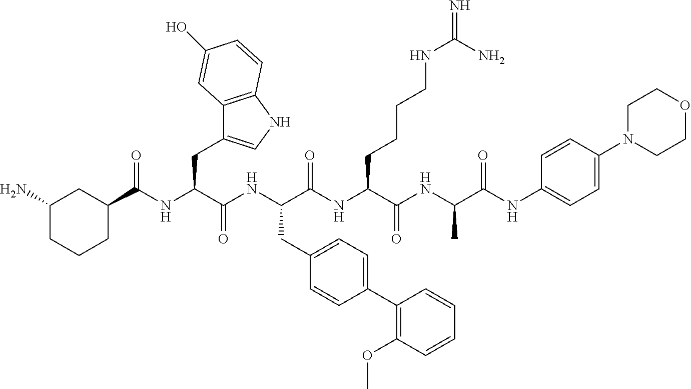 [SMILES: COc1ccccc1-c1ccc(C[C@H](NC(=O)[C@H](Cc2c[nH]c3ccc(O)cc23)NC(=O)[C@H]2CCC[C@H](N)C2)C(=O)N[C@@H](CCCCNC(=N)N)C(=O)N[C@H](C)C(=O)Nc2ccc(N3CCOCC3)cc2)cc1]

or a salt thereof.

Embodiment 131. The peptide of Embodiment 101, that is

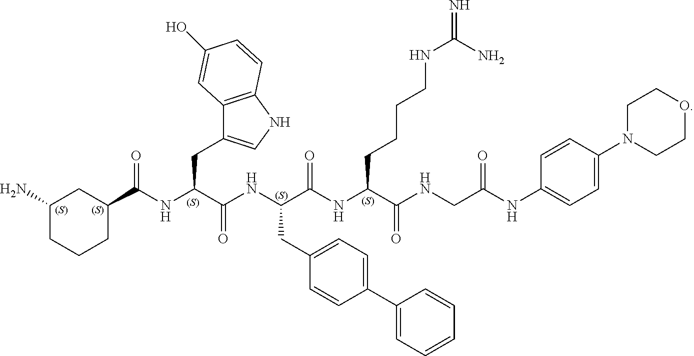

or a salt thereof.

Embodiment 132. A composition comprising a peptide as described in any one of Embodiments 101-131, or a pharmaceutically acceptable salt thereof, and a pharmaceutically acceptable carrier.

Embodiment 133. The composition of Embodiment 132, which is a pharmaceutical composition.

Embodiment 134. A method of activating EphA4 in a motor neuron, the method comprising contacting EphA4 with an effective amount of a peptide as described in any one of Embodiments 101-131, or salt thereof, wherein the peptide is an agonist.

Embodiment 135. The method of Embodiment 134, wherein the EphA4 is activated by at least about 30% when tested with 1 micromolar or less, as compared to non-treated control.

Embodiment 136. The method of Embodiment 134, wherein the EphA4 is activated by at least about 50% when tested with 1 micromolar or less, as compared to non-treated control.

Embodiment 137. A peptide or a pharmaceutically acceptable salt thereof, as described in any one of Embodiments 101-131, for use in medical therapy.

Embodiment 138. A method of treating a disease associated with EphA4 in a mammal in need thereof, comprising administering a therapeutically effective amount of a peptide as described in any one of Embodiments 101-131, or a pharmaceutically acceptable salt thereof, to the mammal.

Embodiment 139. The method of Embodiment 138, wherein the disease associated with EphA4 is cancer.

Embodiment 140. The method of Embodiment 139, wherein the cancer is selected from the group consisting of gastric cancer, breast cancer, pancreatic cancer, multiple myeloma, brain cancer (e.g., glioma), thyroid cancer, urothelial cancer, testis cancer, endometrial cancer, rectal cancer, colon cancer, urothelial cancer, or skin cancer.

Embodiment 141. The method of Embodiment 138, wherein the disease associated with EphA4 is a neurodegenerative disease.

Embodiment 142. The method of Embodiment 141, wherein the neurodegenerative disease is amyotrophic lateral sclerosis (ALS), Alzheimer's disease (AD) or Parkinson's disease (PD).

Embodiment 143. The method of Embodiment 142, wherein the neurodegenerative disease is ALS.

Embodiment 144. The method of Embodiment 143, wherein the ALS is familial ALS (fALS).

Embodiment 145. The method of Embodiment 143, wherein the ALS is sporadic ALS (sALS).

Embodiment 146. The method of any one of Embodiments 143-145, wherein motor neuron degeneration is reduced.

Embodiment 147. The method of any one of Embodiments 143-145, wherein motor neuron degeneration induced by astrocytes is reduced.

Embodiment 148. The method of any one of Embodiments 138-147, wherein the peptide is an EphA4 agonist.

Embodiment 149. The method of Embodiment 148, wherein the peptide activates EphA4 expressed in a brain neuron or a spinal cord neuron.

Embodiment 150. A peptide or a pharmaceutically acceptable salt thereof, as described in any one of Embodiments 101-131, for the prophylactic or therapeutic treatment of a disease associated with EphA4.

Embodiment 151. The use of a peptide or a pharmaceutically acceptable salt thereof, as described in any one of Embodiments 101-131, to prepare a medicament for treating a disease associated with EphA4 in a mammal.

Embodiment 152. A method of treating or preventing motor neuron degeneration in a mammal in need thereof, comprising administering a therapeutically effective amount of a peptide as described in any one of Embodiments 101-131, or a pharmaceutically acceptable salt thereof, to the mammal.

Embodiment 153. The method of Embodiment 152, wherein the motor neuron degeneration is induced by astrocytes.

Embodiment 154. The method of Embodiment 152 or 153, wherein the mammal has familial ALS (fALS) or was determined to have a mutation associated with fALS.

Embodiment 155. The method of Embodiment 154, wherein the peptide is administered to the mammal prophylactically.

Embodiment 156. The method of Embodiment 152 or 153, wherein the mammal has sporadic ALS (sALS).

Embodiment 157. The method of any one of Embodiments 152-156, wherein the peptide is an EphA4 agonist.

Embodiment 158. A peptide or a pharmaceutically acceptable salt thereof, as described in any one of Embodiments 101-131, for treating or preventing motor neuron degeneration.

Embodiment 159. The use of a peptide or a pharmaceutically acceptable salt thereof, as described in any one of Embodiments 101-131, to prepare a medicament for treating or preventing motor neuron degeneration in a mammal.

Embodiment 160. A method for identifying an EphA4 agonist, the method comprising isolating primary motor neurons from the spinal cord of an animal, contacting a test compound with the isolated primary motor neurons, under conditions suitable for binding between the test compound and EphA4, evaluating axon growth cone morphology of the primary motor neurons, and identifying the test compound as an EphA4 agonist when growth cone collapse is detected.

Embodiment 161. A method of identifying an ALS patient that is likely to respond to treatment, the method comprising of a) isolating fibroblasts from the ALS patient, b) culturing the fibroblasts under conditions suitable to generate patient derived astrocytes, c) co-culturing the patient derived astrocytes with mouse motor neurons (MN) in the presence of a peptide as described in any one of Embodiments 101-131, or a pharmaceutically acceptable salt thereof, and d) identifying the patient as being likely to respond to treatment with the peptide, or pharmaceutically acceptable salt thereof, when MN cell degeneration or death is inhibited as compared to a non-treatment control.

### Certain Definitions

The following definitions are used, unless otherwise described: halo or halogen is fluoro, chloro, bromo, or iodo. Alkyl, alkoxy, alkenyl, alkynyl, etc. denote both straight and branched groups; but reference to an individual radical such as propyl embraces only the straight chain radical, a branched chain isomer such as isopropyl being specifically referred to.

The term “alkyl”, by itself or as part of another substituent, means, unless otherwise stated, a straight or branched chain hydrocarbon radical, having the number of carbon atoms designated (i.e., C₁₋₈ means one to eight carbons). Examples include (C₁-C₈)alkyl, (C₂-C₈)alkyl, (C₁-C₆)alkyl, (C₁-C₃)alkyl, (C₂-C₆)alkyl and (C₃-C₆)alkyl. Examples of alkyl groups include methyl, ethyl, n-propyl, iso-propyl, n-butyl, t-butyl, iso-butyl, sec-butyl, n-pentyl, n-hexyl, n-heptyl, n-octyl, and higher homologs and isomers.

The term “alkoxy” refers to the formula —OR or radical thereof, where R is an alkyl as defined.

The term “heteroalkyl” refers to a straight or branched hydrocarbon chain alkyl radical containing no unsaturation, having the number of carbon atoms designated (e.g., C₁-C₈ alkyl) consisting of carbon and hydrogen atoms and one or two heteroatoms selected from O, N, S and Si, wherein the nitrogen or sulfur atoms may be optionally oxidized and the nitrogen atom may be quaternized. The heteroatom(s) may be placed at any position of the heteroalkyl group including between the rest of the heteroalkyl group and the fragment to which it is attached. The heteroalkyl is attached to the rest of the molecule by a single bond.

The term “alkenyl” refers to an unsaturated alkyl radical having one or more double bonds. Examples of such unsaturated alkyl groups include vinyl, 2-propenyl, crotyl, 2-isopentenyl, 2-(butadienyl), 2,4-pentadienyl, 3-(1,4-pentadienyl) and the higher homologs and isomers.

The term “cycloalkyl” refers to a saturated or partially unsaturated (non-aromatic) all carbon ring having 3 to 8 carbon atoms (i.e., (C₃-C₈)carbocycle). The term also includes multiple condensed, saturated all carbon ring systems (e.g., ring systems comprising 2, 3 or 4 carbocyclic rings). Accordingly, carbocycle includes multicyclic carbocyles such as a bicyclic carbocycles (e.g., bicyclic carbocycles having about 3 to 15 carbon atoms, about 6 to 15 carbon atoms, or 6 to 12 carbon atoms such as bicyclo[3.1.0]hexane and bicyclo[2.1.1]hexane), and polycyclic carbocycles (e.g tricyclic and tetracyclic carbocycles with up to about 20 carbon atoms). The rings of the multiple condensed ring system can be connected to each other via fused, spiro and bridged bonds when allowed by valency requirements. For example, multicyclic carbocyles can be connected to each other via a single carbon atom to form a spiro connection (e.g., spiropentane, spiro[4,5]decane, etc), via two adjacent carbon atoms to form a fused connection (e.g., carbocycles such as decahydronaphthalene, norsabinane, norcarane) or via two non-adjacent carbon atoms to form a bridged connection (e.g., norbornane, bicyclo[2.2.2]octane, etc). Non-limiting examples of cycloalkyls include cyclopropyl, cyclobutyl, cyclopentyl, cyclohexyl, bicyclo[2.2.1]heptane, pinane, and adamantane.

The term “heterocycle” or “heterocycloalkyl” refers to a single saturated or partially unsaturated ring that has at least one atom other than carbon in the ring, wherein the atom is selected from the group consisting of oxygen, nitrogen, and sulfur; the term also includes multiple condensed ring systems that have at least one such saturated or partially unsaturated ring, which multiple condensed ring systems are further described below. Thus, the term includes single saturated or partially unsaturated rings (e.g., 3, 4, 5, 6 or 7-membered rings) from about 1 to 6 carbon atoms and from about 1 to 3 heteroatoms selected from the group consisting of oxygen, nitrogen, and sulfur in the ring. The sulfur and nitrogen atoms may also be present in their oxidized forms. Exemplary heterocycles include but are not limited to azetidinyl, tetrahydrofuranyl and piperidinyl. The term “heterocycle” or “heterocycloalkyl” also includes multiple condensed ring systems (e.g., ring systems comprising 2, 3 or 4 rings) wherein a single heterocycle ring (as defined above) can be condensed with one or more groups selected from cycloalkyl, aryl, and heterocycle to form the multiple condensed ring system. The rings of the multiple condensed ring system can be connected to each other via fused, spiro and bridged bonds when allowed by valency requirements. It is to be understood that the individual rings of the multiple condensed ring system may be connected in any order relative to one another. It is also to be understood that the point of attachment of a multiple condensed ring system (as defined above for a heterocycle) can be at any position of the multiple condensed ring system including a heterocycle, aryl and carbocycle portion of the ring. In one embodiment the term heterocycle includes a 3-15 membered heterocycle. In one embodiment the term heterocycle includes a 3-10 membered heterocycle. In one embodiment the term heterocycle includes a 3-8 membered heterocycle. In one embodiment the term heterocycle includes a 3-7 membered heterocycle. In one embodiment the term heterocycle includes a 3-6 membered heterocycle. In one embodiment the term heterocycle includes a 4-6 membered heterocycle. In one embodiment the term heterocycle includes a 3-10 membered monocyclic or bicyclic heterocycle comprising 1 to 4 heteroatoms. In one embodiment the term heterocycle includes a 3-8 membered monocyclic or bicyclic heterocycle heterocycle comprising 1 to 3 heteroatoms. In one embodiment the term heterocycle includes a 3-6 membered monocyclic heterocycle comprising 1 to 2 heteroatoms. In one embodiment the term heterocycle includes a 4-6 membered monocyclic heterocycle comprising 1 to 2 heteroatoms. Exemplary heterocycles include, but are not limited to aziridinyl, azetidinyl, pyrrolidinyl, piperidinyl, homopiperidinyl, morpholinyl, thiomorpholinyl, piperazinyl, tetrahydrofuranyl, dihydrooxazolyl, tetrahydropyranyl, tetrahydrothiopyranyl, 1,2,3,4-tetrahydroquinolyl, benzoxazinyl, dihydrooxazolyl, chromanyl, 1,2-dihydropyridinyl, 2,3-dihydrobenzofuranyl, 1,3-benzodioxolyl, 1,4-benzodioxanyl, spiro[cyclopropane-1,1′-isoindolinyl]-3′-one, isoindolinyl-1-one, 2-oxa-6-azaspiro[3.3]heptanyl, imidazolidin-2-one imidazolidine, pyrazolidine, butyrolactam, valerolactam, imidazolidinone, hydantoin, dioxolane, phthalimide, and 1,4-dioxane.

The term “halo” or “halogen” refers to bromo, chloro, fluoro or iodo. In some embodiments, halogen refers to chloro or fluoro.

The term “aryl” as used herein refers to a single all carbon aromatic ring or a multiple condensed all carbon ring system wherein at least one of the rings is aromatic. For example, in certain embodiments, an aryl group has 6 to 20 carbon atoms, 6 to 14 carbon atoms, 6 to 12 carbon atoms, or 6 to 10 carbon atoms. Aryl includes a phenyl radical. Aryl also includes multiple condensed carbon ring systems (e.g., ring systems comprising 2, 3 or 4 rings) having about 9 to 20 carbon atoms in which at least one ring is aromatic and wherein the other rings may be aromatic or not aromatic (i.e., cycloalkyl. The rings of the multiple condensed ring system can be connected to each other via fused, spiro and bridged bonds when allowed by valency requirements. It is to be understood that the point of attachment of a multiple condensed ring system, as defined above, can be at any position of the ring system including an aromatic or a carbocycle portion of the ring. Non-limiting examples of aryl groups include, but are not limited to, phenyl, indenyl, indanyl, naphthyl, 1, 2, 3, 4-tetrahydronaphthyl, anthracenyl, and the like.

The term “aryl-amino” as used herein refers to an amino group in which one or more hydrogen atom has been replaced with an aryl group as defined above. Non-limiting examples of aryl-amino groups include, but are not limited to, anilinyl that is also referred to as phenylamino (which is (C₆H₅)—NH—), indenylamino, indanylamino, naphthylamino, and the like.

The term “heteroaryl” as used herein refers to a single aromatic ring that has at least one atom other than carbon in the ring, wherein the atom is selected from the group consisting of oxygen, nitrogen and sulfur; “heteroaryl” also includes multiple condensed ring systems that have at least one such aromatic ring, which multiple condensed ring systems are further described below. Thus, “heteroaryl” includes single aromatic rings of from about 1 to 6 carbon atoms and about 1-4 heteroatoms selected from the group consisting of oxygen, nitrogen and sulfur. The sulfur and nitrogen atoms may also be present in an oxidized form provided the ring is aromatic. Exemplary heteroaryl ring systems include but are not limited to pyridyl, pyrimidinyl, oxazolyl or furyl. “Heteroaryl” also includes multiple condensed ring systems (e.g., ring systems comprising 2, 3 or 4 rings) wherein a heteroaryl group, as defined above, is condensed with one or more rings selected from cycloalkyl, aryl, heterocycle, and heteroaryl. It is to be understood that the point of attachment for a heteroaryl or heteroaryl multiple condensed ring system can be at any suitable atom of the heteroaryl or heteroaryl multiple condensed ring system including a carbon atom and a heteroatom (e.g., a nitrogen). Exemplary heteroaryls include but are not limited to pyridyl, pyrrolyl, pyrazinyl, pyrimidinyl, pyridazinyl, pyrazolyl, thienyl, indolyl, imidazolyl, triazolyl, tetrazolyl, oxazolyl, isoxazolyl, thiazolyl, furyl, oxadiazolyl, thiadiazolyl, quinolyl, isoquinolyl, benzothiazolyl, benzoxazolyl, indazolyl, quinoxalyl, and quinazolyl.

As used herein, the term “heteroatom” is meant to include oxygen (O), nitrogen (N), sulfur (S) and silicon (Si).

The term “heteroaryl-amino” as used herein refers to an amino group in which one or more hydrogen atom has been replaced with heteroaryl group as defined above. Non-limiting examples of heteroaryl-amino groups include, but are not limited to, pyridylamino, pyrrolylamino, and the like.

The term “alkyl-amino” as used herein refers to an amino group in which one or more hydrogen atom has been replaced with alkyl group as defined above. Non-limiting examples of alkyl-amino groups include, but are not limited to, methylamino, ethylamino, and the like.

The term “alkoxycarbonyl” as used herein refers to a group (alkyl)-O—C(═O)—, wherein the term alkyl has the meaning defined herein.

The term “alkanoyloxy” as used herein refers to a group (alkyl)-C(═O)—O—, wherein the term alkyl has the meaning defined herein.

The term “amino acid” comprises the residues of the natural amino acids (e.g. Ala (A), Arg (R), Asn (N), Asp (D), Cys (C), Glu (E), Gln (Q), Gly (G), His (H), Ile (I), Leu (L), Lys (K), Met (M), Phe (F), Pro (P), Ser (S), Thr (T), Trp (W), Tyr (Y), Val (V), and Hydroxylysine (Hyl)) in D or L form, as well as unnatural amino acids (e.g., non-limiting examples include, but are not limited to, homoarginine (hArg), 4-phenyl-phenylalanine (Bip or 4-phenyl-Phe), 4-(2-methoxyphenyl)-Phe, 5-hydroxy-tryptophan, 5-methoxy-tryptophan, 2,3-Diaminopropionic acid (Dap), 2,4-Diaminobutyric Acid (Dab), Ornithine (Orn), gamma-amino-butyric acid (GABA), 3-amino-cyclohexanecarboxylic acid (ACHC), homolysine (hLys), 3,5-diiodo-tyrosine, 3,5-Dibromotyrosine, 3-Nitro-tyrosine, Homotyrosine, 2-Hydroxyphenylalanine, meta-Tyrosine, 3-Chlorotyrosine, Fluorophenylalanine, Pentafluorophenylalanine, Acetylphenylalanine, 4-Carboxy-phenylalanine, N-Methyl-proline, 2-Methylproline, 3-Methyl-histidine, 1-Methyl-histidine, 5-Fluoro-tryptophan, PyrAla, ThiAla, (pCl)Phe, (pNO2)Phe, ε-Aminocaproic acid, Met[O₂], dehydPro, (3I)Tyr, norleucine (Nle), para-I-phenylalanine ((pI)Phe), 2-napthylalanine (2-Nal), β-cyclohexylalanine (Cha), β-alanine (β-Ala), phosphoserine, phosphothreonine, phosphotyrosine, hydroxyproline, gamma-carboxyglutamate; hippuric acid, octahydroindole-2-carboxylic acid, statine, 1,2,3,4,-tetrahydroisoquinoline-3-carboxylic acid (Tic), penicillamine, citruline, α-methyl-alanine, para-benzoylphenylalanine, phenylglycine, propargylglycine, sarcosine, and tert-butylglycine) in D or L form. The term “amino acid side chain” refers to the functional group attached to the alpha carbon of an alpha amino acid. For example, the side chain of Ala is CH₃—; the side chain of Cys is HS—CH₂—; the side chain of Ser is HO—CH₂—; the side chain of Phe is benzyl. Amino acid side chains are well known in the art, amino acids are usually categorized based on side chain property into a few groups. For example, amino acid side chain may be polar or nonpolar. Amino acid side chain may be charged (positively or negatively) or noncharged. The term “amino acid” also comprises natural and unnatural amino acids bearing a conventional amino protecting group (e.g. acetyl or benzyloxycarbonyl), as well as natural and unnatural amino acids protected at the carboxy terminus (e.g. as a (C₁-C₆)alkyl, phenyl or benzyl ester or amide; or as an α-methylbenzyl amide). Other suitable amino and carboxy protecting groups are known to those skilled in the art (See for example, T.W. Greene, Protecting Groups In Organic Synthesis; Wiley: New York, 1981, and references cited therein). The term “amino acid” also comprises alpha-amino acids (e.g., glycine: NH₂CH₂COOH), beta-amino acids (e.g., beta-alanine: NH₂(CH₂)₂COOH), gamma-amino acids (e.g., gamma-aminobutyric acid: NH₂(CH₂)₃COOH), delta-amino acids, epsilon-amino acids and zeta-amino acids. The carbon atom next to the carboxyl group is referred to as alpha carbon. Amino acids containing an amino group bonded directly to the alpha carbon are referred to as alpha amino acids. The second carbon atom is referred to as the beta carbon. Amino acids containing an amino group bonded directly to the beta carbon are referred to as beta amino acids. The system continues naming in alphabetical order with Greek letters.

The term “peptide” or “peptidomimetic” describes a sequence of 2 to 25 amino acids (e.g. as defined hereinabove) or peptidyl residues. In certain embodiments, the peptide described herein is 4 amino acids in length. In certain embodiments, the peptide described herein is 5 amino acids in length. In certain embodiments, the peptide described herein is 6 amino acids in length. In certain embodiments, the peptide described herein is 7 amino acids in length. In certain embodiments, the peptide described herein is 8 amino acids in length. Peptide derivatives can be prepared as disclosed in U.S. Pat. Nos. 4,612,302; 4,853,371; and 4,684,620, or as described in the Examples herein below. Peptide sequences specifically recited herein are written with the amino terminus on the left and the carboxy terminus on the right. Each residue of amino acid of the “peptide” or “peptidomimetic” is independently and optionally substituted or unsubstituted. The term “dipeptide” refers to a peptide comprising two amino acids joined through an amide bond. The term “tripeptide” means a peptide comprising three amino acids joined through two amide bonds. The term “tetrapeptide” means a peptide comprising four amino acids joined through three amide bonds. The term “pentapeptide” means a peptide comprising five amino acids joined through four amide bonds. The term “hexapeptide” means a peptide comprising six amino acids joined through five amide bonds. The term “heptapeptide” means a peptide comprising seven amino acids joined through six amide bonds. The term “octapeptide” means a peptide comprising eight amino acids joined through seven amide bonds. It is understood that the N-terminus of a peptide (the amino group of the first amino acid residue in the peptide) could be a free primary amine group NH₂—, which may be positively charged as H₃N⁺— under certain suitable conditions including physiological conditions. Alternatively, the N-terminus of a peptide may be a capped amine group, for example, via acylation or formylation. For instance, the N-terminus may be a capped amine R^{N}—C(═O)—NH—, wherein R^{N} is H, optionally substituted (C₃-C₆)cycloalkyl or (C₁-C₄)alkyl. It is also understood that the C-terminus of a peptide (the carboxy group of the last amino acid residue in the peptide) may be a free carboxyl group —COOH, which may be negatively charged as —COO— under suitable conditions. Alternatively, the C-terminus of a peptide may be capped, for example, via amidation. The C-terminus of a peptide may be amidated as described herein (e.g., amidated with an aryl-amino, heteroaryl-amino, or alkyl-amino group). Hence, the C-terminus of a peptide may be a free carboxyl group —COOH, or its amide thereof.

As used herein, the term “residue of an amino acid, dipeptide or tripeptide” means a portion of an amino acid, dipeptide or tripeptide. For example, scaffold segment may comprise residues of peptides, wherein certain atoms (e.g., H or OH) have been removed to link the amino acids via a peptide bond.

The terms “treat” and “treatment” refer to both therapeutic treatment and prophylactic or preventative measures, wherein the object is to prevent or slow down (lessen) an undesired physiological change or disorder. For purposes of this invention, beneficial or desired clinical results include, but are not limited to, alleviation of symptoms, diminishment of extent of disease, stabilized (i.e., not worsening) state of disease, delay or slowing of disease progression, amelioration or palliation of the disease state, and remission (whether partial or total), whether detectable or undetectable. For example, the onset of a disorder or disease is prevented or delayed. The progression of a disease is slowed or stopped. “Treatment” can also mean prolonging survival as compared to expected survival if not receiving treatment. Those in need of treatment include those already with the condition or disorder as well as those prone to have the condition or disorder or those in which the condition or disorder is to be prevented.

The phrase “therapeutically effective amount” means an amount of a compound (e.g., peptide) of the present invention that (i) treats the particular disease, condition, or disorder, (ii) attenuates, ameliorates, or eliminates one or more symptoms of the particular disease, condition, or disorder, or (iii) prevents or delays the onset of one or more symptoms of the particular disease, condition, or disorder described herein.

The term “prediction” (and variations such as predicting) is used herein to refer to the likelihood that a patient will respond either favorably or unfavorably to a given therapy (e.g., a compound or peptide as described herein). In one embodiment, the prediction relates to the extent of such responses. Predictive methods described herein can be used to make treatment decisions by choosing the most appropriate treatment for a particular patient. The predictive methods may be used in predicting if a patient is likely to respond favorably to a treatment regimen, or whether long-term survival of the patient following a therapeutic regimen is likely.

The term “agonist” or “agonistic agent” as used herein refers to a compound (e.g., peptide) that binds and activates a receptor to produce a biological response. Thus, the biological response triggered by an agonistic compound for receptor EphA4 may be similar to that elicited by the natural ligands (such as ephrinA5). For example, an EphA4 agonist compound as described herein binds and activates EphA4, triggering EphA4 related biological responses, including but not limited to EphA4 receptor phosphorylation and/or initiation of signal transduction along the EphA4 associated signaling pathway(s).

The term “mammal” as used herein refers to, e.g., humans, higher non-human primates, rodents, domestic, cows, horses, pigs, sheep, dogs and cats. In one embodiment, the mammal is a human.

Stereochemical definitions and conventions used herein generally follow S. P. Parker, Ed., McGraw-Hill Dictionary of Chemical Terms (1984) McGraw-Hill Book Company, New York; and Eliel, E. and Wilen, S., “Stereochemistry of Organic Compounds”, John Wiley & Sons, Inc., New York, 1994. The compounds of the invention can contain asymmetric or chiral centers, and therefore exist in different stereoisomeric forms. It is intended that all stereoisomeric forms of the compounds of the invention, including but not limited to, diastereomers, enantiomers and atropisomers, as well as mixtures thereof such as racemic mixtures, form part of the present invention. Many organic compounds exist in optically active forms, i.e., they have the ability to rotate the plane of plane-polarized light. In describing an optically active compound, the prefixes D and L, or R and S, are used to denote the absolute configuration of the molecule about its chiral center(s). The prefixes d and l or (+) and (−) are employed to designate the sign of rotation of plane-polarized light by the compound, with (−) or l meaning that the compound is levorotatory. A compound prefixed with (+) or d is dextrorotatory. For a given chemical structure, these stereoisomers are identical except that they are mirror images of one another. A specific stereoisomer can also be referred to as an enantiomer, and a mixture of such isomers is often called an enantiomeric mixture. A 50:50 mixture of enantiomers is referred to as a racemic mixture or a racemate, which can occur where there has been no stereoselection or stereospecificity in a chemical reaction or process. The terms “racemic mixture” and “racemate” refer to an equimolar mixture of two enantiomeric species, devoid of optical activity.

It will be appreciated by those skilled in the art that certain compounds described herein have a chiral center may exist in and be isolated in optically active and racemic forms. Some compounds may exhibit polymorphism. It is to be understood that the present invention encompasses any racemic, optically-active, polymorphic, or stereoisomeric form, or mixtures thereof, of a compound of the invention, which possess the useful properties described herein, it being well known in the art how to prepare optically active forms (for example, by resolution of the racemic form by recrystallization techniques, by synthesis from optically-active starting materials, by chiral synthesis, or by chromatographic separation using a chiral stationary phase).

When a bond in a compound formula herein is drawn in a non-stereochemical manner (e.g. flat), the atom to which the bond is attached includes all stereochemical possibilities. For example, in certain embodiments, all stereochemical possibilities are included for the following compounds:

| Com- | |
|---|---|
| pound | Structure |
| 1f | 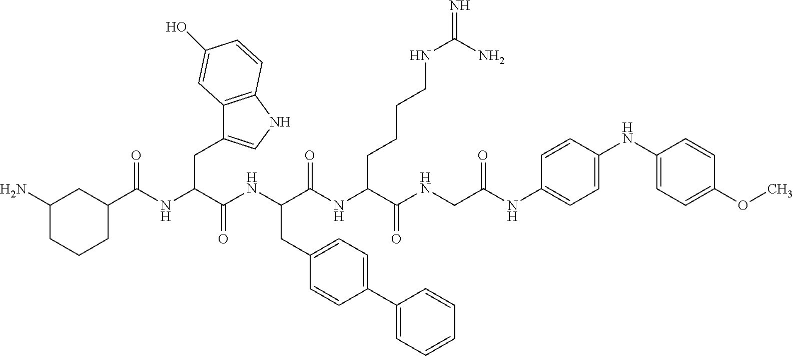 [SMILES: COc1ccc(Nc2ccc(NC(=O)CNC(=O)C(CCCCNC(=N)N)NC(=O)C(Cc3ccc(-c4ccccc4)cc3)NC(=O)C(Cc3c[nH]c4ccc(O)cc34)NC(=O)C3CCCC(N)C3)cc2)cc1] |
| 2f | 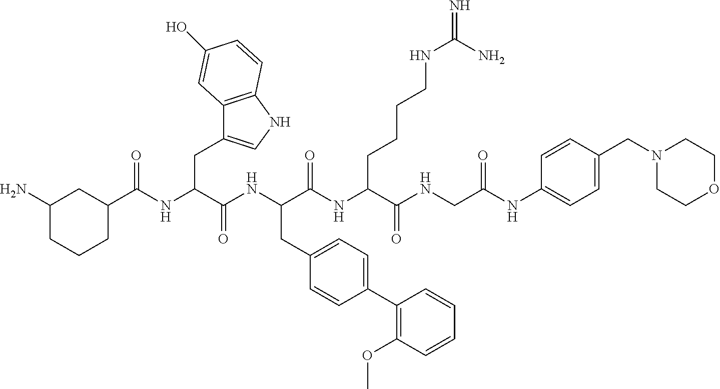 [SMILES: COc1ccccc1-c1ccc(CC(NC(=O)C(Cc2c[nH]c3ccc(O)cc23)NC(=O)C2CCCC(N)C2)C(=O)NC(CCCCNC(=N)N)C(=O)NCC(=O)Nc2ccc(CN3CCOCC3)cc2)cc1] |
| 3f | 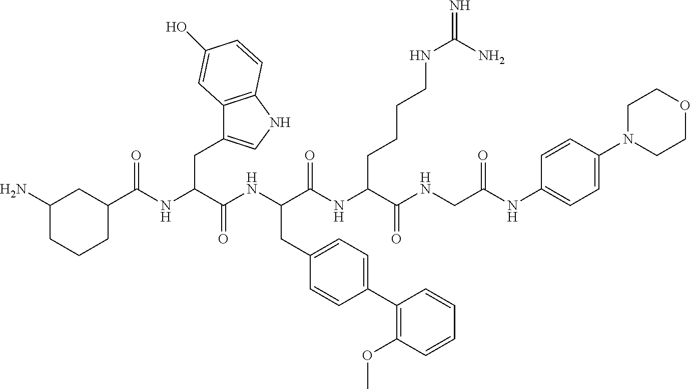 [SMILES: COc1ccccc1-c1ccc(CC(NC(=O)C(Cc2c[nH]c3ccc(O)cc23)NC(=O)C2CCCC(N)C2)C(=O)NC(CCCCNC(=N)N)C(=O)NCC(=O)Nc2ccc(N3CCOCC3)cc2)cc1] |
| 4f | 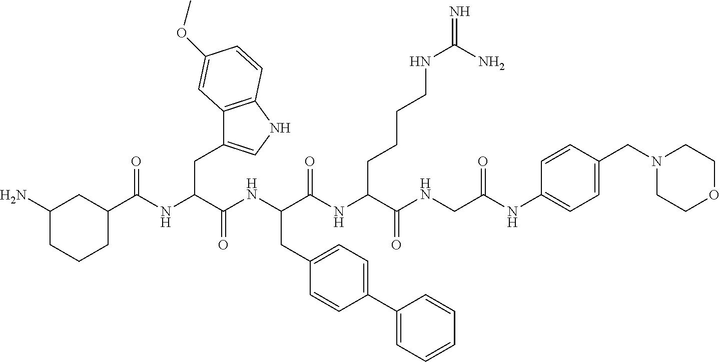 [SMILES: COc1ccc2[nH]cc(CC(NC(=O)C3CCCC(N)C3)C(=O)NC(Cc3ccc(-c4ccccc4)cc3)C(=O)NC(CCCCNC(=N)N)C(=O)NCC(=O)Nc3ccc(CN4CCOCC4)cc3)c2c1] |
| 5f | 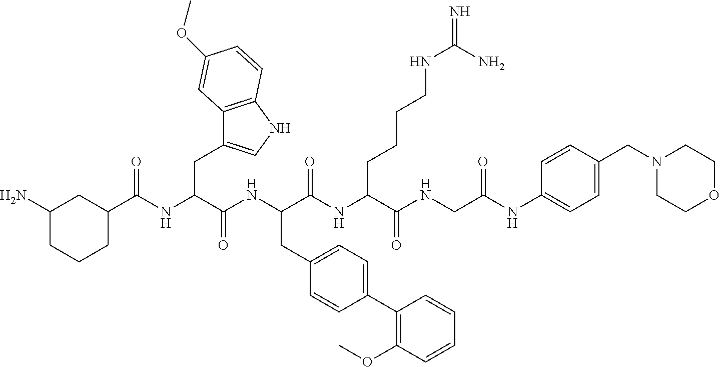 [SMILES: COc1ccc2[nH]cc(CC(NC(=O)C3CCCC(N)C3)C(=O)NC(Cc3ccc(-c4ccccc4OC)cc3)C(=O)NC(CCCCNC(=N)N)C(=O)NCC(=O)Nc3ccc(CN4CCOCC4)cc3)c2c1] |
| 6f | 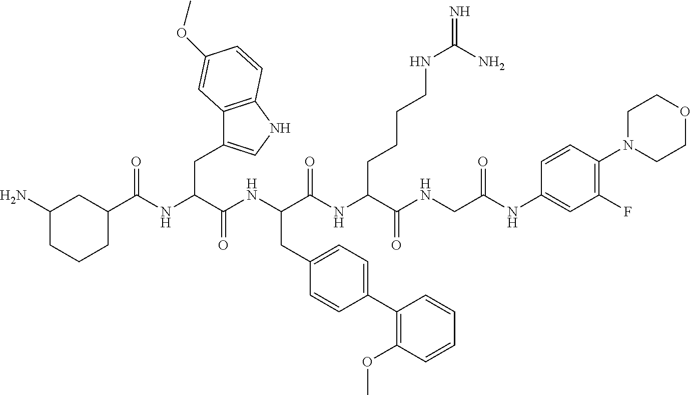 [SMILES: COc1ccc2[nH]cc(CC(NC(=O)C3CCCC(N)C3)C(=O)NC(Cc3ccc(-c4ccccc4OC)cc3)C(=O)NC(CCCCNC(=N)N)C(=O)NCC(=O)Nc3ccc(N4CCOCC4)c(F)c3)c2c1] |
| 7f | 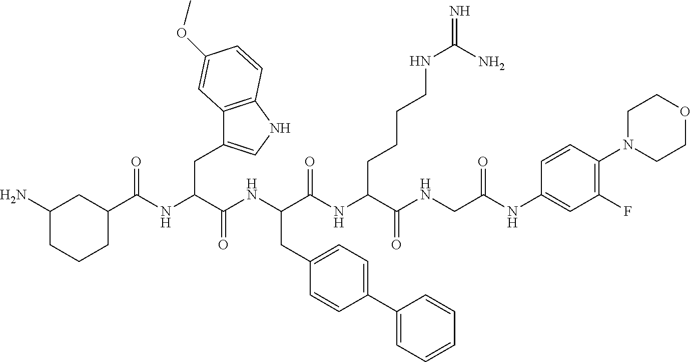 [SMILES: COc1ccc2[nH]cc(CC(NC(=O)C3CCCC(N)C3)C(=O)NC(Cc3ccc(-c4ccccc4)cc3)C(=O)NC(CCCCNC(=N)N)C(=O)NCC(=O)Nc3ccc(N4CCOCC4)c(F)c3)c2c1] |
| 8f | 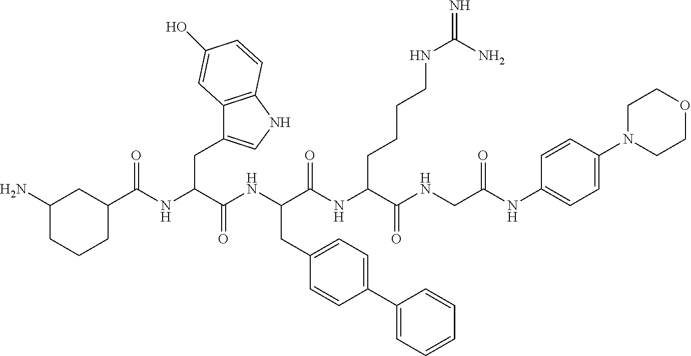 [SMILES: N=C(N)NCCCCC(NC(=O)C(Cc1ccc(-c2ccccc2)cc1)NC(=O)C(Cc1c[nH]c2ccc(O)cc12)NC(=O)C1CCCC(N)C1)C(=O)NCC(=O)Nc1ccc(N2CCOCC2)cc1] |
| 9f | 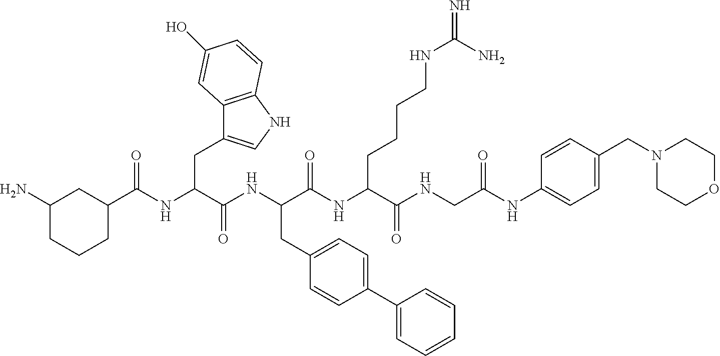 [SMILES: N=C(N)NCCCCC(NC(=O)C(Cc1ccc(-c2ccccc2)cc1)NC(=O)C(Cc1c[nH]c2ccc(O)cc12)NC(=O)C1CCCC(N)C1)C(=O)NCC(=O)Nc1ccc(CN2CCOCC2)cc1] |
| 10f | 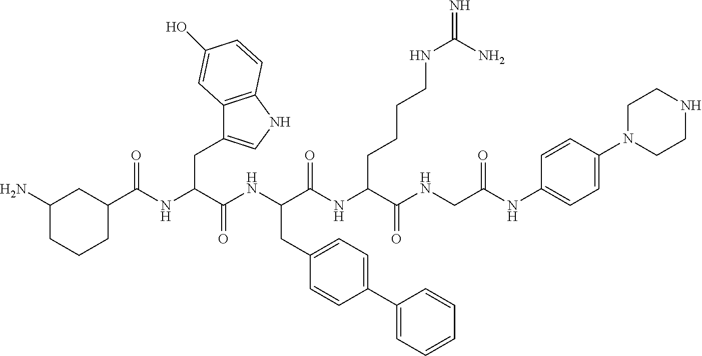 [SMILES: N=C(N)NCCCCC(NC(=O)C(Cc1ccc(-c2ccccc2)cc1)NC(=O)C(Cc1c[nH]c2ccc(O)cc12)NC(=O)C1CCCC(N)C1)C(=O)NCC(=O)Nc1ccc(N2CCNCC2)cc1] |
| 11f | 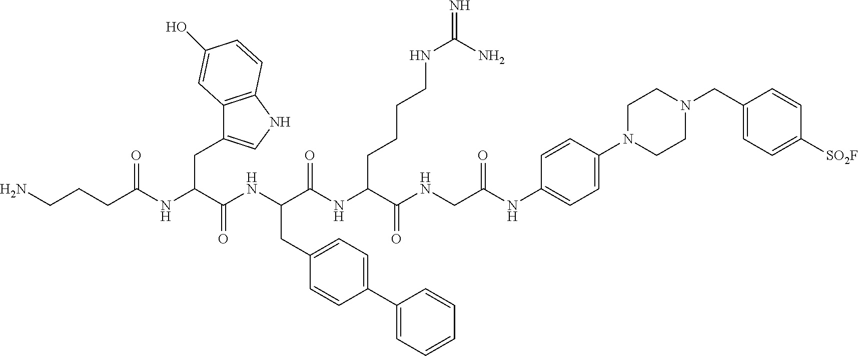 [SMILES: N=C(N)NCCCCC(NC(=O)C(Cc1ccc(-c2ccccc2)cc1)NC(=O)C(Cc1c[nH]c2ccc(O)cc12)NC(=O)CCCN)C(=O)NCC(=O)Nc1ccc(N2CCN(Cc3ccc(S(=O)(=O)F)cc3)CC2)cc1] |
| 12f | 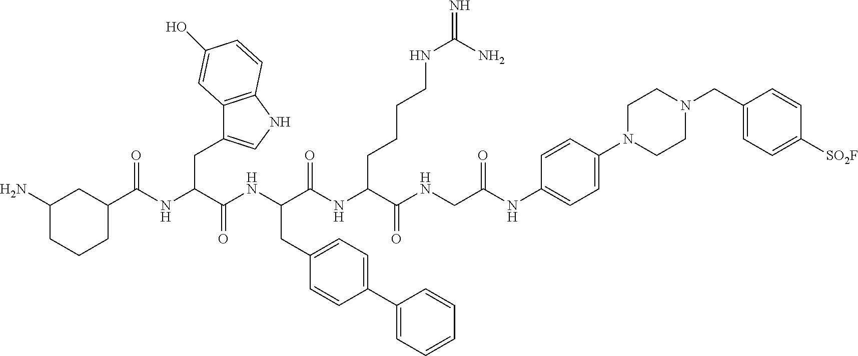 [SMILES: N=C(N)NCCCCC(NC(=O)C(Cc1ccc(-c2ccccc2)cc1)NC(=O)C(Cc1c[nH]c2ccc(O)cc12)NC(=O)C1CCCC(N)C1)C(=O)NCC(=O)Nc1ccc(N2CCN(Cc3ccc(S(=O)(=O)F)cc3)CC2)cc1] |
| 13f | 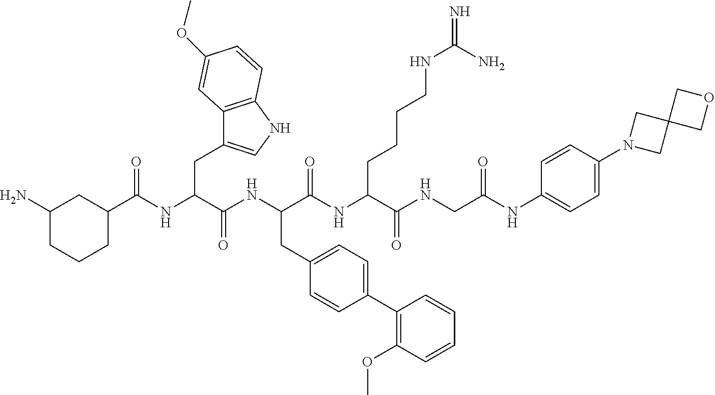 [SMILES: COc1ccc2[nH]cc(CC(NC(=O)C3CCCC(N)C3)C(=O)NC(Cc3ccc(-c4ccccc4OC)cc3)C(=O)NC(CCCCNC(=N)N)C(=O)NCC(=O)Nc3ccc(N4CC5(COC5)C4)cc3)c2c1] |
| 14f | 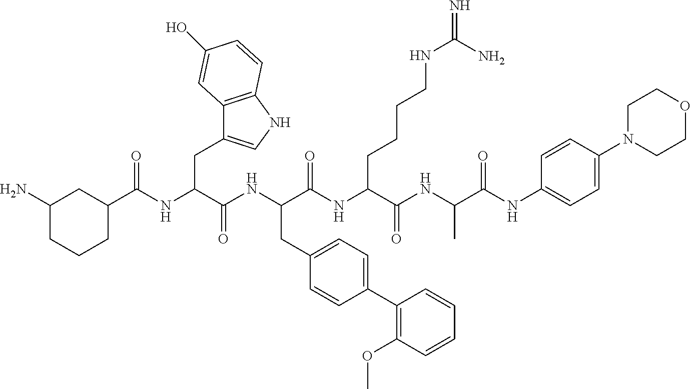 [SMILES: COc1ccccc1-c1ccc(CC(NC(=O)C(Cc2c[nH]c3ccc(O)cc23)NC(=O)C2CCCC(N)C2)C(=O)NC(CCCCNC(=N)N)C(=O)NC(C)C(=O)Nc2ccc(N3CCOCC3)cc2)cc1] |
| 15f | 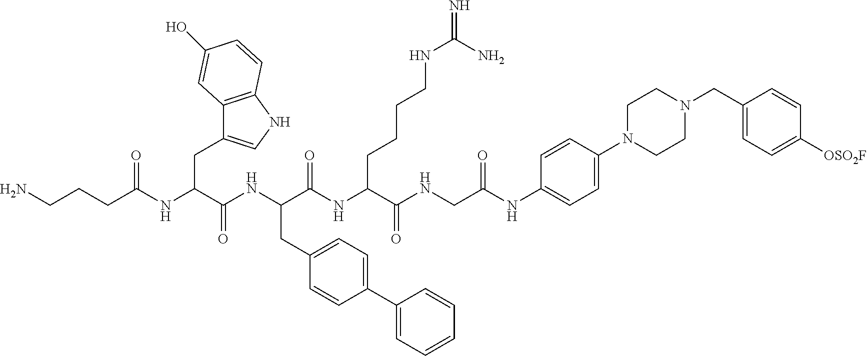 |
| 16f | 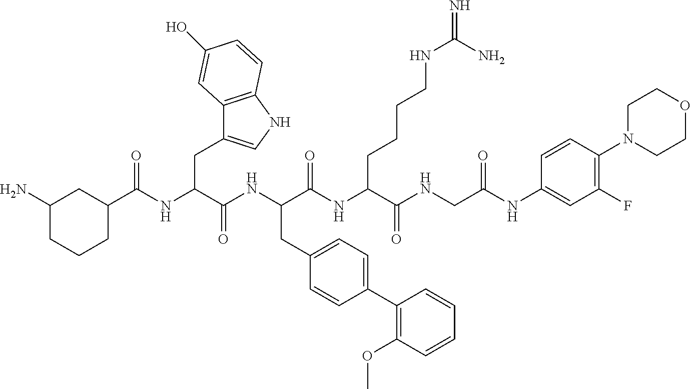 [SMILES: COc1ccccc1-c1ccc(CC(NC(=O)C(Cc2c[nH]c3ccc(O)cc23)NC(=O)C2CCCC(N)C2)C(=O)NC(CCCCNC(=N)N)C(=O)NCC(=O)Nc2ccc(N3CCOCC3)c(F)c2)cc1] |
| 500f | 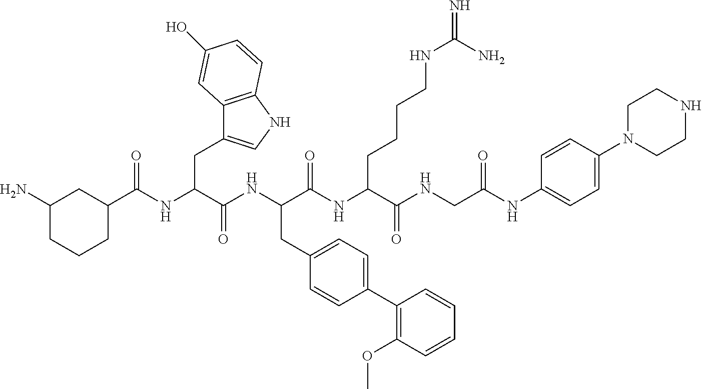 [SMILES: COc1ccccc1-c1ccc(CC(NC(=O)C(Cc2c[nH]c3ccc(O)cc23)NC(=O)C2CCCC(N)C2)C(=O)NC(CCCCNC(=N)N)C(=O)NCC(=O)Nc2ccc(N3CCNCC3)cc2)cc1] |

When a bond in a compound formula herein is drawn in a defined stereochemical manner (e.g. bold, bold-wedge, dashed or dashed-wedge), it is to be understood that the atom to which the stereochemical bond is attached is enriched in the absolute stereoisomer depicted unless otherwise noted. In one embodiment, the compound may be at least 51% the absolute stereoisomer depicted. In another embodiment, the compound may be at least 60% the absolute stereoisomer depicted. In another embodiment, the compound may be at least 80% the absolute stereoisomer depicted. In another embodiment, the compound may be at least 90% the absolute stereoisomer depicted. In another embodiment, the compound may be at least 95 the absolute stereoisomer depicted. In another embodiment, the compound may be at least 99% the absolute stereoisomer depicted. For example, in certain embodiments, the atom to which the stereochemical bond is attached is enriched in the absolute stereoisomer depicted for the following compounds:

| Com- | |
|---|---|
| pound | Structure |
| 1 | 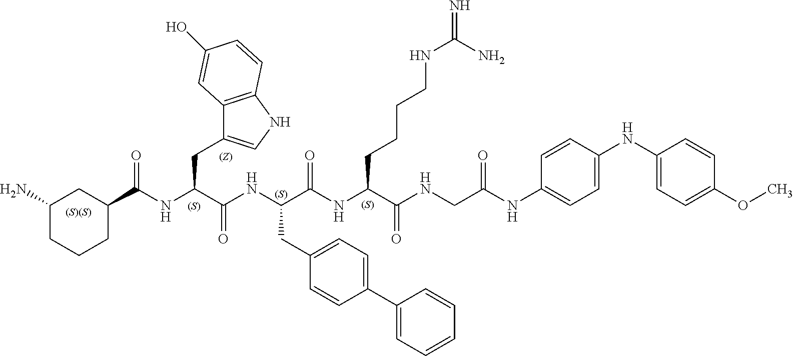 |
| 2 | 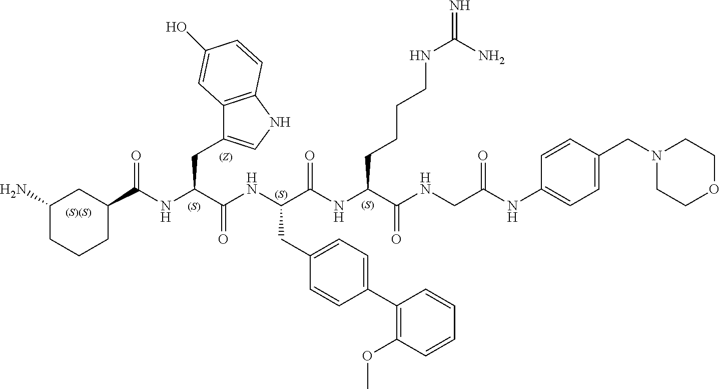 |
| 3 | 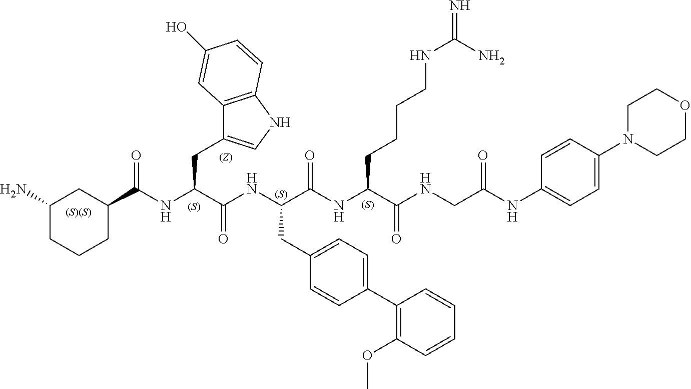 |
| 4 | 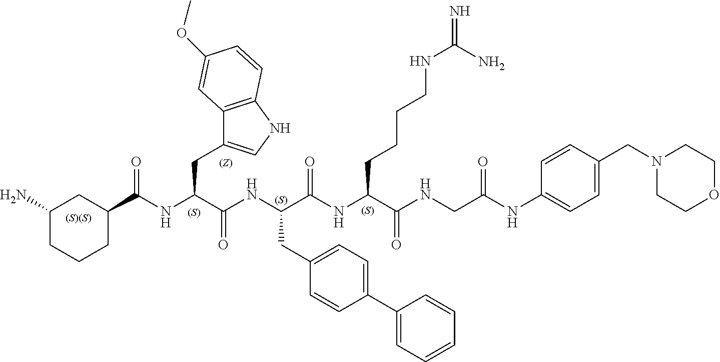 |
| 5 | 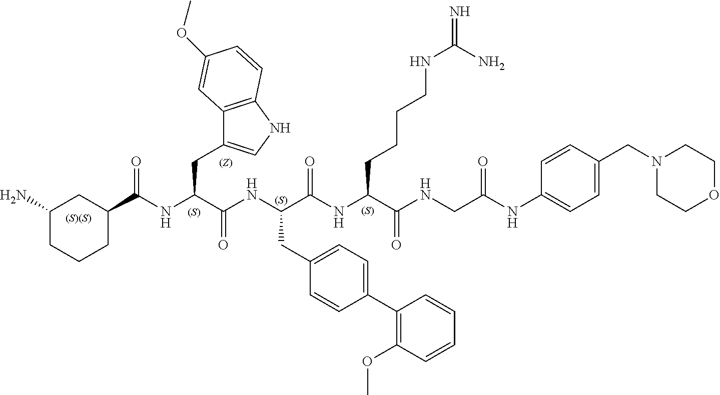 |
| 6 | 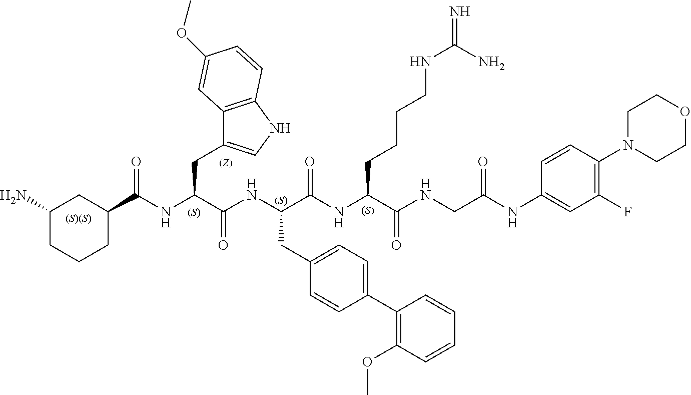 |
| 7 | 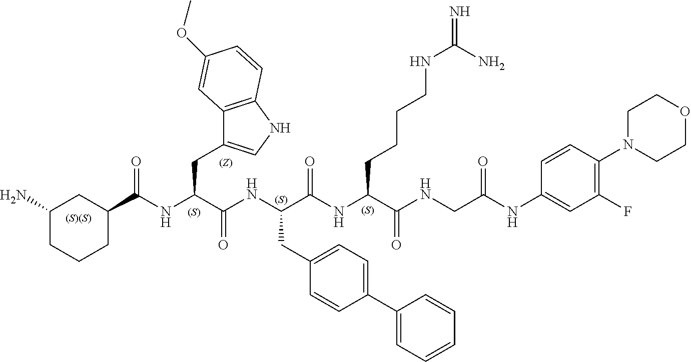 |
| 8 | 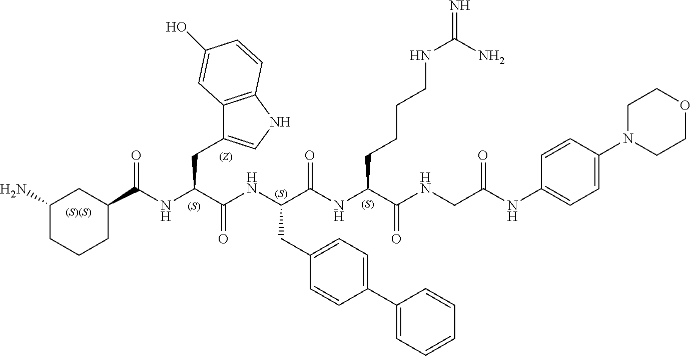 |
| 9 | 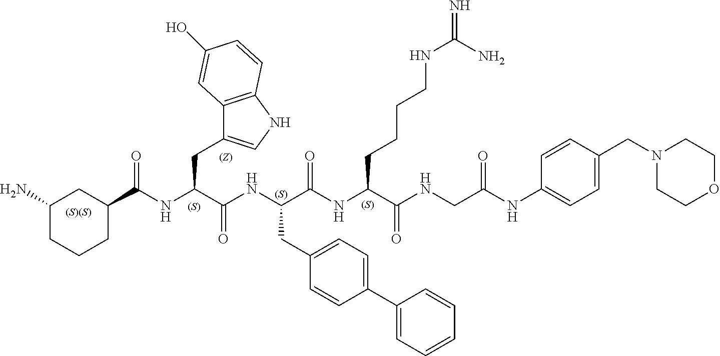 |
| 10 | 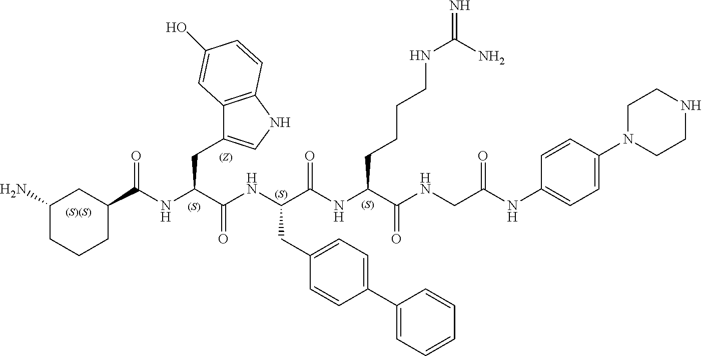 |
| 11 | 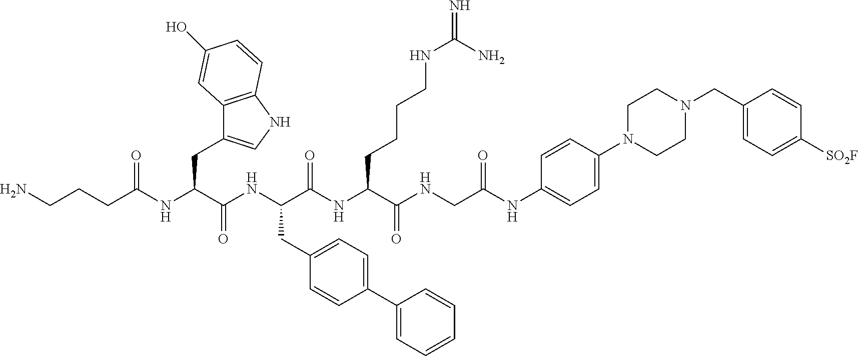 [SMILES: N=C(N)NCCCC[C@H](NC(=O)[C@H](Cc1ccc(-c2ccccc2)cc1)NC(=O)[C@H](Cc1c[nH]c2ccc(O)cc12)NC(=O)CCCN)C(=O)NCC(=O)Nc1ccc(N2CCN(Cc3ccc(S(=O)(=O)F)cc3)CC2)cc1] |
| 12 | 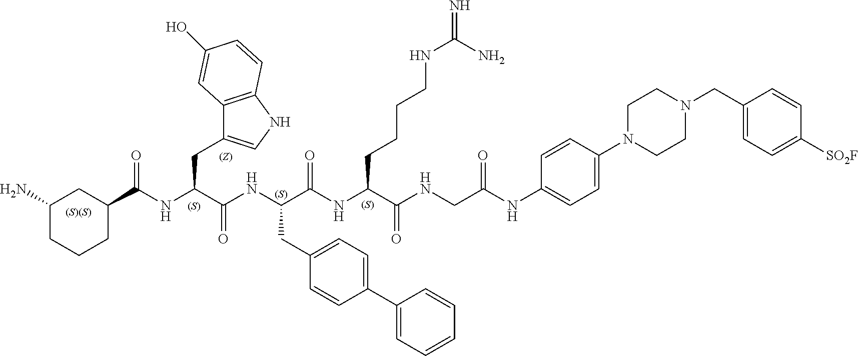 |
| 13 | 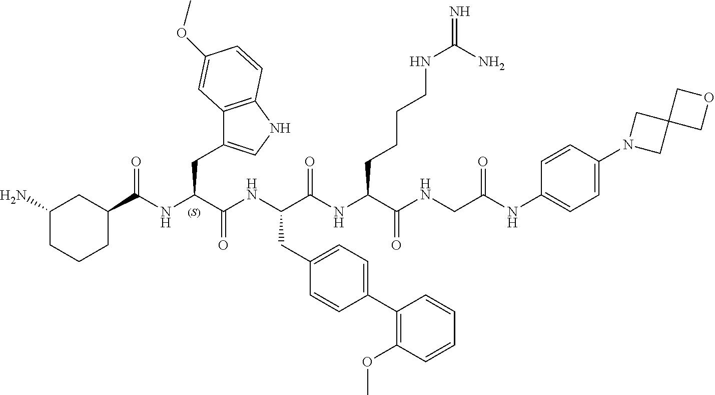 |
| 14 | 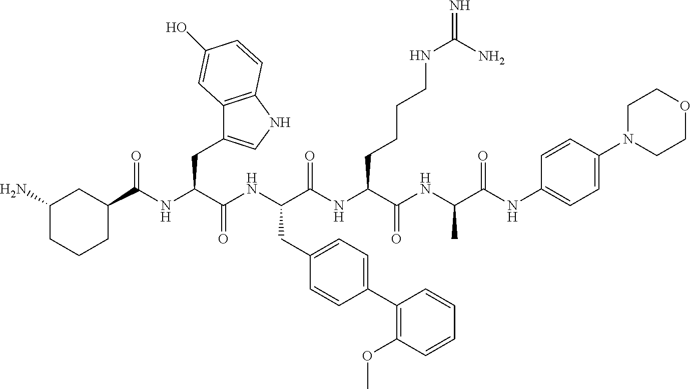 [SMILES: COc1ccccc1-c1ccc(C[C@H](NC(=O)[C@H](Cc2c[nH]c3ccc(O)cc23)NC(=O)[C@H]2CCC[C@H](N)C2)C(=O)N[C@@H](CCCCNC(=N)N)C(=O)N[C@H](C)C(=O)Nc2ccc(N3CCOCC3)cc2)cc1] |
| 15 | 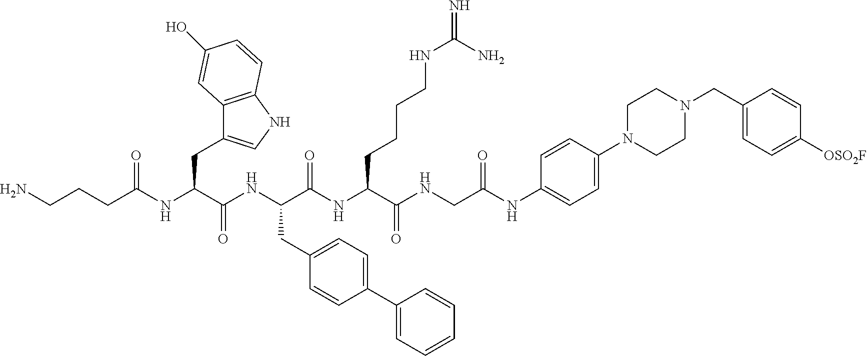 |
| 16 | 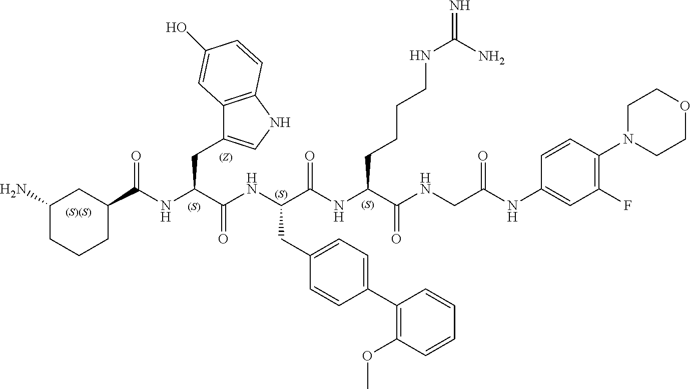 |
| 500 | 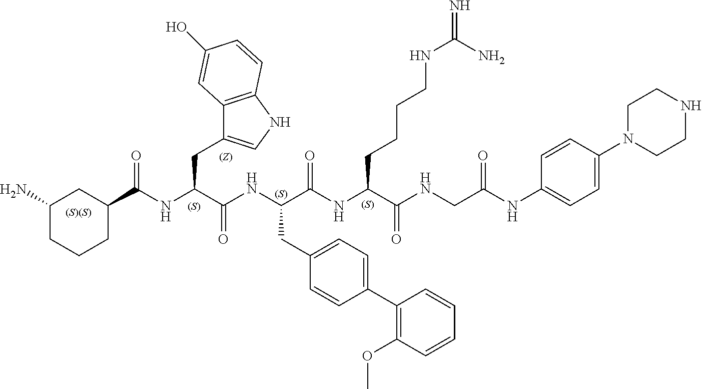 |

Compounds of the invention can be prepared using known starting materials and techniques or using starting materials and techniques that are analogous to those described herein. The invention will now be illustrated by the following non-limiting Examples.

### Example 1

Peptides were synthesized by using standard solid-phase synthesis protocol, using the BAL resin as anchoring for the C-terminal amines. Isothermal Titration Calorimetry (ITC) measurements were performed in a reverse fashion by titrating the protein into the ligand solution. Solution nuclear magnetic resonance (NMR) experiments were conducted on a 700 MHz Bruker Avance spectrometer equipped with a TCI cryoprobe. Each protein sample was dissolved into an NMR tube at a final concentration of 20 μM (1% DMSO-d6) in the presence of 20 μM of each compound.

TABLE 1
Representative agents and dissociation constant and NMR binding properties.

| | | | | | Δδ |
|---|---|---|---|---|---|
| | | | | | ¹³C^{ϵ}/¹H^{ϵ} |
| | | | AH | -TΔS | Met |
| ID | STRUCTURE | Kd (nM)- ITC | (kcal/mol) | (kcal/mol) | 164 |
| 2 | 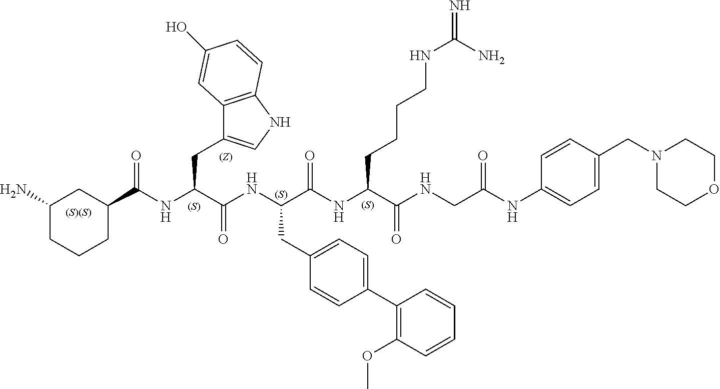 | 75 | −8.74 | −0.97 | + |
| 3 | 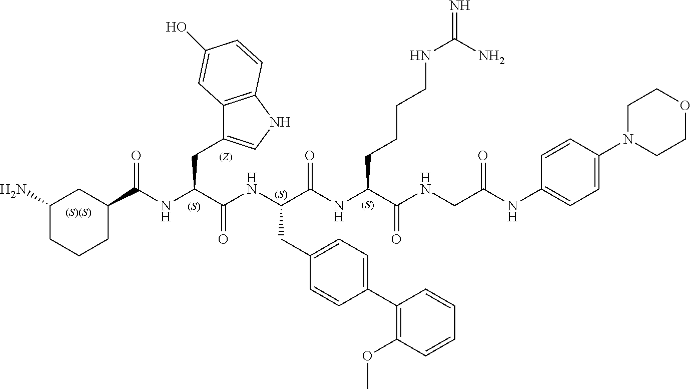 | 106 | −8.92 | −0.60 | + |
| 4 | 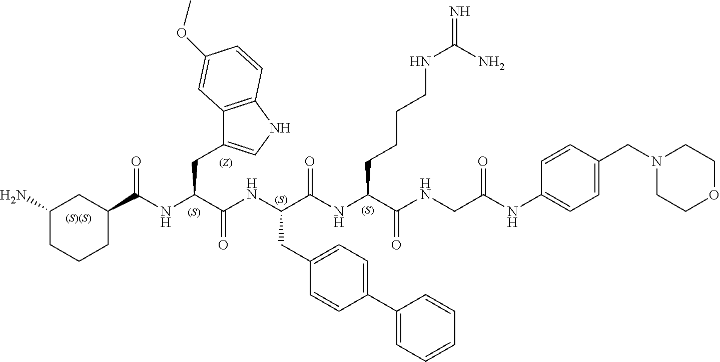 | 100 | −5.99 | −3.57 | +++ |
| 5 | 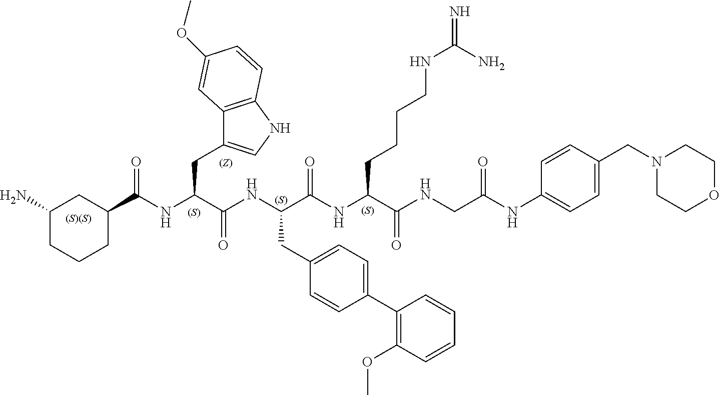 | 163 | −7.00 | −2.25 | ++ |
| 16 | 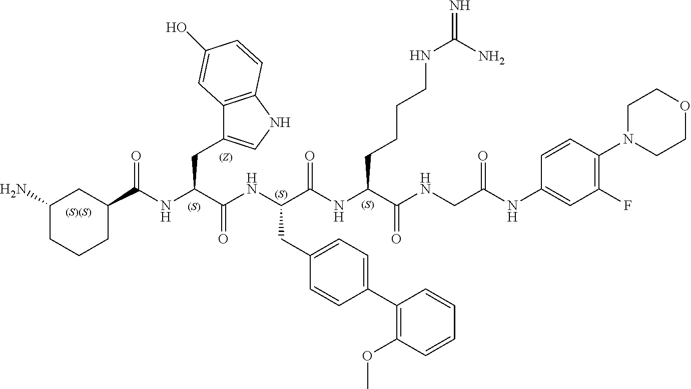 | 127 | −10.00 | 0.58 | + |
| 8 (150D4) | 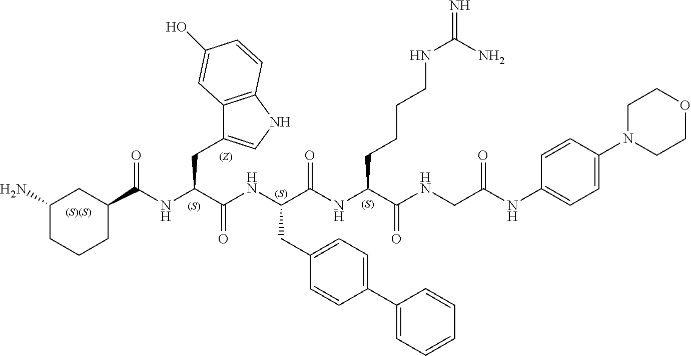 | 113 | −8.49 | −0.88 | ++++ |
| 9 | 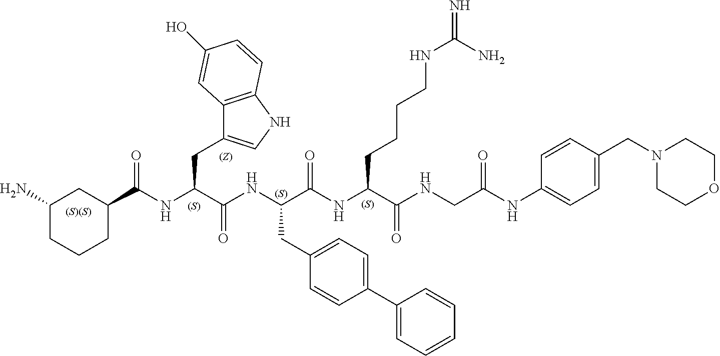 | 144 | −7.44 | −1.90 | +++ |
| 10 | 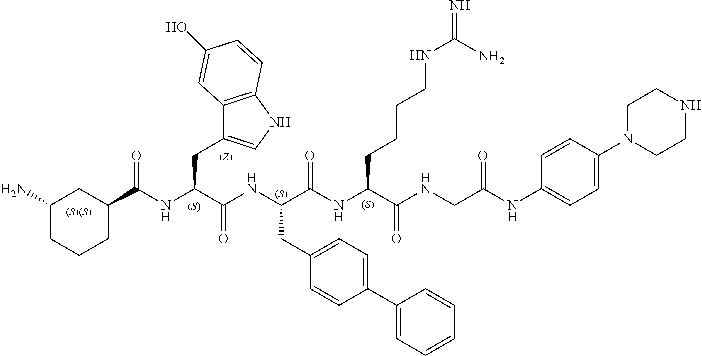 | 90 | −8.27 | −1.35 | ++++ |

Most potent agents bind in the low nanomolar range. The NMR column reflect chemical shift of Met 164 induced in [¹³C,¹H] HSQC experiments with ¹³C^{ϵ}-Met-labeled EphA4-LBD sample, collected in absence and presence of the agent.
Δδ values represent weight average perturbations observed in the ¹H and ¹³C dimensions, as described in the methods.
Δδ: −, no changes; 0< + <0.1 ppm; 0.1 ppm < ++ < 0.2 ppm; 0.2< +++ <0.25; ++++ >0.25 ppm.

### Example 2

### NMR-Guided Design of Potent and Selective EphA4 Agonistic Ligands

Recently High Throughput Screening (HTS) by NMR approach was developed in which a combinatorial library of potential binders is tested by protein NMR spectroscopy methods. A powerful extension of the approach is the focused HTS by NMR (fHTS by NMR) that consists of derivatizing each element of the combinatorial library with a known anchoring chemical moiety. In this Example, the approach was applied to derive potent, low molecular weight ligands capable of mimicking the interactions elicited by the ephrin ligands on the receptor tyrosine kinase EphA4. The agents bind with nanomolar affinity, trigger receptor activation in cellular assays with motor neurons, and provide remarkable motor neuron protection from Amyotrophic Lateral Sclerosis (ALS) patient derived astrocytes. Structural studies on the complex between EphA4 ligand binding domain and a most active agent provide insights on the mechanism of the agents at a molecular level. Taken together, the data form a strong foundation for the translation of these agents for the treatment of ALS and potentially other human diseases.

### Introduction

The focused high throughput screening (fHTS) by NMR method was deployed in this Example to derive potent and selective agents targeting the ligand binding domain of the receptor tyrosine kinase EphA4. The strategy ultimately resulted in the identification of agents binding with enthalpy driven nanomolar affinity for the EphA4 ligand binding domain, as determined by isothermal titration calorimetry, and act as agonists in neuronal cells. Structural studies by solution NMR spectroscopy and X-ray crystallography, including the high-resolution structure of the complex between the most potent agent and the ligand binding domain of EphA4, also provide molecular determinants for the binding of the agents and their agonistic properties. Recent studies linked excessive un-ligated EphA4, that can result both by EphA4 overexpression and/or reduced levels of its ephrinA5 ligand, to the progression of Amyotrophic Lateral Sclerosis (ALS), a degenerative disease that affects motor neurons. Disease in a dish assays with ALS patient derived astrocytes and mice motor neurons, suggest the that new EphA4 agonistic agents, mimicking ephrinA5, could be translated in potentially effective ALS treatments. Reported in this Example are the detailed studies that led to the identification and optimization of this innovative agent, including its biophysical, biochemical, and pharmacological characterizations.

### Results

fHTS by NMR Identifications of Novel EphA4-LBD Ligands

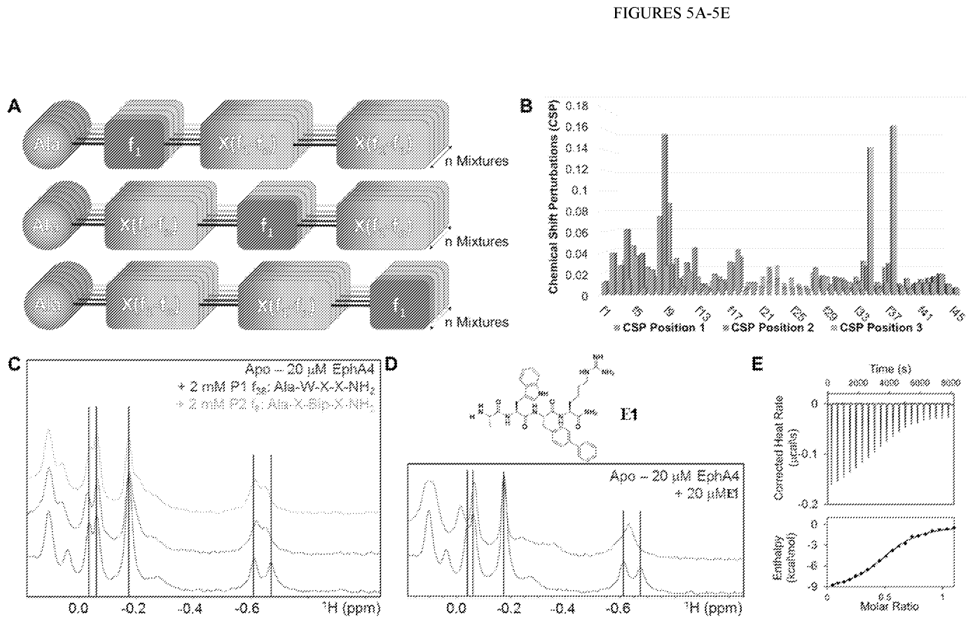

In this Example a positional scanning library of tetrapeptides was derived, all containing a fixed Ala residue at the N-terminus as an initial anchoring moiety. The library, containing 46 natural and non-natural amino-acids at each of the three scanned positions, spanned a chemical space of nearly 100,000 Ala-XXX compounds, arranged in 138 mixtures (FIG. 5) (Baggio et al., ACS Chem Biol, 12 (12): 2981-2989, (2017)). Testing and rank ordering of each mixture was accomplished by measuring sensitive ¹H 1D-aliphatic NMR of recombinant EphA4 ligand binding domain (at 20 μM) and by monitoring eventual chemical shift perturbations induced by each given mixture (at 2 mM total compound concentration). Positive mixtures were therefore identified as those producing a significant perturbation in the aliphatic region of the spectrum (FIG. 5), and correspondingly fixed position amino acids were selected at each of the 3 positions of the Ala-XXX tetrapeptides. Remarkably, the screen and subsequent synthesis and testing of the best combination agent, identified compound E1 with Kd in the low micromolar range as determined by isothermal titration calorimetry (FIG. 5).

Hence, unlike the HTS by NMR approach, in which initial hit molecules presented Kd values for EphA4-LBD in the triple-digit micromolar range (Wu et al., Chemistry & biology, 20 (1): 19-33, (2013)), the fHTS by NMR delivered an agent with single digit micromolar affinity, therefore more directly amenable to further hit-to-lead optimization, as reported below.

### Structure-Activity Relationship Studies Aimed at Hit-to-Lead Optimizations

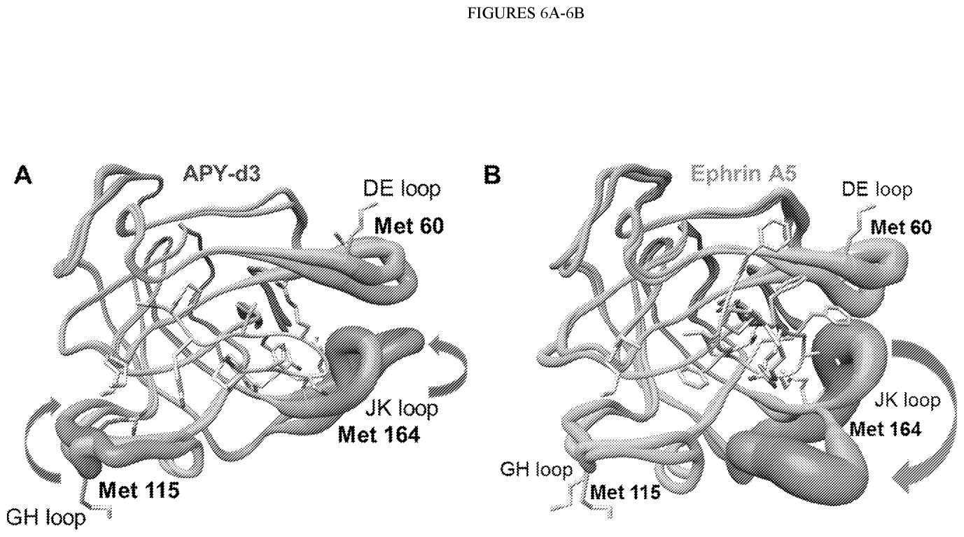

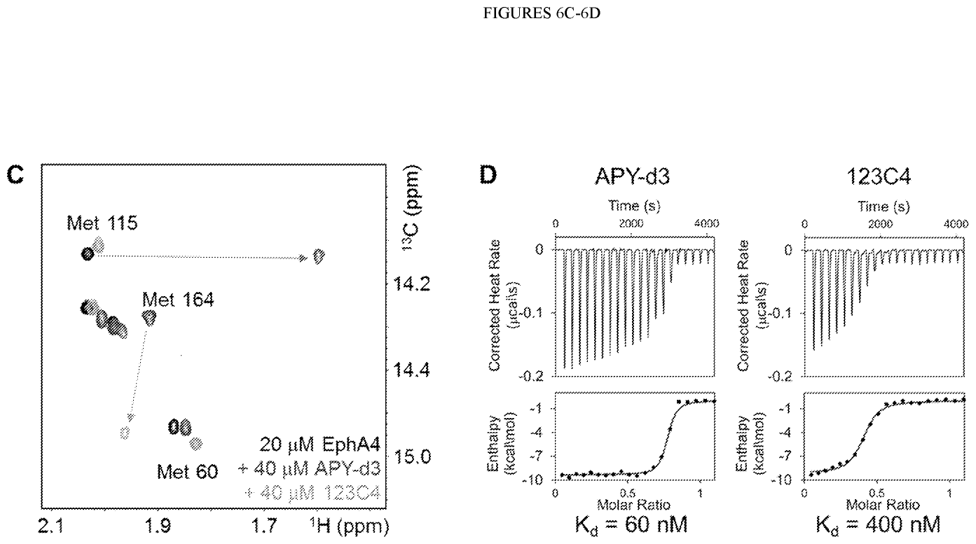

An efficient strategy to monitor ligand binding by protein NMR is the production of protein samples that are uniformly labeled with ¹³C^{ε}-methionine (Wu et al., Cell Chem Biol, 24 (3): 293-305, (2017)). 2D [¹³C,¹H] correlation NMR spectra measured with such protein targets, collected in absence or presence of test ligands can be used to monitor ligand binding and make, albeit qualitative, determinations on possible conformational changes induced by test ligands on the protein target. For example, residues Met 164 and Met 60 are located within the binding site of the EphA4-LBD, in the D-E loop and J-K loop, respectively (FIG. 6A, B). Hence, Met ¹³C^{ε},¹H^{ε} chemical shift perturbations induced by test ligands can be used to monitor and iteratively rank order ligands' binding as illustrated in FIG. 6. The specific resonance assignments of these Met residues have been obtained previously by single point mutations followed by ¹³C^{ε}-methionine labeling and NMR analysis (Wu et al., Cell Chem Biol, 24 (3): 293-305, (2017)). In targeting EphA4, an important aspect regarding the activity of its ligands is whether these agents could be predicted to work as antagonists or agonists. Our previous studies identified 123C4 as a possible agonistic agent (Wu et al., Cell Chem Biol, 24 (3): 293-305, (2017)), while antagonistic compounds have been recently derived by phage display strategies, represented by the 13mer cyclic peptide APY-d3 as the most potent agent reported to date targeting EphA4-LBD (FIG. 6) (Olson et al., ACS Med Chem Lett, 7 (9): 841-846, (2016)). Not surprisingly, comparison of the structures of EphA4-LBD bound to ephrinA5 (a natural EphA4 agonistic ligand) versus EphA4-LBD bound to antagonist APY-d3, revealed differences in the conformational changes induced by the two ligands. Most notably, loop G-H, containing residue Met 115 and located at the EphA4-LBD dimerization interface, assumes two different conformations in the agonist versus antagonist bound structures (FIG. 6A,B). Hence, chemical shift perturbations induced by test ligands to the resonances of Met 115 can be also used to anticipate, albeit qualitatively, whether a ligand caused conformational changes similar to those induced by an agonist or by an antagonist (FIG. 6C). Conversely, structural studies suggest that agonistic agents open the ligand binding domain and cause a large conformational change in the J-K loop, containing Met 164. Hence, antagonists will cause larger chemical shift perturbations of Met 115, while agonists would display larger changes in Met 164, as observed in FIG. 6C. Met 60 chemical shift changes can be more directly attributed to direct interactions of the ligand with the residue, and to some extent, also perhaps to some expected locally induced conformational changes. On the contrary, and as mentioned above, large chemical shift changes for the resonances of residues Met 115 or Met 164 correlated to binding to antagonist agent APY-d3, or by agonistic agent 123C4, respectively. Hence, during the optimizations, the chemical shifts of these residues was monitored to assess whether the test agent would cause conformational changes that are more similar to those caused by the antagonist or by the agonist. Therefore, in carrying out stepwise, iterative structure-activity relationship optimizations studies on initial compound E1, 2D [¹³C,¹H] correlation spectra with ¹³C^{ε}-Met-labeled EphA4-LBD was used to make such qualitative determinations, while isothermal titration calorimetry (ITC) measurements were used for quantitative determinations of the thermodynamics of binding and of the dissociation constants. It was beneficial to use these robust biophysical approaches to iteratively gather detailed information on the binding properties of the agents during the optimization steps, given that biochemical assays have produced false positive agents in the field in the past (Tognolini et al., ACS Chem Neurosci, 5 (12): 1146-1147, (2014); Wu et al., Cell Chem Biol, 24 (3): 293-305, (2017)).

Hence, based on the fHTS by SAR strategy, following the identification of agent E1 (FIG. 5), several agents were designed, synthesized, and tested to optimize each substructure independently. First, the N-terminal Ala position was replaced by other aliphatic amines. These studies are summarized in Table 2 that reports K_{d} measurements by ITC along with ligand induced chemical shifts perturbations on residue Met 164. Of note is that none of the novel agents perturbed Met 115 chemical shifts, suggesting that the series is behaving more like 123C4, hence agonistic, rather than the antagonistic APY-d3.

Replacement of the N-terminal Ala with longer aliphatic chains such as γ-aminobutyric acid (compound E3) increased the affinity of the agent for EphA4 to the sub-micromolar range. Further SAR studies at the same position were also carried out with agents containing a 5 hydroxy tryptophan in P2 (i.e. comparing agent E3, Table 2, with E4 Table 3). These efforts identified either the γ-aminobutyric acid (compound E4) or (1S,3S)-3-aminocyclohexane-1-carboxylic acid (compound E6) as possible preferred replacements of the Ala residue in the P1 position. Compound E6 is particularly interesting based on the larger NMR chemical shifts induced on the resonances of Met 164 and, and based on the reduced losses on entropy upon binding, suggesting the perhaps the constrained cyclohexyl moiety more effectively juxtaposes the primary amine with its binding counterpart.

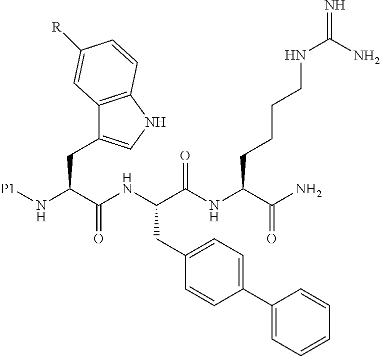

TABLE 2
Compound ID, chemical structures and binding properties of EphA4
targeting agents.

| | | | | | | Δδ |
|---|---|---|---|---|---|---|
| | | | Kd | AH | -TΔS | ¹³C^{ϵ}/¹H^{ϵ} |
| ID | P1 | R | [nM] | [kcal/mol] | [kcal/mol] | Met 164 |
| E1 | 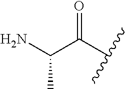 [SMILES: *C(=O)[C@H](C)N] | —H | 3022 | −9.54 | 2.01 | − |
| E2 | 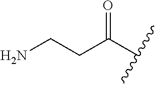 [SMILES: *C(=O)CCN] | —H | 2130 | −11.50 | 3.77 | + |
| E3 | 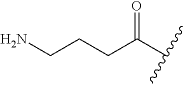 [SMILES: *C(=O)CCCN] | —H | 663 | −10.70 | 2.27 | + |
| E4 | 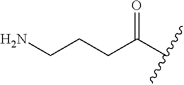 [SMILES: *C(=O)CCCN] | —OH | 352 | −11.59 | 2.79 | + |
| E5 | 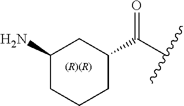 | —OH | 1211 | −9.39 | 1.32 | − |
| E6 | 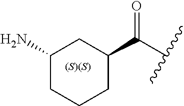 | —OH | 370 | −8.10 | −0.67 | ++ |
| E7 | 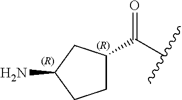 | —OH | 740 | −9.82 | 1.46 | − |
| E8 | 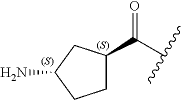 | —OH | 1117 | −8.33 | 0.21 | − |

Kd and thermodynamic binding parameters were obtained by isothermal titration calorimetry measurements against recombinant EphA4 ligand binding domain.
Δδ indicates chemical shift perturbations induced by test ligands (at 40 μM concentration) to ¹H/¹³C^{ϵ} resonances of Met164 in EphA4-LDB (at 20 μM).
Δδ values represent weight average perturbations observed in the ¹H and ¹³C dimensions, as described in the methods.
Δδ: −, no changes; 0< + <0.1 ppm; 0.1 ppm < ++ <0.2 ppm; 0.2< +++ <0.25; ++++ >0.25 ppm.

Further attempts to optimize compound E4 by introducing small modifications of the P2 5OH-Trp residue did not result in agents with improved affinity (Table 3).

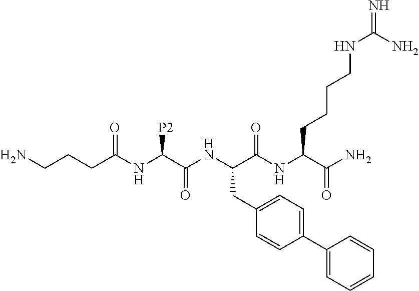

TABLE 3
Compound ID, chemical structures and binding properties of EphA4
targeting agents.

| | | | | | Δδ |
|---|---|---|---|---|---|
| | | Kd | ΔH | -TΔS | ¹³C^{ϵ}/¹H^{ϵ} |
| ID | P2 | [nM] | [kcal/mol] | [kcal/mol] | Met 164 |
| E9 | 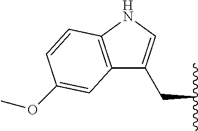 | 444 | −10.56 | 1.90 | + |
| E10 | 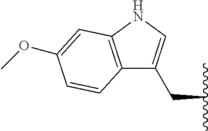 | 2463 | −10.49 | 2.84 | − |
| E11 | 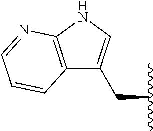 | 16280 | −7.27 | 0.74 | − |

Kd and thermodynamic binding parameters were obtained by isothermal titration calorimetry measurements against recombinant EphA4 ligand binding domain.
Δδ indicates chemical shift perturbations induced by test ligands (at 40 μM concentration) to ¹H/¹³C^{ϵ} resonances of Met 164 in EphA4-LDB (at 20 μM).
Δδ values represent weight average perturbations observed in the ¹H and ¹³C dimensions, as described in the methods.
Δδ: −, no changes; 0< + <0.1 ppm; 0.1 ppm < ++ <0.2 ppm; 0.2< +++ <0.25; ++++ >0.25 ppm.

Hence, further systematic, stepwise optimizations of other positions started with replacements of the P3 phenyl-Phe residue as reported in Table 4. Replacing the P3 position with a α-naphthyl-Ala (compound E12) or a β-naphthyl-Ala (compound E13) reduced the affinity significantly, while smaller substitutions on the biphenyl ring of compound E4 were more tolerated, and in some instances resulted in agents with improved binding affinity for EphA4-LBD (i.e. compounds E16 and E17).

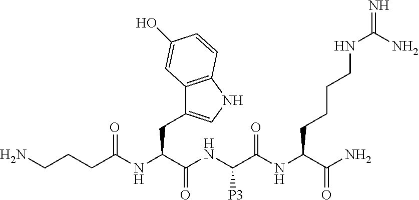

TABLE 4
Compound ID, chemical structures and binding properties of EphA4
targeting agents.

| | | | | | Δδ |
|---|---|---|---|---|---|
| | | Kd | ΔH | -TΔS | ¹³C^{ϵ}/¹H^{ϵ} |
| ID | P3 | [nM] | [kcal/mol] | [kcal/mol] | Met 164 |
| E12 | 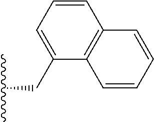 | 11050 | −8.22 | 1.46 | − |
| E13 | 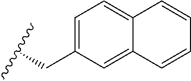 | 5250 | −8.49 | 1.27 | − |
| E14 | 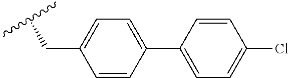 | 749 | −10.36 | 2.00 | ++ |
| E15 | 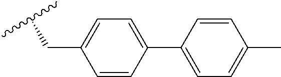 | 708 | −10.16 | 1.77 | − |
| E16 | 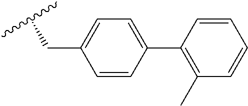 | 362 | −12.08 | 3.29 | + |
| E17 | 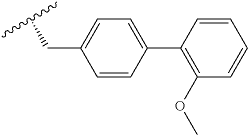 | 196 | −12.29 | 3.14 | ++ |
| E18 | 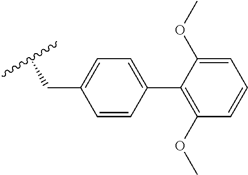 | 1094 | −10.73 | 2.60 | − |

Kd and thermodynamic binding parameters were obtained by isothermal titration calorimetry measurements against recombinant EphA4 ligand binding domain.
Δδ indicates chemical shift perturbations induced by test ligands (at 40 μM concentration) to ¹H/¹³C^{ϵ} resonances of Met 164 in EphA4-LDB (at 20 μM).
Δδ values represent weight average perturbations observed in the ¹H and ¹³C dimensions, as described in the methods.
Δδ: −, no changes; 0< + <0.1 ppm; 0.1 ppm < ++ <0.2 ppm; 0.2< +++ <0.25; ++++ >0.25 ppm.

Likewise, the role of h-Arg in P4 was probed by synthesizing and testing agents with various positively charged residues as listed in Table 5. These efforts suggest that replacements of the D-hArg are more tolerated as typified by agents E21 or E22 containing L-Arg or L-Lys in that position. These limited SAR studies in P4 suggest that perhaps this residue is not intimately in direct contact with EphA4-LBD, as also corroborated by the relatively flat results for the fHTS by NMR in P4 that did not identify a clearly preferred amino acid at that position over others (FIG. 5).

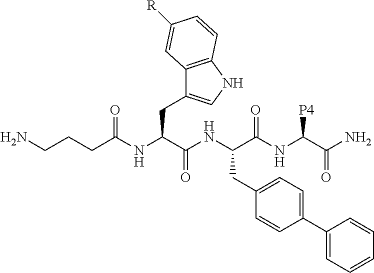

TABLE 5
Compound ID, chemical structures and binding properties of EphA4
targeting agents.

| | | | | | | Δδ |
|---|---|---|---|---|---|---|
| | | | Kd | ΔH | -TΔS | ¹³C^{ϵ}/¹H^{ϵ} |
| ID | P4 | R | [nM] | [kcal/mol] | [kcal/mol] | Metl64 |
| E19 | 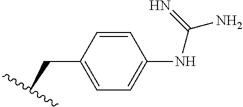 | —H | 1156 | −9.43 | 1.33 | − |
| E20 | 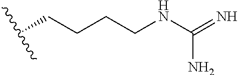 | —H | 270 | −9.26 | 0.30 | − |
| E21 | 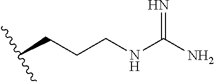 | —OH | 459 | −10.99 | 2.35 | + |
| E22 | 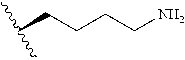 | —OH | 437 | −11.21 | 2.54 | + |

Kd and thermodynamic binding parameters were obtained by isothermal titration calorimetry measurements against recombinant EphA4 ligand binding domain.
Δδ indicates chemical shift perturbations induced by test ligands (at 40 μM concentration) to ¹H/¹³C^{ϵ} resonances of Met 164 in EphA4-LDB (at 20 μM).
Δδ values represent weight average perturbations observed in the ¹H and ¹³C dimensions, as described in the methods.
Δδ: −, no changes; 0< + <0.1 ppm; 0.1 ppm < ++ <0.2 ppm; 0.2< +++ <0.25; ++++ >0.25 ppm.

Hence, keeping as an upper limit of the molecular weight of the final agents less than 1000 Da (Ran et al., Curr Opin Chem Biol, 44 75-86, (2018)) and following our previous optimization strategies, it was probed whether the binding affinity of the agents could be further improved by elongating the molecules with one additional P5 element. Interestingly, only derivatization with Gly or D-Ala resulted in agents with similar or slightly improved affinity, while elongation with other amino acids resulted in agents with significantly reduced affinity (Table 6).

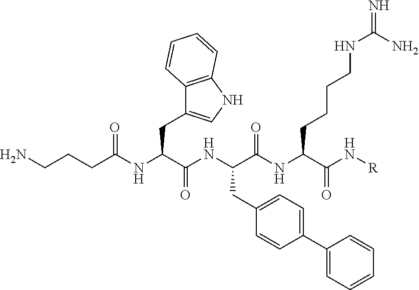

TABLE 6
Compound ID, chemical structures and binding properties of EphA4 targeting agents.

| | | | | | Δδ |
|---|---|---|---|---|---|
| | | | | | 1D |
| | | Kd | ΔH | -TΔS | Aliph-¹H |
| ID | R | [nM] | [kcal/mol] | [kcal/mol] | NMR |
| E3 | —H | 663 | −10.70 | 2.27 | +++ |
| E34 | 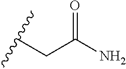 [SMILES: *CC(N)=O] | 563 | −11.29 | 2.76 | +++ |
| E35 | 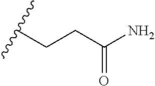 [SMILES: *CCC(N)=O] | 846 | −9.94 | 1.65 | ++ |
| E36 | 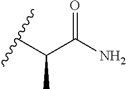 [SMILES: *[C@H](C)C(N)=O] | 348 | −11.58 | 2.77 | +++ |
| E37 | 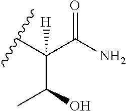 [SMILES: *[C@@H](C(N)=O)[C@H](C)O] | 1970 | −9.13 | 1.35 | ++ |
| E38 | 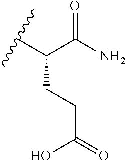 [SMILES: *[C@@H](CCC(=O)O)C(N)=O] | ND | ND | ND | − |
| E39 | 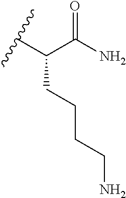 [SMILES: *[C@@H](CCCCN)C(N)=O] | ND | ND | ND | − |
| E40 | 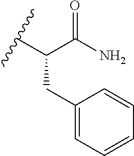 [SMILES: *[C@@H](Cc1ccccc1)C(N)=O] | ND | ND | ND | + |
| E41 | 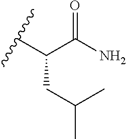 [SMILES: *[C@@H](CC(C)C)C(N)=O] | ND | ND | ND | + |
| E42 | 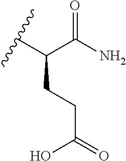 [SMILES: *[C@H](CCC(=O)O)C(N)=O] | ND | ND | ND | ++ |
| E43 | 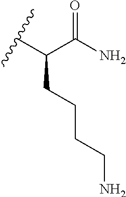 [SMILES: *[C@H](CCCCN)C(N)=O] | ND | ND | ND | ++ |
| E44 | 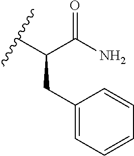 [SMILES: *[C@H](Cc1ccccc1)C(N)=O] | 696 | −9.73 | 1.33 | +++ |
| E45 | 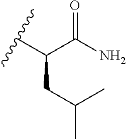 [SMILES: *[C@H](CC(C)C)C(N)=O] | 788 | −8.12 | −0.20 | +++ |
| E46 | 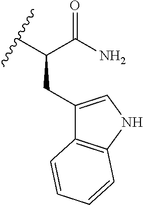 [SMILES: *[C@H](Cc1c[nH]c2ccccc12)C(N)=O] | 439 | −8.28 | −0.40 | ++ |

Kd and thermodynamic binding parameters were obtained by isothermal titration calorimetry assays against recombinant Epha4 ligand binding domains.
Δδ indicates chemical shift perturbations induced by test ligands (at 20 μM concentration) to ID ¹H aliphatic region of the EphA4-LDB as illustrated in FIG. 5.
Δδ: 0< − <0.1 ppm; 0.1 ppm < + <0.2 ppm; 0.2< ++ <0.3; +++ >0.3 ppm.

Therefore, fixing Gly at the C-terminus, it was further tested whether agents could be further elongated with small amines, again trying to keep the MW within 1000 Da. These efforts are summarized in Table 7, where several aromatic amines were introduced and found to have improved the binding affinity and caused a larger shift of the resonances of Met 164.

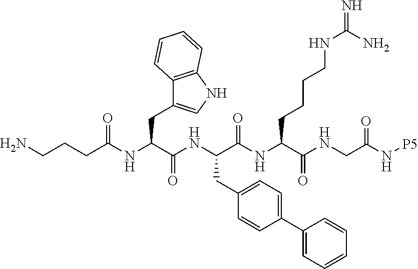

TABLE 7
Compound ID, chemical structures and binding properties of EphA4
targeting agents.

| | | | | | Δδ |
|---|---|---|---|---|---|
| | | Kd | AH | -TΔS | ¹³C^{ϵ}/¹H^{ϵ} |
| ID | P5 | [nM] | [kcal/mol] | [kcal/mol] | Met 164 |
| E23 | 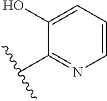 [SMILES: *c1ncccc1O] | 488 | −9.48 | 0.86 | + |
| E24 | 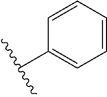 [SMILES: *c1ccccc1] | 132 | −12.26 | 2.87 | ++ |
| E25 | 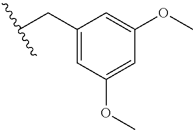 [SMILES: *Cc1cc(OC)cc(OC)c1] | 307 | −11.27 | 2.39 | + |
| E26 | 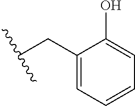 [SMILES: *Cc1ccccc1O] | 258 | −11.98 | 3.00 | + |
| E27 | 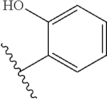 [SMILES: *c1ccccc1O] | 191 | −12.04 | 2.88 | ++ |
| E28 | 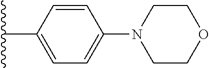 [SMILES: *c1ccc(N2CCOCC2)cc1] | 94 | −11.22 | 1.63 | ++++ |
| E29 | 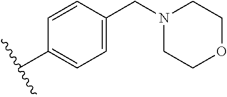 [SMILES: *c1ccc(CN2CCOCC2)cc1] | 106 | −10.56 | 1.05 | +++ |

Kd and thermodynamic binding parameters were obtained by isothermal titration calorimetry measurements against recombinant EphA4 ligand binding domain.
Δδ indicates chemical shift perturbations induced by test ligands (at 40 μM concentration) to ¹H/¹³C^{ϵ} resonances of Met 164 in EphA4-LDB (at 20 μM).
Δδ values represent weight average perturbations observed in the ¹H and ¹³C dimensions, as described in the methods.
Δδ: −, no changes; 0< + <0.1 ppm; 0.1 ppm < ++ <0.2 ppm; 0.2< +++ <0.25; ++++ >0.25 ppm.

Finally, based on the data from Tables 2-7, additional agents were synthesized presenting various combinations of the most active substituents at the P1-P5 positions (Table 8).

TABLE 8
Compound ID, chemical structures, and binding properties of EphA4
targeting agents.

| | | | K_{d} (nM) | K_{d} (nM) | K_{d} (nM) | | Δδ | Δδ | Δδ |
|---|---|---|---|---|---|---|---|---|---|
| | | | vs | vs | vs | Aqueous | Met | Met | Met |
| ID | Structure | MW | EphA4 | EphA3 | EphA2 | Solubility | 164 | 115 | 60 |
| 2 | 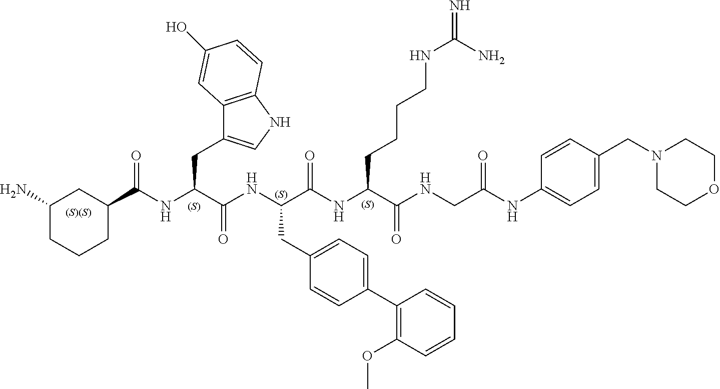 | 1000 | 77 | ~9000 | N.A. | >50 mM | + | + | ++ |
| 9 | 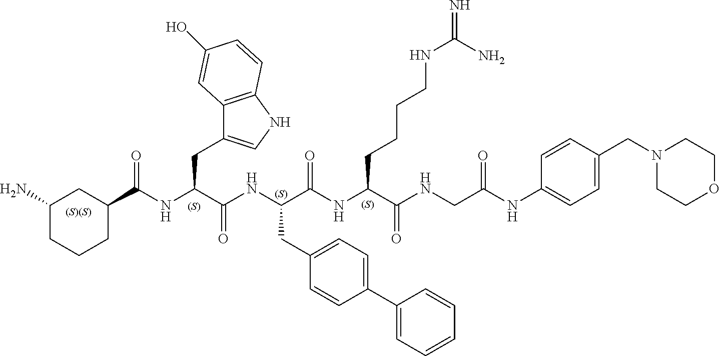 | 970 | 144 | ~7000 | N.A. | >50 mM | +++ | + | ++ |
| 3 | 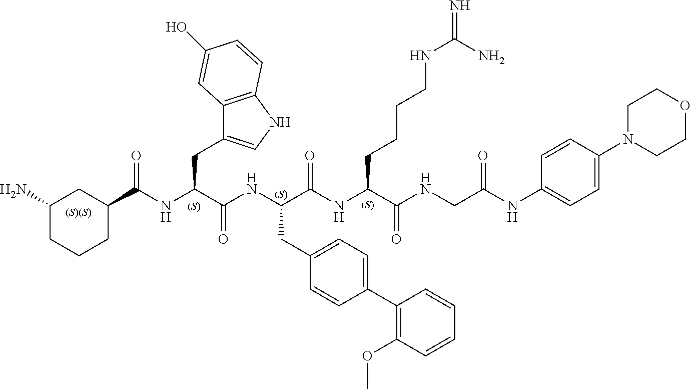 | 986 | 106 | −7000 | N.A. | >50 mM | + | + | + |
| 8 (150D4) | 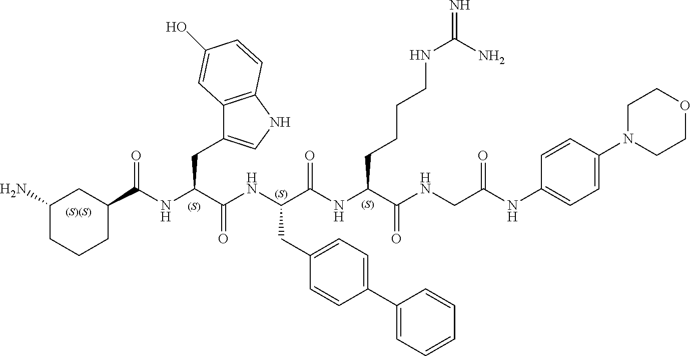 | 956 | 113 | ~4000 | N.A. | >50 mM | ++++ | + | ++ |
| 123C4 | 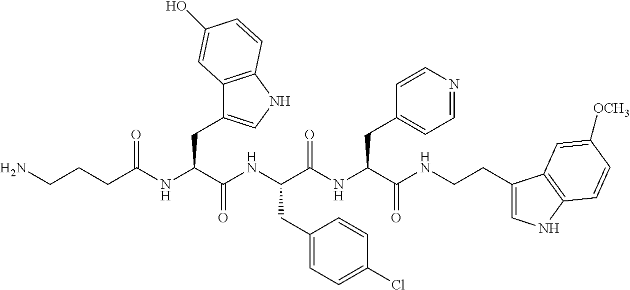 [SMILES: COc1ccc2[nH]cc(CCNC(=O)[C@H](Cc3ccncc3)NC(=O)[C@H](Cc3ccc(Cl)cc3)NC(=O)[C@H](Cc3c[nH]c4ccc(O)cc34)NC(=O)CCCN)c2c1] | 807 | 400 | >10000 | N.A. | ~100 μM | ++ | + | + |
| APY-d3 | βA-PYCVYR-βA-SWSC-CONH₂ | 1402 | 60 | ~1500 | ND. | N.D. | + | ++++ | + |

Kd and thermodynamic binding parameters were obtained by isothermal titration calorimetry measurements with the respective ligand binding domains.
Δδ indicates chemical shift perturbations induced by test ligands (at 40 μM concentration) to ¹H/¹³C^{ϵ} resonances of the indicated residues in EphA4-LDB (at 20 μM).
Δδ values represent weight average perturbations observed in the ¹H and ¹³C dimensions, as described in the methods.
Δδ: −, no changes; 0< + <0.1 ppm; 0.1 ppm < ++ <0.2 ppm; 0.2< +++ <0.25; ++++ >0.25 ppm.
N.D., not determined; N.A., no affinity detectable under the tested experimental conditions.

Noteworthy is that the resulting agents, all within MW˜1000 Da, displayed a binding affinity for EphA4-LBD that is comparable to that of the phage display derived (and extensively optimized) antagonistic cyclic 13mer peptide APY-d3 (MW=1402; FIG. 6; Table 8). Moreover, the agents are very soluble in buffer, that may turn out particularly useful if their administration as therapeutics would require intrathecal delivery.

In summary, the fHTS by NMR approach of the Ala-XXX positional scanned tetra-peptide library, followed by stepwise and iterative optimizations of the P1-P4 positions, and the introduction of a P5 amine at the C-terminal, resulted in agents that are as potent as those derived from an extensively optimized phage display derived APY-d3 peptide. Perhaps most importantly, unlike APY-d3, the agents are predicted to work as agonists based on chemical shift perturbations detected via the ¹³C^{ε}/¹H^{ε} resonances of Met 115, Met 164, and Met 60 (Table 8).

To assess the selectivity of the final agents (Table 8), they were tested against the two most closely related Eph ligand binding domains, namely EphA3 (˜73% sequence identity with EphA4 within the LBD) and EphA2 (˜55% sequence identity with EphA4 within the LBD) (Table 8). Under the same experimental conditions, the agents appeared inactive against the EphA2, while displayed micromolar affinities (at best) against the EphA3. Hence the agents are >ten-fold selective for the EphA4 compared to its most closely related receptor, the EphA3 (Table 8). These data identified compound 8 (named 150D4; Table 8) as a potent agonistic agent targeting EphA4.

### Molecular Basis for the Affinity and the Selectivity of 150D4 for EphA4-LBD

To further investigate at the molecular level the basis of binding and selectivity, these agents, a high-resolution X-ray structure of the complex between EphA4-LBD and the representative compound 150D4 was obtained.

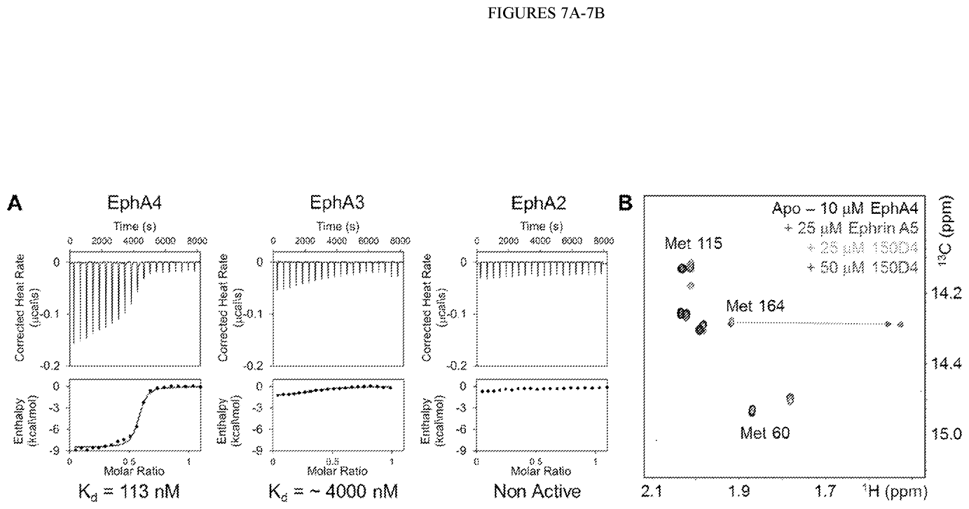

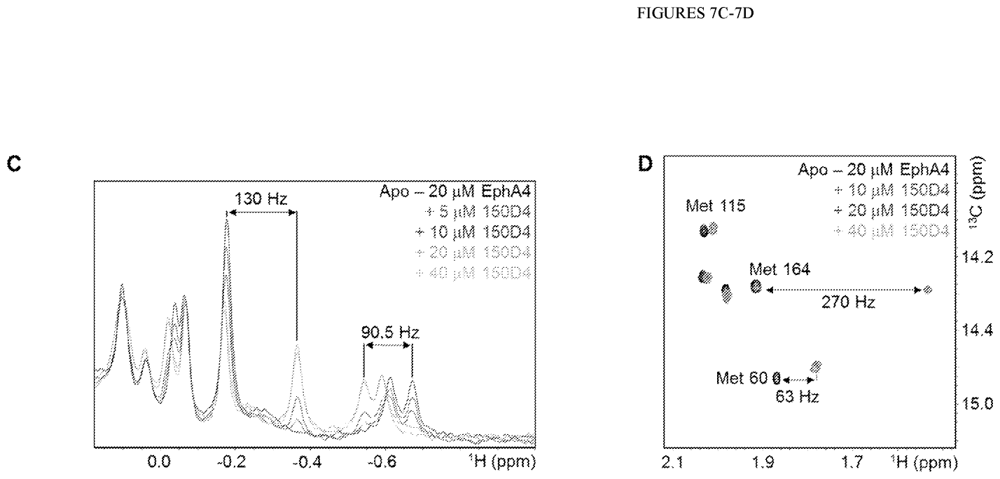

As mentioned, sequence specific resonance assignments of the Met residues have been obtained in previous studies by single point mutations followed by ¹³C^{ε}-methionine labeling and NMR analysis (Wu et al., Cell Chem Biol, 24 (3): 293-305, (2017)). Chemical shifts perturbations upon ligand titration to the ¹³C^{ε}-Met labeled EphA4-LBD occurred in slow-exchange in the NMR-time scale, that is upon titration of 150D4 the cross-peaks corresponding to both ¹³C^{ε}/¹H^{ε} resonances of Met 60 and Met 164 progressively disappeared, while two new cross-peaks appeared, which is typical of tight binding affinities (Pellecchia et al., Nat Rev Drug Discov, 1 (3): 211-219, (2002); Pellecchia, Chemistry & biology, 12 (9): 961-971, (2005); Pellecchia et al., Nat Rev Drug Discov, 7 (9): 738-745, (2008); Barile et al., Chem Rev, 114 (9): 4749-4763, (2014)). Two methionine residues, Met 164 and Met 60, are located in the ephrin binding site of EphA4-LBD, while small or no significant perturbations are observed for residue Met 115, in the G-H loop at the dimerization interface in the ephrin-bound structures (FIG. 7). Chemical shifts differences of Met 60 resonances between the free versus 150D4 bound form provide an estimated upper limit for the off rate for the complex of k_{off}<60 s⁻¹, that assuming a diffusion limited on rate of 10⁹ M⁻¹ s⁻¹, would correspond to a dissociation constant Kd<60 nM (FIG. 7B), thus in good agreement with the ITC data, that indicated a Kd of 113 nM for 150D4 versus EphA4-LBD. On the contrary, limited binding was observed in a similar assay against EphA3 or EphA2 (FIG. 7A).

Furthermore, and in agreement with our binding and NMR data, it was found that 150D4 efficiently displaces the binding of ephrinA5 (the most potent endogenous ephrin ligand for EphA4) (Bowden et al., Structure, 17 (10): 1386-1397, (2009)) using displacement 2D [¹³C, ¹H] correlation spectra (FIG. 7D).

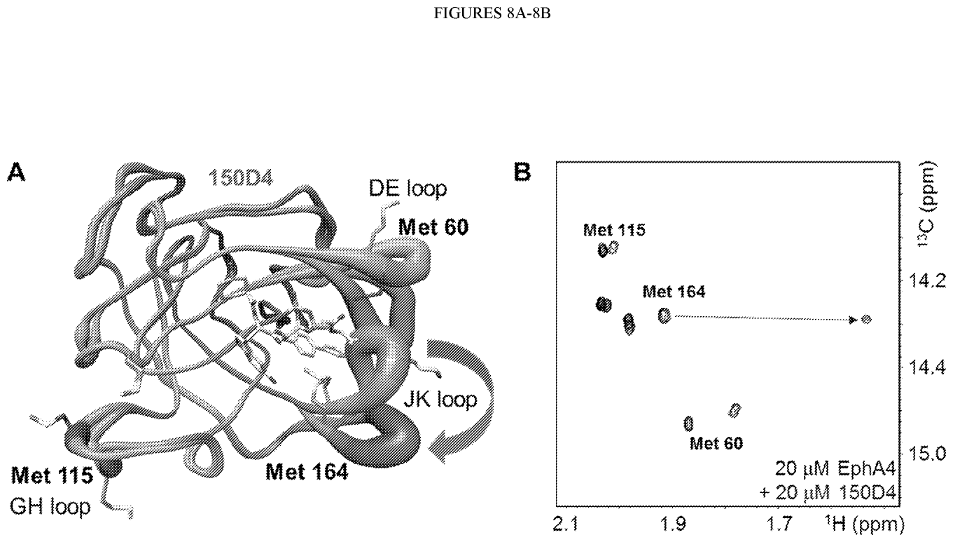

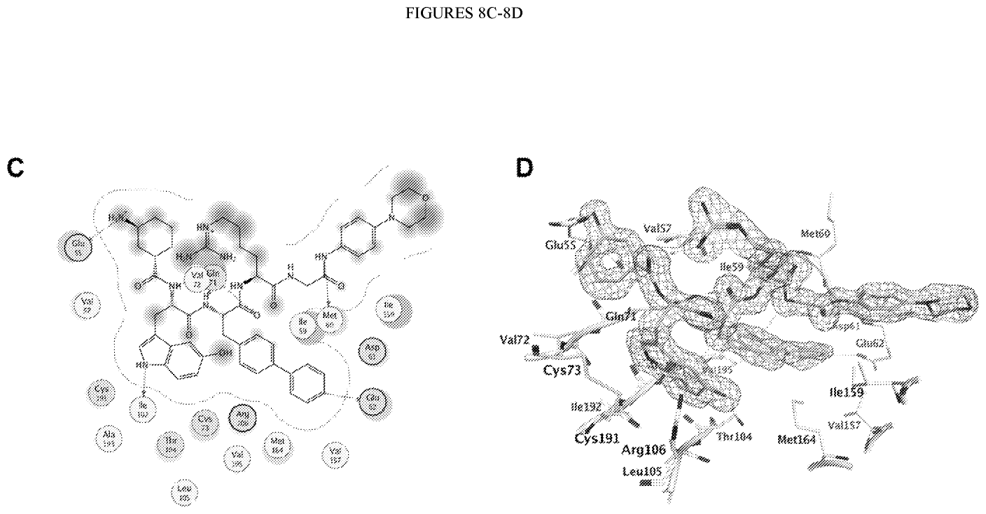

Next, the X-ray structure of EphA4-LBD in complex with 150D4 at 1.43 Å resolution was determined (FIG. 8). The crystals contained one monomer of EphA4-LBD in the asymmetric unit comprising EphA4-LBD residues Asn 29 to Arg 209 and the ligand. The structure of the ligand binding domain of EphA4-LDB in complex with ligand 150D4 adopts a typical bi-lobal architecture that is characteristic of other members of the eukaryotic protein kinase family (FIG. 8). The resulting electron density showed an unambiguous binding mode for the ligand 150D4, including the orientation and conformation of the ligand. Based on a distance of less than 3.5 Å of the donor and acceptor atoms, several specific hydrogen bonds of the ligand 150D4 could be identified, namely to the main chain atoms of Ile 192 and Met 60, as well as the side chain atoms of Glu 55 and Gln 71. According to the above distance criteria, the presence of additional hydrophilic interactions to the main chain atoms of Met 60 as well as the side chain atoms of Thr 104 and Arg 162 could also be identified. The following residues can be found in the vicinity of the ligand with a maximum distance of 3.9 Å (FIG. 8): Glu 55, Ile 59, Met 60, Asp 61, Glu 62, Gln 71, Val 72, Cys 73, Thr 104, Leu 105, Arg 106, Ile 159, Met 164, Cys 191, Ile 192, and Ala 193.

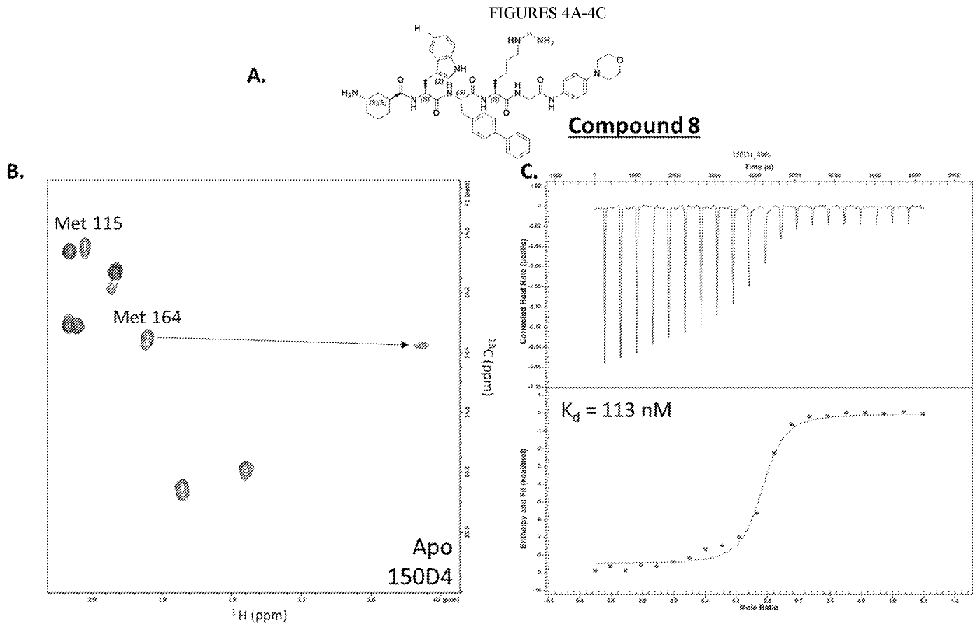

Several of such structural details of the binding mode of 150D4 can fully explain the observed SAR studies of Tables 2, 3, 4, 5 and 7. For example, the N-terminal amine is involved in hydrogen bonding with EphA4 Glu 55, the tryptophan in position P2 occupies a shallow hydrophobic sub pocket, with the 5-hydroxyl group involved in hydrogen bonding with Arg 106 mediated by a water molecule, the bi-phenyl group of position P3 occupies a deep pocket, juxtaposing the aromatic rings in proximity of Met 164 (FIG. 8). This geometry likely justifies the large perturbations induced by these agents on the chemical shifts of Met 164. The C-terminal portion of the molecule, with the P4 homo-Arg does not seem to be intimately involved intermolecular interactions, in close agreement with the fHTS by NMR and SAR data (FIG. 4, Table 4), where the P4 position appeared to be relatively less critical for binding. On the contrary, phenyl-morpholino in P5, also conferring increased solubility to the agent, is likely causing the large conformational change in the J-K loop (FIG. 8), in agreement with Met 164 NMR chemical shift perturbation studies, presumably further promoting an agonist-like activity of the agent.

In this regard, analysis of the conformations of the loop regions D-E, J-K, and G-H in 150D4 bound EphA4-LBD suggests that these loops adopt a conformation that is more similar to that adopted by the target when bound to the ephrin ligand (PDB ID 2WO1)(Bowden et al., Structure, 17 (10): 1386-1397, (2009)) compared to that of antagonist bound APY-d3 (PDB ID 5JR2) (Olson et al., ACS Med Chem Lett, 7 (9): 841-846, (2016)). In particular, residue Met 115 within loop G-H did not display the large conformational rearrangement as observed in the complex with APY-d3, in agreement with NMR measurements (FIG. 8).

The molecular basis for the selectivity of the compound can also be deduced by analyses of the X-ray structure of EphA4 in complex with 150D4 and the X-ray structure of EphA3 in complex with ephrinA5 (PDB ID 4LOP) (Forse et al., PLoS One, 10 (5): e0127081, (2015)). Comparing EphA4-LBD and EphA3-LBD structures confirmed that there are only few significant differences in the ligand binding regions of these proteins, amounting to 8 mutations. Hence, a construct representing the ligand binding domain of EphA3 was prepared by introducing these 8 mutations and in a EphA4-LBD construct (see experimental session). Isothermal titration calorimetry measurements with 150D4 and this EphA3-LBD chimera indicated that the agent presented a markedly reduced affinity for this construct (FIG. 7, Table 8), despite its high similarity with EphA4-LBD (>95% sequence identity).

Taken together, these structural data strongly suggest that 150D4 and other related agents in Table 8 represent potent and selective binders for EphA4-LBD, that induce conformational changes upon binding that resemble more closely those induced by agonistic agents.

### Cellular Studies

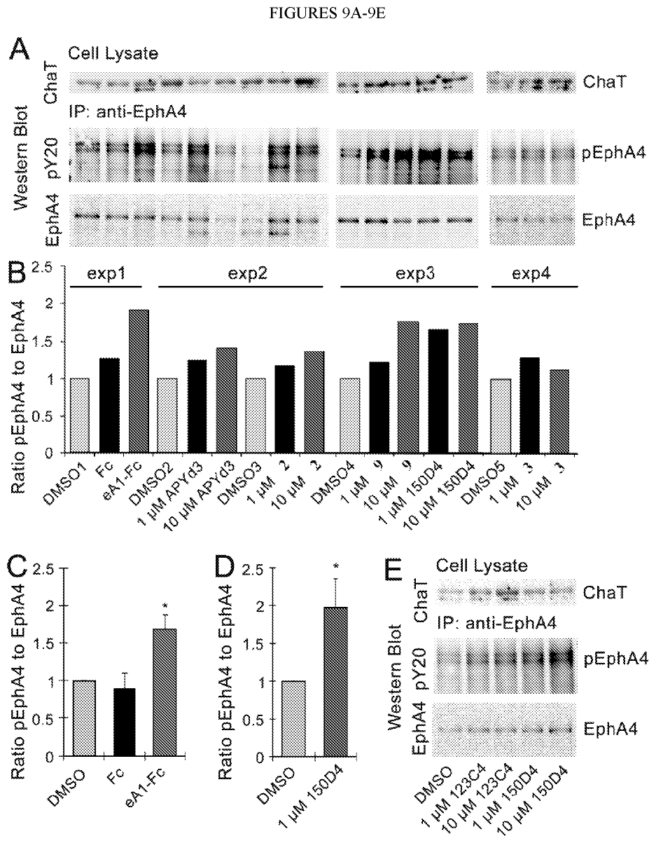

An indirect measure of agonism by EphA4 ligands can be assessed by monitoring the phosphorylation of its cytosolic kinase domain upon ligand binding. Hence, primary motor neurons were isolated from postnatal day (P) 0-P2 mouse spinal cords of B6.Cg-Tg(Hlxb9-GFP)1Tmj/J (Hb9-GFP) mice. Subsequently, cells were treated with ephrinA1-Fc (R&D Systems, #602-A1) or human Fc (R&D Systems, #110-HG) as controls. EphrinA1-Fc needs clustering for maximal agonistic activity, which was accomplished by the incubation with goat anti-human IgG (Jackson ImmunoResearch, #109-005-003) for 1 h at 4° C. At 2 days in vitro (DIV) primary motor neurons were then treated with pre-clustered Fc (2 μg/mL, as negative control), pre-clustered ephrinA1-Fc (2 μg/mL, as positive control), and various agents including antagonist APY-d3, and agents listed in Table 8, namely 123C4, compound 2, compound 9, compound 3, and 150D4, each at concentrations 1 μM or 10 μM, for 30 min at 37° C. under 5% CO₂/10% O₂ atmosphere, and then processed for western blotting. After cell lysis, cells were exposed to protein-A agarose beads (Sigma, #P1406) and anti-EphA4 antibody (Invitrogen, #371600), for 2 h at 4° C., and subsequently boiled in reducing conditions, spun down, and the supernatant was subjected to WB analysis with an anti-phosphotyrosine antibody and re-probed with EphA4 antibody (FIG. 9). These data suggest that most agents induced receptor phosphorylation (FIG. 9A,B), with 150D4 resulting more significantly active than others (FIG. 9C,D). Direct comparison between 150D4 and the previously derived agonistic agent 123C4 (Table 8) in the same assay, also revealed a significant increased phosphorylation induced by the newest compound (FIG. 9E).

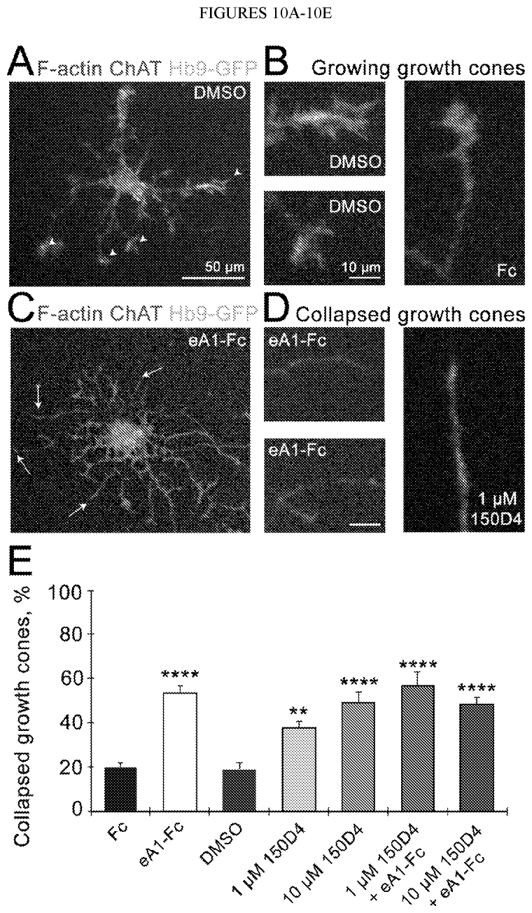

To further verify agonism in a functional assay, growth cone analysis was conducted using 2 DIV primary spinal cord motor neurons treated as described above with Fc, ephrinA1-Fc, 1 μM 150D4, 10 μM 150D4, 1 μM 150D4 plus ephrinA1-Fc, or 10 μM 150D4 plus ephrinA1-Fc. After images collection (100 images were collected per treatment group), growth cones were assessed based on filamentous (F)-actin labeling and classified into collapsed and growing based on their morphology. The percentage of neurons with collapsed growth cones was determined. Representative images are reported in FIG. 10A-D, including statistical analysis based on differences for multiple groups, as assessed by one-way ANOVA followed by Bonferroni's post hoc tests (FIG. 10E). In this experimental setting, 150D4 is effective in inducing growth cone collapse similar to the agonistic clustered ligand ephrinA1-Fc (FIG. 10), further corroborating the potential agonism induced by the tested agents.

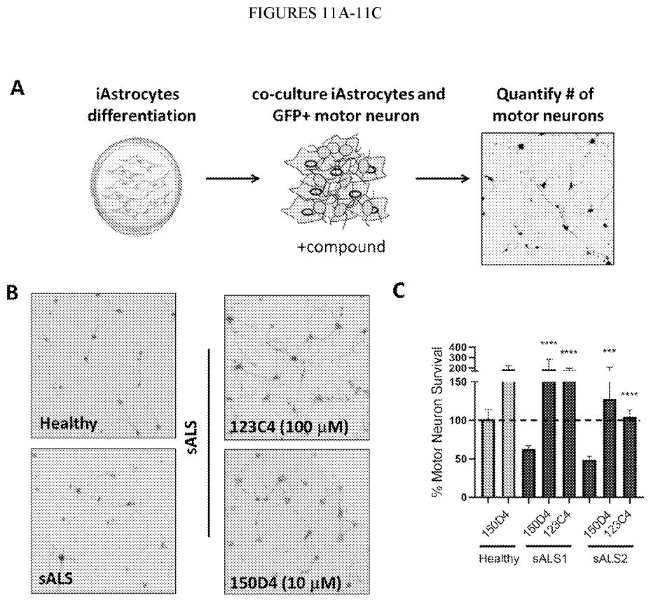

EphA4 has been directly implicated in the progression of ALS in mice models and in human genetic studies. While the mechanisms for onset and progression of ALS remain largely undetermined, astrocytes have been implicated as significant contributors to motor neuron death in both in familial ALS (fALS), that is driven by inactivating mutations within the superoxide dismutase 1 (SOD1) gene and that account for less than 2% of all ALS cases, (Di Giorgio et al., Nat Neurosci, 10 (5): 608-614, (2007); Nagai et al., Nat Neurosci, 10 (5): 615-622, (2007); Yamanaka et al., Nat Neurosci, 11 (3): 251-253, (2008)) and the more common sporadic form of ALS (sALS). (Di Giorgio et al., Nat Neurosci, 10 (5): 608-614, (2007); Nagai et al., Nat Neurosci, 10 (5): 615-622, (2007); Di Giorgio et al., Cell Stem Cell, 3 (6): 637-648, (2008); Marchetto et al., Cell Stem Cell, 3 (6): 649-657, (2008); Yamanaka et al., Nat Neurosci, 11 (3): 251-253, (2008)). Recently, it was shown that astrocytes derived from both patient groups are similarly toxic to motor neurons (Haidet-Phillips et al., Nat Biotechnol, 29 (9): 824-828, (2011)). Hence, a co-culture assay was developed to provide a meaningful and a more general in vitro model system to evaluate potential experimental therapeutics for sALS and fALS. Preliminarily, the ability of agonistic agent 150D4 (at 10 μM) was probed to rescue sALS astrocytes-induced motor neuron death, side by side with first generation agent 123C4. The latter resulted active only at higher concentrations, hence it was tested at 100 μM. Both 150D4 and, to a lesser extent, 123C4 (at a higher concentration) were able to protect mouse motor neurons from iAstrocytes derived from sALS patients (FIG. 11) (Meyer et al., Proc Natl Acad Sci USA, 111 (2): 829-832, (2014)). Most previously known agents tested using this assay for unrelated targets did not result in any significant protection, making 150D4 potentially a viable lead agent for further drug development studies for ALS therapeutics.

In addition, brain exposure to 150D4 was determined in pharmacokinetics study (Table 9), suggesting 150D4 may be able to cross blood brain barrier.

TABLE 9
In vivo pharmacokinetics and brain exposure data. Mice (n = 4 for each route of
administration) were administrated with 150D4 at the indicated doses either IV or IP and plasma
and brain drug concentrations were determined via LC/MS at the time points indicated. Amount
of 150D4 in the whole brain at the indicated time points was obtained by extraction in
acetonitrile/water (~1 ml) from homogenized brain tissue followed by LC/MS analysis.

| | | | | | | | Amount of | |
|---|---|---|---|---|---|---|---|---|
| | Plasma drug | | Brain | | Total extracted | | compound per mg | |
| Dose/route | concentration | | concentration | | brain compound | | of wet brain tissue | |
| 10 mg/kg | 484 | +/− 250 ng/ml | 189 | +/− 1 ng/ml | 205.6 | +/− 3 ng | 4.73 | +/− 0.1 ng/mg |
| IV | (30 min) | | (30 min) | | (30 min) | | (30 min) | |
| | 14.2 | +/− 5.0 ng/ml | 54 | +/− 3 ng/ml | 60 | +/− 8 ng | 1.36 | +/− 0.1 ng/mg |
| | (24 hr) | | (24 hr) | | (24 hr) | | (24 hr) | |
| 50 mg/Kg | 7735 | +/− 120 ng/ml | 184 | +/− 87 ng/ml | 185 | +/− 38 ng | 4.6 | +/− 2 ng/mg |
| IP | (1 hr) | | (1 hr) | | (30 min) | | (30 min) | |
| | 45 | +/− 3 ng/ml | 45.5 | +/− 0.3 ng/ml | 34.7 | +/− 6 ng | 1.1 | +/− 0.01 ng/mg |

| | | | |
|---|---|---|---|
| (24 hr) | (24 hr) | (24 hr) | (24 hr) |

These data collectively suggest that the agents function as EphA4 agonistic ligands in vitro and in relevant cellular and functional assays. Hence, agents such as 150D4 could find immediate applications in disease areas where activation of EphA4 by the ephrin ligands may be beneficial, including amyotrophic lateral sclerosis (ALS), Parkinson's disease (PD), Alzheimer's disease, and potentially other diseases.

### Discussion

EphA4 belongs to large family of receptor tyrosine kinases, which together with their ephrin ligands, is involved in bi-directional signaling events that control several cellular processes during development and in disease (Pasquale, Cell, 133 (1): 38-52, (2008)). The ephrin mediated signaling occur by interactions with the extracellular Eph ligand binding domain (LBD), which in turn activates intracellular domains including a kinase domain, in addition to a sterile alpha motif (SAM) domain, and a PDZ binding motif that are believed to propagate the cell signaling cascade. In recent years EphA4 went under scrutiny for its possible role critical role in several disease conditions (Boyd et al., Nat Rev Drug Discov, 13 (1): 39-62, (2014)), including abnormal blood clotting, spinal cord injury, and Alzheimer's disease (AD), in addition to amyotrophic lateral sclerosis, and potentially other diseases (Tognolini et al., ACS Chem Neurosci, 5 (12): 1146-1147, (2014)).

Numerous structural studies identified the molecular determinants for EphA4-LBD/ephrin interactions (Bowden et al., Structure, 17 (10): 1386-1397, (2009)). In addition, several phage display derived short peptide binders (12-mers) that selectively block ephrin ligands from interacting with the EphA4 have been reported (Murai et al., Molecular and cellular neurosciences, 24 (4): 1000-1011, (2003)), that bind to EphA4-LBD with Kd values in the low micromolar range (Murai et al., Molecular and cellular neurosciences, 24 (4): 1000-1011, (2003); Lamberto et al., The Biochemical journal, 445 (1): 47-56, (2012)). More recently a cyclic peptide termed APY-d3 (FIG. 5) was also reported that displayed antagonistic behavior for the receptor, displacing ephrinA5 mediated phosphorylation and growth cone collapse in neuronal cells (Lamberto et al., ACS chemical biology, 9 (12): 2787-2795, (2014)). More recently, agent 123C4 (FIG. 5) was reported as a first synthetic agonistic ligand with a dissociation constant for EphA4-LBD of ˜ 400 nM, that was derived by a de novo HTS by NMR campaign, testing a library of ˜ 100,000 tri-peptides, including non-natural amino acids, followed by SAR studies (Wu et al., Chemistry & biology, 20 (1): 19-33, (2013); Wu et al., Curr Top Med Chem, 15 (20): 2032-2042, (2015); Bottini et al., Chem Med Chem, 11 (8): 919-927, (2016); Baggio et al., ACS Chem Biol, 12 (12): 2981-2989, (2017); Wu et al., Cell Chem Biol, 24 (3): 293-305, (2017)). In cellular assays, 123C4 was found to have agonistic activity for EphA4 in primary cortical neurons, hence, acting similarly to ephrin ligands, induced receptor phosphorylation and growth cone collapse (Wu et al., Cell Chem Biol, 24 (3): 293-305, (2017)), but the molecular determinants at the basis for such activity have not yet been elucidated.

In this Example, the initial hit molecule compound E1 was identified that was able to bind to EphA4-LBD already with low micromolar affinity (FIG. 5, Table 2). Iterative optimizations of each of the positions of the tetra peptide (Tables 2-5), including further derivatizations at the C-terminus with a 5^{th} substituent (Table 7), resulted in the design of the final agents reported in Table 8. Potency, selectivity, and solubility of the resulting agents have been the main guiding criteria during the optimization steps keeping the molecular weight well below 1000 Da (Table 8). During each iterative optimization steps, binding was monitored by NMR spectroscopy with ¹³C^{ε}-Met labeled EphA4-LBD and sensitive isothermal titration calorimetry studies, including testing the agents against the most closely related Eph receptors, namely the EphA3, and EphA2, to assess selectivity. These efforts culminated in a series of agents that bind potently and selectively to EphA4-LBD in the nanomolar range, with minimal targeting of EphA3, and non-significant interactions with EphA2 (Table 8).

To further elucidate at the molecular level the basis for the observed activity and selectivity, the X-ray structure of 150D4 in complex with EphA4-LBD was also determined (FIG. 8), where a dense network of favorable intermolecular interactions can be observed that are consistent the observed SAR, potency, and selectivity.

Recent X-ray crystallography studies with the EphA4 receptor in complex with an ephrin ligand (Xu et al., Proc Natl Acad Sci USA, 110 (36): 14634-14639, (2013)) suggested that ligand binding to the EphA4 receptor induces a conformational changes in the D-E and J-K loops in EphA4-LBD that favor receptor activation. Similar studies with antagonist APY-d3 reveal instead conformational changes induced at the loop G-H that perhaps could preclude dimer formation, which is believed to the first step for receptor activation. Interestingly, loop G-H contains residue Met 115, hence ¹³C^{ε}-Met labeled samples of EphA4-LBD was used to monitor and compare the binding of the antagonist agent APY-d3, and the agents described herein. It was found that while both classes of agents caused widespread changes in the chemical shifts of binding site Met residues 60 and 164 (located in the D-E and J-K loops, respectively), only the antagonist caused very large chemical shift perturbations in G-H loop Met 115 resonances, well in agreement with X-ray studies on that complex. At the same time, these observations would suggest that certain agents described herein, similar to 123C4, could act as agonists for the EphA4. Accordingly, the agents acted as agonists towards EphA4 in primary motor neurons, both by activating receptor phosphorylation and by inducing growth cone collapse, similar to ephrinA1-Fc.

Recent studies on the role of EphA4 in disease were focused on ALS, where until now SOD1 mutant transgenic mice models have been used to evaluate the potential therapeutic benefit of experimental therapeutics for this specific form of fALS. While deletion of the EphA4 gene (heterozygous) in a SOD1(G93A) mouse model of ALS resulted in improved survival (Van Hoecke et al., Nature medicine, 18 (9): 1418-1422, (2012)), more recent studies seemed to be contradictory on how and when to target EphA4 in ALS. For example, ubiquitous reduction of EphA4 levels to 50% in the same SOD1(G93A) mice at 60 days of age, did not improve disease onset or survival (Rue et al., Sci Rep, 9 (1): 14112, (2019)), suggesting that specific knockdown in EphA4 in adulthood may have a limited therapeutic potential for ALS. Moreover, pharmacological inhibition of EphA4 also produced non-conclusive results using transgenic SOD1(G93A) animal models. For example, earlier studies with an EphA4 antagonistic peptide KYL (Murai et al., Molecular and cellular neurosciences, 24 (4): 1000-1011, (2003)), targeting its ligand binding domain similar to APY-d3, apparently improved onset and survival in a rat model for ALS (Van Hoecke et al., Nature medicine, 18 (9): 1418-1422, (2012)). However, the study also reported a similar in vivo effect with a previously identified small molecule pyrrole-salicylate agent (Noberini et al., The Journal of biological chemistry, 283 (43): 29461-29472, (2008)) that researchers (Tognolini et al., A(CS Chem Neurosci, 5 (12): 1146-1147, (2014); Wu et al., Cell Chem Biol, 24 (3): 293-305, (2017)) later recognized to be a false positive, and not targeting EphA4 potently nor specifically, perhaps further corroborating the variability of the transgenic models in evaluating the potential efficacy of experimental therapeutics. A more controlled study with the optimized EphA4 antagonist agent APY-d3 (Olson et al., ACS Med Chem Lett, 7 (9): 841-846, (2016)) was also recently reported and the studies concluded there was no difference between treatment groups and controls in disease onset or survival (ephrins.org/doc/libro_abstract_2018.pdf). More recently, a fusion protein combining the extracellular domain of wild-type EphA4 with an IgG Fc fragment (EphA4-Fc) has been proposed as a decoy to suppress EphA4 signaling, and showed in SOD1(G93A) mice models only a modest improvement in survival but a more significant improvement on delaying disease onset (Zhao et al., Sci Rep, 8 (1): 11393, (2018)).

EphA4 binds to their natural ligands, the ephrins, inducing bidirectional signaling, and the ligands can interact with the receptor in cis (i.e from the same cells) or from adjacent cells (i.e. the astrocytes) (Pasquale, Cell, 133 (1): 38-52, (2008)). When EphA4 is aberrantly overexpressed, the unbound receptor can exert a pro-apoptotic activity in motor neurons (MNs) (Furne et al., Biochim Biophys Acta, 1793 (2): 231-238, (2009)), while the ephrin bound receptor is not pro-apoptotic, suggesting that ephrin-mimetics (or agonistic agents) may be needed to ameliorate MNs cell death induced by overexpression of EphA4 in ALS patients. Accordingly, it was recently found that reduction of ephrin-A5 aggravates disease progression in amyotrophic lateral sclerosis, perhaps because eliminating its ephrin ligand, unbound EphA4 can exert its pro-apoptotic effect in MNs (Rue et al., Acta Neuropathol Commun, 7 (1): 114, (2019)). These observations collectively would suggest that ephrinA5 mimetics, hence EphA4 agonistic agents, rather than antagonists, or EphA4-decoys, or genetic suppression of EphA4 expression, may provide a benefit for ALS patients. For example, it was shown that in vivo administration of agonistic agent 123C4 daily in SOD1(G93A) transgenic mice significantly prolonged survival in the treated cohort (Wu et al., Cell Chem Biol, 24 (3): 293-305, (2017)).

To preliminarily evaluate the agents described herein, a more disease specific cell-based assay was adopted to monitor the cytoprotective ability of test agents in motor neurons, and also assess their ability to rescue motor neuron death induced by sALS patients derived astrocytes (Haidet-Phillips et al., Nat Biotechnol, 29 (9): 824-828, (2011); Meyer et al., Proc Natl Acad Sci USA, 111 (2): 829-832, (2014)). The data clearly suggest that the agents exert a marked cytoprotective activity and can effectively protect motor neurons from sALS derived iAstrocytes (FIG. 11), providing a clear path for the development of these agents as ALS therapeutics.

The bidirectional signaling mediated by ephrinA5 and EphA4 offers also other possible therapeutic opportunities. For example, ephrinA5 mimetics, like 150D4, could find applications also in oncology, including glioma and colon cancers (Li et al., Oncogene, 28 (15): 1759-1768, (2009); Wang et al., FEBS J, 279 (2): 251-263, (2012); Pensold et al., Int J Mol Sci, 22 (3): (2021)), where ephrinA5 was found to act as a tumor suppressor, interfering with EGFR. Unlike ephrinA5, 150D4 is more selective for the EphA4 subtype, and being a synthetic agent, it is readily available for translation into a therapeutic agent.

This new series of potent and selective EphA4 agonistic agents and 150D4 in particular, represent powerful and unprecedented pharmacological tools to further evaluate and validate the therapeutic potential of the EphA4 signaling for the onset and progression of ALS and possibly other diseases. The agent could be deployed for detailed in vivo efficacy evaluations in models of ALS and potentially other human diseases, including Alzheimer disease (AD) (Fu et al., Proceedings of the National Academy of Sciences of the United States of America, 111 (27): 9959-9964, (2014)) spinal cord injury, (Spanevello et al., J Neurotrauma, 30 (12): 1023-1034, (2013)), brain injury (Frugier et al., J Neuropathol Exp Neurol, 71 (3): 242-250, (2012); Hanell et al., J Neurotrauma, 29 (17): 2660-2671, (2012)), and some type of cancers (Iiizumi et al., Cancer Sci, 97 (11): 1211-1216, (2006); Fukai et al., Mol Cancer Ther, 7 (9): 2768-2778, (2008); Oshima et al., Int J Oncol, 33 (3): 573-577, (2008); Miyazaki et al., BMC Clin Pathol, 13 (1): 19, (2013)). In conclusion, the described agents and related data form the basis for the immediate development of novel therapeutics.

### Experimental Procedures

### Chemistry

General. All reagents and solvents were obtained from commercial sources, including the majority of Fmoc-protected amino acids and resins for solid phase synthesis. NMR spectra were used to evaluate the concentration of stock solutions and were recorded on Bruker Avance III 700 MHz equipped with a TCI cryo-probe. High resolution mass spectral data were acquired on an Agilent LC-TOF instrument. RP-HPLC purifications were performed on a JASCO preparative system equipped with a PDA detector and a fraction collector controlled by a ChromNAV system (JASCO) on a XTerra C18 10μ 10×250 mm (Waters). Purity of tested compounds was assessed by HPLC using an Atlantis T3 3 μm 4.6×150 mm² column (H₂O/acetonitrile gradient from 5 to 100% in 45 min). All compounds have a purity >95%. APY-d3 was synthesized by Innopep (San Diego), while all other agents were synthesized in house by standard solid-phase Fmoc peptide synthesis protocols on BAL resin. Briefly, for each coupling reaction, 3 eq. of Fmoc-AA, 3 eq. of HATU, 3 eq. of OximaPure, and 5 eq. of DIPEA in 1 ml of DMF were used. The coupling reaction was allowed to proceed for 1 h. Fmoc deprotection was performed by treating the resin-bound peptide with 20% piperidine in DMF twice. Peptides were cleaved from Rink amide resin with a cleavage cocktail containing TFA/TIS/water/phenol (94:2:2:2) for 5 h. The cleaving solution was filtered from the resin, evaporated under reduced pressure and the peptides precipitated in Et₂O, centrifuged and dried in high vacuum.

For the synthesis of Fmoc-amino acids that were not commercially available, 1 equivalent of the unprotected amino acid and 3.75 equivalents of Na₂CO₃ were dissolved in tetrahydrofuran (THF)/H₂O (1:1) and cooled to 0° C. 1.1 equivalent of Fmoc chloride was dissolved in THE and added dropwise to the mixture. The reaction was stirred for 2 h at 0° C. The organic solvent was evaporated under reduced pressure and the pH was lowered to 0 using concentrated HCl. The aqueous phase was extracted 3 times with AcOEt and the collected organic phase was dried with Na₂SO₄, filtered, and evaporated. The resulting crude was purified using a CombiFlash Rf (teledyne ISCO) using cyclohexane/ethyl acetate (10-100%).

Compound 2: (1S,3S)-3-amino-N—((S)-1-(((S)-1-(((S)-6-guanidino-1-((2-((4-(morpholinomethyl)phenyl)amino)-2-oxoethyl)amino)-1-oxohexan-2-yl)amino)-3-(2′-methoxy-[1,1′-biphenyl]-4-yl)-1-oxopropan-2-yl)amino)-3-(5-hydroxy-1H-indol-3-yl)-1-oxopropan-2-yl)cyclohexane-1-carboxamide. BAL resin was used as solid-phase support (0.05 mmol scale). Briefly, a BAL resin was loaded using a solution of 4-(Morpholinomethyl)aniline (3 eq.) in DMF added to the reactor and shaken for 30 min, followed by reduction using Sodium triacetoxyborohydride (3 eq., overnight reaction at room temperature). The resin was subsequently filtered, washed three times with DMF, three times with DCM (3×) and again three times with DMF. For the coupling of Fmoc-Glycine on the secondary amine, reaction time was increased to 2 h. Fmoc deprotection and peptide elongation then followed standard procedures described in the general chemistry section. After cleavage, the crude was purified by preparative RP-HPLC using a XTerra C18 (Waters) and water/acetonitrile gradient (5% to 100%) containing 0.1% TFA. HRMS: calcd 999.53 (M+H)⁺; obs 1000.54 (M+H)*.

Compound 9: (1S,3S)—N—((S)-1-(((S)-3-([1,1′-biphenyl]-4-yl)-1-(((S)-6-guanidino-1-((2-((4-(morpholinomethyl)phenyl)amino)-2-oxoethyl)amino)-1-oxohexan-2-yl)amino)-1-oxopropan-2-yl)amino)-3-(5-hydroxy-1H-indol-3-yl)-1-oxopropan-2-yl)-3-aminocyclohexane-1-carboxamide. BAL resin was used as solid-phase support (0.05 mmol scale), and the previously described conditions for compound 2 were used to obtain the peptidic part of the agent. After cleavage, the crude was purified by preparative RP-HPLC using a XTerra C18 (Waters) and water/acetonitrile gradient (5% to 100%) containing 0.1% TFA. HRMS: calcd 969.52 (M+H)+; obs 970.53 (M+H)+.

General scheme for the synthesis of compounds 9 and 2. Conditions: (a) DMF, 30 min, rt; (b) sodium triacetoxyborohydride (3 eq.), o/n, rt; (c) Fmoc-Gly-COOH (3 equiv), HATU (3 eq.), Oxyma Pure (3 eq.), DIPEA (5 eq.), 2 h, rt; (d) 20% 4-methylpiperidine in DMF, rt; (e) Fmoc-L-HomoArg(Pbf)-OH (3 eq.), HATU (3 eq.), Oxyma Pure (3 eq.), DIPEA (5 eq.), 1 h, rt; (f) Fmoc-p-phenyl-L-phenylalanine or Fmoc-4-(2-methoxyphenyl)-L-phenylalanine (3 eq.), HATU (3 eq.), Oxyma Pure (3 eq.), DIPEA (5 eq.), 1 h, rt; (g) Fmoc-5-Hydroxy-L-tryptophan (3 eq.), HATU (3 eq.), Oxyma Pure (3 eq.), DIPEA (5 eq.), 1 h, rt; (h) (1S,3S)-3-(Boc-amino)cyclohexanecarboxylic acid (3 eq.), HATU (3 eq.), Oxyma Pure (3 eq.), DIPEA (5 eq.), 1 h, rt; (g) TFA/TIS/water/phenol (94:2:2:2), 5 h, rt.

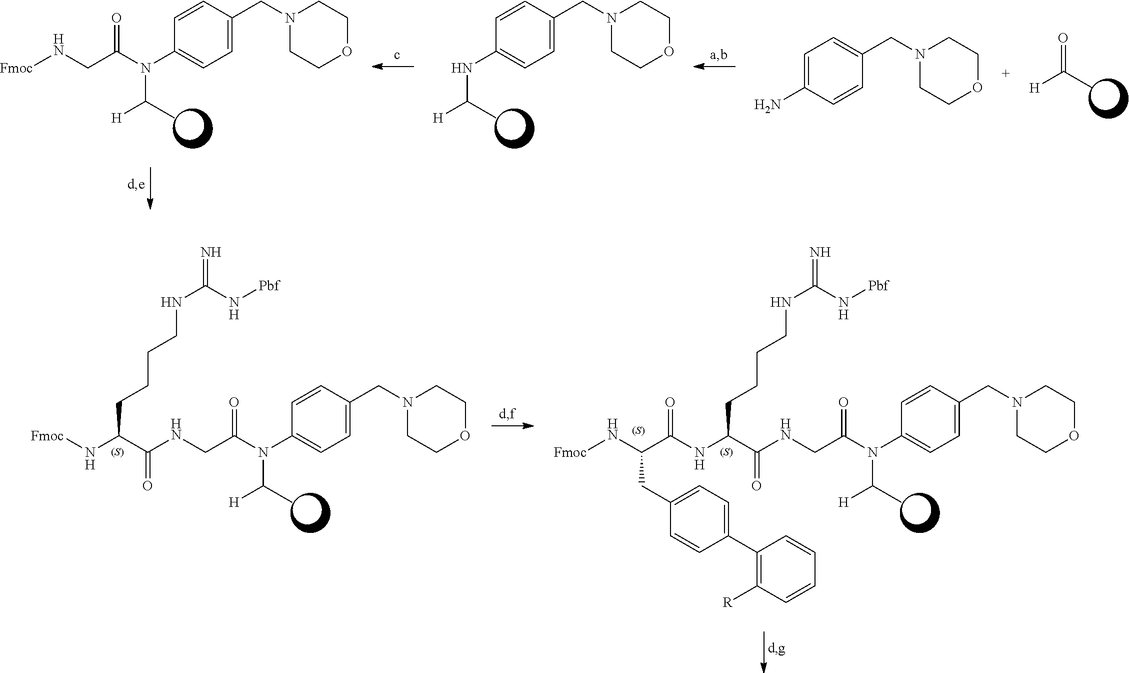

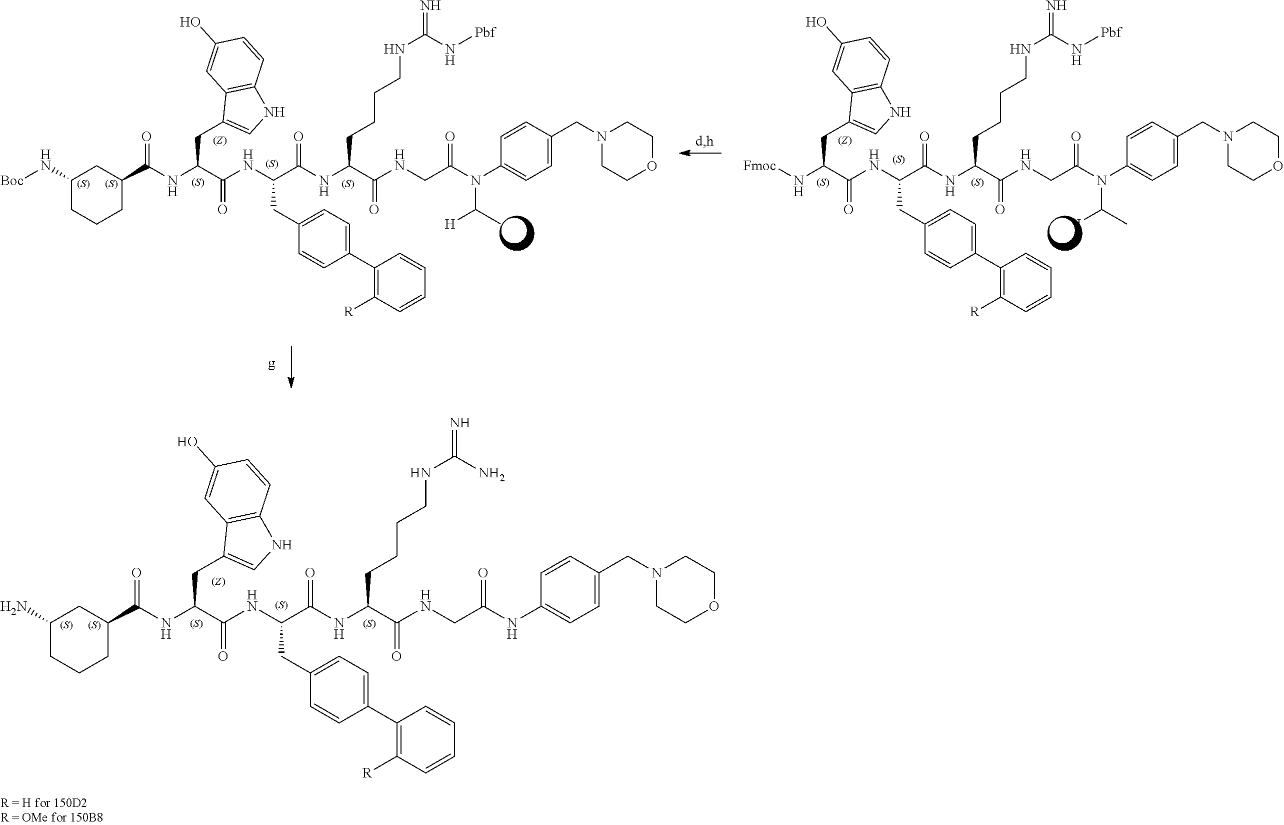

Compound 8 (150D4): (1S,3S)—N—((S)-1-(((S)-3-([1,1′-biphenyl]-4-yl)-1-(((S)-6-guanidino-1-((2-((4-morpholinophenyl)amino)-2-oxoethyl)amino)-1-oxohexan-2-yl)amino)-1-oxopropan-2-yl)amino)-3-(5-hydroxy-1H-indol-3-yl)-1-oxopropan-2-yl)-3-aminocyclohexane-1-carboxamide. BAL resin was used as solid-phase support (0.05 mmol scale). Briefly, a BAL resin was loaded using a solution of 4-Morpholinoaniline (3 eq.) in DMF added to the reactor and shaken for 30 min, followed by reduction using Sodium triacetoxyborohydride (3 eq., overnight reaction at room temperature). The resin was subsequently filtered, washed three times with DMF, three times with DCM (3×) and again three times with DMF. For the coupling of Fmoc-Glycine on the secondary amine reaction time was increased to 2 h. Fmoc deprotection and peptide elongation then followed standard procedures described in the general chemistry section. After cleavage, the crude was purified by preparative RP-HPLC using a XTerra C18 (Waters) and water/acetonitrile gradient (5% to 100%) containing 0.1% TFA. HRMS: calcd 955.51 (M+H)⁺; obs 956.51 (M+H)+, 978.49 (M+Na)+.

Compound 3: (1S,3S)-3-amino-N—((S)-1-(((S)-1-(((S)-6-guanidino-1-((2-((4-morpholinophenyl)amino)-2-oxoethyl)amino)-1-oxohexan-2-yl)amino)-3-(2′-methoxy-[1,1′-biphenyl]-4-yl)-1-oxopropan-2-yl)amino)-3-(5-hydroxy-1H-indol-3-yl)-1-oxopropan-2-yl)cyclohexane-1-carboxamide. BAL resin was used as solid-phase support (0.05 mmol scale), and the previously described conditions for compound 8 (150D4) were used to obtain the peptidic part of the agent. After cleavage, the crude was purified by preparative RP-HPLC using a XTerra C18 (Waters) and water/acetonitrile gradient (5% to 100%) containing 0.1% TFA. HRMS: calcd 985.52 (M+H)+; obs 986.53 (M+H)+, 1008.51 (M+Na)+.

General scheme for the synthesis of compounds 150D4 and 3. Conditions: (a) DMF, 30 min, rt; (b) sodium triacetoxyborohydride (3 eq.), o/n, rt; (c) Fmoc-Gly-COOH (3 equiv), HATU (3 eq.), Oxyma Pure (3 eq.), DIPEA (5 eq.), 2 h, rt; (d) 20% 4-methylpiperidine in DMF, rt; (e) Fmoc-L-HomoArg(Pbf)-OH (3 eq.), HATU (3 eq.), Oxyma Pure (3 eq.), DIPEA (5 eq.), 1 h, rt; (f) Fmoc-p-phenyl-L-phenylalanine or Fmoc-4-(2-methoxyphenyl)-L-phenylalanine (3 eq.), HATU (3 eq.), Oxyma Pure (3 eq.), DIPEA (5 eq.), 1 h, rt; (g) Fmoc-5-Hydroxy-L-tryptophan (3 eq.), HATU (3 eq.), Oxyma Pure (3 eq.), DIPEA (5 eq.), 1 h, rt; (h) (1S,3S)-3-(Boc-amino)cyclohexanecarboxylic acid (3 eq.), HATU (3 eq.), Oxyma Pure (3 eq.), DIPEA (5 eq.), 1 h, rt; (g) TFA/TIS/water/phenol (94:2:2:2), 5 h, rt.

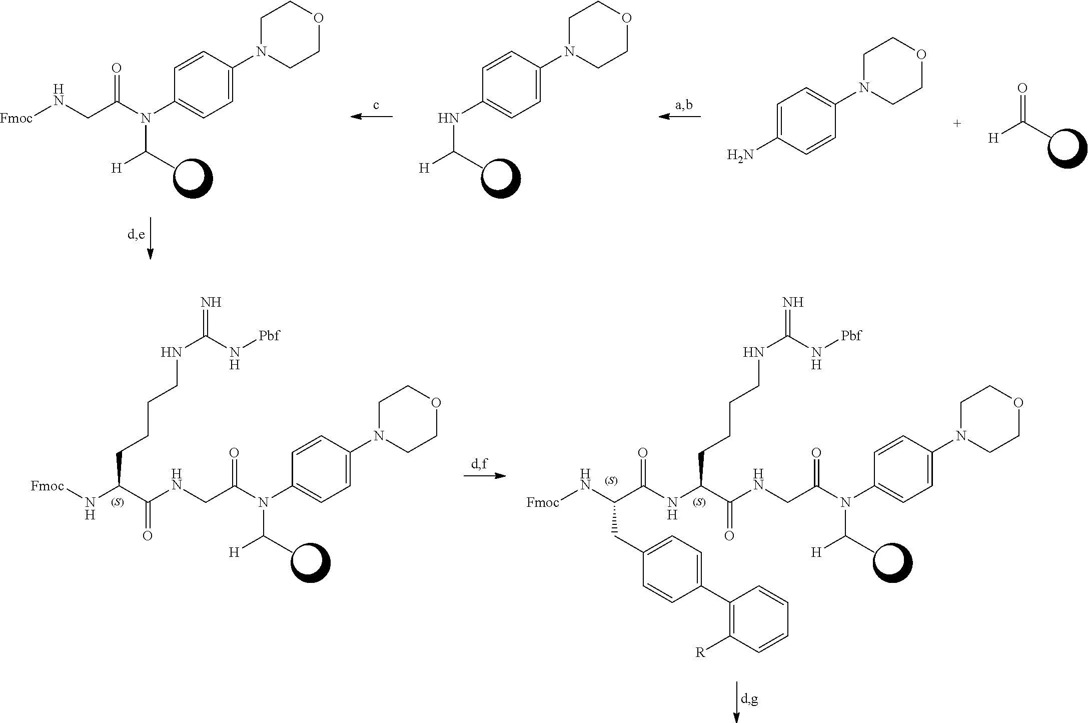

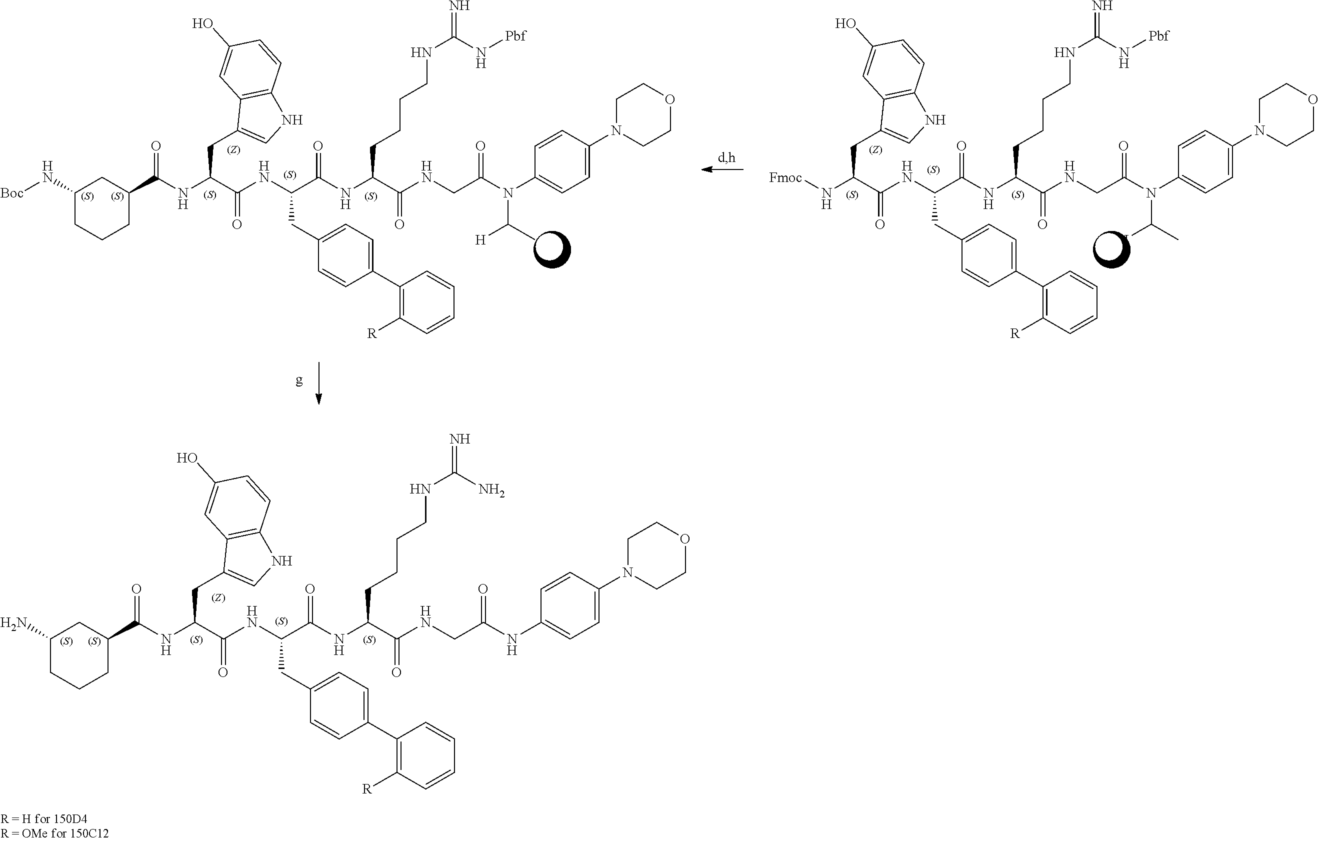

Protein Expression and Purification. cDNA fragments encoding the ligand binding domain of EphA4 (residues 29-209) cloned into a pET15b vector with the Cysteine 204 mutated to Alanine for stability and an N-terminal His tag were used in the expressions of EphA4-LBD (Baggio et al., J Med Chem, (2018)). The fragments were transformed into Rosetta-Gami B(DE3) competent cells and grown in LB medium at 37° C. with 100 μg/mL of ampicillin until reaching an OD₆₀₀ of 0.6-0.7 followed by induction with 0.4 mM IPTG overnight at 20° C. Bacteria were then collected by centrifugation and lysed by sonication at 4° C. Proteins were purified using Ni²⁺ affinity chromatography, eluted in 25 mM Tris at pH 7.5, 500 mM NaCl, and 500 mM imidazole. Finally, the protein was further purified, and buffer exchanged, through a size-exclusion chromatography with a HiLoad 26/60 Superdex 75 preparative-grade column into an aqueous buffer composed of 25 mM Tris at pH 7.5, 150 mM NaCl. EphA4 LBD with ¹³C^{ε}/¹H^{ε}-Met labeled was expressed like previously described but adding a suspension of 100 mg of ¹³C^{ε}/¹H^{ε}-Methionine in 1 mL of DMSO per liter of LB medium, 10 minutes before induction. EphA3 chimera was produced by introduction the following mutations in EphA4-LBD (29-209): Arg37Lys, Gly52His, Ile59Gly, Met60Val, Glu77Asp, Val157Met, Ile159Leu, Met164Leu, Cys204Ala. Transformation, expression, and purification of EphA3-LBD chimera was performed as described above for EphA4-LBD.

For the expression of EphA2-LBD, a pET15b vector encoding for the EphA2 ligand binding domain (residues 27-200) and an N-terminal His tag was transformed into Origami (DE3) competent cells. The transformed cells were transferred to LB medium at 37° C. with 100 g/L of ampicillin until reaching an OD₆₀₀ of 0.6-0.7, followed by induction with 0.5 mM IPTG overnight at 20° C. Bacteria were collected and lysed by sonication at 4° C. The overexpressed protein was purified using Ni²⁺ affinity chromatography and further purified and buffer exchanged through a size-exclusion chromatography with a HiLoad 26/60 Superdex 75 preparative-grade column into an aqueous buffer composed of 25 mM Tris at pH 7.5, 150 mM NaCl. Human Ephrin-A5 was obtained from Sino Biological US Inc (Chesterbrook, Pa.).

### Isothermal Titration Calorimetry (ITC) Measurements.

Isothermal titration calorimetry (ITC) measurements were performed using the Affinity ITC Autosampler from TA Instruments (New Castle, Del.). The titrations were performed in a reverse fashion by titrating the protein into the ligand solution. All the measurements were performed at 25° C. dissolving the agents in 25 mM Tris at pH 7.5, 150 mM NaCl, and a final DMSO concentration of 1%. The syringe was filled with a 100 μM solution of EphA4-LBD, EphA3-LBD Chimera, or EphA2-LBD, and 20 injections of 2.5 μL each were performed into the cell containing a 20 μM solution of the compounds. The injections were made at 200 s or 400 s intervals with a stirring speed of 75 rpm. All the solutions were kept in the autosampler at 4° C. The analysis of the thermodynamics signatures and for dissociation constant determination was performed by the NanoAnalyze software (TA Instruments, New Castle, Del.) and subsequently exported into Microsoft Excel.

Nuclear Magnetic Resonance (NMR) spectroscopy.

NMR spectra were acquired on Bruker Avance III 700 MHz spectrometer equipped with a TCI cryoprobe. All NMR data were processed and analyzed using TOPSPIN 3.6.1 (Bruker, Billerica, Mass., USA). 2D-[¹³C,¹H]-HSQC experiments were acquired with 20 μM proteins using 8 scans with 2,048 and 256 complex data points in the ¹H and ¹⁵N dimensions, respectively, at 298 K. For the 150D4 NMR titrations in FIG. 8 2D-[¹³C,¹H]-HSQC experiments were acquired using 16 scans per increment, and for ephrinA5 binding studies (FIG. 8) with 10 μM protein, using 32 scans per increment.

For the fHTS by NMR screening, each of the 138 mixtures (3×46) was dissolved into a 5 mm NMR tube to a final concentration of 2 mM in the presence of 20 μM of EphA4-LBD in a buffer containing 25 mM TRIS pH=7.5, 150 mM NaCl. For each mixture 2D [¹³C,¹H] HSQC and 1D ¹H-aliph experiments were acquired. For ranking purposes (FIG. 5) a total chemical shift perturbation generated by each mixtures to the 5 peaks below 0 ppm in the 1D ¹H-aliph spectra of EphA4-LBD were considered.

Chemical shift changes (A) in the 2D [¹³C, ¹H] spectra were calculated as weight average perturbations observed in the ¹H and ¹³C dimensions using the following Equation:

$$Δδ = \sqrt{\frac{1}{2} * [ {( {Δ}^{1} H )}^{2} + {( 0.3 * {Δ}^{13} C )}^{2} ]}$$

### Molecular Modeling

Molecular models were analyzed using MOE 2019.0101 (Chemical Computing Group) or Chimera (cgl.ucsf.edu/chimera). Structural comparisons were carried out between the X-ray structure of 150D4 in complex with EphA4-LBD and the structures of the complexes between EphA4 and APY-D3 (PDB-ID 5JR2), EphA4-ephrinA5 (PDB-ID 4BKA), and apo Eph4-LBD (PDB-ID 2WO1).

### X-Ray Structure Determination.

EphA4-LBD was used in crystallisation trials employing both a standard screen with approximately 1200 different conditions, as well as crystallisation conditions identified using literature data. Conditions initially obtained have been optimised using standard strategies, systematically varying parameters critically influencing crystallisation, such as temperature, protein concentration, drop ratio, and others. These conditions were also refined by systematically varying pH or precipitant concentrations. A cryo-protocol was established using PROTEROS Standard Protocols. Crystals have been flash-frozen and measured at a temperature of 100 K. The X-ray diffraction data have been collected from complex crystals of EphA4-LBD mutant C204A with the ligand 150D4 at the Deutsches Elektronen-Synchrotron (DESY, Hamburg, Germany) using cryogenic conditions. The crystals belong to space group P 43 21 2. Data were processed using the programmes autoPROC, XDS and autoPROC, AIMLESS. The phase information necessary to determine and analyze the structure was obtained by molecular replacement. Model building and refinement was performed according to standard protocols with COOT and the software package CCP4, respectively. For the calculation of the free R-factor, a measure to cross-validate the correctness of the final model, about 4.9% of measured reflections were excluded from the refinement procedure (see Table 10). Anisotropic B-factor refinement (using REFMAC5, CCP4) has been carried out, which resulted in lower R-factors and higher quality of the electron density map. The ligand parameterization and generation of the corresponding library files were carried out with CORINA. The water model was built with the “Find waters”-algorithm of COOT by putting water molecules in peaks of the Fo-Fc map contoured at 3.0 followed by refinement with REFMAC5 and checking all waters with the validation tool of COOT. The criteria for the list of suspicious waters were: B-factor greater 80 A2, 2Fo-Fc map less than 1.2 s, distance to closest contact less than 2.3 Å or more than 3.5 Å. The suspicious water molecules and those in the ligand binding site (distance to ligand less than 10 Å) were checked manually. The Ramachandran Plot of the final model shows 90.3% of all residues in the most favored region, 9.1% in the additionally allowed region, and 0.6% in the generously allowed region. No residues are found in the disallowed region. Statistics of the final structure and the refinement process are listed in Table 10.

TABLE 10
Data collection and Refinement statistics for 150D4

| Ligand | 150D4 | |
|---|---|---|
| Data collection | | |
| X-ray source | P13 | (DESY) |
| Wavelength [Å] | 1.0332 | |
| Detector | PILATUS 6M | |
| Temperature [K] | 100 | |
| Space group | P 4₃ 2₁ 2 | |
| Cell: | | |
| a; b; c; [Å] | 55.03; 55.03; 147.30 | |
| α; β; γ; [°] | 90.0; 90.0; 90.0 | |
| Resolution [Å] | 1.43 | (1.54-1.43) |
| Unique reflections | 34710 | (1736) |
| Multiplicity | 12.9 | (4.0) |
| Spherical completeness [%] | 81.0 | (20.1) |
| Ellipsoidal completeness [%] | 93.8 | (57.1) |
| Rₚᵢₘ [%] | 1.5 | (48.0) |
| R_{sym} [%] | 5.3 | (88.3) |
| Rₘₑₐₛ [%] | 5.5 | (101.2) |
| CC½ [%] | 100.00 | (53.40) |
| Mean(I)/sd | 24.9 | (1.2) |

| | |
|---|---|
| Refinement statistics | |
| Resolution [Å] | 51.55-1.43 |
| Number of reflections (working/test) | 32995/1715 |
| R_{cryst} [%] | 15.3 |
| R_{free} [%]² | 20.8 |
| Total number of atoms: | |
| Protein | 1517 |
| Water | 303 |
| Ligand | 70 |
| Acetate | 4 |
| Deviation from ideal geometry: | |
| Bond lengths [Å] | 0.010 |
| Bond angles [°] | 1.64 |
| Bonded B’s [Å²] | 3.4 |
| Ramachandran plot: | |
| Most favoured regions [%] | 90.3 |
| Additional allowed regions [%] | 9.1 |
| Generously allowed regions [%] | 0.6 |
| Disallowed regions [%] | 0.0 |

### Primary Motor Neurons

Primary motor neurons were isolated from spinal cords of B6.Cg-Tg(Hlxb9-GFP)1Tmj/J (Hb9-GFP) mice at postnatal day (P) 0-2. Tissues were dissected, cut into 1-2 mm pieces and treated with a papain/DNase I (0.1 M PBS/0.1% BSA/25 mM glucose/5% papain/1×DNase I [Sigma, #D5025-15K]) solution for 20 min at 37° C. Cells were mechanically dissociated, filtered using a 100 m cell strainer and further purified using OptiPrep gradient centrifugation as described in (Wang, 2017). Neurons were plated on poly-D-lysine (0.5 mg/mL) and laminin (5 μg/mL) coated 6- or 24-well plates (350,000 cells per well of 6-well plate and 75,000 cells per well of 24-well plate) in Neurobasal media with 25 mM glutamine, 1% penicillin-streptomycin, B27 supplement (Invitrogen, #17504-044). After 2 h media was changed to a fresh media containing 5% horse serum (Gibco, #26050-070) and 10 ng CTNF (Sino Biological, #11841-H-07E-5). Cells were maintained under 5% CO₂/10% 02 atmosphere at 37° C. for two days.

### EphA4 Receptor Activation

EphrinA1-Fc (R&D Systems, #602-A1) and human Fc (R&D Systems, #110-HG) were pre-clustered by the incubation with goat anti-human IgG (Jackson ImmunoResearch, #109-005-003) for 1 h at 4° C. At 2 days in vitro (DIV) primary motor neurons were treated with pre-clustered Fc (2 μg/mL), pre-clustered ephrinA1-Fc (2 μg/mL), APYd3, 123C4, compound 2, compound 9, compound 3 or 150D4 (at concentrations 1 μM and 10 μM) for 30 min at 37° C. under 5% CO₂/10% O₂ atmosphere and then processed for western blotting. For growth cone analysis 2 DIV primary spinal cord motor neurons were treated as described above with Fc, ephrinA1-Fc, 1 μM 150D4, 10 μM 150D4, 1 μM 150D4 plus ephrinA1-Fc or 10 μM 150D4 plus ephrinA1-Fc. 0.1% DMSO was used as a negative control in both experiments.

### Immunoprecipitation and Western Blot Analysis

Cells were collected and lysed in the lysis buffer (25 mM Tris-HCl, 150 mM NaCl, 5 mM EDTA, 1% Triton X-100, 1 mM sodium pervanadate, and protease inhibitor cocktail [1:100, Sigma, #P8340]) at 4° C. for 30 min. Cell lysates were cleared by centrifugation at 13,500 rpm for 20 min at 4° C., then incubated with protein-A agarose beads (Sigma, #P1406) and anti-EphA4 antibody (Invitrogen, #371600), for 2 h at 4° C. Beads and cell lysate were boiled in reducing sample buffer (Laemmli 2×concentrate, Sigma, #S3401). Samples were briefly spun down and the supernatant was run on an 8%-16% Tris-glycine SDS-PAGE (Invitrogen, #XP08160BOX). Proteins were transferred onto Protran BA 85 nitrocellulose membrane (GE Healthcare) and blocked for 1 h at room temperature in 5% BSA. The blots were incubated with anti-phosphotyrosine antibody (BD Transduction, #610000) in Tris-buffered saline (TBS)/0.1% Tween 20/1% BSA at 4° C. overnight. Membranes then were washed 3×10 min with TBS/0.1% Tween-20/1% BSA and incubated with HRP-conjugated anti-mouse secondary antibodies at 1:5000 (Jackson ImmunoResearch, #715-035-150) for 2 h at room temperature in a TBS/0.1% Tween-20/1% BSA solution. Blots were further incubated with ECL Detection reagent (Thermo Scientific, #32106) and imaged using ChemiDoc imaging system (Bio-Rad). For reprobing, membrane blots were washed in stripping buffer (2% SDS, 100 mM β-mercaptoethanol, 50 mM Tris-HCl [pH 6.8]) for 30 min at 55° C. and then washed 5×5 min with TBST, blocked with 5% skim milk, and re-probed for EphA4 (Invitrogen, #371600). Blots were washed 3×10 min with TBS/0.1% Tween 20 and then incubated with anti-mouse HRP-conjugated secondary antibodies in TBS/0.1% Tween 20/1% BSA (Jackson ImmunoResearch, #715-035-150) for 2 h at room temperature. After the incubation, blots were washed 3×10 min with TBS/0.1% Tween 20 and developed as described above. Blots with cell lysate samples were probed for ChAT (rabbit anti-mouse, Millipore-Sigma, #AB143, 1:1000) overnight and then incubated with corresponding secondary HRP-conjugated goat anti-rabbit antibody (Thermo Fisher Scientific, #G-21234) for 2 h as described above. Band density was analyzed by measuring band and background intensity using Adobe Photoshop CS5.1 software.

### Immunocytochemistry

Primary neurons were fixed with 2% paraformaldehyde in 0.1 M PBS for 30 min at room temperature, then washed 3×10 min with 0.1 M PBS. Cells were permeabilized for 10 min at room temperature with 0.1% Triton X-100 in 0.1 M PBS and then washed 3×10 min in 0.1 M PBS. Cells were blocked with 5% NDS in 0.1 M PBS for 1 h at room temperature. For visualization of axon growth cones, cells were stained with phalloidin-rhodamine (1:40, Invitrogen, #R415) in a blocking buffer for 1 h at room temperature and motor neuron marker was immunolabeled with anti-ChAT antibody (1:500, Millipore-Sigma, #AB143) in 0.1 M PBS containing 1% NDS overnight at 4° C. Coverslips then were washed 3×10 min with 0.1 M PBS at room temperature followed by the incubation with Alexa Fluor 350-conjugated goat anti-rabbit immunoglobulin G (IgG) (1:500, Invitrogen, #A21068) for 2 h at room temperature. Coverslips were mounted on slides with Vectashield mounting medium (Vector Laboratories, #H100010).

### Image Analysis

For growth cone analysis, images were captured using a Nikon Eclipse TE2000-U inverted fluorescent microscope with a 20×air objective and a Hamamatsu ORCA-AG 12-bit CCD camera using Image-Pro software. For analysis, 100 images were collected (2 coverslips/group, 3 experiments, 1-3 neurons/image) per treatment group. Growth cones were assessed based on filamentous (F)-actin labeling and classified into collapsed and growing based on their morphology. The percentage of neurons with collapsed growth cones was determined. Statistical differences for multiple groups were assessed by one-way ANOVA followed by Bonferroni's post hoc tests.

Co-Culture Studies with Human Astrocytes and Motor Neurons

Patient fibroblasts were reprogrammed directly into neuronal progenitor cells (NPCs) as previously described (Meyer et al., Proc Natl Acad Sci USA, 111 (2): 829-832, (2014)). Induced Astrocytes were generated by seeding a low quantity of NPCs into astrocyte medium (DMEM media containing 10% FBS and 0.2% N2) for five days. Following differentiation, iAstrocytes were lifted and seeded into a 96 well (10,000 cells per well).

Motor neurons expressing GFP under an HB9 promotor were differentiated from mouse embryonic bodies as previously described (Meyer et al., Proc Natl Acad Sci USA, 111 (2): 829-832, (2014)), EBs were dissociated with papain and sorted using Becton-Dickenson Influx sorter using software. Cells are sorted through a 100 micron tip with sheath pressure of 27. GFP+ motor neurons were seeded in a 96 well plate (10,000 cells per well). Co-cultures were imaged with InCell for up to three days. Motor neurons with neurite outgrowth of greater than 50 um were counted as alive.

Abbreviations used: BAL, 5-(4-Formyl-3,5-dimethoxyphenoxy)pentanoyl amido (4-methylphenyl)methyl polystyrene resin; HATU, 1-[Bis(dimethylamino)methylene]-1H-1,2,3-triazolo[4,5-b]pyridinium 3-oxid hexafluorophosphate; DIPEA, N,N-Diisopropylethylamine; DMF, Dimethylformamide.

All publications, patents, and patent documents are incorporated by reference herein, as though individually incorporated by reference. The invention has been described with reference to various specific and preferred embodiments and techniques. However, it should be understood that many variations and modifications may be made while remaining within the spirit and scope of the invention.

What is claimed is:

1. A peptide of Formula (I) X₀—X₁—X₂—X₃—X₄ from N terminal to C terminal, wherein:
X₀ is a residue of an amino acid, wherein the N-terminus is a primary amine group NH₂—, or a capped amine R^{N}—C(═O)—NH—, wherein R^{N} is H, (C₃-C₆)cycloalkyl, or (C₁-C₄)alkyl;
X₁ is a residue of Trp, wherein Trp is optionally substituted with one or more groups independently selected from the group consisting of halo, hydroxy, cyano, carboxyl, —CONH₂, —NO₂, —SH, —SO₃H, —SO₄H, —SO₂NH₂, —OSO₂F, —SO₂F, —NHNH₂, —ONH₂, —NHC(═O)NHNH₂, —NHC(═O)NH₂, —NHC(═O)H, —NHC(═O)OH, —NHOH, (C₁-C₆)alkyl, (C₁-C₆)alkoxy, (C₁-C₆)alkoxycarbonyl, (C₁-C₆)alkanoyl, (C₁-C₆)alkanoyloxy, (C₁-C₆)heteroalkyl, (C₃-C₆)cycloalkyl, heterocycloalkyl, heterocycloalkyl(C₁-C₆)alkyl, aryl, heteroaryl, and —NR^{r}R^{s}, wherein any (C₁-C₆)alkyl, (C₁-C₆)alkoxy, (C₁-C₆)alkoxycarbonyl, (C₁-C₆)alkanoyl, (C₁-C₆)alkanoyloxy, (C₁-C₆)heteroalkyl, (C₃-C₆)cycloalkyl, heterocycloalkyl, heterocycloalkyl(C₁-C₆)alkyl, aryl, and heteroaryl is optionally substituted with one or more groups independently selected from the group consisting of halo, hydroxy, and (C₁-C₆)alkoxy;
X₂ is a residue of Bip, wherein Bip is optionally substituted on one or both phenyl groups with one or more groups independently selected from the group consisting of halo, hydroxy, cyano, carboxyl, —CONH₂, —NO₂, —SH, —SO₃H, —SO₄H, —SO₂NH₂, —OSO₂F, —SO₂F, —NHNH₂, —ONH₂, —NHC(═O)NHNH₂, —NHC(═O)NH₂, —NHC(═O)H, —NHC(═O)OH, —NHOH, (C₁-C₆)alkyl, (C₁-C₆)alkoxy, (C₁-C₆)alkoxycarbonyl, (C₁-C₆)alkanoyl, (C₁-C₆)alkanoyloxy, (C₁-C₆)heteroalkyl, (C₃-C₆)cycloalkyl, heterocycloalkyl, heterocycloalkyl(C₁-C₆)alkyl, aryl, heteroaryl, and —NR^{r}R^{s}, wherein any (C₁-C₆)alkyl, (C₁-C₆)alkoxy, (C₁-C₆)alkoxycarbonyl, (C₁-C₆)alkanoyl, (C₁-C₆)alkanoyloxy, (C₁-C₆)heteroalkyl, (C₃-C₆)cycloalkyl, heterocycloalkyl, heterocycloalkyl(C₁-C₆)alkyl, aryl, and heteroaryl is optionally substituted with one or more groups independently selected from the group consisting of halo, hydroxy, and (C₁-C₆)alkoxy;
X₃ is a residue of an amino acid;
X₄ is absent or a residue of an amino acid, wherein the C-terminus is a carboxyl group —COOH, or the C-terminus is amidated to form —C(═O)NR^{x}R^{y};
wherein each R^{x} and R^{y} is independently selected from the group consisting of H, (C₁-C₆)alkyl, (C₃-C₆)cycloalkyl, aryl, heteroaryl, (C₃-C₆)cycloalkyl(C₁-C₆)alkyl, aryl(C₁-C₆)alkyl, heteroaryl(C₁-C₆)alkyl, aryl-heterocycloalkyl-aryl, aryl-heterocycloalkyl-heteroaryl, aryl-heterocycloalkyl-(C₁-C₆)alkyl-aryl, aryl-heterocycloalkyl-(C₁-C₆)alkyl-heteroaryl, and aryl-(C₁-C₆)alkyl-heterocycloalkyl-aryl,
wherein any (C₁-C₆)alkyl, (C₃-C₆)cycloalkyl, aryl, heteroaryl, (C₃-C₆)cycloalkyl(C₁-C₆)alkyl, aryl(C₁-C₆)alkyl, heteroaryl(C₁-C₆)alkyl, aryl-heterocycloalkyl-aryl, aryl-heterocycloalkyl-heteroaryl, aryl-heterocycloalkyl-(C₁-C₆)alkyl-aryl, aryl-heterocycloalkyl-(C₁-C₆)alkyl-heteroaryl, and aryl-(C₁-C₆)alkyl-heterocycloalkyl-aryl is optionally substituted with one or more groups independently selected from the group consisting of halo, hydroxy, cyano, carboxyl, —CONH₂, —NO₂, —SH, —SO₃H, —SO₄H, —SO₂NH₂, —OSO₂F, —SO₂F, —NHNH₂, —ONH₂, —NHC(═O)NHNH₂, —NHC(═O)NH₂, —NHC(═O)H, —NHC(═O)OH, —NHOH, (C₁-C₆)alkyl, (C₁-C₆)alkoxy, (C₁-C₆)alkoxycarbonyl, (C₁-C₆)alkanoyl, (C₁-C₆)alkanoyloxy, (C₁-C₆)heteroalkyl, (C₃-C₆)cycloalkyl, heterocycloalkyl, heterocycloalkyl(C₁-C₆)alkyl, aryl, heteroaryl, and —NR^{r}R^{s}, wherein any (C₁-C₆)alkyl, (C₁-C₆)alkoxy, (C₁-C₆)alkoxycarbonyl, (C₁-C₆)alkanoyl, (C₁-C₆)alkanoyloxy, (C₁-C₆)heteroalkyl, (C₃-C₆)cycloalkyl, heterocycloalkyl, heterocycloalkyl(C₁-C₆)alkyl, aryl, and heteroaryl is optionally substituted with one or more groups independently selected from the group consisting of halo, hydroxy, and (C₁-C₆)alkoxy;
each R^{r} and R^{s} is independently selected from the group consisting of H, (C₁-C₆)alkyl, aryl, and (C₁-C₆)alkoxyaryl;
or a salt thereof.

2. The peptide of claim 1, wherein the N-terminus of the peptide is a primary amine group.

3. The peptide of any one of claims 1-2, wherein X₀ is a residue of a gamma-amino acid.

4. The peptide of claim 1, wherein X₀ is a residue of gamma-amino-butyric acid (GABA).

5. The peptide of claim 1, wherein X₀ is a residue of 3-amino-cyclohexanecarboxylic acid (ACHC).

6. The peptide of claim 1, wherein X₀ is a residue of a beta-amino acid, gamma-amino acid, or delta-amino acid, wherein the N-terminus is a primary amine group NH₂—, or a capped amine R^{N}—C(═O)—NH—, wherein R^{N} is H, (C₃-C₆)cycloalkyl, or (C₁-C₄)alkyl.

7. The peptide of any one of claims 1-6, wherein X₁ is a residue of 5-hydroxy-Trp or 5-methoxy-Trp.

8. The peptide of any one of claims 1-7, wherein X₂ is a residue of Bip or 4-(2-methoxyphenyl)-Phe.

9. The peptide of any one of claims 1-8, wherein X₃ is a positively charged amino acid residue.

10. The peptide of any one of claims 1-9, wherein X₃ is a residue of hArg, Arg, hLys, Lys, Orn, Dab, Dap, or 4-guanidino Phe.

11. The peptide of any one of claims 1-10, wherein X₄ is a residue of Gly, Ala, Thr or Ser.

12. The peptide of any one of claims 1-10, wherein X₄ is a residue of an amino acid, wherein the C-terminus is a carboxyl group —COOH, or the C-terminus is amidated to form —C(═O)NR^{x}R^{y}.

13. The peptide of any one of claims 1-10, wherein X₄ is absent.

14. The peptide of any one of claims 1-13, wherein the C-terminus of the peptide of Formula (I) is amidated and R^{x} is H; and R^{y} is aryl or heteroaryl, wherein the aryl and heteroaryl is optionally substituted with one or more groups independently selected from the group consisting of halo, hydroxy, cyano, carboxyl, —CONH₂, —NO₂, —SH, —SO₃H, —SO₄H, —SO₂NH₂, —OSO₂F, —SO₂F, —NHNH₂, —ONH₂, —NHC(═O)NHNH₂, —NHC(═O)NH₂, —NHC(═O)H, —NHC(═O)OH, —NHOH, (C₁-C₆)alkyl, (C₁-C₆)alkoxy, (C₁-C₆)alkoxycarbonyl, (C₁-C₆)alkanoyl, (C₁-C₆)alkanoyloxy, (C₁-C₆)heteroalkyl, (C₃-C₆)cycloalkyl, heterocycloalkyl, heterocycloalkyl(C₁-C₆)alkyl, aryl, heteroaryl, and —NR^{r}R^{s}, wherein any (C₁-C₆)alkyl, (C₁-C₆)alkoxy, (C₁-C₆)alkoxycarbonyl, (C₁-C₆)alkanoyl, (C₁-C₆)alkanoyloxy, (C₁-C₆)heteroalkyl, (C₃-C₆)cycloalkyl, heterocycloalkyl, heterocycloalkyl(C₁-C₆)alkyl, aryl, and heteroaryl is optionally substituted with one or more groups independently selected from the group consisting of halo, hydroxy, and (C₁-C₆)alkoxy.

15. The peptide of any one of claims 1-13, wherein the C-terminus of the peptide of Formula (I) is amidated and R^{x} is H; and R^{y} is phenyl that is optionally substituted with one or more groups independently selected from halo, hydroxy, cyano, carboxyl, —CONH₂, —NO₂, —SH, —SO₃H, —SO₄H, —SO₂NH₂, —OSO₂F, —SO₂F, —NHNH₂, —ONH₂, —NHC(═O)NHNH₂, —NHC(═O)NH₂, —NHC(═O)H, —NHC(═O)OH, —NHOH, (C₁-C₆)alkyl, (C₁-C₆)alkoxy, (C₁-C₆)alkoxycarbonyl, (C₁-C₆)alkanoyl, (C₁-C₆)alkanoyloxy, (C₁-C₆)heteroalkyl, (C₃-C₆)cycloalkyl, heterocycloalkyl, heterocycloalkyl(C₁-C₆)alkyl, aryl, heteroaryl, and —NR^{r}R^{s}, wherein any (C₁-C₆)alkyl, (C₁-C₆)alkoxy, (C₁-C₆)alkoxycarbonyl, (C₁-C₆)alkanoyl, (C₁-C₆)alkanoyloxy, (C₁-C₆)heteroalkyl, (C₃-C₆)cycloalkyl, heterocycloalkyl, heterocycloalkyl(C₁-C₆)alkyl, aryl, and heteroaryl is optionally substituted with one or more groups independently selected from the group consisting of halo, hydroxy, and (C₁-C₆)alkoxy.

16. The peptide of any one of claims 1-13, wherein the C-terminus of the peptide of Formula (I) is amidated and R^{x} is H; and R^{y} is 2-pyridyl that is optionally substituted with one or more groups independently selected from halo, hydroxy, cyano, carboxyl, —CONH₂, —NO₂, —SH, —SO₃H, —SO₄H, —SO₂NH₂, —OSO₂F, —SO₂F, —NHNH₂, —ONH₂, —NHC(═O)NHNH₂, —NHC(═O)NH₂, —NHC(═O)H, —NHC(═O)OH, —NHOH, (C₁-C₆)alkyl, (C₁-C₆)alkoxy, (C₁-C₆)alkoxycarbonyl, (C₁-C₆)alkanoyl, (C₁-C₆)alkanoyloxy, (C₁-C₆)heteroalkyl, (C₃-C₆)cycloalkyl, heterocycloalkyl, heterocycloalkyl(C₁-C₆)alkyl, aryl, heteroaryl, and —NR^{r}R^{s}, wherein any (C₁-C₆)alkyl, (C₁-C₆)alkoxy, (C₁-C₆)alkoxycarbonyl, (C₁-C₆)alkanoyl, (C₁-C₆)alkanoyloxy, (C₁-C₆)heteroalkyl, (C₃-C₆)cycloalkyl, heterocycloalkyl, heterocycloalkyl(C₁-C₆)alkyl, aryl, and heteroaryl is optionally substituted with one or more groups independently selected from the group consisting of halo, hydroxy, and (C₁-C₆)alkoxy.

17. The peptide of claim 1, wherein:
X₀ is a residue of GABA or ACHC,
X₁ is a residue of Trp, 5-hydroxy-Trp or 5-methoxy-Trp,
X₂ is a residue of Bip or 4-(2-methoxyphenyl)-Phe,
X₃ is a residue of hArg, Arg, hLys, Lys, Orn, Dab or Dap,
X₄ is a residue of Gly, Ala, Thr or Ser,
wherein the C-terminus of the peptide of Formula (I) is amidated and R^{x} is H; and R^{y} is 2-pyridyl or phenyl that is optionally substituted with one or more groups independently selected from the group consisting of halo, hydroxy, cyano, carboxyl, —CONH₂, —NO₂, —SH, —SO₃H, —SO₄H, —SO₂NH₂, —OSO₂F, —SO₂F, —NHNH₂, —ONH₂, —NHC(═O)NHNH₂, —NHC(═O)NH₂, —NHC(═O)H, —NHC(═O)OH, —NHOH, (C₁-C₆)alkyl, (C₁-C₆)alkoxy, (C₁-C₆)alkoxycarbonyl, (C₁-C₆)alkanoyl, (C₁-C₆)alkanoyloxy, (C₁-C₆)heteroalkyl, (C₃-C₆)cycloalkyl, heterocycloalkyl, heterocycloalkyl(C₁-C₆)alkyl, aryl, heteroaryl, and —NR^{r}R^{s}, wherein any (C₁-C₆)alkyl, (C₁-C₆)alkoxy, (C₁-C₆)alkoxycarbonyl, (C₁-C₆)alkanoyl, (C₁-C₆)alkanoyloxy, (C₁-C₆)heteroalkyl, (C₃-C₆)cycloalkyl, heterocycloalkyl, heterocycloalkyl(C₁-C₆)alkyl, aryl, and heteroaryl is optionally substituted with one or more groups independently selected from the group consisting of halo, hydroxy, and (C₁-C₆)alkoxy;
or a salt thereof.

18. The peptide of any one of claims 1-17, wherein X₄ is a residue of Gly or Ala.

19. The peptide of claim 1, that has structure of Formula (Ia) 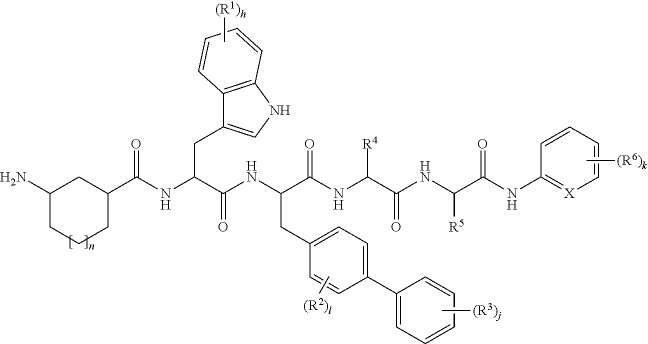
wherein
n is 0, 1, or 2;
X is C or N;
h, i, j and k are each independent 0, 1, 2 or 3;
R¹, R², R³, and R⁶ are each independently selected from the group consisting of absent, halo, hydroxy, cyano, carboxyl, —CONH₂, —NO₂, —SH, —SO₃H, —SO₄H, —SO₂NH₂, —OSO₂F, —SO₂F, —NHNH₂, —ONH₂, —NHC(═O)NHNH₂, —NHC(═O)NH₂, —NHC(═O)H, —NHC(═O)OH, —NHOH, (C₁-C₆)alkyl, (C₁-C₆)alkoxy, (C₁-C₆)alkoxycarbonyl, (C₁-C₆)alkanoyl, (C₁-C₆)alkanoyloxy, (C₁-C₆)heteroalkyl, (C₃-C₆)cycloalkyl, heterocycloalkyl, heterocycloalkyl(C₁-C₆)alkyl, aryl, heteroaryl, and —NR^{r}R^{s}, wherein any (C₁-C₆)alkyl, (C₁-C₆)alkoxy, (C₁-C₆)alkoxycarbonyl, (C₁-C₆)alkanoyl, (C₁-C₆)alkanoyloxy, (C₁-C₆)heteroalkyl, (C₃-C₆)cycloalkyl, heterocycloalkyl, heterocycloalkyl(C₁-C₆)alkyl, aryl, and heteroaryl is optionally substituted with one or more groups independently selected from the group consisting of halo, hydroxy, and (C₁-C₆)alkoxy;
R⁴, and R⁵ are each independently an amino acid side chain;
or a salt thereof.

20. The peptide of claim 19, wherein R⁴ is the side chain of hArg, Arg, hLys, Lys, Orn, Dab, or Dap.

21. The peptide of claim 19, wherein R⁴ is the side chain of L-hArg.

22. The peptide of any one of claims 19-21, wherein R^{s} is —H, —CH₃, —CH₂OH, or —CH(OH)CH₃.

23. The peptide of claim 1, that has structure of Formula (Ib) 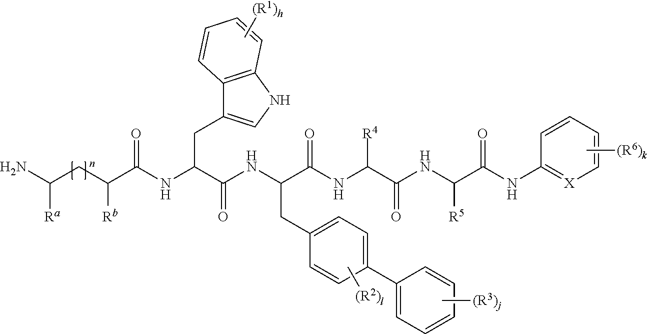
wherein
n is 0, 1, or 2;
X is C or N;
h, i, j and k are each independent 0, 1, 2 or 3;
R^{a}, R^{b}, R¹, R², R³, and R⁶ are each independently selected from the group consisting of absent, hydrogen, halo, hydroxy, cyano, carboxyl, —CONH₂, —NO₂, —SH, —SO₃H, —SO₄H, —SO₂NH₂, —OSO₂F, —SO₂F, —NHNH₂, —ONH₂, —NHC(═O)NHNH₂, —NHC(═O)NH₂, —NHC(═O)H, —NHC(═O)OH, —NHOH, (C₁-C₆)alkyl, (C₁-C₆)alkoxy, (C₁-C₆)alkoxycarbonyl, (C₁-C₆)alkanoyl, (C₁-C₆)alkanoyloxy, (C₁-C₆)heteroalkyl, (C₃-C₆)cycloalkyl, heterocycloalkyl, heterocycloalkyl(C₁-C₆)alkyl, aryl, heteroaryl, and —NR^{r}R^{s}, wherein any (C₁-C₆)alkyl, (C₁-C₆)alkoxy, (C₁-C₆)alkoxycarbonyl, (C₁-C₆)alkanoyl, (C₁-C₆)alkanoyloxy, (C₁-C₆)heteroalkyl, (C₃-C₆)cycloalkyl, heterocycloalkyl, heterocycloalkyl(C₁-C₆)alkyl, aryl, and heteroaryl is optionally substituted with one or more groups independently selected from the group consisting of halo, hydroxy, and (C₁-C₆)alkoxy;
R⁴, and R⁵ are each independently an amino acid side chain;
or a salt thereof.

24. The peptide of claim 23, wherein R⁶ is —OSO₂F, or —SO₂F.

25. The peptide of claim 1, that has structure of Formula (Ic) 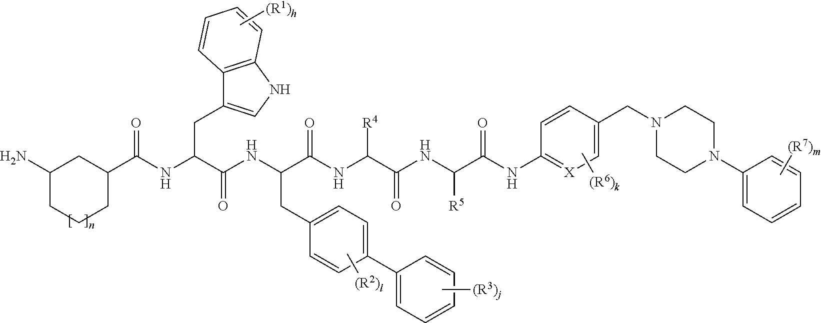
wherein
n is 0, 1, or 2;
X is C or N;
h, i, j, k and m are each independent 0, 1, 2 or 3;
R¹, R², R³, R⁶ and R⁷ are each independently selected from the group consisting of absent, halo, hydroxy, cyano, carboxyl, —CONH₂, —NO₂, —SH, —SO₃H, —SO₄H, —SO₂NH₂, —OSO₂F, —SO₂F, —NHNH₂, —ONH₂, —NHC(═O)NHNH₂, —NHC(═O)NH₂, —NHC(═O)H, —NHC(═O)OH, —NHOH, (C₁-C₆)alkyl, (C₁-C₆)alkoxy, (C₁-C₆)alkoxycarbonyl, (C₁-C₆)alkanoyl, (C₁-C₆)alkanoyloxy, (C₁-C₆)heteroalkyl, (C₃-C₆)cycloalkyl, heterocycloalkyl, heterocycloalkyl(C₁-C₆)alkyl, aryl, heteroaryl, and —NR^{r}R^{s}, wherein any (C₁-C₆)alkyl, (C₁-C₆)alkoxy, (C₁-C₆)alkoxycarbonyl, (C₁-C₆)alkanoyl, (C₁-C₆)alkanoyloxy, (C₁-C₆)heteroalkyl, (C₃-C₆)cycloalkyl, heterocycloalkyl, heterocycloalkyl(C₁-C₆)alkyl, aryl, and heteroaryl is optionally substituted with one or more groups independently selected from the group consisting of halo, hydroxy, and (C₁-C₆)alkoxy;
R⁴, and R⁵ are each independently an amino acid side chain;
or a salt thereof.

26. The peptide of claim 25, wherein R⁷ is —OSO₂F, or —SO₂F.

27. The peptide of claim 1, that has structure of Formula (Id) 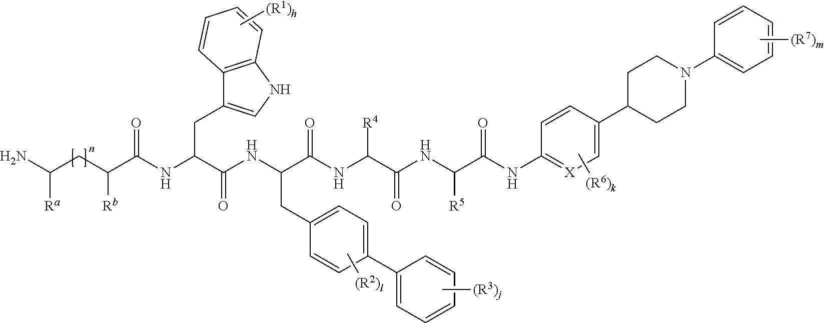
wherein
n is 0, 1, or 2;
X is C or N;
h, i, j, k and m are each independent 0, 1, 2 or 3;
R^{a}, R^{b}, R¹, R², R³, R⁶ and R⁷ are each independently selected from the group consisting of absent, hydrogen, halo, hydroxy, cyano, carboxyl, —CONH₂, —NO₂, —SH, —SO₃H, —SO₄H, —SO₂NH₂, —OSO₂F, —SO₂F, —NHNH₂, —ONH₂, —NHC(═O)NHNH₂, —NHC(═O)NH₂, —NHC(═O)H, —NHC(═O)OH, —NHOH, (C₁-C₆)alkyl, (C₁-C₆)alkoxy, (C₁-C₆)alkoxycarbonyl, (C₁-C₆)alkanoyl, (C₁-C₆)alkanoyloxy, (C₁-C₆)heteroalkyl, (C₃-C₆)cycloalkyl, heterocycloalkyl, heterocycloalkyl(C₁-C₆)alkyl, aryl, heteroaryl, and —NR^{r}R^{s}, wherein any (C₁-C₆)alkyl, (C₁-C₆)alkoxy, (C₁-C₆)alkoxycarbonyl, (C₁-C₆)alkanoyl, (C₁-C₆)alkanoyloxy, (C₁-C₆)heteroalkyl, (C₃-C₆)cycloalkyl, heterocycloalkyl, heterocycloalkyl(C₁-C₆)alkyl, aryl, and heteroaryl is optionally substituted with one or more groups independently selected from the group consisting of halo, hydroxy, and (C₁-C₆)alkoxy;
R⁴, and R⁵ are each independently an amino acid side chain;
or a salt thereof.

28. The peptide of claim 27, wherein R⁷ is —OSO₂F, or —SO₂F.

29. The peptide of claim 1, that is selected from the group consisting of: 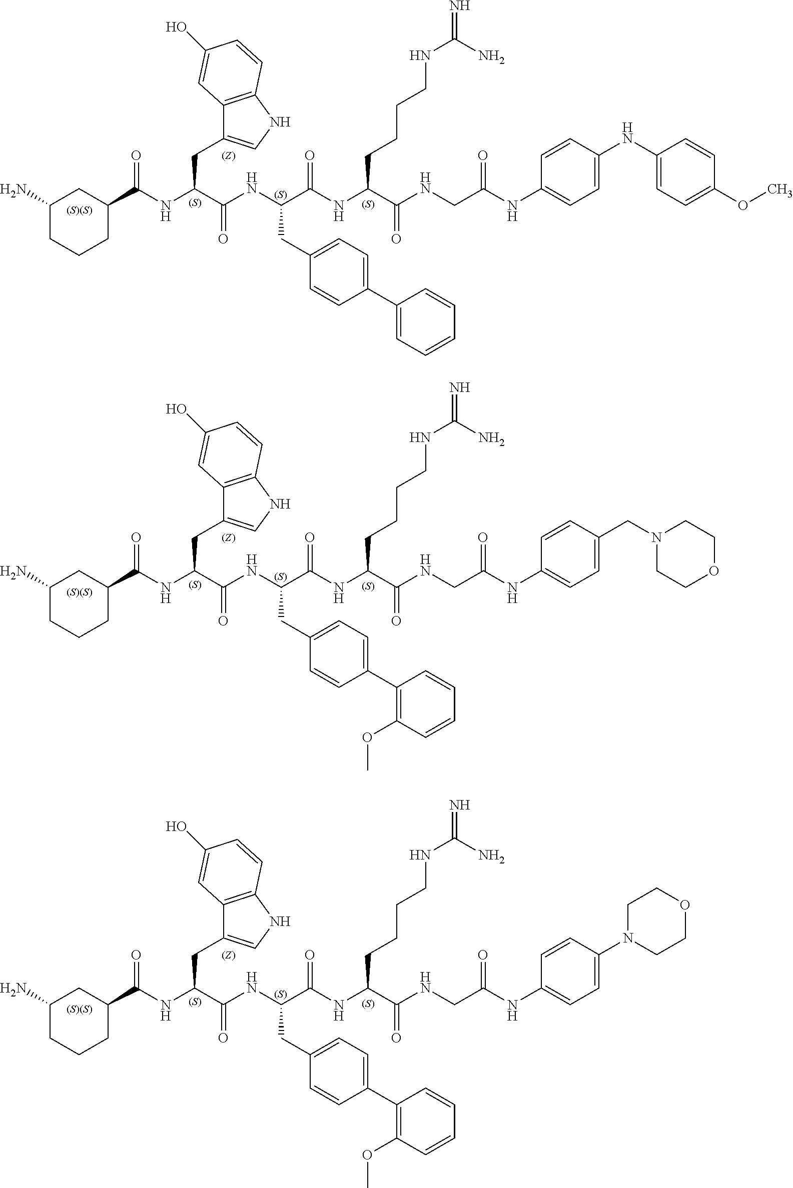 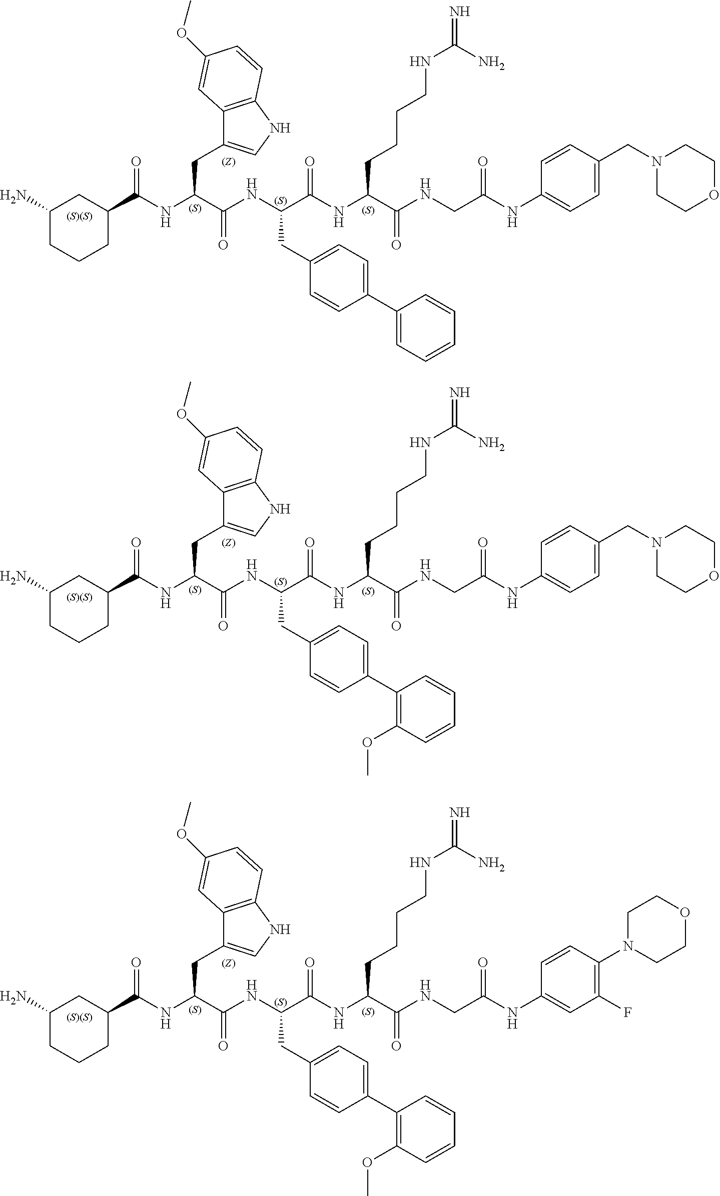 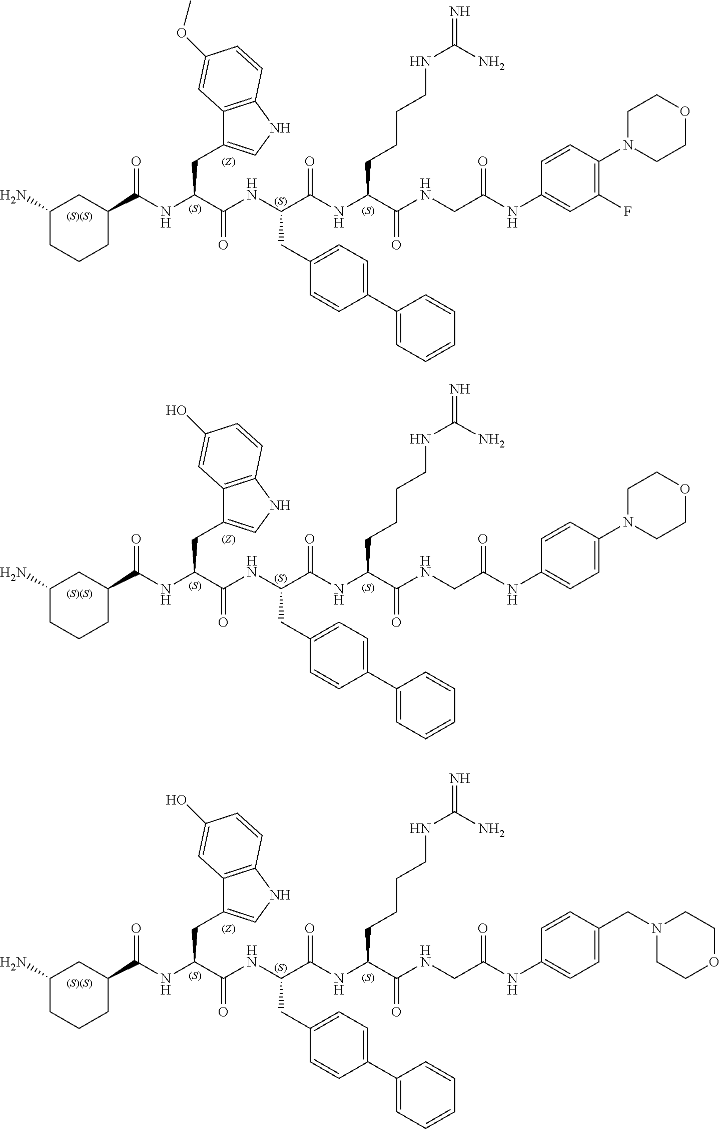 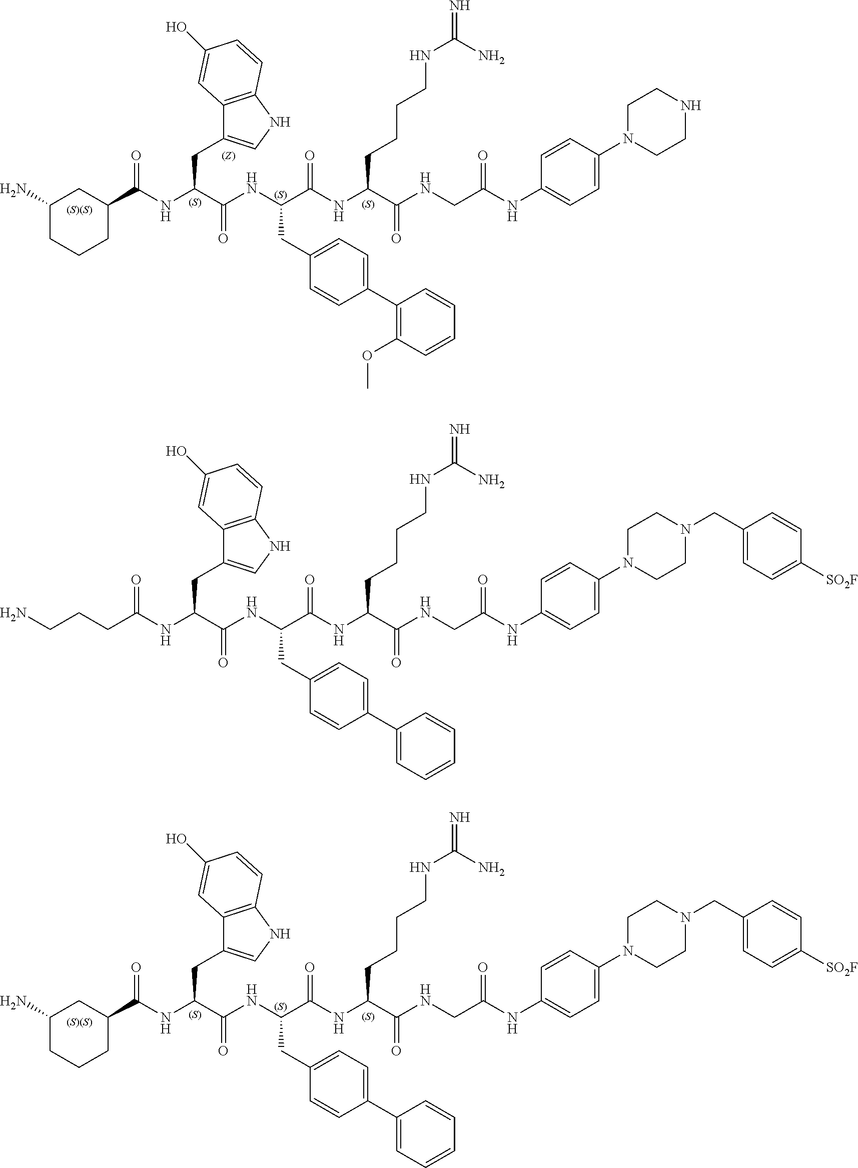 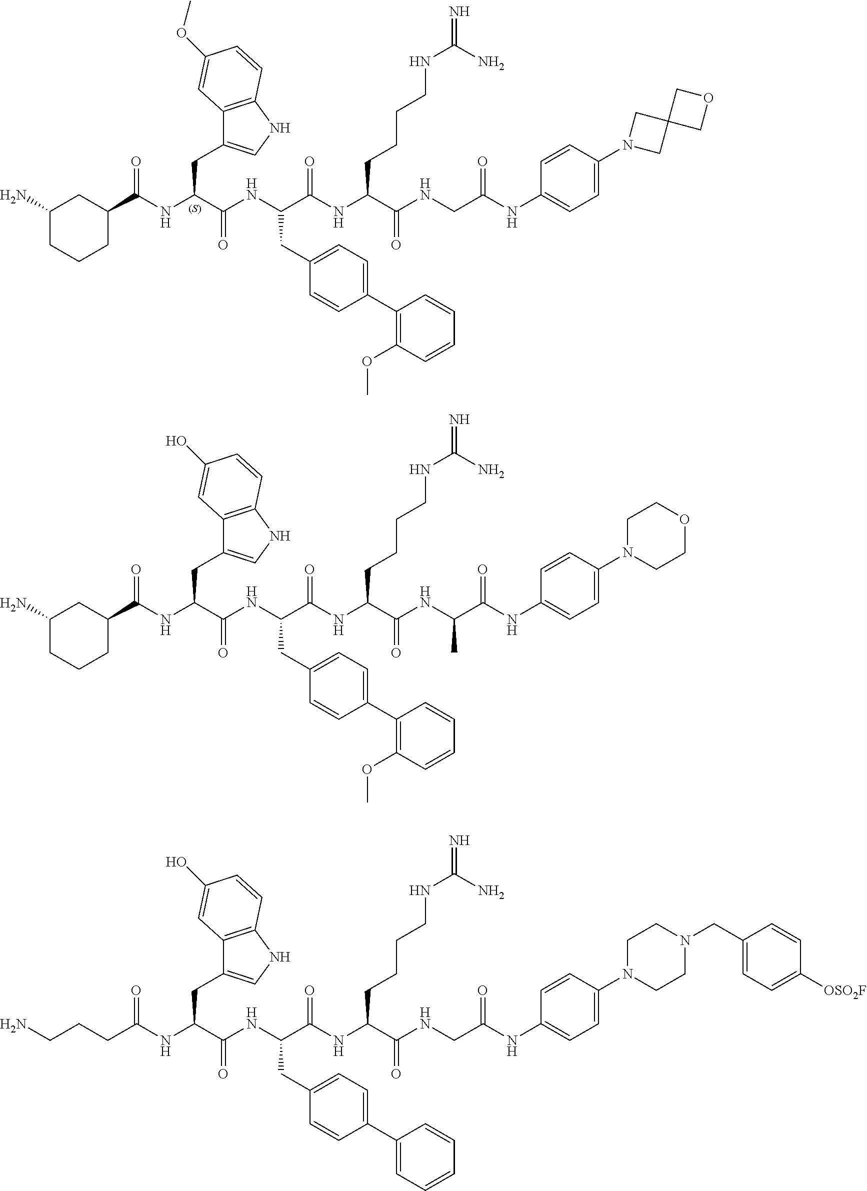 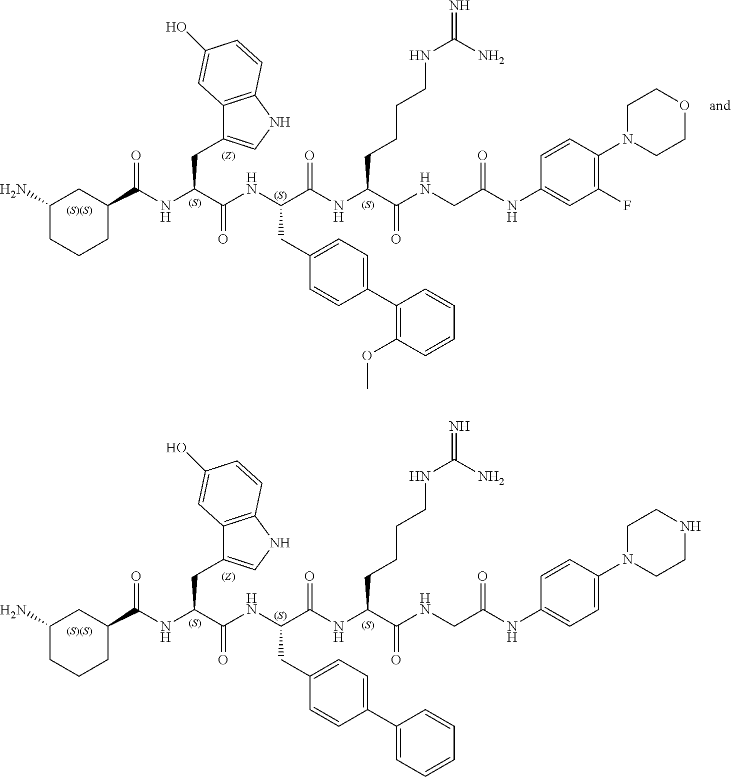
or a salt thereof.

30. The peptide of claim 1, that is 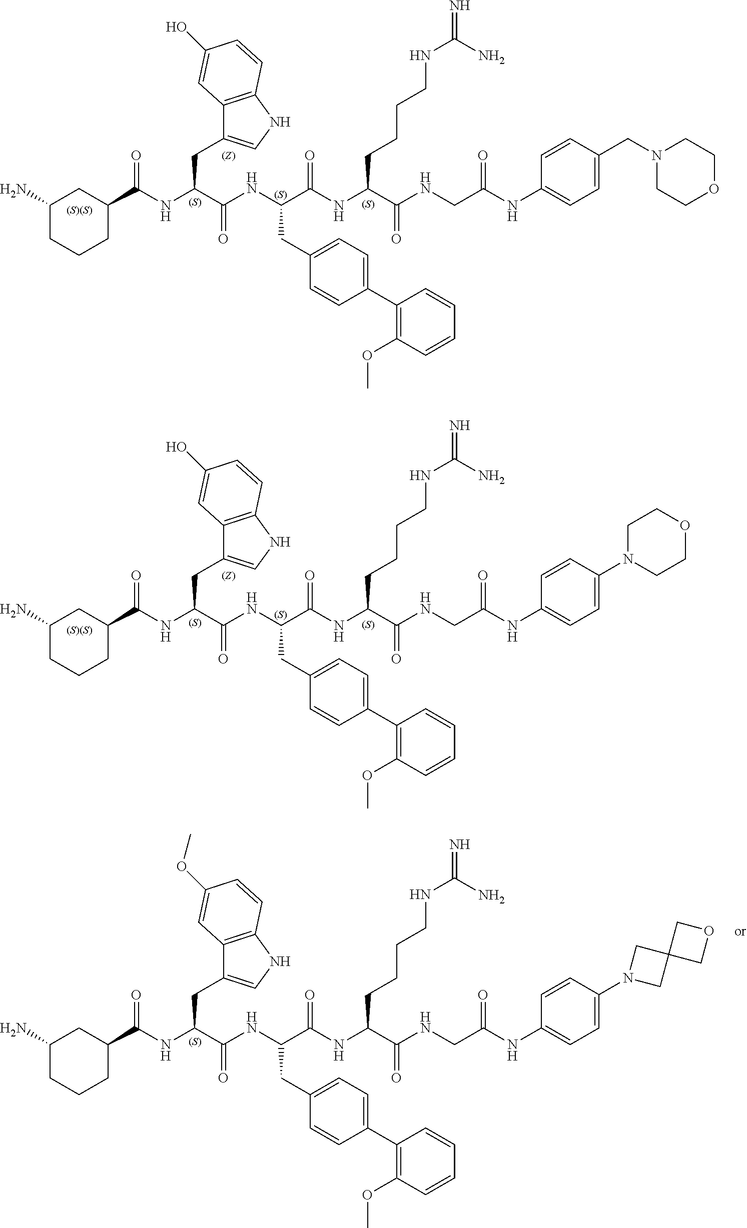 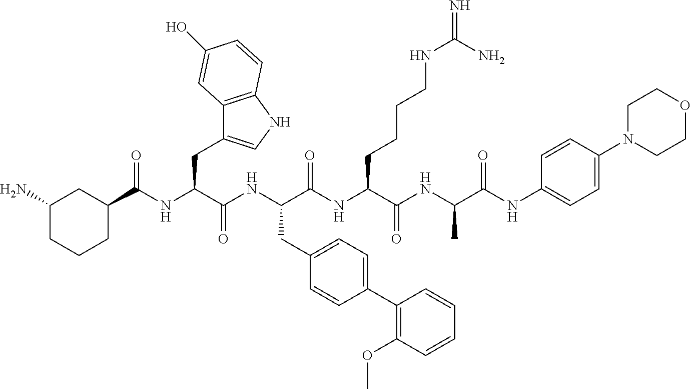 [SMILES: COc1ccccc1-c1ccc(C[C@H](NC(=O)[C@H](Cc2c[nH]c3ccc(O)cc23)NC(=O)[C@H]2CCC[C@H](N)C2)C(=O)N[C@@H](CCCCNC(=N)N)C(=O)N[C@H](C)C(=O)Nc2ccc(N3CCOCC3)cc2)cc1]
or a salt thereof.

31. The peptide of claim 1, that is 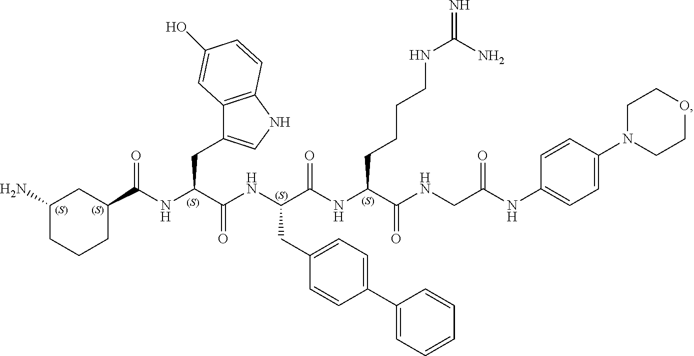
or a salt thereof.

32. A composition comprising a peptide as described in any one of claims 1-31, or a pharmaceutically acceptable salt thereof, and a pharmaceutically acceptable carrier.

33. The composition of claim 32, which is a pharmaceutical composition.

34. A method of activating EphA4 in a motor neuron, the method comprising contacting EphA4 with an effective amount of a peptide as described in any one of claims 1-31, or salt thereof, wherein the peptide is an agonist.

35. The method of claim 34, wherein the EphA4 is activated by at least about 30% when tested with 1 micromolar or less, as compared to non-treated control.

36. The method of claim 34, wherein the EphA4 is activated by at least about 50% when tested with 1 micromolar or less, as compared to non-treated control.

37. A peptide or a pharmaceutically acceptable salt thereof, as described in any one of claims 1-31, for use in medical therapy.

38. A method of treating a disease associated with EphA4 in a mammal in need thereof, comprising administering a therapeutically effective amount of a peptide as described in any one of claims 1-31, or a pharmaceutically acceptable salt thereof, to the mammal.

39. The method of claim 38, wherein the disease associated with EphA4 is cancer.

40. The method of claim 39, wherein the cancer is selected from the group consisting of gastric cancer, breast cancer, pancreatic cancer, multiple myeloma, brain cancer (e.g., glioma), thyroid cancer, urothelial cancer, testis cancer, endometrial cancer, rectal cancer, colon cancer, urothelial cancer, and skin cancer.

41. The method of claim 38, wherein the disease associated with EphA4 is a neurodegenerative disease.

42. The method of claim 41, wherein the neurodegenerative disease is amyotrophic lateral sclerosis (ALS), Alzheimer's disease (AD) or Parkinson's disease (PD).

43. The method of claim 42, wherein the neurodegenerative disease is ALS.

44. The method of claim 43, wherein the ALS is familial ALS (fALS).

45. The method of claim 43, wherein the ALS is sporadic ALS (sALS).

46. The method of any one of claims 43-45, wherein motor neuron degeneration is reduced.

47. The of method of any one of claims 43-45, wherein motor neuron degeneration induced by astrocytes is reduced.

48. The method of any one of claims 38-47, wherein the peptide is an EphA4 agonist.

49. The method of claim 48, wherein the peptide activates EphA4 expressed in a brain neuron or a spinal cord neuron.

50. A peptide or a pharmaceutically acceptable salt thereof, as described in any one of claims 1-31, for the prophylactic or therapeutic treatment of a disease associated with EphA4.

51. The use of a peptide or a pharmaceutically acceptable salt thereof, as described in any one of claims 1-31, to prepare a medicament for treating a disease associated with EphA4 in a mammal.

52. A method of treating or preventing motor neuron degeneration in a mammal in need thereof, comprising administering a therapeutically effective amount of a peptide as described in any one of claims 1-31, or a pharmaceutically acceptable salt thereof, to the mammal.

53. The method of claim 52, wherein the motor neuron degeneration is induced by astrocytes.

54. The method of claim 52 or 53, wherein the mammal has familial ALS (fALS) or was determined to have a mutation associated with fALS.

55. The method of claim 54, wherein the peptide is administered to the mammal prophylactically.

56. The method of claim 52 or 53, wherein the mammal has sporadic ALS (sALS).

57. The method of any one of claims 52-56, wherein the peptide is an EphA4 agonist.

58. A peptide or a pharmaceutically acceptable salt thereof, as described in any one of claims 1-31, for treating or preventing motor neuron degeneration.

59. The use of a peptide or a pharmaceutically acceptable salt thereof, as described in any one of claims 1-31, to prepare a medicament for treating or preventing motor neuron degeneration in a mammal.

60. A method for identifying an EphA4 agonist, the method comprising isolating primary motor neurons from the spinal cord of an animal, contacting a test compound with the isolated primary motor neurons, under conditions suitable for binding between the test compound and EphA4, evaluating axon growth cone morphology of the primary motor neurons, and identifying the test compound as an EphA4 agonist when growth cone collapse is detected.

61. A method of identifying an ALS patient that is likely to respond to treatment, the method comprising of a) isolating fibroblasts from the ALS patient, b) culturing the fibroblasts under conditions suitable to generate patient derived astrocytes, c) co-culturing the patient derived astrocytes with mouse motor neurons (MN) in the presence of a peptide as described in any one of claims 1-31, or a pharmaceutically acceptable salt thereof, and d) identifying the patient as being likely to respond to treatment with the peptide, or pharmaceutically acceptable salt thereof, when MN cell degeneration or death is inhibited as compared to a non-treatment control.
